SEQUENCE LISTING (163 sequences)

SEQ ID NO: 1 - protein, 169 aa, Mus musculus
MWVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGM

SEQ ID NO: 2 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 3 - protein, 223 aa, Mus musculus
MACLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDFLLWILVAVSLGLFFYSFLVTAVSLSKMLKKRSPLTTGVYVKMPPTEPECEKQFQPYFIPIN

SEQ ID NO: 4 - protein, 223 aa, Homo sapiens
MACLGFQRHKAQLNLATRTWPCTLLFFLLFIPVFCKAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 5 - protein, 468 aa, Mus musculus
MGEDLLLGFLLLGLLWEAPVVSSGPGKELPVVWAQEGAPVHLPCSLKSPNLDPNFLRRGGVIWQHQPDSGQPTPIPALDLHQGMPSPRQPAPGRYTVLSVAPGGLRSGRQPLHPHVQLEERGLQRGDFSLWLRPALRTDAGEYHATVRLPNRALSCSLRLRVGQASMIASPSGVLKLSDWVLLNCSFSRPDRPVSVHWFQGQNRVPVYNSPRHFLAETFLLLPQVSPLDSGTWGCVLTYRDGFNVSITYNLKVLGLEPVAPLTVYAAEGSRVELPCHLPPGVGTPSLLIAKWTPPGGGPELPVAGKSGNFTLHLEAVGLAQAGTYTCSIHLQGQQLNATVTLAVITVTPKSFGLPGSRGKLLCEVTPASGKERFVWRPLNNLSRSCPGPVLEIQEARLLAERWQCQLYEGQRLLGATVYAAESSSGAHSARRISGDLKGGHLVLVLILGALSLFLLVAGAFGFHKRIQ

SEQ ID NO: 6 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 7 - protein, 184 aa, Mus musculus
MKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLLYT

SEQ ID NO: 8 - protein, 289 aa, Homo sapiens
MKTLPAMLGTGKLFWVFFLIPYLDIWNIHGKESCDVQLYIKRQSEHSILAGDPFELECPVKYCANRPHVTWCKLNGTTCVKLEDRQTSWKEEKNISFFILHFEPVLPNDNGSYRCSANFQSNLIESHSTTLYVTDVKSASERPSKDEMASRPWLLYRLLPLGGLPLLITTCFCLFCCLRRHQGKQNELSDTAGREINLVDAHLKSEQTEASTRQNSQVLLSETGIYDNDPDLCFRMQEGSEVYSNPCLEENKPGIVYASLNHSVIGPNSRLARNVKEAPTEYASICVRS

SEQ ID NO: 9 - protein, 187 aa, Mus musculus
MERILMAPGQSCCALAILLAIVNFQHGGCIHVTSSASQKGGRLDLTCTLWHKKDEAEGLILFWCKDNPWNCSPETNLEQLRVKRDPETDGITEKSSQLVFTIEQATPSDSGTYQCCARSQKPEIYIHGHFLSVLVTGNHTEIRQRQRSHPDFSHINGTLSSGFLQVKAWGMLVTSLVALQALYTLAA

SEQ ID NO: 10 - protein, 181 aa, Homo sapiens
MLLEPGRGCCALAILLAIVDIQSGGCINITSSASQEGTRLNLICTVWHKKEEAEGFVVFLCKDRSGDCSPETSLKQLRLKRDPGIDGVGEISSQLMFTISQVTPLHSGTYQCCARSQKSGIRLQGHFFSILFTETGNYTVTGLKQRQHLEFSHNEGTLSSGFLQEKVWVMLVTSLVALQAL

SEQ ID NO: 11 - protein, 255 aa, Mus musculus
MIGQAVLFTTFLLLRAHQGQDCPDSSEEVVGVSGKPVQLRPSNIQTKDVSVQWKKTEQGSHRKIEILNWYNDGPSWSNVSFSDIYGFDYGDFALSIKSAKLQDSGHYLLEITNTGGKVCNKNFQLLILDHVETPNLKAQWKPWTNGTCQLFLSCLVTKDDNVSYALYRGSTLISNQRNSTHWENQIDASSLHTYTCNVSNRASWANHTLNFTHGCQSVPSNFRFLPFGVIIVILVTLFLGAIICFCVWTKKRKAA

SEQ ID NO: 12 - protein, 365 aa, Homo sapiens
MLGQVVTLILLLLLKVYQGKGCQGSADHVVSISGVPLQLQPNSIQTKVDSIAWKKLLPSQNGFHHILKWENGSLPSNTSNDRFSFIVKNLSLLIKAAQQQDSGLYCLEVTSISGKVQTATFQVFVFDKVEKPRLQGQGKILDRGRCQVALSCLVSRDGNVSYAWYRGSKLIQTAGNLTYLDEEVDINGTHTYTCNVSNPVSWESHTLNLTQDCQNAHQEFRFWPFLVIIVILSALFLGTLACFCVWRRKRKEKQSETSPKEFLTIYEDVKDLKTRRNHEQEQTFPGGGSTIYSMIQSQSSAPTSQEPAYTLYSLIQPSRKSGSRKRNHSPSFNSTIYEVIGKSQPKAQNPARLSRKELENFDVYS

SEQ ID NO: 13 - protein, 188 aa, Mus musculus
MADSSIYSTLELPEAPQVQDESRWKLKAVLHRPHLSRFAMVALGLLTVILMSLLMYQRILCCGSKDSTCSHCPSCPILWTRNGSHCYYFSMEKKDWNSSLKFCADKGSHLLTFPDNQGVKLFGEYLGQDFYWIGLRNIDGWRWEGGPALSLRILTNSLIQRCGAIHRNGLQASSCEVALQWICKKVLY

SEQ ID NO: 14 - protein, 195 aa, Homo sapiens
MTDSVIYSMLELPTATQAQNDYGPQQKSSSSRPSCSCLVAIALGLLTAVLLSVLLYQWILCQGSNYSTCASCPSCPDRWMKYGNHCYYFSVEEKDWNSSLEFCLARDSHLLVITDNQEMSLLQVFLSEAFCWIGLRNNSGWRWEDGSPLNFSRISSNSFVQTCGAINKNGLQASSCEVPLHWVCKKCPFADQALF

SEQ ID NO: 15 - protein, 333 aa, Mus musculus
MAYVTWLLAILHVHKALCEETLWDTTVRLSETMTLECVYPLTHNLTQVEWTKNTGTKTVSIAVYNPNHNMHIESNYLHRVHFLNSTVGFRNMSLSFYNASEADIGIYSCLFHAFPNGPWEKKIKVVWSDSFEIAAPSDSYLSAEPGQDVTLTCQLPRTWPVQQVIWEKVQPHQVDILASCNLSQETRYTSKYLRQTRSNCSQGSMKSILIIPNAMAADSGLYRCRSEAITGKNKSFVIRLIITDGGTNKHFILPIVGGLVSLLLVILIIIIFILYNRKRRRQVRIPLKEPRDKQSKVATNCRSPTSPIQSTDDEKEDIYVNYPTFSRRPKPRL

SEQ ID NO: 16 - protein, 336 aa, Homo sapiens
MDYPTLLLALLHVYRALCEEVLWHTSVPFAENMSLECVYPSMGILTQVEWFKIGTQQDSIAIFSPTHGMVIRKPYAERVYFLNSTMASNNMTLFFRNASEDDVGYYSCSLYTYPQGTWQKVIQVVQSDSFEAAVPSNSHIVSEPGKNVTLTCQPQMTWPVQAVRWEKIQPRQIDLLTYCNLVHGRNFTSKFPRQIVSNCSHGRWSVIVIPDVTVSDSGLYRCYLQASAGENETFVMRLTVAEGKTDNQYTLFVAGGTVLLLLFVISITTIIVIFLNRRRRRERRDLFTESWDTQKAPNNYRSPISTSQPTNQSMDDTREDIYVNYPTFSRRPKTRV

SEQ ID NO: 17 - protein, 165 aa, Mus musculus
MKGWLLLVWVQGLIQAAFLATGATAGTIDTKRNISAEEGGSVILQCHFSSDTAEVTQVDWKQQDQLLAIYSVDLGWHVASVFSDRVVPGPSLGLTFQSLTMNDTGEYFCTYHTYPGGIYKGRIFLKVQESSVAQFQTAPLGGTMAAVLGLICLMVTGVTVLARKK

SEQ ID NO: 18 - protein, 244 aa, Homo sapiens
MRWCLLLIWAQGLRQAPLASGMMTGTIETTGNISAEKGGSIILQCHLSSTTAQVTQVNWEQQDQLLAICNADLGWHISPSFKDRVAPGPGLGLTLQSLTVNDTGEYFCIYHTYPDGTYTGRIFLEVLESSVAEHGARFQIPLLGAMAATLVVICTAVIVVVALTRKKKALRIHSVEGDLRRKSAGQEEWSPSAPSPPGSCVQAEAAPAGLCGEQRGEDCAELHDYFNVLSYRSLGNCSFFTETG

SEQ ID NO: 19 - protein, 316 aa, Mus musculus
MLRGWGGPSVGVCVRTALGVLCLCLTGAVEVQVSEDPVVALVDTDATLRCSFSPEPGFSLAQLNLIWQLTDTKQLVHSFTEGRDQGSAYSNRTALFPDLLVQGNASLRLQRVRVTDEGSYTCFVSIQDFDSAAVSLQVAAPYSKPSMTLEPNKDLRPGNMVTITCSSYQGYPEAEVFWKDGQGVPLTGNVTTSQMANERGLFDVHSVLRVVLGANGTYSCLVRNPVLQQDAHGSVTITGQPLTFPPEALWVTVGLSVCLVVLLVALAFVCWRKIKQSCEEENAGAEDQDGDGEGSKTALRPLKPSENKEDDGQEIA

SEQ ID NO: 20 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 21 - protein, 308 aa, Mus musculus
MGVPAVPEASSPRWGTLLLAIFLAASRGLVAAFKVTTPYSLYVCPEGQNATLTCRILGPVSKGHDVTIYKTWYLSSRGEVQMCKEHRPIRNFTLQHLQHHGSHLKANASHDQPQKHGLELASDHHGNFSITLRNVTPRDSGLYCCLVIELKNHHPEQRFYGSMELQVQAGKGSGSTCMASNEQDSDSITAAALATGACIVGILCLPLILLLVYKQRQVASHRRAQELVRMDSNTQGIENPGFETTPPFQGMPEAKTRPPLSYVAQRQPSESGRYLLSDPSTPLSPPGPGDVFFPSLDPVPDSPNSEAI

SEQ ID NO: 22 - protein, 311 aa, Homo sapiens
MGVPTALEAGSWRWGSLLFALFLAASLGPVAAFKVATPYSLYVCPEGQNVTLTCRLLGPVDKGHDVTFYKTWYRSSRGEVQTCSERRPIRNLTFQDLHLHHGGHQAANTSHDLAQRHGLESASDHHGNFSITMRNLTLLDSGLYCCLVVEIRHHHSEHRVHGAMELQVQTGKDAPSNCVVYPSSSQDSENITAAALATGACIVGILCLPLILLLVYKQRQAASNRRAQELVRMDSNIQGIENPGFEASPPAQGIPEAKVRHPLSYVAQRQPSESGRHLLSEPSTPLSPPGPGDVFFPSLDPVPDSPNFEVI

SEQ ID NO: 23 - protein, 281 aa, Mus musculus
MFSGLTLNCVLLLLQLLLARSLENAYVFEVGKNAYLPCSYTLSTPGALVPMCWGKGFCPWSQCTNELLRTDERNVTYQKSSRYQLKGDLNKGDVSLIIKNVTLDDHGTYCCRIQFPGLMNDKKLELKLDIKAAKVTPAQTAHGDSTTASPRTLTTERNGSETQTLVTLHNNNGTKISTWADEIKDSGETIRTAIHIGVGVSAGLTLALIIGVLILKWYSCKKKKLSSLSLITLANLPPGGLANAGAVRIRSEENIYTIEENVYEVENSNEYYCYVNSQQPS

SEQ ID NO: 24 - protein, 301 aa, Homo sapiens
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIGIYIGAGICAGLALALIFGALIFKWYSHSKEKIQNLSLISLANLPPSGLANAVAEGIRSEENIYTIEENVYEVEEPNEYYCYVSSRQQPSQPLGCRFAMP

SEQ ID NO: 25 - protein, 290 aa, Mus musculus
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRTHWVLLGSILLFLIVVSTVLLFLRKQVRMLDVEKCGVEDTSSKNRNDTQFEET

SEQ ID NO: 26 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 27 - protein, 247 aa, Mus musculus
MLLLLPILNLSLQLHPVAALFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRTWPLHVFIPACTIALIFLAIVIIQRKRI

SEQ ID NO: 28 - protein, 273 aa, Homo sapiens
MIFLLLMLSLELQLHQIAALFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPTWLLHIFIPFCIIAFIFIATVIALRKQLCQKLYSSKDTTKRPVTTTKREVNSAI

SEQ ID NO: 29 - protein, 276 aa, Mus musculus
MEPLPGWGSAPWSQAPTDNTFRLVPCVFLLNLLQRISAQPSCRQEEFLVGDECCPMCNPGYHVKQVCSEHTGTVCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVVYYVVSILLPLVIVGVGIAGFLICTRRHLHTSSVAKELEPFQQEQQENTIRFPVTEVGFAETEEETASN

SEQ ID NO: 30 - protein, 283 aa, Homo sapiens
MEPPGDWGPPPWRSTPKTDVLRLVLYLTFLGAPCYAPALPSCKEDEYPVGSECCPKCSPGYRVKEACGELTGTVCEPCPPGTYIAHLNGLSKCLQCQMCDPAMGLRASRNCSRTENAVCGCSPGHFCIVQDGDHCAACRAYATSSPGQRVQKGGTESQDTLCQNCPPGTFSPNGTLEECQHQTKCSWLVTKAGAGTSSSHWVWWFLSGSLVIVIVCSTVGLIICVKRRKPRGDVVKVIVSVQRKRQEAEGEATVIEALQAPPDVTTVAVEETIPSFTGRSPNH

SEQ ID NO: 31 - protein, 240 aa, Mus musculus
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARSSGVCWTATWLVVTTLIIHRILLT

SEQ ID NO: 32 - protein, 243 aa, Homo sapiens
MCSRGWDSCLALELLLLPLSLLVTSIQGHLVHMTVVSGSNVTLNISESLPENYKQLTWFYTFDQKIVEWDSRKSKYFESKFKGRVRLDPQSGALYISKVQKEDNSTYIMRVLKKTGNEQEWKIKLQVLDPVPKPVIKIEKIEDMDDNCYLKLSCVIPGESVNYTWYGDKRPFPKELQNSVLETTLMPHNYSRCYTCQVSNSVSSKNGTVCLSPPCTLARSFGVEWIASWLVVTVPTILGLLLT

SEQ ID NO: 33 - protein, 530 aa, Mus musculus
MARAAVLPPSRLSPTLPLLPLLLLLLQETGAQDVRVRVLPEVRGRLGGTVELPCHLLPPTTERVSQVTWQRLDGTVVAAFHPSFGVDFPNSQFSKDRLSFVRARPETNADLRDATLAFRGLRVEDEGNYTCEFATFPNGTRRGVTWLRVIAQPENHAEAQEVTIGPQSVAVARCVSTGGRPPARITWISSLGGEAKDTQEPGIQAGTVTIISRYSLVPVGRADGVKVTCRVEHESFEEPILLPVTLSVRYPPEVSISGYDDNWYLGRSEAILTCDVRSNPEPTDYDWSTTSGVFPASAVAQGSQLLVHSVDRMVNTTFICTATNAVGTGRAEQVILVRESPSTAGAGATGGIIGGIIAAIIATAVAGTGILICRQQRKEQRLQAADEEEELEGPPSYKPPTPKAKLEEPEMPSQLFTLGASEHSPVKTPYFDAGVSCADQEMPRYHELPTLEERSGPLLLGATGLGPSLLVPPGPNVVEGVSLSLEDEEEDDEEEDFLDKINPIYDALSYPSPSDSYQSKDFFVSRAMYV

SEQ ID NO: 34 - protein, 538 aa, Homo sapiens
MARAAALLPSRSPPTPLLWPLLLLLLLETGAQDVRVQVLPEVRGQLGGTVELPCHLLPPVPGLYISLVTWQRPDAPANHQNVAAFHPKMGPSFPSPKPGSERLSFVSAKQSTGQDTEAELQDATLALHGLTVEDEGNYTCEFATFPKGSVRGMTWLRVIAKPKNQAEAQKVTFSQDPTTVALCISKEGRPPARISWLSSLDWEAKETQVSGTLAGTVTVTSRFTLVPSGRADGVTVTCKVEHESFEEPALIPVTLSVRYPPEVSISGYDDNWYLGRTDATLSCDVRSNPEPTGYDWSTTSGTFPTSAVAQGSQLVIHAVDSLFNTTFVCTVTNAVGMGRAEQVIFVRETPNTAGAGATGGIIGGIIAAIIATAVAATGILICRQQRKEQTLQGAEEDEDLEGPPSYKPPTPKAKLEAQEMPSQLFTLGASEHSPLKTPYFDAGASCTEQEMPRYHELPTLEERSGPLHPGATSLGSPIPVPPGPPAVEDVSLDLEDEEGEEEEEYLDKINPIYDALSYSSPSDSYQGKGFVMSRAMYV

SEQ ID NO: 35 - protein, 408 aa, Mus musculus
MAQLARATRSPLSWLLLLFCYALRKAGGDIRVLVPYNSTGVLGGSTTLHCSLTSNENVTITQITWMKKDSGGSHALVAVFHPKKGPNIKEPERVKFLAAQQDLRNASLAISNLSVEDEGIYECQIATFPRGSRSTNAWLKVQARPKNTAEALEPSPTLILQDVAKCISANGHPPGRISWPSNVNGSHREMKEPGSQPGTTTVTSYLSMVPSRQADGKNITCTVEHESLQELDQLLVTLSQPYPPENVSISGYDGNWYVGLTNLTLTCEAHSKPAPDMAGYNWSTTTGDFPNSVKRQGNMLLISTVEDGLNNTVIVCEVTNALGSGQGQVHIIVKEKPENMQQNTRLHLGYIFLIVFVLAVVIIIAALYTIRRCRHGRALQSNPSERENVQYSSVNGDCRLNMEPNSTR

SEQ ID NO: 36 - protein, 417 aa, Homo sapiens
MARAMAAAWPLLLVALLVLSWPPPGTGDVVVQAPTQVPGFLGDSVTLPCYLQVPNMEVTHVSQLTWARHGESGSMAVFHQTQGPSYSESKRLEFVAARLGAELRNASLRMFGLRVEDEGNYTCLFVTFPQGSRSVDIWLRVLAKPQNTAEVQKVQLTGEPVPMARCVSTGGRPPAQITWHSDLGGMPNTSQVPGFLSGTVTVTSLWILVPSSQVDGKNVTCKVEHESFEKPQLLTVNLTVYYPPEVSISGYDNNWYLGQNEATLTCDARSNPEPTGYNWSTTMGPLPPFAVAQGAQLLIRPVDKPINTTLICNVTNALGARQAELTVQVKEGPPSEHSGISRNAIIFLVLGILVFLILLGIGIYFYWSKCSREVLWHCHLCPSSTEHASASANGHVSYSAVSRENSSSQDPQTEGTR

SEQ ID NO: 37 - protein, 521 aa, Mus musculus
MELASAHLHKGQVPWGGLLLTASLLASWSPATTAEVTIEAVPPQVAEDNNVLLLVHNLPLALGAFAWYKGNTTAIDKEIARFVPNSNMNFTGQAYSGREIIYSNGSLLFQMITMKDMGVYTLDMTDENYRRTQATVRFHVHPILLKPNITSNNSNPVEGDDSVSLTCDSYTDPDNINYLWSRNGESLSEGDRLKLSEGNRTLTLLNVTRNDTGPYVCETRNPVSVNRSDPFSLNIIYGPDTPIISPSDIYLHPGSNLNLSCHAASNPPAQYFWLINEKPHASSQELFIPNITTNNSGTYTCFVNNSVTGLSRTTVKNITVLEPVTQPFLQVTNTTVKELDSVTLTCLSNDIGANIQWLFNSQSLQLTERMTLSQNNSILRIDPIKREDAGEYQCEISNPVSVRRSNSIKLDIIFDPTQGGLSDGAIAGIVIGVVAGVALIAGLAYFLYSRKSGGGSDQRDLTEHKPSTSNHNLAPSDNSPNKVDDVAYTVLNFNSQQPNRPTSAPSSPRATETVYSEVKKK

SEQ ID NO: 38 - protein, 526 aa, Homo sapiens
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGAIAGIVIGVVALVALIAVALACFLHFGKTGRASDQRDLTEHKPSVSNHTQDHSNDPPNKMNEVTYSTLNFEAQQPTQPTSASPSLTATEIIYSEVKKQ

SEQ ID NO: 39 - protein, 264 aa, Mus musculus
MADSFSLNDALAGSGNPNPQGYPGAWGNQPGAGGYPGAAYPGAYPGQAPPGAYPGQAPPGAYPGQAPPSAYPGPTAPGAYPGPTAPGAYPGQPAPGAFPGQPGAPGAYPQCSGGYPAAGPYGVPAGPLTVPYDLPLPGGVMPRMLITIMGTVKPNANRIVLDFRRGNDVAFHFNPRFNENNRRVIVCNTKQDNNWGKEERQSAFPFESGKPFKIQVLVEADHFKVAVNDAHLLQYNHRMKNLREISQLGISGDITLTSANHAMI

SEQ ID NO: 40 - protein, 250 aa, Homo sapiens [sequence omitted]

SEQ ID NO: 41 - protein, 314 aa, Mus musculus
MGKIYSFVLVAIALMMGREGWALESESCLREQVRLRAQVHQLETRVKQQQTMIAQLLHEKEVQFLDKGSENSFIDLGGKRQYADCSEIYNDGFKQSGFYKIKPLQSLAEFSVYCDMSDGGGWTVIQRRSDGSENFNRGWNDYENGFGNFVQNNGEYWLGNKNINLLTIQGDYTLKIDLTDFEKNSSFAQYQSFKVGDKKSFYELNIGEYSGTAGDSLSGTFHPEVQWWASHQRMKFSTWDRDNDNYQGNCAEEEQSGWWFNRCHSANLNGVYYRGSYRAETDNGVVWYTWHGWWYSLKSVVMKIRPSDFIPNII

SEQ ID NO: 42 - protein, 312 aa, Homo sapiens
MAKVFSFILVTTALTMGREISALEDCAQEQMRLRAQVRLLETRVKQQQVKIKQLLQENEVQFLDKGDENTVIDLGSKRQYADCSEIFNDGYKLSGFYKIKPLQSPAEFSVYCDMSDGGGWTVIQRRSDGSENFNRGWKDYENGFGNFVQKHGEYWLGNKNLHFLTTQEDYTLKIDLADFEKNSRYAQYKNFKVGDEKNFYELNIGEYSGTAGDSLAGNFHPEVQWWASHQRMKFSTWDRDHDNYEGNCAEEDQSGWWFNRCHSANLNGVYYSGPYTAKTDNGIVWYTWHGWWYSLKSVVMKIRPNDFIPNVI

SEQ ID NO: 43 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 44 - protein, 306 aa, Mus musculus
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLVLFGAGFGAVITVVVIVVIIKCFCKHRSCFRRNEASRETNNSLTFGPEEALAEQTVFL

SEQ ID NO: 45 - protein, 323 aa, Homo sapiens
MGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 46 - protein, 309 aa, Mus musculus
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEITASVTVALLLVMLLIIVCHKKPNQPSRPSNTASKLERDSNADRETINLKELEPQIASAKPNAE

SEQ ID NO: 47 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 48 - protein, 309 aa, Mus musculus
MDQHTLDVEDTADARHPAGTSCPSDAALLRDTGLLADAALLSDTVRPTNAALPTDAAYPAVNVRDREAAWPPALNFCSRHPKLYGLVALVLLLLIAACVPIFTRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 49 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 50 - protein, 256 aa, Mus musculus
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 51 - protein, 277 aa, Homo sapiens
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 52 - protein, 272 aa, Mus musculus
MYVWVQQPTALLLLGLTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPPTLVTPEGPAFAVLLGLGLGLLAPLTVLLALYLLRKAWRLPNTPKPCWGNSFRTPIQEEHTDAHFTLAKI

SEQ ID NO: 53 - protein, 133 aa, Homo sapiens
MVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 54 - protein, 198 aa, Mus musculus
MEGEGVQPLDENLENGSRPRFKWKKTLRLVVSGIKGAGMLLCFIYVCLQLSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 55 - protein, 277 aa, Homo sapiens
MVRLPLQCVLWGCLLTAVHPEPPTACREKQYLINSQCCSLCQPGQKLVSDCTEFTETECLPCGESEFLDTWNRETHCHQHKYCDPNLGLRVQQKGTSETDTICTCEEGWHCTSEACESCVLHRSCSPGFGVKQIATGVSDTICEPCPVGFFSNVSSAFEKCHPWTSCETKDLVVQQAGTNKTDVVCGPQDRLRALVVIPIIFGILFAILLVLVFIKKVAKKPTNKAPHPKQEPQEINFPDDLPGSNTAAPVQETLHGCQPVTQEDGKESRISVQERQ

SEQ ID NO: 56 - protein, 289 aa, Mus musculus
MVSLPRLCALWGCLLTAVHLGQCVTCSDKQYLHDGQCCDLCQPGSRLTSHCTALEKTQCHPCDSGEFSAQWNREIRCHQHRHCEPNQGLRVKKEGTAESDTVCTCKEGQHCTSKDCEACAQHTPCIPGFGVMEMATETTDTVCHPCPVGFFSNQSSLFEKCYPWTSCEDKNLEVLQKGTSQTNVICGLKSRMRALLVIPVVMGILITIFGVFLYIKKVVKKPKDNEILPPAARRQDPQEMEDYPGHNTAAPVQETLHGCQPVTQEDGKESRISVQERQVTDSIALRPLV

SEQ ID NO: 57 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 58 - protein, 260 aa, Mus musculus
MIETYSQPSPRSVATGLPASMKIFMYLLTVFLITQMIGSVLFAVYLHRRLDKVEEEVNLHEDFVFIKKLKRCNKGEGSLSLLNCEEMRRQFEDLVKDITLNKEEKKENSFEMQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 59 - protein, 302 aa, Homo sapiens
MRLGSPGLLFLLFSSLRADTQEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSILAVLCLLVVVAVAIGWVCRDRCLQHSYAGAWAVSPETELTGHV

SEQ ID NO: 60 - protein, 322 aa, Mus musculus
MQLKCPCFVSLGTRQPVWKKLHVSSGFFSGLGLFLLLLSSLCAASAETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELKVLVPVLAVLAAAAFVSFIIYRRTRPHRSYTGPKTVQLELTDHA

SEQ ID NO: 61 - protein, 199 aa, Homo sapiens
MKSGLWYFFLFCLRIKVLTGEINGSANYEMFIFHNGGVQILCKYPDIVQQFKMQLLKGGQILCDLTKTKGSGNTVSIKSLKFCHSQLSNNSVSFFLYNLDHSHANYYFCNLSIFDPPPFKVTLTGGYLHIYESQLCCQLKFWLPIGCAAFVVVCILGCILICWLTKKKYSSSVHDPNGEYMFMRAVNTAKKSRLTDVTL

SEQ ID NO: 62 - protein, 200 aa, Mus musculus
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLWLPVGCAAFVVVLLFGCILIIWFSKKKYGSSVHDPNSEYMFMAAVNTNKKSRLAGVTS

SEQ ID NO: 63 - protein, 495 aa, Vesiculovirus indiana
KFTIVFPHNQKGNWKNVPSNYHYCPSSSDLNWHNDLIGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDITFFSEDGELSSLGKEGTGFRSNYFAYETGGKACKMQYCKHWGVRLPSGVWFEMADKDLFAAARFPECPEGSSISAPSQTSVDVSLIQDVERILDYSLCQETWSKIRAGLPISPVDLSYLAPKNPGTGPAFTIINGTLKYFETRYIRVDIAAPILSRMVGMISGTTTERELWDDWAPYEDVEIGPNGVLRTSSGYKFPLYMIGHGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 64 - protein, 1273 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 65 - protein, 150 aa, Artificial Sequence
  Description of Artificial Sequence Synthetic polypeptide
IGTALQVKMPKSHKAIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLISMDI

SEQ ID NO: 66 - protein, 130 aa, Artificial Sequence
  Description of Artificial Sequence Synthetic polypeptide
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNL

SEQ ID NO: 67 - protein, 115 aa, Artificial Sequence
  Description of Artificial Sequence Synthetic polypeptide
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTT

SEQ ID NO: 68 - protein, 100 aa, Artificial Sequence
  Description of Artificial Sequence Synthetic polypeptide
IQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFING

SEQ ID NO: 69 - protein, 84 aa, Artificial Sequence
  Description of Artificial Sequence Synthetic polypeptide
IQADGWMCHASKWVTTCDFRWYGPKYITHSIRSFTPSVEQCKESIEQTKQGTWLNPGFPPQSCGYATVTDAEAVIVQVTPHHVL

SEQ ID NO: 70 - protein, 27 aa, Artificial Sequence
  Description of Artificial Sequence Synthetic peptide
GYIPEAPRDGQAYVRKDGEWVLLSTFL

SEQ ID NO: 71 - protein, 33 aa, Artificial Sequence
  Description of Artificial Sequence Synthetic polypeptide
RMKQLEDKVEELLSKQYHLENEVARLKKLVGER

SEQ ID NO: 72 - protein, 33 aa, Artificial Sequence
  Description of Artificial Sequence Synthetic polypeptide
RMKQLEDKVEELLSKNYHLENEVARLKKLVGER

SEQ ID NO: 73 - protein, 40 aa, Artificial Sequence
  Description of Artificial Sequence Synthetic polypeptide
MNPNQKIITIGSICLVVGLISLILQIGNIISIWISHSIQT

SEQ ID NO: 74 - protein, 75 aa, Artificial Sequence
  Description of Artificial Sequence Synthetic polypeptide
MNPNQKIITIGSICMVTGIVSLMLQIGNMISIWVSHSIHTGNQHQSEPISNTNFLTEKAVASVKLAGNSSLCPIN

SEQ ID NO: 75 - protein, 68 aa, Artificial Sequence
  Description of Artificial Sequence Synthetic polypeptide
KLCIEAKISNTTTDSRCPTQGEATLVEEQDTNFVCRRTFVDRGHGNGCGLFGKGSLITCAKFKCVTKL

SEQ ID NO: 76 - protein, 83 aa, Artificial Sequence
  Description of Artificial Sequence Synthetic polypeptide
IELLKTEVTNPAVLRKLCIEAKISNTTTDSRCPTQGEATLVEEQDTNFVCRRTFVDRGHGNGCGLFGKGSLITCAKFKCVTKL

SEQ ID NO: 77 - protein, 83 aa, Artificial Sequence
  Description of Artificial Sequence Synthetic polypeptide
KLCIEAKISNTTTDSRCPTQGEATLVEEQDTNFVCRRTFVDRGHGNGCGLFGKGSLITCAKFKCVTKLEGKIVQYENLKYSVI

SEQ ID NO: 78 - protein, 59 aa, Artificial Sequence
  Description of Artificial Sequence Synthetic polypeptide
EAKISNTTTDSRCPTQGEATLVEEQDTNFVCRRTFVDRGHGNGCGLFGKGSLITCAKFK

SEQ ID NO: 79 - protein, 22 aa, Vesiculovirus indiana
IASFFFIIGLIIGLFLVLRVGI

SEQ ID NO: 80 - protein, 37 aa, Vesiculovirus indiana
PIELVEGWFSSWKSSIASFFFIIGLIIGLFLVLRVGI

SEQ ID NO: 81 - protein, 53 aa, Vesiculovirus indiana
DDESLFFGDTGLSKNPIELVEGWFSSWKSSIASFFFIIGLIIGLFLVLRVGIH

SEQ ID NO: 82 - protein, 81 aa, Vesiculovirus indiana
GMLDSDLHLSSKAQVFEHPHIQDAASQLPDDESLFFGDTGLSKNPIELVEGWFSSWKSSIASFFFIIGLIIGLFLVLRVGI

SEQ ID NO: 83 - protein, 25 aa, Vesiculovirus indiana
HLCIKLKHTKKRQIYTDIEMNRLGK

SEQ ID NO: 84 - protein, 23 aa, Influenza A virus
IITIGSVCMTIGMANLILQIGNI

SEQ ID NO: 85 - protein, 23 aa, Influenza A virus
LAIYSTVASSLVLVVSLGAISFW

SEQ ID NO: 86 - protein, 52 aa, Dengue virus
AYGVLFSGVSWTMKIGIGILLTWLGLNSRSTSLSMTCIAVGMVTLYLGVMVQ

SEQ ID NO: 87 - protein, 43 aa, Human immunodeficiency virus 1
LFIMIVGGLVGLRIVFAVLSIVNRVRQGYSPLSFQTHLPTPRG

SEQ ID NO: 88 - protein, 60 aa, Unknown
  Description of Unknown Spike protein S1 sequence
WYVWLGFIAGLIAIVMVTILLCCMTSCCSCLKGACSCGSCCKFDEDDSEPVLKGVKLHYT

SEQ ID NO: 89 - protein, 60 aa, Unknown
  Description of Unknown Spike protein S2 sequence
WYIWLGFIAGLIAIVMVTIMLCCMTSCCSCLKGCCSCGSCCKFDEDDSEPVLKGVKLHYT

SEQ ID NO: 90 - protein, 31 aa, Sindbis virus
WLFALFGGASSLLIIGLMIFACSMMLTSTRR

SEQ ID NO: 91 - protein, 58 aa, Measles morbillivirus
MSPQRDRINAFYKDNPHPKGSRIVINREHLMIDRPYVLLAVLFVMFLSLIGLLAIAGI

SEQ ID NO: 92 - protein, 26 aa, Measles morbillivirus
IVYILIAVCLGGLIGIPALICCCRGR

SEQ ID NO: 93 - protein, 49 aa, Unknown
  Description of Unknown RD114 TM sequence
LLTLLLILTIGPCVFSRLMAFINDRLNVVHAMVLAQQYQALKAEEEAQD

SEQ ID NO: 94 - protein, 49 aa, Unknown
  Description of Unknown BaEV TM sequence
LLTLLLLLTIGPCIFNRLTAFINDKLNIIHAMVLTQQYQVLRTDEEAQD

SEQ ID NO: 95 - protein, 182 aa, Unknown
  Description of Unknown GP120 TM sequence
IKIFIMIVGGLVGLRIIFAVLSIVNRVRQGYSPLSFQIHSHHQREPDRPEGIEEGGGEQGKDRSVRLVSGFLALAWDDLRSLCLFSYHRLRDFILVAARTVELLGHSSLKGLRLGWEGLKYLGNLLIYWSQELKTSAISLFDTIAIAVAGWTDRVIEIAQRAGRAIIHIPRRIRQGLERALQ

SEQ ID NO: 96 - protein, 585 aa, Homo sapiens
MEKKWKYCAVYYIIQIHFVKGVWEKTVNTEENVYATLGSDVNLTCQTQTVGFFVQMQWSKVTNKIDLIAVYHPQYGFYCAYGRPCESLVTFTETPENGSKWTLHLRNMSCSVSGRYECMLVLYPEGIQTKIYNLLIQTHVTADEWNSNHTIEIEINQTLEIPCFQNSSSKISSEFTYAWSVENSSTDSWVLLSKGIKEDNGTQETLISQNHLISNSTLLKDRVKLGTDYRLHLSPVQIFDDGRKFSCHIRVGPNKILRSSTTVKVFAKPEIPVIVENNSTDVLVERRFTCLLKNVFPKANITWFIDGSFLHDEKEGIYITNEERKGKDGFLELKSVLTRVHSNKPAQSDNLTIWCMALSPVPGNKVWNISSEKITFLLGSEISSTDPPLSVTESTLDTQPSPASSVSPARYPATSSVTLVDVSALRPNTTPQPSNSSMTTRGFNYPWTSSGTDTKKSVSRIPSETYSSSPSGAGSTLHDNVFTSTARAFSEVPTTANGSTKTNHVHITGIVVNKPKDGMSWPVIVAALLFCCMILFGLGVRKWCQYQKEIMERPPPFKPPPPPIKYTCIQEPNESDLPYHEMETL

SEQ ID NO: 97 - protein, 602 aa, Mus sp.
MGRKWTYCVVYTIIQIQFFRGVWEELFNVGDDVYALPGSDINLTCQTKEKNFLVQMQWSKVTDKNDMIALYHPQYGLYCGQEHACESQVAATETEKGVTNWTLYLRNISSALGGKYECIFTLYPEGIKTTVYNLIVEPYTQDEHNYTIEIETNRTLEIPCFQNTSSEIPPRFTFSWLVEKDGVEEVLFTHHHHVNNSTSFKGRIRLGGDYRLHLSPVQIQDDGRTFSCHLTVNPLKAWKMSTTVKVFAKPEILMTVENSTMDVLGERVFTCLLKNVFPKANITWFIDGRFLQGNEEGIYITNEEKNCSSGFWELKSVLTRMHSGPSQSNNMTAWCMALSPGPRNKMWNTSSQPITVSFDSVIAPTKHLPTVTGSTLGTQPFSDAGVSPTGYLATPSVTIVDENGLTPDATPQTSNSSMTTKDGNYLEASSGTDAKNSSRAAASSKSGSWPFPFTSPPEWHSLPGTSTGPQEPDSPVSWIPSEVHTSAPLDASLAPHDTIISTTTEFPNVLTTANGTTKIDHGPITSIIVNQPSDGMSWPVLVAALLFFCTLLFGLGVRKWYRYQNEIMERPPPFKPPPPPIKYTYIQEPIGCDLCCHEMEVL

SEQ ID NO: 98 - protein, 282 aa, Homo sapiens
MASLGQILFWSIISIIIILAGAIALIIGFGISGRHSITVTTVASAGNIGEDGILSCTFEPDIKLSDIVIQWLKEGVLGLVHEFKEGKDELSEQDEMFRGRTAVFADQVIVGNASLRLKNVQLTDAGTYKCYIITSKGKGNANLEYKTGAFSMPEVNVDYNASSETLRCEAPRWFPQPTVVWASQVDQGANFSEVSNTSFELNSENVTMKVVSVLYNVTINNTYSCMIENDIAKATGDIKVTESEIKRRSHLQLLNSKASLCVSSFFAISWALLPLSPYLMLK

SEQ ID NO: 99 - protein, 283 aa, Mus sp.
MASLGQIIFWSIINIIIILAGAIALIIGFGISGKHFITVTTFTSAGNIGEDGTLSCTFEPDIKLNGIVIQWLKEGIKGLVHEFKEGKDDLSQQHEMFRGRTAVFADQVVVGNASLRLKNVQLTDAGTYTCYIRTSKGKGNANLEYKTGAFSMPEINVDYNASSESLRCEAPRWFPQPTVAWASQVDQGANFSEVSNTSFELNSENVTMKVVSVLYNVTINNTYSCMIENDIAKATGDIKVTDSEVKRRSQLQLLNSGPSPCVFSSAFVAGWALLSLSCCLMLR

SEQ ID NO: 100 - protein, 467 aa, Homo sapiens
MLLLLLLPLLWGRERVEGQKSNRKDYSLTMQSSVTVQEGMCVHVRCSFSYPVDSQTDSDPVHGYWFRAGNDISWKAPVATNNPAWAVQEETRDRFHLLGDPQTKNCTLSIRDARMSDAGRYFFRMEKGNIKWNYKYDQLSVNVTALTHRPNILIPGTLESGCFQNLTCSVPWACEQGTPPMISWMGTSVSPLHPSTTRSSVLTLIPQPQHHGTSLTCQVTLPGAGVTTNRTIQLNVSYPPQNLTVTVFQGEGTASTALGNSSSLSVLEGQSLRLVCAVDSNPPARLSWTWRSLTLYPSQPSNPLVLELQVHLGDEGEFTCRAQNSLGSQHVSLNLSLQQEYTGKMRPVSGVLLGAVGGAGATALVFLSFCVIFIVVRSCRKKSARPAADVGDIGMKDANTIRGSASQGNLTESWADDNPRHHGLAAHSSGEEREIQYAPLSFHKGEPQDLSGQEATNNEYSEIKIPK

SEQ ID NO: 101 - protein, 463 aa, Homo sapiens
MLLLLLPLLWGRERAEGQTSKLLTMQSSVTVQEGLCVHVPCSFSYPSHGWIYPGPVVHGYWFREGANTDQDAPVATNNPARAVWEETRDRFHLLGDPHTKNCTLSIRDARRSDAGRYFFRMEKGSIKWNYKHHRLSVNVTALTHRPNILIPGTLESGCPQNLTCSVPWACEQGTPPMISWIGTSVSPLDPSTTRSSVLTLIPQPQDHGTSLTCQVTFPGASVTTNKTVHLNVSYPPQNLTMTVFQGDGTVSTVLGNGSSLSLPEGQSLRLVCAVDAVDSNPPARLSLSWRGLTLCPSQPSNPGVLELPWVHLRDAAEFTCRAQNPLGSQQVYLNVSLQSKATSGVTQGVVGGAGATALVFLSFCVIFVVVRSCRKKSARPAAGVGDTGIEDANAVRGSASQGPLTEPWAEDSPPDQPPPASARSSVGEGELQYASLSFQMVKPWDSRGQEATDTEYSEIKIHR

SEQ ID NO: 102 - protein, 260 aa, Homo sapiens
MARPHPWWLCVLGTLVGLSATPAPKSCPERHYWAQGKLCCQMCEPGTFLVKDCDQHRKAAQCDPCIPGVSFSPDHHTRPHCESCRHCNSGLLVRNCTITANAECACRNGWQCRDKECTECDPLPNPSLTARSSQALSPHPQPTHLPYVSEMLEARTAGHMQTLADFRQLPARTLSTHWPPQRSLCSSDFIRILVIFSGMFLVFTLAGALFLHQRRKYRSNKGESPVEPAEPCHYSCPREEEGSTIPIQEDYRKPEPACSP

SEQ ID NO: 103 - protein, 250 aa, Mus sp.
MAWPPPYWLCMLGTLVGLSATLAPNSCPDKHYWTGGGLCCRMCEPGTFFVKDCEQDRTAAQCDPCIPGTSFSPDYHTRPHCESCRHCNSGFLIRNCTVTANAECSCSKNWQCRDQECTECDPPLNPALTRQPSETPSPQPPPTHLPHGTEKPSWPLHRQLPNSTVYSQRSSHRPLCSSDCIRIFVTFSSMFLIFVLGAILFFHQRRNHGPNEDRQAVPEEPCPYSCPREEEGSAIPIQEDYRKPEPAFYP

SEQ ID NO: 104 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 105 - protein, 218 aa, Mus sp.
MTLRLLFLALNFFSVQVTENKILVKQSPLLVVDSNEVSLSCRYSYNLLAKEFRASLYKGVNSDVEVCVGNGNFTYQPQFRSNAEFNCDGDFDNETVTFRLWNLHVNHTDIYFCKIEFMYPPPYLDNERSNGTIIHIKEKHLCHTQSSPKLFWALVVVAGVLFCYGLLVTVALCVIWTNSRRNRLLQSDYMNMTPRRPGLTRKPYQPYAPARDFAAYRP

SEQ ID NO: 106 - protein, 552 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLAQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 107 - protein, 539 aa, Mus sp.
MATIALPWSLSLYVFLLLLATPWASAAVKNCSHLECFYNSRANVSCMWSHEEALNVTTCHVHAKSNLRHWNKTCELTLVRQASWACNLILGSFPESQSLTSVDLLDINVVCWEEKGWRRVKTCDFHPFDNLRLVAPHSLQVLHIDTQRCNISWKVSQVSHYIEPYLEFEARRRLLGHSWEDASVLSLKQRQQWLFLEMLIPSTSYEVQVRVKAQRNNTGTWSPWSQPLTFRTRPADPMKEILPMSWLRYLLLVLGCFSGFFSCVYILVKCRYLGPWLKTVLKCHIPDPSEFFSQLSSQHGGDLQKWLSSPVPLSFFSPSGPAPEISPLEVLDGDSKAVQLLLLQKDSAPLPSPSGHSQASCFTNQGYFFFHLPNALEIESCQVYFTYDPCVEEEVEEDGSRLPEGSPHPPLLPLAGEQDDYCAFPPRDDLLLFSPSLSTPNTAYGGSRAPEERSPLSLHEGLPSLASRDLMGLQRPLERMPEGDGEGLSANSSGEQASVPEGNLHGQDQDRGQGPILTLNTDAYLSLQELQAQDSVHLI

SEQ ID NO: 108 - protein, 241 aa, Homo sapiens
MAQHGAMGAFRALCGLALLCALSLGQRPTGGPGCGPGRLLLGTGTDARCCRVHTTRCCRDYPGEECCSEWDCMCVQPEFHCGDPCCTTCRHHPCPPGQGVQSQGKFSFGFQCIDCASGTFSGGHEGHCKPWTDCTQFGFLTVFPGNKTHNAVCVPGSPPAEPLGWLTVVLLAVAACVLLLTSAQLGLHIWQLRSQCMWPRETQLLLEVPPSTEDARSCQFPEEERGERSAEEKGRLGDLWV

SEQ ID NO: 109 - protein, 228 aa, Mus sp.
MGAWAMLYGVSMLCVLDLGQPSVVEEPGCGPGKVQNGSGNNTRCCSLYAPGKEDCPKERCICVTPEYHCGDPQCKICKHYPCQPGQRVESQGDIVFGFRCVACAMGTFSAGRDGHCRLWTNCSQFGFLTMFPGNKTHNAVCIPEPLPTEQYGHLTVIFLVMAACIFFLTTVQLGLHIWQLRRQHMCPRETQPFAEVQLSAEDACSFQFPEEERGEQTEEKCHLGGRWP

SEQ ID NO: 110 - protein, 193 aa, Homo sapiens
MPEEGSGCSVRRRPYGCVLRAALVPLVAGLVICLVVCIQRFAQAQQQLPLESLGWDVAELQLNHTGPQQDPRLYWQGGPALGRSFLHGPELDKGQLRIHRDGIYMVHIQVTLAICSSTTASRHHPTTLAVGICSPASRSISLLRLSFHQGCTIASQRLTPLARGDTLCTNLTGTLLPSRNTDETFFGVQWVRP

SEQ ID NO: 111 - protein, 195 aa, Mus sp.
MPEEGRPCPWVRWSGTAFQRQWPWLLLVVFITVFCCWFHCSGLLSKQQQRLLEHPEPHTAELQLNLTVPRKDPTLRWGAGPALGRSFTHGPELEEGHLRIHQDGLYRLHIQVTLANCSSPGSTLQHRATLAVGICSPAAHGISLLRGRFGQDCTVALQRLTYLVHGDVLCTNLTLPLLPSRNADETFFGVQWICP

SEQ ID NO: 112 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 113 - protein, 169 aa, Mus sp.
MYSMQLASCVTLTLVLLVNSAPTSSSTSSSTAEAQQQQQQQQQQQQHLEQLLMDLQELLSRMENYRNLKLPRMLTFKFYLPKQATELKDLQCLEDELGPLRHVLDLTQSKSFQLEDAENFISNIRVTVVKLKGSDNTFECQFDDESATVVDFLRRWIAFCQSIISTSPQ

SEQ ID NO: 114 - protein, 199 aa, Homo sapiens
MTLHPSPITCEFLFSTALISPKMCLSHLENMPLSHSRTQGAQRSSWKLWLFCSIVMLLFLCSFSWLIFIFLQLETAKEPCMAKFGPLPSKWQMASSEPPCVNKVSDWKLEILQNGLYLIYGQVAPNANYNDVAPFEVRLYKNKDMIQTLTNKSKIQNVGGTYELHVGDTIDLIFNSEHQVLKNNTYWGIILLANPQFIS

SEQ ID NO: 115 - protein, 173 aa, Mus sp.
MEEMPLRESSPQRAERCKKSWLLCIVALLLMLLCSLGTLIYTSLKPTAIESCMVKFELSSSKWHMTSPKPHCVNTTSDGKLKILQSGTYLIYGQVIPVDKKYIKDNAPFVVQIYKKNDVLQTLMNDFQILPIGGVYELHAGDNIYLKFNSKDHIQKTNTYWGIILMPDLPFIS

SEQ ID NO: 116 - protein, 428 aa, Artificial Sequence
  Description of Artificial Sequence Synthetic polypeptide
MGVRQVPWSFTWAVLQLSWQSGWLLEVPNGPWRSLTFYPAWLTVSEGANATFTCSLSNWSEDLMLNWNRLSPSNQTEKQAAFCNGLSQPVQDARFQIIQLPNRHDFHMNILDTRRNDSGIYLCGAISLHPKAKIEESPGAELVVTERILETSTRYPSPSPKPEGRFQGMVIGIMSALVGIPVLLLLAWALDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 117 - protein, 475 aa, Artificial Sequence
  Description of Artificial Sequence Synthetic polypeptide
MRIFAGIIFTACCHLLRAFTITAPKDLYVVEYGSNVTMECRFPVERELDLLALVVYWEKEDEQVIQFVAGEEDLKPQHSNFRGRASLPKDQLLKGNAALQITDVKLQDAGVYCCIISYGGADYKRITLKVNAPYRKINQRISVDPATSEHELICQAEGYPEAEVIWTNSDHQPVSGKRSVTTSRTEGMLLNVTSSLRVNATANDVFYCTFWRSQPGQNHTAELIIPELPATHPPQNRDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 118 - protein, 464 aa, Artificial Sequence
  Description of Artificial Sequence Synthetic polypeptide
MIGQAVLFTTFLLLRAHQGQDCPDSSEEVVGVSGKPVQLRPSNIQTKDVSVQWKKTEQGSHRKIEILNWYNDGPSWSNVSFSDIYGFDYGDFALSIKSAKLQDSGHYLLEITNTGGKVCNKNFQLLILDHVETPNLKAQWKPWTNGTCQLFLSCLVTKDDNVSYALYRGSTLISNQRNSTHWENQIDASSLHTYTCNVSNRASWANHTLNFTHGCQSVPSNFRFLPDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 119 - protein, 457 aa, Artificial Sequence
  Description of Artificial Sequence Synthetic polypeptide
MLLLLPILNLSLQLHPVAALFTVTAPKEVYTVDVGSSVSLECDFDRRECTELEGIRASLQKVENDTSLQSERATLLEEQLPLGKALFHIPSVQVRDSGQYRCLVICGAAWDYKYLTVKVKASYMRIDTRILEVPGTGEVQLTCQARGYPLAEVSWQNVSVPANTSHIRTPEGLYQVTSVLRLKPQPSRNFSCMFWNAHMKELTSAIIDPLSRMEPKVPRDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 120 - protein, 429 aa, Artificial Sequence
  Description of Artificial Sequence Synthetic polypeptide
MTCLGLRRYKAQLQLPSRTWPFVALLTLLFIPVFSEAIQVTQPSVVLASSHGVASFPCEYSPSHNTDEVRVTVLRQTNDQMTEVCATTFTEKNTVGFLDYPFCSGTFNESRVNLTIQGLRAVDTGLYLCKVELMYPPPYFVGMGNGTQIYVIDPEPCPDSDFLLWILVAVSLGLFFYSFLVTAVSLSKRIQDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 121 - protein, 501 aa, Artificial Sequence
  Description of Artificial Sequence Synthetic polypeptide
MADSFSLNDALAGSGNPNPQGYPGAWGNQPGAGGYPGAAYPGAYPGQAPPGAYPGQAPPGAYPGQAPPSAYPGPTAPGAYPGPTAPGAYPGQPAPGAFPGQPGAPGAYPQCSGGYPAAGPYGVPAGPLTVPYDLPLPGGVMPRMLITIMGTVKPNANRIVLDFRRGNDVAFHFNPRFNENNRRVIVCNTKQDNNWGKEERQSAFPFESGKPFKIQVLVEADHFKVAVNDAHLLQYNHRMKNLREISQLGISGDITLTSANHAMIIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 122 - protein, 678 aa, Artificial Sequence
  Description of Artificial Sequence Synthetic polypeptide
MGEDLLLGFLLLGLLWEAPVVSSGPGKELPVVWAQEGAPVHLPCSLKSPNLDPNFLRRGGVIWQHQPDSGQPTPIPALDLHQGMPSPRQPAPGRYTVLSVAPGGLRSGRQPLHPHVQLEERGLQRGDFSLWLRPALRTDAGEYHATVRLPNRALSCSLRLRVGQASMIASPSGVLKLSDWVLLNCSFSRPDRPVSVHWFQGQNRVPVYNSPRHFLAETFLLLPQVSPLDSGTWGCVLTYRDGFNVSITYNLKVLGLEPVAPLTVYAAEGSRVELPCHLPPGVGTPSLLIAKWTPPGGGPELPVAGKSGNFTLHLEAVGLAQAGTYTCSIHLQGQQLNATVTLAVITVTPKSFGLPGSRGKLLCEVTPASGKERFVWRPLNNLSRSCPGPVLEIQEARLLAERWQCQLYEGQRLLGATVYAAESSSGAHSARRISGDLKGGDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 123 - protein, 551 aa, Artificial Sequence
  Description of Artificial Sequence Synthetic polypeptide
MGKIYSFVLVAIALMMGREGWALESESCLREQVRLRAQVHQLETRVKQQQTMIAQLLHEKEVQFLDKGSENSFIDLGGKRQYADCSEIYNDGFKQSGFYKIKPLQSLAEFSVYCDMSDGGGWTVIQRRSDGSENFNRGWNDYENGFGNFVQNNGEYWLGNKNINLLTIQGDYTLKIDLTDFEKNSSFAQYQSFKVGDKKSFYELNIGEYSGTAGDSLSGTFHPEVQWWASHQRMKFSTWDRDNDNYQGNCAEEEQSGWWFNRCHSANLNGVYYRGSYRAETDNGVVWYTWHGWWYSLKSVVMKIRPSDFIPNIIIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 124 - protein, 445 aa, Artificial Sequence
  Description of Artificial Sequence Synthetic polypeptide
MEPLPGWGSAPWSQAPTDNTFRLVPCVFLLNLLQRISAQPSCRQEEFLVGDECCPMCNPGYHVKQVCSEHTGTVCAPCPPQTYTAHANGLSKCLPCGVCDPDMGLLTWQECSSWKDTVCRCIPGYFCENQDGSHCSTCLQHTTCPPGQRVEKRGTHDQDTVCADCLTGTFSLGGTQEECLPWTNCSAFQQEVRRGTNSTDTTCSSQVDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 125 - protein, 419 aa, Artificial Sequence
  Description of Artificial Sequence Synthetic polypeptide
MKTVPAMLGTPRLFREFFILHLGLWSILCEKATKRNDEECPVQLTITRNSKQSARTGELFKIQCPVKYCVHRPNVTWCKHNGTICVPLEVSPQLYTSWEENQSVPVFVLHFKPIHLSDNGSYSCSTNFNSQVINSHSVTIHVTERTQNSSEHPLITVSDIPDATNASGPSTMEERPGRTWLDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 126 - protein, 425 aa, Artificial Sequence
  Description of Artificial Sequence Synthetic polypeptide
MERILMAPGQSCCALAILLAIVNFQHGGCIHVTSSASQKGGRLDLTCTLWHKKDEAEGLILFWCKDNPWNCSPETNLEQLRVKRDPETDGITEKSSQLVFTIEQATPSDSGTYQCCARSQKPEIYIHGHFLSVLVTGNHTEIRQRQRSHPDFSHINGTLSSGFLQVKAWGMLVTSLVALQALYTLAADIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 127 - protein, 477 aa, Artificial Sequence
  Description of Artificial Sequence Synthetic polypeptide
MCFIKQGWCLVLELLLLPLGTGFQGHSIPDINATTGSNVTLKIHKDPLGPYKRITWLHTKNQKILEYNYNSTKTIFESEFKGRVYLEENNGALHISNVRKEDKGTYYMRVLRETENELKITLEVFDPVPKPSIEINKTEASTDSCHLRLSCEVKDQHVDYTWYESSGPFPKKSPGYVLDLIVTPQNKSTFYTCQVSNPVSSKNDTVYFTLPCDLARSSGVCWTATWLVVTTLIIHRILLTIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 128 - protein, 588 aa, Artificial Sequence
  Description of Artificial Sequence Synthetic polypeptide
MARAAVLPPSRLSPTLPLLPLLLLLLQETGAQDVRVRVLPEVRGRLGGTVELPCHLLPPTTERVSQVTWQRLDGTVVAAFHPSFGVDFPNSQFSKDRLSFVRARPETNADLRDATLAFRGLRVEDEGNYTCEFATFPNGTRRGVTWLRVIAQPENHAEAQEVTIGPQSVAVARCVSTGGRPPARITWISSLGGEAKDTQEPGIQAGTVTIISRYSLVPVGRADGVKVTCRVEHESFEEPILLPVTLSVRYPPEVSISGYDDNWYLGRSEAILTCDVRSNPEPTDYDWSTTSGVFPASAVAQGSQLLVHSVDRMVNTTFICTATNAVGTGRAEQVILVRESPSTAGAGATGGIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 129 - protein, 377 aa, Artificial Sequence
  Description of Artificial Sequence Synthetic polypeptide
MKGWLLLVWVQGLIQAAFLATGATAGTIDTKRNISAEEGGSVILQCHFSSDTAEVTQVDWKQQDQLLAIYSVDLGWHVASVFSDRVVPGPSLGLTFQSLTMNDTGEYFCTYHTYPGGIYKGRIFLKVQESSVAQFQTAPDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 130 - protein, 585 aa, Artificial Sequence
  Description of Artificial Sequence Synthetic polypeptide
MAQLARATRSPLSWLLLLFCYALRKAGGDIRVLVPYNSTGVLGGSTTLHCSLTSNENVTITQITWMKKDSGGSHALVAVFHPKKGPNIKEPERVKFLAAQQDLRNASLAISNLSVEDEGIYECQIATFPRGSRSTNAWLKVQARPKNTAEALEPSPTLILQDVAKCISANGHPPGRISWPSNVNGSHREMKEPGSQPGTTTVTSYLSMVPSRQADGKNITCTVEHESLQELDQLLVTLSQPYPPENVSISGYDGNWYVGLTNLTLTCEAHSKPAPDMAGYNWSTTTGDFPNSVKRQGNMLLISTVEDGLNNTVIVCEVTNALGSGQGQVHIIVKEKPENMQQNTRLHLIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 131 - protein, 439 aa, Artificial Sequence
  Description of Artificial Sequence Synthetic polypeptide
MFSHLPFDCVLLLLLLLLTRSSEVEYRAEVGQNAYLPCFYTPAAPGNLVPVCWGKGACPVFECGNVVLRTDERDVNYWTSRYWLNGDFRKGDVSLTIENVTLADSGIYCCRIQIPGIMNDEKFNLKLVIKPAKVTPAPTRQRDFTAAFPRMLTTRGHGPAETQTLGSLPDINLTQISTLANELRDSRLANDLRDSGATIRIDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 132 - protein, 665 aa, Artificial Sequence
  Description of Artificial Sequence Synthetic polypeptide
MGHLSAPLHRVRVPWQGLLLTASLLTFWNPPTTAQLTTESMPFNVAEGKEVLLLVHNLPQQLFGYSWYKGERVDGNRQIVGYAIGTQQATPGPANSGRETIYPNASLLIQNVTQNDTGFYTLQVIKSDLVNEEATGQFHVYPELPKPSISSNNSNPVEDKDAVAFTCEPETQDTTYLWWINNQSLPVSPRLQLSNGNRTLTLLSVTRNDTGPYECEIQNPVSANRSDPVTLNVTYGPDTPTISPSDTYYRPGANLSLSCYAASNPPAQYSWLINGTFQQSTQELFIPNITVNNSGSYTCHANNSVTGCNRTTVKTIIVTELSPVVAKPQIKASKTTVTGDKDSVNLTCSTNDTGISIRWFFKNQSLPSSERMKLSQGNTTLSINPVKREDAGTYWCEVFNPISKNQSDPIMLNVNYNALPQENGLSPGIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 133 - protein, 483 aa, Artificial Sequence
  Description of Artificial Sequence Synthetic polypeptide
MDPRCTMGLAILIFVTVLLISDAVSVETQAYFNGTAYLPCPFTKAQNISLSELVVFWQDQQKLVLYEHYLGTEKLDSVNAKYLGRTSFDRNNWTLRLHNVQIKDMGSYDCFIQKKPPTGSIILQQTLTELSVIANFSEPEIKLAQNVTGNSGINLTCTSKQGHPKPKKMYFLITNSTNEYGDNMQISQDNVTELFSISNSLSLSFPDGVWHMTVVCVLETESMKISSKPLNFTQEFPSPQTYWKEDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 134 - protein, 485 aa, Artificial Sequence
  Description of Artificial Sequence Synthetic polypeptide
MGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 135 - protein, 486 aa, Artificial Sequence
  Description of Artificial Sequence Synthetic polypeptide
MACNCQLMQDTPLLKFPCPRLILLFVLLIRLSQVSSDVDEQLSKSVKDKVLLPCRYNSPHEDESEDRIYWQKHDKVVLSVIAGKLKVWPEYKNRTLYDNTTYSLIILGLVLSDRGTYSCVVQKKERGTYEVKHLALVKLSIKADFSTPNITESGNPSADTKRITCFASGGFPKPRFSWLENGRELPGINTTISQDPESELYTISSQLDFNTTRNHTIKCLIKYGDAHVSEDFTWEKPPEDPPDSKNTLDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 136 - protein, 484 aa, Artificial Sequence
  Description of Artificial Sequence Synthetic polypeptide
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 137 - protein, 293 aa, Artificial Sequence
  Description of Artificial Sequence Synthetic polypeptide
MYTDIEMNRLGKNPNQKIITIGSICMVTGIVSLMLQIGNMISIWVSHSIHTGNQHQSEPISNTNFLTEKAVASVKLAGNSSLCPINDRTEPRPALTITTSPNLGTRENNADQVTPVSHIGCPNTTQQGSPVFAKLLAKNQASLCNTTLNWHSQDGAGSSYLSQGLRYEEDKKELVVDSPGLYYVFLELKLSPTFTNTGHKVQGWVSLVLQAKPQVDDFDNLALTVELFPCSMENKLVDRSWSQLLLLKAGHRLSVGLRAYLHGAQDAYRDWELSYPNTTSFGLFLVKPDNPWE

SEQ ID NO: 138 - protein, 292 aa, Artificial Sequence
  Description of Artificial Sequence Synthetic polypeptide
MYTDIEMNRLGKNPNQKIITIGSICMVTGIVSLMLQIGNMISIWVSHSIHTGNQHQSEPISNTNFLTEKAVASVKLAGNSSLCPINDACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 139 - protein, 235 aa, Artificial Sequence
  Description of Artificial Sequence Synthetic polypeptide
MYTDIEMNRLGKNPNQKIITIGSICMVTGIVSLMLQIGNMISIWVSHSIHTGNQHQSEPISNTNFLTEKAVASVKLAGNSSLCPINDSSSPAKDPPIQRLRGAVTRCEDGQLFISSYKNEYQTMEVQNNSVVIKCDGLYIIYLKGSFFQEVKIDLHFREDHNPISIPMLNDGRRIVFTVVASLAFKDKVYLTVNAPDTLCEHLQINDGELIVVQLTPGYCAPEGSYHSTVNQVPL

SEQ ID NO: 140 - protein, 220 aa, Artificial Sequence
  Description of Artificial Sequence Synthetic polypeptide
MYTDIEMNRLGKNPNQKIITIGSICMVTGIVSLMLQIGNMISIWVSHSIHTGNQHQSEPISNTNFLTEKAVASVKLAGNSSLCPINDMVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 141 - protein, 268 aa, Artificial Sequence
  Description of Artificial Sequence Synthetic polypeptide
MYTDIEMNRLGKNPNQKIITIGSICMVTGIVSLMLQIGNMISIWVSHSIHTGNQHQSEPISNTNFLTEKAVASVKLAGNSSLCPINDLHQRLGDIVAHLPDGGKGSWEKLIQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMV

SEQ ID NO: 142 - protein, 524 aa, Artificial Sequence
  Description of Artificial Sequence Synthetic polypeptide
MSALLTAAGLLFLGMLQAFPTDRPLKTTCAGDLSHYPGEAARNCCYQCPSGLSPTQPCPRGPAHCRKQCAPDYYVNEDGKCTACVTCLPGLVEKAPCSGNSPRICECQPGMHCCTPAVNSCARCKLHCSGEEVVKSPGTAKKDTICELPSSGSGPNCSNPGDRKTLTSHATPQAMPTLESPANDSARSLLPMRVTNLVQEDATELVKVPESSSSKAREPSPDPGNAEKNMTLELPSPGTLPDISTSENSKEPASTASTLSLVVDAWTSSRMQPTSPLSTGTPFLDPGIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 143 - protein, 259 aa, Artificial Sequence
  Description of Artificial Sequence Synthetic polypeptide
MYTDIEMNRLGKNPNQKIITIGSICMVTGIVSLMLQIGNMISIWVSHSIHTGNQHQSEPISNTNFLTEKAVASVKLAGNSSLCPINDQKKDSTPNTTEKAPLKGGNCSEDLFCTLKSTPSKKSWAYLQVSKHLNNTKLSWNEDGTIHGLIYQDGNLIVQFPGLYFIVCQLQFLVQCSNHSVDLTLQLLINSKIKKQTLVTVCESGVQSKNIYQNLSQFLLHYLQVNSTISVRVDNFQYVDTNTFPLDNVLSVFLYSSSD

SEQ ID NO: 144 - protein, 443 aa, Artificial Sequence
  Description of Artificial Sequence Synthetic polypeptide
MKCKFLGSFFLLFSLSGKGADCRDNETIWGVLGHGITLNIPNFQMTDDIDEVRWVRRGTLVAEFKRKKPPFLISETYEVLANGSLKIKKPMMRNDSGTYNVMVYGTNGMTRLEKDLDVRILERVSKPMIHWECPNTTLTCAVLQGTDFELKLYQGETLLNSLPQKNMSYQWTNLNAPFKCEAINPVSKESKMEVVNCPEKGLSFYDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 145 - protein, 419 aa, Artificial Sequence
  Description of Artificial Sequence Synthetic polypeptide
MAWPPPYWLCMLGTLVGLSATLAPNSCPDKHYWTGGGLCCRMCEPGTFFVKDCEQDRTAAQCDPCIPGTSFSPDYHTRPHCESCRHCNSGFLIRNCTVTANAECSCSKNWQCRDQECTECDPPLNPALTRQPSETPSPQPPPTHLPHGTEKPSWPLHRQLPNSTVYSQRSSHRPLCSSDCIRIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 146 - protein, 238 aa, Artificial Sequence
  Description of Artificial Sequence Synthetic polypeptide
MYTDIEMNRLGKNPNQKIITIGSICMVTGIVSLMLQIGNMISIWVSHSIHTGNQHQSEPISNTNFLTEKAVASVKLAGNSSLCPINDSKQQQRLLEHPEPHTAELQLNLTVPRKDPTLRWGAGPALGRSFTHGPELEEGHLRIHQDGLYRLHIQVTLANCSSPGSTLQHRATLAVGICSPAAHGISLLRGRFGQDCTVALQRLTYLVHGDVLCTNLTLPLLPSRNADETFFGVQWICP

SEQ ID NO: 147 - protein, 517 aa, Artificial Sequence
  Description of Artificial Sequence Synthetic polypeptide
MQLKCPCFVSLGTRQPVWKKLHVSSGFFSGLGLFLLLLSSLCAASAETEVGAMVGSNVVLSCIDPHRRHFNLSGLYVYWQIENPEVSVTYYLPYKSPGINVDSSYKNRGHLSLDSMKQGNFSLYLKNVTPQDTQEFTCRVFMNTATELVKILEEVVRLRVAANFSTPVISTSDSSNPGQERTYTCMSKNGYPEPNLYWINTTDNSLIDTALQNNTVYLNKLGLYDVISTLRLPWTSRGDVLCCVENVALHQNITSISQAESFTGNNTKNPQETHNNELKDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 148 - protein, 380 aa, Artificial Sequence
  Description of Artificial Sequence Synthetic polypeptide
MKPYFCRVFVFCFLIRLLTGEINGSADHRMFSFHNGGVQISCKYPETVQQLKMRLFREREVLCELTKTKGSGNAVSIKNPMLCLYHLSNNSVSFFLNNPDSSQGSYYFCSLSIFDPPPFQERNLSGGYLHIYESQLCCQLKLDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 149 - protein, 219 aa, Artificial Sequence
  Description of Artificial Sequence Synthetic polypeptide
MYTDIEMNRLGKNPNQKIITIGSICMVTGIVSLMLQIGNMISIWVSHSIHTGNQHQSEPISNTNFLTEKAVASVKLAGNSSLCPINDTSLKPTAIESCMVKFELSSSKWHMTSPKPHCVNTTSDGKLKILQSGTYLIYGQVIPVDKKYIKDNAPFVVQIYKKNDVLQTLMNDFQILPIGGVYELHAGDNIYLKFNSKDHIQKTNTYWGIILMPDLPFIS

SEQ ID NO: 150 - protein, 390 aa, Artificial Sequence
  Description of Artificial Sequence Synthetic polypeptide
MGAWAMLYGVSMLCVLDLGQPSVVEEPGCGPGKVQNGSGNNTRCCSLYAPGKEDCPKERCICVTPEYHCGDPQCKICKHYPCQPGQRVESQGDIVFGFRCVACAMGTFSAGRDGHCRLWTNCSQFGFLTMFPGNKTHNAVCIPEPLPTEQYGHIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 151 - protein, 426 aa, Artificial Sequence
  Description of Artificial Sequence Synthetic polypeptide
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTDIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 152 - protein, 450 aa, Artificial Sequence
  Description of Artificial Sequence Synthetic polypeptide
MYVWVQQPTALLLLGLTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPPTLVTPEGPADIIQADGWMCHASKWVTTCDFRWYGPKYITHSIRSFTPSVEQCKESIEQTKQGTWLNPGFPPQSCGYATVTDAEAVIVQVTPHHVLVDEYTGEWVDSQFINGKCSNYICPTVHNSTTWHSDYKVKGLCDSNLGMLDSDLHLSSKAQVFEHPHIQDAASQLPDDESLFFGDTGLSKNPIELVEGWFSSWKSSIASFFFIIGLIIGLFLVLRVGIHLCIKLKHTKKRQIYTDIEMNRLGK

SEQ ID NO: 153 - protein, 595 aa, Homo sapiens
MRVLLAALGLLFLGALRAFPQDRPFEDTCHGNPSHYYDKAVRRCCYRCPMGLFPTQQCPQRPTDCRKQCEPDYYLDEADRCTACVTCSRDDLVEKTPCAWNSSRVCECRPGMFCSTSAVNSCARCFFHSVCPAGMIVKFPGTAQKNTVCEPASPGVSPACASPENCKEPSSGTIPQAKPTPVSPATSSASTMPVRGGTRLAQEAASKLTRAPDSPSSVGRPSSDPGLSPTQPCPEGSGDCRKQCEPDYYLDEAGRCTACVSCSRDDLVEKTPCAWNSSRTCECRPGMICATSATNSCARCVPYPICAAETVTKPQDMAEKDTTFEAPPLGTQPDCNPTPENGEAPASTSPTQSLLVDSQASKTLPIPTSAPVALSSTGKPVLDAGPVLFWVILVLVVVVGSSAFLLCHRRACRKRIRQKLHLCYPVQTSQPKLELVDSRPRRSSTQLRSGASVTEPVAEERGLMSQPLMETCHSVGAAYLESLPLQDASPAGGPSSPRDLPEPRVSTEHTNNKIEKIYIMKADTVIVGTVKAELPEGRGLAGPAEPELEEELEADHTPHYPEQETEPPLGSCSDVMLSVEEEGKEDPLPTAASGK

SEQ ID NO: 154 - protein, 498 aa, Mus sp.
MSALLTAAGLLFLGMLQAFPTDRPLKTTCAGDLSHYPGEAARNCCYQCPSGLSPTQPCPRGPAHCRKQCAPDYYVNEDGKCTACVTCLPGLVEKAPCSGNSPRICECQPGMHCCTPAVNSCARCKLHCSGEEVVKSPGTAKKDTICELPSSGSGPNCSNPGDRKTLTSHATPQAMPTLESPANDSARSLLPMRVTNLVQEDATELVKVPESSSSKAREPSPDPGNAEKNMTLELPSPGTLPDISTSENSKEPASTASTLSLVVDAWTSSRMQPTSPLSTGTPFLDPGPVLFWVAMVVLLVGSGSFLLCYWKACRRRFQQKFHLDYLVQTFQPKMEQTDSCPTEKLTQPQRSGSVTDPSTGHKLSPVSPPPAVETCASVGATYLENLPLLDDSPAGNPFSPREPPEPRVSTEHTNNRIEKIYIMKADTVIVGSVKTEVPEGRAPAGSTESELEAELEVDHAPHYPEQETEPPLGSCTEVMFSVEEGGKEDHGPTTVSEK

SEQ ID NO: 155 - protein, 261 aa, Homo sapiens; identical to SEQ ID NO: 57

SEQ ID NO: 156 - protein, 260 aa, Mus sp.; identical to SEQ ID NO: 58

SEQ ID NO: 157 - protein, 240 aa, Homo sapiens
MEESVVRPSVFVVDGQTDIPFTRLGRSHRRQSCSVARVGLGLLLLLMGAGLAVQGWFLLQLHWRLGEMVTRLPDGPAGSWEQLIQERRSHEVNPAAHLTGANSSLTGSGGPLLWETQLGLAFLRGLSYHDGALVVTKAGYYYIYSKVQLGGVGCPLGLASTITHGLYKRTPRYPEELELLVSQQSPCGRATSSSRVWWDSSFLGGVVHLEAGEKVVVRVLDERLVRLRDGTRSYFGAFMV

SEQ ID NO: 158 - protein, 239 aa, Mus sp.
MESVVQPSVFVVDGQTDIPFRRLEQNHRRRRCGTVQVSLALVLLLGAGLATQGWFLLRLHQRLGDIVAHLPDGGKGSWEKLIQDQRSHQANPAAHLTGANASLIGIGGPLLWETRLGLAFLRGLTYHDGALVTMEPGYYYVYSKVQLSGVGCPQGLANGLPITHGLYKRTSRYPKELELLVSRRSPCGRANSSRVWWDSSFLGGVVHLEAGEEVVVRVPGNRLVRPRDGTRSYFGAFMV

SEQ ID NO: 159 - protein, 234 aa, Homo sapiens
MDPGLQQALNGMAPPGDTAMHVPAGSVASHLGTTSRSYFYLTTATLALCLVFTVATIMVLVVQRTDSIPNSPDNVPLKGGNCSEDLLCILKRAPFKKSWAYLQVAKHLNKTKLSWNKDGILHGVRYQDGNLVIQFPGLYFIICQLQFLVQCPNNSVDLKLELLINKHIKKQALVTVCESGMQTKHVYQNLSQFLLDYLQVNTTISVNVDTFQYIDTSTFPLENVLSIFLYSNSD

SEQ ID NO: 160 - protein, 239 aa, Mus sp.
MEPGLQQAGSCGAPSPDPAMQVQPGSVASPWRSTRPWRSTSRSYFYLSTTALVCLVVAVAIILVLVVQKKDSTPNTTEKAPLKGGNCSEDLFCTLKSTPSKKSWAYLQVSKHLNNTKLSWNEDGTIHGLIYQDGNLIVQFPGLYFIVCQLQFLVQCSNHSVDLTLQLLINSKIKKQTLVTVCESGVQSKNIYQNLSQFLLHYLQVNSTISVRVDNFQYVDTNTFPLDNVLSVFLYSSSD

SEQ ID NO: 161 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 162 - protein, 344 aa, Mus sp.
MKCKFLGSFFLLFSLSGKGADCRDNETIWGVLGHGITLNIPNFQMTDDIDEVRWVRRGTLVAEFKRKKPPFLISETYEVLANGSLKIKKPMMRNDSGTYNVMVYGTNGMTRLEKDLDVRILERVSKPMIHWECPNTTLTCAVLQGTDFELKLYQGETLLNSLPQKNMSYQWTNLNAPFKCEAINPVSKESKMEVVNCPEKGLSFYVTVGVGAGGLLLVLLVALFIFCICKRRKRNRRRKDEELEIKASRTSTVERGPKPHSTPAAAAQNSVALQAPPPPGHHLQTPGHRPLPPGHRTREHQQKKRPPPSGTQIHQQKGPPLPRPRVQPKPPCGSGDGVSLPPPN

SEQ ID NO: 163 - protein, 9 aa, Artificial Sequence
  Description of Artificial Sequence Synthetic peptide
EGSRNQDWL

(19) United States
(12) Patent Application Publication
(10) Pub. No.: US 2024/0252613 A1
(43) Pub. Date: Aug. 1, 2024

(54) IMMUNE CHECKPOINT MULTIVALENT PARTICLES COMPOSITIONS AND METHODS OF USE

(71) Applicant: Achelois BioPharma, Inc., Redwood City, CA (US)

(72) Inventors: Chang-Zheng CHEN, Palo Alto, CA (US); Yiling LUO, South San Francisco, CA (US); Michael CHEN, Palo Alto, CA (US); Hua ZHOU, San Mateo, CA (US); Tian-Qiang SUN, San Francisco, CA (US); Michael LINCOLN, Redwood City, CA (US)

(21) Appl. No.: 18/560,623

(22) PCT Filed: May 19, 2022
(86) PCT No.: PCT/US2022/030012

§ 371 Date: Nov. 13, 2023

Related U.S. Application Data
(60) Provisional application No. 63/191,031, filed on May 20, 2021.

(51) Int. Cl. A61K 39/145; A61K 39/00; A61K 39/12; A61K 39/165; A61K 39/205; A61K 39/21; A61P 35/00; A61P 37/04

(52) U.S. Cl. CPC A61K 39/145; A61K 39/12; A61K 39/165; A61K 39/205; A61K 39/21; A61P 35/00; A61P 37/04; A61K 2039/5258; A61K 2039/55516; A61K 2039/585; A61K 2039/70

(57) ABSTRACT

Provided herein are multivalent particles and compositions of multivalent particles expressing immune checkpoint molecules.

### CROSS-REFERENCE

This application claims the benefit of U.S. Provisional Application No. 63/191,031 filed May 20, 2021, which is incorporated herein by reference in its entirety.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF SUMMARY

Disclosed herein, in some embodiments, is a multivalent particle comprising a fusion protein that comprises a mammalian immune checkpoint polypeptide and a transmembrane polypeptide wherein the fusion protein is expressed at a valency of at least about 10 copies on a surface of the multivalent particle. In some embodiments, the mammalian immune checkpoint polypeptide comprises a polypeptide expressed on T cells. In some embodiments, the mammalian immune checkpoint polypeptide comprises an immune inhibitory checkpoint polypeptide. In some embodiments, the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9. In some embodiments, the immune inhibitory checkpoint polypeptide is expressed on antigen presenting cells, tumor cells, or normal cells. In some embodiments, the immune inhibitory checkpoint polypeptide comprises PD-L1, PD-L2, CD80, CD86, HVEM, CD48, CD112, CD155, Ceacam1, FGL1, or Galectin-3. In some embodiments, the mammalian immune checkpoint polypeptide comprises an immune stimulatory checkpoint polypeptide. In some embodiments, the immune stimulatory checkpoint polypeptide comprises a polypeptide expressed on T cells. In some embodiments, the immune stimulatory checkpoint polypeptide comprises CD27, CD28, CD40, CD122, 4-1BB, ICOS, OX40, CD2, CD30, or GITR. In some embodiments, the immune stimulatory checkpoint polypeptide comprises CD70, CD80, CD86, CD40L, GITRL, 4-1BBL, OX40L, LIGHT, CD30L, CD48, or ICOSL. In some embodiments, the immune stimulatory checkpoint polypeptide is expressed on antigen presenting cells, tumor cells, or normal cells. In some embodiments, the immune inhibitory checkpoint polypeptide comprises an amino acid sequence of at least 90% sequence identity to an amino acid sequence according to any one of SEQ ID NOs: 1-42, or 96-101. In some embodiments, the immune stimulatory checkpoint polypeptide comprises an amino acid sequence of at least 90% sequence identity to an amino acid sequence according to any one of SEQ ID NOs: 43-62, 102-115 or 153-162. In some embodiments, the transmembrane polypeptide anchors the fusion protein to a bilayer of the multivalent particle. In some embodiments, the transmembrane polypeptide comprises a spike glycoprotein, a mammalian membrane protein, an envelope protein, a nucleocapsid protein, or a cellular transmembrane protein. In some embodiments, the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120. In some embodiments, the VSVG comprises full length VSVG or a truncated VSVG. In some embodiments, the VSVG comprises a transmembrane domain and cytoplasmic tail. In some embodiments, the fusion protein further comprises an oligomerization domain. In some embodiments, the oligomerization domain comprises a dimerization domain, a trimerization domain, or a tetramerization domain. In some embodiments, the dimerization domain comprises a leucine zipper dimerization domain. In some embodiments, the fusion protein further comprises a cytosolic domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the transmembrane polypeptide comprises an amino acid sequence at least about 90% identical to that set forth in any one of SEQ ID NOs: 63, 64, or 79-95. In some embodiments, the tetramerization domain comprises an influenza neuraminidase stem domain. In some embodiments, the oligomerization domain comprises an amino acid sequence that has at least 95% sequence identity to an amino acid sequence according to SEQ ID NOs: 65-78. In some embodiments, when the fusion protein is expressed on the surface of the multivalent particle, the oligomerization domain is outside of the multivalent particle. In some embodiments, when the fusion protein is expressed on the surface of the multivalent particle, the oligomerization domain is outside of the multivalent particle and adjacent to a signal peptide. In some embodiments, when the fusion protein is expressed on the surface of the multivalent particle, the oligomerization domain is inside of the multivalent particle. In some embodiments, when the fusion protein is expressed on the surface of the multivalent particle, the oligomerization domain is inside of the multivalent particle and adjacent to the transmembrane polypeptide. In some embodiments, the fusion protein comprises a signal peptide. In some embodiments, domains of the fusion protein are arranged from the N-terminus to the C-terminus in the following orders: signal peptide, mammalian immune checkpoint polypeptide, oligomerization domain, transmembrane polypeptide, and cytosolic domain; signal peptide, mammalian immune checkpoint polypeptide, transmembrane polypeptide, oligomerization domain, and cytosolic domain; or signal peptide, oligomerization domain, mammalian immune checkpoint polypeptide, transmembrane polypeptide, and cytosolic domain. In some embodiments, the fusion protein is expressed at a valency of about 10 copies on a surface of the multivalent particle. In some embodiments, the fusion protein is expressed at a valency of about 10 to about 15 copies on a surface of the multivalent particle. In some embodiments, the fusion protein is expressed at a valency of at least about 25 copies on a surface of the multivalent particle. In some embodiments, the fusion protein is expressed at a valency of at least about 50 copies on a surface of the multivalent particle. In some embodiments, the fusion protein is expressed at a valency of at least about 75 copies on a surface of the multivalent particle. In some embodiments, the fusion protein is expressed at a valency of at least about 100 copies on a surface of the multivalent particle. In some embodiments, the fusion protein is expressed at a valency of at least about 150 copies on a surface of the multivalent particle. In some embodiments, the fusion protein is expressed at a valency of at least about 200 copies on a surface of the multivalent particle. In some embodiments, the multivalent particle does not comprise viral genetic material. In some embodiments, the multivalent particle is a viral-like a particle. In some embodiments, the multivalent particle is an extracellular vesicle (EV). In some embodiments, the multivalent particle is an exosome. In some embodiments, the multivalent particle is an ectosome. In some embodiments, (a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; and (b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120. In some embodiments, (a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; and (b) the transmembrane polypeptide comprises an amino acid sequence at least about 75%, 80%, 85%, 90%, 95%, or 99% identical to that set forth in any one of SEQ ID NOs: 63, 64, or 79-95. In some embodiments, (a) the immune checkpoint polypeptide comprises an amino acid sequence of at least 75%, 80%, 85%, 90%, 95%, or 99% sequence identity to an amino acid sequence according to any one of SEQ ID NOs: 1-62, 96-115, or 153-162; and (b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120. In some embodiments, (a) the immune checkpoint polypeptide comprises an amino acid sequence of at least 75%, 80%, 85%, 90%, 95%, or 99% sequence identity to an amino acid sequence according to any one of SEQ ID NOs: 1-62, 96-115, or 153-162; and (b) the transmembrane polypeptide comprises an amino acid sequence at least about 75%, 80%, 85%, 90%, 95%, or 99% identical to that set forth in any one of SEQ ID NOs: 63, 64, or 79-95. In some embodiments, (a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; (b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120; and (c) the oligomerization domain comprises a leucine zipper dimerization domain, a post-fusion oligomerization domain of viral surface protein, a D4 post-fusion trimerization domain of VSV-G protein, a Dengue E protein post-fusion trimerization domain, a foldon trimerization domain, or an influenza neuraminidase stem domain. In some embodiments, (a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; (b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120; and (c) the oligomerization domain comprises an amino acid sequence that has at least 75%, 80%, 85%, 90%, 95%, or 99% sequence identity to an amino acid sequence according to SEQ ID NOs: 65-78. In some embodiments, (a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; (b) the transmembrane polypeptide comprises an amino acid sequence at least about 75%, 80%, 85%, 90%, 95%, or 99% identical to that set forth in any one of SEQ ID NOs: 63, 64, or 79-95; and (c) the oligomerization domain comprises a leucine zipper dimerization domain, a post-fusion oligomerization domain of viral surface protein, a D4 post-fusion trimerization domain of VSV-G protein, a Dengue E protein post-fusion trimerization domain, a foldon trimerization domain, or an influenza neuraminidase stem domain. In some embodiments, (a) the immune checkpoint polypeptide comprises an amino acid sequence of at least 75%, 80%, 85%, 90%, 95%, or 99% sequence identity to an amino acid sequence according to any one of SEQ ID NOs: 1-62, 96-115, or 153-162; (b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120; and (c) the oligomerization domain comprises a leucine zipper dimerization domain, a post-fusion oligomerization domain of viral surface protein, a D4 post-fusion trimerization domain of VSV-G protein, a Dengue E protein post-fusion trimerization domain, a foldon trimerization domain, or an influenza neuraminidase stem domain. In some embodiments, (a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; (b) the transmembrane polypeptide comprises an amino acid sequence at least about 75%, 80%, 85%, 90%, 95%, or 99% identical to that set forth in any one of SEQ ID NOs: 63, 64, or 79-95; and (c) the oligomerization domain comprises an amino acid sequence that has at least 75%, 80%, 85%, 90%, 95%, or 99% sequence identity to an amino acid sequence according to SEQ ID NOs: 65-78.

Disclosed herein, in some embodiments, is a composition comprising a first nucleic acid sequence encoding a multivalent particle comprising a fusion protein that comprises a mammalian immune checkpoint polypeptide and a transmembrane polypeptide wherein the fusion protein is expressed at a valency of at least about 10 copies on a surface of the multivalent particle when the multivalent particle is expressed; and an excipient. In some embodiments, the composition further comprises a second nucleic acid sequence that encodes one or more viral proteins. In some embodiments, the one or more viral proteins is a lentiviral protein, a retroviral protein, an adenoviral protein, or combinations thereof. In some embodiments, the one or more viral proteins comprises gag, pol, pre, tat, rev, or combinations thereof. In some embodiments, the composition further comprises a third nucleic acid sequence that encodes a replication incompetent viral genome, a reporter, a therapeutic molecule, or combinations thereof. In some embodiments, the viral genome is derived from vesicular stomatitis virus, measles virus, Hepatitis virus, influenza virus, or combinations thereof. In some embodiments, the reporter is a fluorescent protein or luciferase. In some embodiments, the fluorescent protein is green fluorescent protein. In some embodiments, the therapeutic molecule is a cellular signal modulating molecule, a proliferation modulating molecule, a cell death modulating molecule, or combinations thereof. In some embodiments, the mammalian immune checkpoint polypeptide comprises a polypeptide expressed on T cells. In some embodiments, the mammalian immune checkpoint polypeptide comprises an immune inhibitory checkpoint polypeptide. In some embodiments, the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9. In some embodiments, the immune inhibitory checkpoint polypeptide is expressed on antigen presenting cells, tumor cells, or normal cells. In some embodiments, the immune inhibitory checkpoint polypeptide comprises PD-L1, PD-L2, CD80, CD86, HVEM, CD48, CD112, CD155, Ceacam1, FGL1, or Galectin-3. In some embodiments, the mammalian immune checkpoint polypeptide comprises an immune stimulatory checkpoint polypeptide. In some embodiments, the immune stimulatory checkpoint polypeptide comprises a polypeptide expressed on T cells. In some embodiments, the immune stimulatory checkpoint polypeptide comprises CD27, CD28, CD40, CD122, 4-1BB, ICOS, OX40, CD2, CD30, or GITR. In some embodiments, the immune stimulatory checkpoint polypeptide comprises CD70, CD80, CD86, CD40L, GITRL, 4-1BBL, OX40L, LIGHT, CD30L, CD48, or ICOSL. In some embodiments, the immune stimulatory checkpoint polypeptide is expressed on antigen presenting cells, tumor cells, or normal cells. In some embodiments, the immune inhibitory checkpoint polypeptide comprises an amino acid sequence of at least 90% sequence identity to an amino acid sequence according to any one of SEQ ID NOs: 1-42, or 96-101. In some embodiments, the immune stimulatory checkpoint polypeptide comprises an amino acid sequence of at least 90% sequence identity to an amino acid sequence according to any one of SEQ ID NOs: 43-62, 102-115, or 153-162. In some embodiments, the transmembrane polypeptide anchors the fusion protein to a bilayer of the multivalent particle. In some embodiments, the transmembrane polypeptide comprises a spike glycoprotein, a mammalian membrane protein, an envelope protein, a nucleocapsid protein, or a cellular transmembrane protein. In some embodiments, the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120. In some embodiments, the VSVG comprises full length VSVG or a truncated VSVG. In some embodiments, the VSVG comprises a transmembrane domain and cytoplasmic tail. In some embodiments, the transmembrane polypeptide comprises an amino acid sequence at least about 90% identical to that set forth in any one of SEQ ID NOs: 63, 64, or 79-95. In some embodiments, the fusion protein further comprises an oligomerization domain. In some embodiments, the oligomerization domain comprises a dimerization domain, a trimerization domain, or a tetramerization domain. In some embodiments, the dimerization domain comprises a leucine zipper dimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the fusion protein further comprises a cytosolic domain. In some embodiments, the tetramerization domain comprises an influenza neuraminidase stem domain. In some embodiments, the oligomerization domain comprises an amino acid sequence that has at least 95% sequence identity to an amino acid sequence according to SEQ ID NOs: 65-78. In some embodiments, when the fusion protein is expressed on the surface of the multivalent particle, the oligomerization domain is outside of the multivalent particle. In some embodiments, when the fusion protein is expressed on the surface of the multivalent particle, the oligomerization domain is outside of the multivalent particle and adjacent to a signal peptide. In some embodiments, when the fusion protein is expressed on the surface of the multivalent particle, the oligomerization domain is inside of the multivalent particle. In some embodiments, when the fusion protein is expressed on the surface of the multivalent particle, the oligomerization domain is inside of the multivalent particle and adjacent to the transmembrane polypeptide. In some embodiments, the fusion protein comprises a signal peptide. In some embodiments, domains of the fusion protein are arranged from the N-terminus to the C-terminus in the following orders: signal peptide, mammalian immune checkpoint polypeptide, oligomerization domain, transmembrane polypeptide, and cytosolic domain; signal peptide, mammalian immune checkpoint polypeptide, transmembrane polypeptide, oligomerization domain, and cytosolic domain; or signal peptide, oligomerization domain, mammalian immune checkpoint polypeptide, transmembrane polypeptide, and cytosolic domain. In some embodiments, the fusion protein is expressed at a valency of at about 10 copies on a surface of the multivalent particle when the multivalent particle is expressed. In some embodiments, the fusion protein is expressed at a valency of at about 10 copies to about 15 copies on a surface of the multivalent particle when the multivalent particle is expressed. In some embodiments, the fusion protein is expressed at a valency of at least about 25 copies on a surface of the multivalent particle when the multivalent particle is expressed. In some embodiments, the fusion protein is expressed at a valency of at least about 50 copies on a surface of the multivalent particle when the multivalent particle is expressed. In some embodiments, the fusion protein is expressed at a valency of at least about 75 copies on a surface of the multivalent particle when the multivalent particle is expressed. In some embodiments, the fusion protein is expressed at a valency of at least about 100 copies on a surface of the multivalent particle when the multivalent particle is expressed. In some embodiments, the fusion protein is expressed at a valency of at least about 150 copies on a surface of the multivalent particle when the multivalent particle is expressed. In some embodiments, the fusion protein is expressed at a valency of at least about 200 copies on a surface of the multivalent particle when the multivalent particle is expressed. In some embodiments, the fusion protein is expressed at a valency of at least about 500 copies on a surface of the multivalent particle when the multivalent particle is expressed. In some embodiments, the fusion protein is expressed at a valency of at least about 1000 copies on a surface of the multivalent particle when the multivalent particle is expressed. In some embodiments, the fusion protein is expressed at a valency of at least about 2000 copies on a surface of the multivalent particle when the multivalent particle is expressed. In some embodiments, the multivalent particle does not comprise viral genetic material. In some embodiments, the multivalent particle is a viral-like a particle. In some embodiments, the multivalent particle is an extracellular vesicle (EV). In some embodiments, the multivalent particle is an exosome. In some embodiments, the multivalent particle is an ectosome. In some embodiments, the first nucleic acid sequence, the second nucleic acid sequence, and the third nucleic acid sequence are within a same vector. In some embodiments, the first nucleic acid sequence, the second nucleic acid sequence, and the third nucleic acid sequence are within different vectors. In some embodiments, the vector is a lentivirus vector, an adenovirus vector, or an adeno-associated virus vector. In some embodiments, the vectors comprise a lentivirus vector, an adenovirus vector, or an adeno-associated virus vector. In some embodiments, (a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; and (b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120. In some embodiments, (a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; and (b) the transmembrane polypeptide comprises an amino acid sequence at least about 75%, 80%, 85%, 90%, 95%, or 99% identical to that set forth in any one of SEQ ID NOs: 63, 64, or 79-95. In some embodiments, (a) the immune checkpoint polypeptide comprises an amino acid sequence of at least 75%, 80%, 85%, 90%, 95%, or 99% sequence identity to an amino acid sequence according to any one of SEQ ID NOs: 1-62, 96-115, or 153-162; and (b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120. In some embodiments, (a) the immune checkpoint polypeptide comprises an amino acid sequence of at least 75%, 80%, 85%, 90%, 95%, or 99% sequence identity to an amino acid sequence according to any one of SEQ ID NOs: 1-62, 96-115, or 153-162; and (b) the transmembrane polypeptide comprises an amino acid sequence at least about 75%, 80%, 85%, 90%, 95%, or 99% identical to that set forth in any one of SEQ ID NOs: 63, 64, or 79-95. In some embodiments, (a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; (b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120; and (c) the oligomerization domain comprises a leucine zipper dimerization domain, a post-fusion oligomerization domain of viral surface protein, a D4 post-fusion trimerization domain of VSV-G protein, a Dengue E protein post-fusion trimerization domain, a foldon trimerization domain, or an influenza neuraminidase stem domain. In some embodiments, (a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; (b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120; and (c) the oligomerization domain comprises an amino acid sequence that has at least 75%, 80%, 85%, 90%, 95%, or 99% sequence identity to an amino acid sequence according to SEQ ID NOs: 65-78. In some embodiments, (a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; (b) the transmembrane polypeptide comprises an amino acid sequence at least about 75%, 80%, 85%, 90%, 95%, or 99% identical to that set forth in any one of SEQ ID NOs: 63, 64, or 79-95; and (c) the oligomerization domain comprises a leucine zipper dimerization domain, a post-fusion oligomerization domain of viral surface protein, a D4 post-fusion trimerization domain of VSV-G protein, a Dengue E protein post-fusion trimerization domain, a foldon trimerization domain, or an influenza neuraminidase stem domain. In some embodiments, (a) the immune checkpoint polypeptide comprises an amino acid sequence of at least 75%, 80%, 85%, 90%, 95%, or 99% sequence identity to an amino acid sequence according to any one of SEQ ID NOs: 1-62, 96-115, or 153-162; (b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120; and (c) the oligomerization domain comprises a leucine zipper dimerization domain, a post-fusion oligomerization domain of viral surface protein, a D4 post-fusion trimerization domain of VSV-G protein, a Dengue E protein post-fusion trimerization domain, a foldon trimerization domain, or an influenza neuraminidase stem domain. In some embodiments, (a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; (b) the transmembrane polypeptide comprises an amino acid sequence at least about 75%, 80%, 85%, 90%, 95%, or 99% identical to that set forth in any one of SEQ ID NOs: 63, 64, or 79-95; and (c) the oligomerization domain comprises an amino acid sequence that has at least 75%, 80%, 85%, 90%, 95%, or 99% sequence identity to an amino acid sequence according to SEQ ID NOs: 65-78.

Disclosed herein, in some embodiments, is a pharmaceutical composition comprising a multivalent particle disclosed herein and a pharmaceutically acceptable excipient.

Disclosed herein, in some embodiments, is a method of treating a cancer, an autoimmune disease, an infection, or an inflammatory disease, comprising administering a multivalent particle disclosed herein. In some embodiments, the multivalent particle is administered intravenously. In some embodiments, the multivalent particle is administered through inhalation. In some embodiments, the multivalent particle is administered by intraperitoneal injection. In some embodiments, the multivalent particle is administered by subcutaneous injection.

Disclosed herein, in some embodiments, is a composition comprising a multivalent particle (MVP) wherein the MVP comprises an enveloped particle that displays at least about 10 copies of an immune checkpoint polypeptide on a surface of the MVP, wherein the immune checkpoint polypeptide forms multivalent interactions with a ligand on a target immune cell when displayed on the surface of the enveloped particle.

Disclosed herein, in some embodiments, is a method of using a multivalent particle (MVP) displaying an immune checkpoint polypeptide to mimic multivalent interactions between a first immune cell expressing the immune checkpoint polypeptide and a second immune cell expressing a target of the immune checkpoint polypeptide, wherein the immune checkpoint polypeptide is displayed at least about 10 copies on a surface of the MVP.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A illustrates vector design for monomeric display of immune checkpoints on MVPs.

FIG. 1B illustrates vector design for trimeric display of immune checkpoints on MVPs.

FIG. 1C illustrates vector design for displaying type II immune checkpoints on MVPs.

FIG. 2A illustrates production of monomeric IC-MVPs as VLPs containing RNA genomes.

FIG. 2B illustrates production of monomeric IC-MVPs as VLPs without RNA genomes.

FIG. 2C illustrates production of monomeric IC-MVPs as EVs.

FIG. 3A illustrates production of trimeric IC-MVPs as VLPs containing RNA genomes.

FIG. 3B illustrates production of trimeric IC-MVPs as VLPs without RNA genomes.

FIG. 3C illustrates production of trimeric IC-MVPs as EVs.

FIG. 4A illustrates production of mixed monomeric and trimeric IC-MVPs as VLPs containing RNA genomes.

FIG. 4B illustrates production of mixed monomeric and trimeric IC-MVPs as VLPs without RNA genomes.

FIG. 4C illustrates production of mixed monomeric and trimeric IC-MVPs as EVs.

FIGS. 5A-5C illustrate various D4 configurations.

FIGS. 6A-6C illustrate various oligomerization domain configurations.

FIG. 7A illustrates FACS-based assay to measure specific binding between dye-labelled IC-MVPs and target cells expressing the cognate receptor or ligand.

FIG. 7B illustrates FACS-based assay to measure specific binding between unlabeled IC-MVPs and target cells expressing the cognate receptor or ligand.

FIG. 8A illustrates quantitative western blot analysis of PD-1-MVPs.

FIG. 8B illustrates FACS analysis of specific binding of dye-labeled PD-1-MVPs to target cells expressing cognate receptor PD-L1.

FIG. 8C illustrates FACS analysis of specific binding of unlabeled PD-1-MVPs to target cells expressing cognate receptor PD-L1.

FIG. 8D illustrates FACS analysis of specific binding of dye-labeled PD-1-MVPs to target cells expressing cognate receptor PD-L2.

FIG. 8E illustrates FACS analysis of specific binding of unlabeled PD-1-MVPs to target cells expressing cognate receptor PD-L2.

FIG. 9A illustrates the inhibitory immune checkpoints on T cells and their ligands on antigen presenting cells including tumor cells.

FIG. 9B illustrates the PD-L1 and PD-1 mediated inhibitory checkpoint signaling for antigen-specific T cells.

FIG. 9C illustrates blockade of PD-L1 and PD-1 mediated inhibitory checkpoint signaling by anti-PD-1 antibodies

FIG. 9D illustrates blockade of PD-L1 and PD-1 mediated inhibitory checkpoint signaling by PD-1-MVPs.

FIG. 10A illustrates FACS analysis of PD-L1 expression on B16F0 melanoma cells.

FIG. 10B illustrates FACS analysis of PD-L1 expression on B16F10 melanoma cells.

FIG. 10C illustrates FACS analysis of specific binding of dye-labeled PD-1-MVPs to B16F0 melanoma cells expressing cognate receptor PD-L1.

FIG. 10D illustrates FACS analysis of specific binding of dye-labeled PD-1-MVPs to B16F10 melanoma cells expressing cognate receptor PD-L1.

FIG. 11A illustrates study design to determine the effects of PD-1-MVPs on mouse B16F0 melanoma.

FIG. 11B illustrates the effects of PD-1-MVPs on mice B16F0 melanoma tumor growth.

FIG. 11C illustrates the effects of PD-1-MVPs on survival of mice bearing B16F0 melanoma tumors.

FIG. 12A illustrates study design to determine the effects of PD-1-MVPs on mouse B16F10 melanoma.

FIG. 12B illustrates the effects of PD-1-MVPs on mice B16F10 melanoma tumor growth.

FIG. 13A illustrates FACS analysis PD-L1 expression on MC38 colon adenocarcinoma cells.

FIG. 13B illustrates FACS analysis of specific binding of dye-labeled PD-1-MVPs to MC38 colon adenocarcinoma cells expressing cognate receptor PD-L1.

FIG. 13C illustrates study design to determine the effects of PD-1-MVPs on mouse MC38 colon adenocarcinoma.

FIG. 13D illustrates the effects of PD-1-MVPs on mouse MC38 colon adenocarcinoma tumor growth.

FIG. 14A illustrates the lack of engagement between PD-L1 on antigen presenting cells and PD-1 on antigen-specific T cells.

FIG. 14B illustrates the use of PD-L1-MVPs or PD-L2-MVPs as an agonist to turn on PD-1 mediated inhibitory checkpoint signaling in antigen-specific T cells.

FIG. 15A illustrates quantitative western blot analysis of PDL-1-MVPs.

FIG. 15B illustrates FACS analysis of specific binding of dye-labeled PDL-1-MVPs to target cells expressing cognate receptor PD-1.

FIG. 15C illustrates FACS analysis of specific binding of unlabeled PDL-1-MVPs to target cells expressing cognate receptor PD-1.

FIG. 16A illustrates study design to determine the effects of PDL-1-MVPs on ARDS in mice.

FIG. 16B illustrates the effects of PDL-1-MVPs on the survival of mice with ARDS.

FIG. 17A illustrates quantitative western blot analysis of 2B4-MVPs.

FIG. 17B illustrates FACS analysis of specific binding of unlabeled 2B4-MVPs to target cells expressing cognate receptor CD48.

FIG. 18A illustrates study design to determine the effects of 2B4-MVPs on ARDS in mice.

FIG. 18B illustrates the effects of 2B4-MVPs on the survival of mice with ARDS.

FIG. 19A illustrates quantitative western blot analysis of PDL-2-MVPs.

FIG. 19B illustrates FACS analysis of specific binding of dye-labeled PDL-2-MVPs to target cells expressing cognate receptor PD-1.

FIG. 19C illustrates FACS analysis of specific binding of unlabeled PDL-2-MVPs to target cells expressing cognate receptor PD-1.

FIG. 20A illustrates quantitative western blot analysis of CTLA4-MVPs.

FIG. 20B illustrates FACS analysis of specific binding of dye-labeled CTLA4-MVPs to target cells expressing cognate receptor CD80.

FIG. 20C illustrates FACS analysis of specific binding of unlabeled CTLA4-MVPs to target cells expressing cognate receptor CD80.

FIG. 20D illustrates FACS analysis of specific binding of dye-labeled CTLA4-MVPs to target cells expressing cognate receptor CD86.

FIG. 20E illustrates FACS analysis of specific binding of unlabeled CTLA4-MVPs to target cells expressing cognate receptor CD86.

FIG. 21A illustrates quantitative western blot analysis of CD80-MVPs.

FIG. 21B illustrates FACS analysis of specific binding of dye-labeled CD80-MVPs to target cells expressing cognate receptor CTLA-4.

FIG. 21C illustrates FACS analysis of specific binding of unlabeled CD80-MVPs to target cells expressing cognate receptor CTLA-4.

FIG. 22A illustrates quantitative western blot analysis of CD86-MVPs.

FIG. 22B illustrates FACS analysis of specific binding of dye-labeled CD86-MVPs to target cells expressing cognate receptor CTLA-4.

FIG. 22C illustrates FACS analysis of specific binding of unlabeled CD86-MVPs to target cells expressing cognate receptor CTLA-4.

FIG. 23A illustrates quantitative western blot analysis of Galectin3-MVPs.

FIG. 23B illustrates FACS analysis of specific binding of dye-labeled Galectin3-MVPs to target cells expressing cognate receptor LAG-3.

FIG. 23C illustrates FACS analysis of specific binding of unlabeled Galectin3-MVPs to target cells expressing cognate receptor LAG-3.

FIG. 24A illustrates quantitative western blot analysis of LAG3-MVPs.

FIG. 24B illustrates FACS analysis of specific binding of dye-labeled LAG3-MVPs to target cells expressing cognate receptor Galectin-3.

FIG. 24C illustrates FACS analysis of specific binding of unlabeled LAG3-MVPs to target cells expressing cognate receptor Galectin-3.

FIG. 25A illustrates quantitative western blot analysis of FGL1-MVPs.

FIG. 25B illustrates FACS analysis of specific binding of dye-labeled FGL1-MVPs to target cells expressing cognate receptor LAG-3.

FIG. 25C illustrates FACS analysis of specific binding of unlabeled FGL1-MVPs to target cells expressing cognate receptor LAG-3.

FIG. 26A illustrates quantitative western blot analysis of LAG3-MVPs.

FIG. 26B illustrates FACS analysis of specific binding of dye-labeled LAG3-MVPs to target cells expressing cognate receptor FGL1.

FIG. 26C illustrates FACS analysis of specific binding of unlabeled LAG3-MVPs to target cells expressing cognate receptor FGL1.

FIG. 27A illustrates quantitative western blot analysis of HVEM-MVPs.

FIG. 27B illustrates FACS analysis of specific binding of unlabeled HVEM-MVPs to target cells expressing cognate receptor BTLA.

FIG. 28A illustrates quantitative western blot analysis of BTLA-MVPs.

FIG. 28B illustrates FACS analysis of specific binding of dye-labeled BTLA-MVPs to target cells expressing cognate receptor HVEM.

FIG. 28C illustrates FACS analysis of specific binding of unlabeled BTLA-MVPs to target cells expressing cognate receptor HVEM.

FIG. 29A illustrates quantitative western blot analysis of CD160-MVPs.

FIG. 29B illustrates FACS analysis of specific binding of dye-labeled CD160-MVPs to target cells expressing cognate receptor HVEM.

FIG. 29C illustrates FACS analysis of specific binding of unlabeled CD160-MVPs to target cells expressing cognate receptor HVEM.

FIG. 30A illustrates quantitative western blot analysis of CD48-MVPs.

FIG. 30B illustrates FACS analysis of specific binding of dye-labeled CD48-MVPs to target cells expressing cognate receptor 2B4.

FIG. 30C illustrates FACS analysis of specific binding of unlabeled CD48-MVPs to target cells expressing cognate receptor 2B4.

FIG. 31A illustrates quantitative western blot analysis of CD112-MVPs.

FIG. 31B illustrates FACS analysis of specific binding of dye-labeled CD112-MVPs to target cells expressing cognate receptor TIGIT.

FIG. 32A illustrates quantitative western blot analysis of TIGIT-MVPs.

FIG. 32B illustrates FACS analysis of specific binding of dye-labeled TIGIT-MVPs to target cells expressing cognate receptor CD112.

FIG. 32C illustrates FACS analysis of specific binding of unlabeled TIGIT-MVPs to target cells expressing cognate receptor CD112.

FIG. 33A illustrates quantitative western blot analysis of CD155-MVPs.

FIG. 33B illustrates FACS analysis of specific binding of dye-labeled CD155-MVPs to target cells expressing cognate receptor TIGIT.

FIG. 33C illustrates FACS analysis of specific binding of unlabeled CD155-MVPs to target cells expressing cognate receptor TIGIT.

FIG. 34A illustrates quantitative western blot analysis of TIGIT-MVPs.

FIG. 34B illustrates FACS analysis of specific binding of dye-labeled TIGIT-MVPs to target cells expressing cognate receptor CD155.

FIG. 34C illustrates FACS analysis of specific binding of unlabeled TIGIT-MVPs to target cells expressing cognate receptor CD155.

FIG. 35A illustrates quantitative western blot analysis of human TIM3-MVPs.

FIG. 35B illustrates FACS analysis of specific binding of dye-labeled human TIM3-MVPs to target cells expressing cognate receptor human Ceacam-1.

FIG. 35C illustrates FACS analysis of specific binding of unlabeled human TIM3-MVPs to target cells expressing cognate receptor human Ceacam-1.

FIG. 36A illustrates quantitative western blot analysis of human Ceacam1-MVPs.

FIG. 36B illustrates FACS analysis of specific binding of dye-labeled human Ceacam1-MVPs to target cells expressing cognate receptor human TIM-3.

FIG. 36C illustrates FACS analysis of specific binding of unlabeled human Ceacam1-MVPs to target cells expressing cognate receptor human TIM-3.

FIG. 37A illustrates activating immune checkpoints, including co-stimulatory signals, on T cells and their ligands on antigen presenting cells.

FIG. 37B illustrates the use of co-stimulatory MVPs to supplement T cell receptor (TCR) activation signaling.

FIG. 37C illustrates the use of anti-CD3 antibodies together with co-stimulatory MVPs for T cell activation.

FIG. 38A illustrates the effects of co-stimulatory murine CD86-MVPs on mouse spleen T cell activation based on the expression of CD69 and CD25 at day-2 post activation.

FIG. 38B illustrates the effects of co-stimulatory murine CD86-MVPs on T cell proliferation.

FIG. 39A illustrates western-blot analysis of human CD86-MVPs under non-reducing or reducing conditions.

FIG. 39B illustrates FACS analysis of the effects of human CD86-MVPs on human peripheral blood T cell activation based on the expression of CD69 and CD25 at day-2 post activation.

FIG. 39C illustrates FACS analysis of the effects of human CD86-MVPs on human peripheral blood T cell differentiation status based on the expression of CD45RO and CD62L at day-8 post activation.

FIG. 40A illustrates FACS analysis of the effects of murine CD80-MVPs on mouse spleen T cell activation based on the expression of CD69 and CD25 at day-2 post activation.

FIG. 40B illustrates the effects of murine CD80-MVPs on T cell proliferation.

FIG. 41A illustrates western-blot analysis of human CD80-MVPs under non-reducing or reducing conditions.

FIG. 41B illustrates FACS analysis of the effects of human CD80-MVPs on human peripheral blood T cell activation based on the expression of CD69 and CD25 at day-2 post activation.

FIG. 41C illustrates the effects of human CD80-MVPs on human peripheral blood T cell differentiation status based on the expression of CD45RO and CD62L at day-8 post activation.

FIG. 42A illustrates FACS analysis of the effects of co-stimulatory murine 4-1BBL-MVPs on mouse spleen T cells activation based on the expression of CD69 and CD25 at day-2 post activation.

FIG. 42B illustrates the effects of co-stimulatory murine 4-1BBL-MVPs on mouse spleen T cell proliferation.

FIG. 43A illustrates quantitative western-blot analysis of human 4-1BBL-MVPs.

FIG. 43B illustrates FACS analysis of binding of unlabeled human 4-1BBL-MVPs to target cells expressing cognate receptor 4-1BB.

FIG. 43C illustrates FACS analysis of the effects of human 4-1BBL-MVPs on human peripheral blood T cells activation based on the expression of CD69 and CD25 at day-2 post activation.

FIG. 43D illustrates FACS analysis of the effects of human 4-1BBL-MVPs on human peripheral blood T cell differentiation status based on the expression of CD45RO and CD62L at day-8 post activation.

FIG. 44A illustrates FACS analysis of the effects of co-stimulatory murine OX40L-MVPs on mouse spleen T cell activation based on the expression of CD69 and CD25 at day-2 post activation.

FIG. 44B illustrates the effects of co-stimulatory murine OX40L-MVPs on mouse spleen T cell proliferation.

FIG. 45A illustrates quantitative western-blot analysis of human OX40L-MVPs.

FIG. 45B illustrates western-blot analysis of human OX40L-MVPs under a non-reducing condition.

FIG. 45C illustrates FACS analysis of specific binding of dye-labeled human OX40L-MVPs to target cells expressing cognate receptor OX40.

FIG. 45D illustrates FACS analysis of the effects of human OX40L-MVPs on human peripheral blood T cell activation based on the expression of CD69 and CD25 at day-2 post activation.

FIG. 45E illustrates FACS analysis of the effects of human OX40L-MVPs on human peripheral blood T cell differentiation status based on the expression of CD45RO and CD62L at day-8 post activation.

FIG. 46A illustrates quantitative western-blot analysis of murine LIGHT-MVPs.

FIG. 46B illustrates FACS analysis of specific binding of dye-labeled murine LIGHT-MVPs to target cells expressing cognate receptor HVEM.

FIG. 47A illustrates quantitative western-blot analysis of CD30-MVPs.

FIG. 47B illustrates FACS analysis of specific binding of dye-labeled CD30-MVPs to target cells expressing cognate receptor CD30 ligand.

FIG. 47C illustrates FACS analysis of specific binding of unlabeled CD30-MVPs to target cells expressing cognate receptor CD30 ligand.

FIG. 48A illustrates quantitative western-blot analysis of CD30L-MVPs.

FIG. 48B illustrates FACS analysis of specific binding of dye-labeled CD30L-MVPs to target cells expressing cognate receptor CD30.

FIG. 48C illustrates FACS analysis of specific binding of unlabeled CD30L-MVPs to target cells expressing cognate receptor CD30.

FIG. 49A illustrates quantitative western-blot analysis of CD48-MVPs.

FIG. 49B illustrates FACS analysis of specific binding of dye-labeled CD48-MVPs to target cells expressing cognate receptor CD2.

FIG. 49C illustrates FACS analysis of specific binding of unlabeled CD48-MVPs to target cells expressing cognate receptor CD2.

FIG. 50A illustrates quantitative western-blot analysis of CD2-MVPs.

FIG. 50B illustrates FACS analysis of specific binding of dye-labeled CD2-MVPs to target cells expressing cognate receptor CD48.

FIG. 50C illustrates FACS analysis of specific binding of unlabeled CD2-MVPs to target cells expressing cognate receptor CD48.

FIG. 51A illustrates quantitative western-blot analysis of CD27-MVPs.

FIG. 51B illustrates FACS analysis of specific binding of dye-labeled CD27-MVPs to target cells expressing cognate receptor CD70.

FIG. 51C illustrates FACS analysis of specific binding of unlabeled CD27-MVPs to target cells expressing cognate receptor CD70.

FIG. 52A illustrates quantitative western-blot analysis of CD70-MVPs.

FIG. 52B illustrates FACS analysis of specific binding of dye-labeled CD70-MVPs to target cells expressing cognate receptor CD27.

FIG. 52C illustrates FACS analysis of specific binding of unlabeled CD70-MVPs to target cells expressing cognate receptor CD27.

FIG. 53A illustrates quantitative western-blot analysis of ICOSL-MVPs.

FIG. 53B illustrates FACS analysis of specific binding of dye-labeled ICOSL-MVPs to target cells expressing cognate receptor ICOS.

FIG. 53C illustrates FACS analysis of specific binding of unlabeled ICOSL-MVPs to target cells expressing cognate receptor ICOS.

FIG. 54A illustrates quantitative western-blot analysis of ICOS-MVPs.

FIG. 54B illustrates FACS analysis of specific binding of dye-labeled ICOS-MVPs to target cells expressing cognate receptor ICOS ligand.

FIG. 55A illustrates quantitative western-blot analysis of GITRL-MVPs.

FIG. 55B illustrates FACS analysis of specific binding of dye-labeled GITRL-MVPs to target cells expressing cognate receptor GITR.

FIG. 55C illustrates FACS analysis of specific binding of unlabeled GITRL-MVPs to target cells expressing cognate receptor GITR.

FIG. 56A illustrates quantitative western-blot analysis of GITR-MVPs.

FIG. 56B illustrates FACS analysis of specific binding of dye-labeled GITR-MVPs to target cells expressing cognate receptor GITR ligand.

FIG. 56C illustrates FACS analysis of specific binding of unlabeled GITR-MVPs to target cells expressing cognate receptor GITR ligand.

FIG. 57A illustrates quantitative western-blot analysis of 4-1BB-MVPs.

FIG. 57B illustrates FACS analysis of specific binding of dye-labeled 4-1BB-MVPs to target cells expressing cognate receptor 4-1BB ligand.

FIG. 58A illustrates quantitative western-blot analysis of OX40-MVPs.

FIG. 58B illustrates FACS analysis of specific binding of dye-labeled OX40-MVPs to target cells expressing cognate receptor OX40 ligand.

FIG. 58C illustrates FACS analysis of specific binding of unlabeled OX40-MVPs to target cells expressing cognate receptor OX40 ligand.

### DETAILED DESCRIPTION

The present disclosure employs, unless otherwise indicated, conventional molecular biology techniques, which are within the skill of the art. Unless defined otherwise, all technical and scientific terms used herein have the same meaning as is commonly understood by one of ordinary skill in the art.

### Definitions

Throughout this disclosure, various embodiments are presented in a range format. It should be understood that the description in range format is merely for convenience and brevity and should not be construed as an inflexible limitation on the scope of any embodiments. Accordingly, the description of a range should be considered to have specifically disclosed all the possible subranges as well as individual numerical values within that range to the tenth of the unit of the lower limit unless the context clearly dictates otherwise. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual values within that range, for example, 1.1, 2, 2.3, 5, and 5.9. This applies regardless of the breadth of the range. The upper and lower limits of these intervening ranges may independently be included in the smaller ranges, and are also encompassed within the disclosure, subject to any specifically excluded limit in the stated range. Where the stated range includes one or both of the limits, ranges excluding either or both of those included limits are also included in the disclosure, unless the context clearly dictates otherwise.

The terminology used herein is for the purpose of describing particular embodiments only and is not intended to be limiting of any embodiment. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” in reference to a number or range of numbers is understood to mean the stated number and numbers +/−10% thereof, or 10% below the lower listed limit and 10% above the higher listed limit for the values listed for a range.

### Multivalent Particles

Direct cell-cell interaction plays critical roles in regulating T cell development and function. For example, antigen presenting cells, such as dendritic cells, somatic cells, or tumor cells, can control T cell activation and development through cell-cell interaction mediated by peptide:MHC complexes and T cell receptors (TCR) on their surface. Moreover, T cells express immune checkpoint molecules on their surfaces to provide additional activating or inhibitory controls. These molecules can be stimulatory immune checkpoints that promote immune cell activation, protecting the host from invading pathogens and developing malignancies, or inhibitory checkpoints that suppress immune cells to dampen inflammation, maintain immune homeostasis, and prevent tissue damage. Tumor cells frequently exploit immune checkpoint pathways by up-regulating expression of ligands that engage inhibitory checkpoints on different immune cell types, allowing them to evade destruction by the immune system. Dysregulation of checkpoint expression may also contribute to the development and persistence of autoimmune diseases and chronic infection.

Researchers have developed cancer immunotherapies targeting immune checkpoint molecules by using either antibody-based agonists of stimulatory immune checkpoints or antibody-based antagonists of inhibitory immune checkpoints. However, these checkpoint blockade therapies are only effective in 10% to 20% of cancer patients. Moreover, some patients that initially respond to checkpoint blockade therapies can develop resistance or relapse due to an up-regulation of other immune checkpoint pathways. Therefore, it is critical to develop more effective immune checkpoint therapies so that more patients with cancer, autoimmune, or chronic infections can benefit from these transformative therapies.

Described herein are novel compositions and methods for immune checkpoint modulation. Compositions and methods as described herein are multivalent particle-based immune checkpoints (IC-MVPs). In some embodiments, IC-MVPs are genetically encoded vesicles, such as viral-like particles (VLPs), exosomes, or ectosomes, displaying multiple copies of immune checkpoint molecules. IC-MVPs can mimic checkpoint-regulation through particle-cell interactions, and form high affinity multivalent interactions with immune cell targets, such as T cells and other immune cells, effectively controlling their activation, development and function. IC-MVPs can function as activating or inhibitory switches to control the activation, development and function of T cells and other target cells depending on the displayed checkpoint molecules. For example, IC-MVPs displaying activating immune checkpoints can block the activation of T cells or other target cells through the same activating immune checkpoints, whereas IC-MVPs displaying inhibitory immune checkpoints can block the inhibition of T cells or other target cells through the same inhibitory immune checkpoints. Alternatively, IC-MVPs displaying ligands for activating immune checkpoints can be used to activate T cells or other target cells, whereas IC-MVPs displaying ligands for inhibitory immune checkpoints can be used to inhibit T cells or other target cells. Finally, IC-MVPs can be genetically programmed to display combination of checkpoint molecules to enable combinatorial activation and inhibition of T cells and other target cells.

Described herein, in some embodiments, are multivalent particles comprising a fusion protein that comprises a mammalian immune checkpoint polypeptide and a transmembrane polypeptide. Described herein, in some embodiments, are multivalent particles comprising a fusion protein that comprises the extracellular domain of a mammalian immune checkpoint polypeptide linked to an oligomerization polypeptide, and a transmembrane polypeptide. In some embodiments, the mammalian immune checkpoint polypeptide is an immune inhibitory checkpoint polypeptide. In some embodiments, the mammalian immune checkpoint polypeptide is an immune stimulatory checkpoint polypeptide. In some embodiments, the mammalian immune checkpoint polypeptide comprises a polypeptide expressed on T cells. In some embodiments, the mammalian immune checkpoint polypeptide comprises a polypeptide expressed on antigen presenting cells, such as dendritic cells, somatic cells, or tumor cells.

In some embodiments, the immune inhibitory checkpoint polypeptide comprises Programmed cell death protein 1 (PD-1), cluster of differentiation 152 (also known as CTLA4), Lymphocyte Activating 3 (LAG3), B and T lymphocyte attenuator (BTLA), CD160, Natural Killer Cell Receptor 2B4 (2B4), Cluster of Differentiation 226 (CD226), T cell immunoreceptor with Ig and ITIM domains (TIGIT), cluster of differentiation 96 (CD96), B7 homolog 3 protein (B7-H3), B7 homolog 4 protein (B7-H4), V-domain Ig suppressor of T cell activation (VISTA), T-cell immunoglobulin and mucin-domain containing-3 (TIM3), Sialic Acid Binding Ig Like Lectin 7 (SIGLEC7), Killer cell lectin-like receptor subfamily G member 1 (KLRG1), or Sialic Acid Binding Ig Like Lectin 9 (SIGLEC9). In some embodiments, the immune inhibitory checkpoint polypeptide comprises Programmed death-ligand 1 (PD-L1), Programmed death-ligand 2 (PD-L2), Cluster of differentiation 80 (CD80), Cluster of Differentiation 86 (CD86), Herpesvirus entry mediator (HVEM), Cluster of Differentiation 48 (CD48), cluster of differentiation 112 (CD112), cluster of differentiation 155 (CD155), CEA Cell Adhesion Molecule 1 (Ceacam1), Fibrinogen Like 1 (FGL1), or Galectin-3.

In some embodiments, the immune stimulatory checkpoint polypeptide comprises CD27 Molecule (CD27), Cluster of Differentiation 28 (CD28), Cluster of differentiation 40 (CD40), Interleukin-2 receptor subunit beta (CD122), 4-1BB (also known as CD137), Inducible T cell costimulatory (ICOS), OX40, cluster of differentiation 2 (CD2), CD30 (also known as TNFRSF8), or Glucocorticoid-induced TNFR-related protein (GITR). In some embodiments, the immune stimulatory checkpoint polypeptide comprises Cluster of Differentiation 70 (CD70), Cluster of Differentiation 80 (CD80), Cluster of Differentiation 86 (CD86), CD40 ligand (CD40L), Interleukin-2 (IL-2), GITR ligand (GITRL), 4-1BB ligand (4-1BBL), OX40 ligand (OX40L), LIGHT (also known as TNFSF14), CD30 ligand (CD30L), Cluster of Differentiation 48 (CD48), or ICOS ligand (ICOSL).

Various immune checkpoint multivalent particles are contemplated herein. In some embodiments, the immune checkpoint multivalent particle is recombinant. In some embodiments, the immune checkpoint multivalent particle does not comprise viral genetic material. In some embodiments, the immune checkpoint multivalent particle is a viral-like particle or virus-like particle. As used herein, viral-like particle and virus-like particle interchangeably. In some embodiments, the viral-like particle does not comprise viral genetic material. In some embodiments, the immune checkpoint multivalent particle is an extracellular vesicle. In some embodiments, the immune checkpoint multivalent particle is an exosome. In some embodiments, the immune checkpoint multivalent particle is an ectosome.

The immune checkpoint multivalent particles as described herein, in some embodiments, comprise a fusion protein, wherein the fusion protein is expressed at multiple copies on a surface of the multivalent particle. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, 3000, 3025, 3050, 3075, 3100, 3125, 3150, 3175, 3200, 3225, 3250, 3275, 3300, 3325, 3350, 3375, 3400, 3425, 3450, 3475, 3500, 3525, 3550, 3575, 3600, 3625, 3650, 3675, 3700, 3725, 3750, 3775, 3800, 3825, 3850, 3875, 3900, 3925, 3950, 3975, 4000, or more than 4000 copies on a surface of the multivalent particle. In some embodiments, the fusion protein is expressed at a valency of at least or about 5 to about 400, about 20 to about 400, about 10 to about 300, about 20 to about 300, about 20 to about 200, about 50 to about 150, about 20 to about 100, about 50 to about 100, or about 10 to about 15 copies on a surface of the multivalent particle. In some embodiments, the fusion protein is expressed at a valency of at least or about 10 copies on a surface of the multivalent particle. In some embodiments, the fusion protein is expressed at a valency of at least or about 15 copies on a surface of the multivalent particle. In some embodiments, the fusion protein is expressed at a valency of at least or about 25 copies on a surface of the multivalent particle. In some embodiments, the fusion protein is expressed at a valency of at least or about 50 copies on a surface of the multivalent particle. In some embodiments, the fusion protein is expressed at a valency of at least or about 75 copies on a surface of the multivalent particle. In some embodiments, the fusion protein is expressed at a valency of at least or about 100 copies on a surface of the multivalent particle. In some embodiments, the fusion protein is expressed at a valency of at least or about 125 copies on a surface of the multivalent particle. In some embodiments, the fusion protein is expressed at a valency of at least or about 150 copies on a surface of the multivalent particle. In some embodiments, the fusion protein is expressed at a valency of at least or about 175 copies on a surface of the multivalent particle. In some embodiments, the fusion protein is expressed at a valency of at least or about 200 copies on a surface of the multivalent particle. In some embodiments, the fusion protein is expressed at a valency of at least or about 225 copies on a surface of the multivalent particle. In some embodiments, the fusion protein is expressed at a valency of at least or about 250 copies on a surface of the multivalent particle. In some embodiments, the fusion protein is expressed at a valency of at least or about 275 copies on a surface of the multivalent particle. In some embodiments, the fusion protein is expressed at a valency of at least or about 300 copies on a surface of the multivalent particle. In some embodiments, the fusion protein is expressed at a valency of at least or about 350 copies on a surface of the multivalent particle. In some embodiments, the fusion protein is expressed at a valency of at least or about 400 copies on a surface of the multivalent particle. In some embodiments, the fusion protein is expressed at a valency of at least or about 450 copies on a surface of the multivalent particle. In some embodiments, the fusion protein is expressed at a valency of at least or about 500 copies on a surface of the multivalent particle. In some embodiments, the fusion protein is expressed at a valency of at least or about 600 copies on a surface of the multivalent particle. In some embodiments, the fusion protein is expressed at a valency of at least or about 700 copies on a surface of the multivalent particle. In some embodiments, the fusion protein is expressed at a valency of at least or about 800 copies on a surface of the multivalent particle. In some embodiments, the fusion protein is expressed at a valency of at least or about 900 copies on a surface of the multivalent particle. In some embodiments, the fusion protein is expressed at a valency of at least or about 1000 copies on a surface of the multivalent particle. In some embodiments, the fusion protein is expressed at a valency of at least or about 1100 copies on a surface of the multivalent particle. In some embodiments, the fusion protein is expressed at a valency of at least or about 1200 copies on a surface of the multivalent particle. In some embodiments, the fusion protein is expressed at a valency of at least or about 1300 copies on a surface of the multivalent particle. In some embodiments, the fusion protein is expressed at a valency of at least or about 1400 copies on a surface of the multivalent particle. In some embodiments, the fusion protein is expressed at a valency of at least or about 1500 copies on a surface of the multivalent particle. In some embodiments, the fusion protein is expressed at a valency of at least or about 1600 copies on a surface of the multivalent particle. In some embodiments, the fusion protein is expressed at a valency of at least or about 1700 copies on a surface of the multivalent particle. In some embodiments, the fusion protein is expressed at a valency of at least or about 1800 copies on a surface of the multivalent particle. In some embodiments, the fusion protein is expressed at a valency of at least or about 1900 copies on a surface of the multivalent particle. In some embodiments, the fusion protein is expressed at a valency of at least or about 2000 copies on a surface of the multivalent particle. In some embodiments, the fusion protein is expressed at a valency of at least or about 2100 copies on a surface of the multivalent particle. In some embodiments, the fusion protein is expressed at a valency of at least or about 2200 copies on a surface of the multivalent particle. In some embodiments, the fusion protein is expressed at a valency of at least or about 2300 copies on a surface of the multivalent particle. In some embodiments, the fusion protein is expressed at a valency of at least or about 2400 copies on a surface of the multivalent particle. In some embodiments, the fusion protein is expressed at a valency of at least or about 2500 copies on a surface of the multivalent particle. In some embodiments, the fusion protein is expressed at a valency of at least or about 2600 copies on a surface of the multivalent particle. In some embodiments, the fusion protein is expressed at a valency of at least or about 2700 copies on a surface of the multivalent particle. In some embodiments, the fusion protein is expressed at a valency of at least or about 2800 copies on a surface of the multivalent particle. In some embodiments, the fusion protein is expressed at a valency of at least or about 2900 copies on a surface of the multivalent particle. In some embodiments, the fusion protein is expressed at a valency of at least or about 3000 copies on a surface of the multivalent particle.

In some embodiments, the immune checkpoint multivalent particle is a viral-like particle. The viral-like particle as described herein, in some embodiments, comprise a fusion protein, wherein the fusion protein is expressed at multiple copies on a surface of the viral-like particle. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, 3000, 3025, 3050, 3075, 3100, 3125, 3150, 3175, 3200, 3225, 3250, 3275, 3300, 3325, 3350, 3375, 3400, 3425, 3450, 3475, 3500, 3525, 3550, 3575, 3600, 3625, 3650, 3675, 3700, 3725, 3750, 3775, 3800, 3825, 3850, 3875, 3900, 3925, 3950, 3975, 4000, or more than 4000 copies on a surface of the viral-like particle. In some embodiments, the fusion protein is expressed at a valency of at least or about 5 to about 400, about 20 to about 400, about 10 to about 300, about 20 to about 300, about 20 to about 200, about 50 to about 150, about 20 to about 100, about 50 to about 100, or about 10 to about 15 copies on a surface of the viral-like particle. In some embodiments, the fusion protein is expressed at a valency of at least or about 10 copies on a surface of the viral-like particle. In some embodiments, the fusion protein is expressed at a valency of at least or about 15 copies on a surface of the viral-like particle. In some embodiments, the fusion protein is expressed at a valency of at least or about 25 copies on a surface of the viral-like particle. In some embodiments, the fusion protein is expressed at a valency of at least or about 50 copies on a surface of the viral-like particle. In some embodiments, the fusion protein is expressed at a valency of at least or about 75 copies on a surface of the viral-like particle. In some embodiments, the fusion protein is expressed at a valency of at least or about 100 copies on a surface of the viral-like particle. In some embodiments, the fusion protein is expressed at a valency of at least or about 125 copies on a surface of the viral-like particle. In some embodiments, the fusion protein is expressed at a valency of at least or about 150 copies on a surface of the viral-like particle. In some embodiments, the fusion protein is expressed at a valency of at least or about 175 copies on a surface of the viral-like particle. In some embodiments, the fusion protein is expressed at a valency of at least or about 200 copies on a surface of the viral-like particle. In some embodiments, the fusion protein is expressed at a valency of at least or about 225 copies on a surface of the viral-like particle. In some embodiments, the fusion protein is expressed at a valency of at least or about 250 copies on a surface of the viral-like particle. In some embodiments, the fusion protein is expressed at a valency of at least or about 275 copies on a surface of the viral-like particle. In some embodiments, the fusion protein is expressed at a valency of at least or about 300 copies on a surface of the viral-like particle.

In some embodiments, the immune checkpoint multivalent particle is an extracellular vesicle. The extracellular vesicle as described herein, in some embodiments, comprise a fusion protein, wherein the fusion protein is expressed at multiple copies on a surface of the extracellular vesicle. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, or more than 400 copies on a surface of the extracellular vesicle. In some embodiments, the fusion protein is expressed at a valency of at least or about 5 to about 400, about 20 to about 400, about 10 to about 300, about 20 to about 300, about 20 to about 200, about 50 to about 150, about 20 to about 100, about 50 to about 100, or about 10 to about 15 copies on a surface of the extracellular vesicle. In some embodiments, the fusion protein is expressed at a valency of at least or about 10 copies on a surface of the extracellular vesicle. In some embodiments, the fusion protein is expressed at a valency of at least or about 15 copies on a surface of the extracellular vesicle. In some embodiments, the fusion protein is expressed at a valency of at least or about 25 copies on a surface of the extracellular vesicle. In some embodiments, the fusion protein is expressed at a valency of at least or about 50 copies on a surface of the extracellular vesicle. In some embodiments, the fusion protein is expressed at a valency of at least or about 75 copies on a surface of the extracellular vesicle. In some embodiments, the fusion protein is expressed at a valency of at least or about 100 copies on a surface of the extracellular vesicle. In some embodiments, the fusion protein is expressed at a valency of at least or about 125 copies on a surface of the extracellular vesicle. In some embodiments, the fusion protein is expressed at a valency of at least or about 150 copies on a surface of the extracellular vesicle. In some embodiments, the fusion protein is expressed at a valency of at least or about 175 copies on a surface of the extracellular vesicle. In some embodiments, the fusion protein is expressed at a valency of at least or about 200 copies on a surface of the extracellular vesicle. In some embodiments, the fusion protein is expressed at a valency of at least or about 225 copies on a surface of the extracellular vesicle. In some embodiments, the fusion protein is expressed at a valency of at least or about 250 copies on a surface of the extracellular vesicle. In some embodiments, the fusion protein is expressed at a valency of at least or about 275 copies on a surface of the extracellular vesicle. In some embodiments, the fusion protein is expressed at a valency of at least or about 300 copies on a surface of the extracellular vesicle. In some embodiments, the fusion protein is expressed at a valency of at least or about 350 copies on a surface of the extracellular vesicle. In some embodiments, the fusion protein is expressed at a valency of at least or about 400 copies on a surface of the extracellular vesicle. In some embodiments, the fusion protein is expressed at a valency of at least or about 450 copies on a surface of the extracellular vesicle. In some embodiments, the fusion protein is expressed at a valency of at least or about 500 copies on a surface of the extracellular vesicle. In some embodiments, the fusion protein is expressed at a valency of at least or about 600 copies on a surface of the extracellular vesicle. In some embodiments, the fusion protein is expressed at a valency of at least or about 700 copies on a surface of the extracellular vesicle. In some embodiments, the fusion protein is expressed at a valency of at least or about 800 copies on a surface of the extracellular vesicle. In some embodiments, the fusion protein is expressed at a valency of at least or about 900 copies on a surface of the extracellular vesicle. In some embodiments, the fusion protein is expressed at a valency of at least or about 1000 copies on a surface of the extracellular vesicle. In some embodiments, the fusion protein is expressed at a valency of at least or about 1100 copies on a surface of the extracellular vesicle. In some embodiments, the fusion protein is expressed at a valency of at least or about 1200 copies on a surface of the extracellular vesicle. In some embodiments, the fusion protein is expressed at a valency of at least or about 1300 copies on a surface of the extracellular vesicle. In some embodiments, the fusion protein is expressed at a valency of at least or about 1400 copies on a surface of the extracellular vesicle. In some embodiments, the fusion protein is expressed at a valency of at least or about 1500 copies on a surface of the extracellular vesicle. In some embodiments, the fusion protein is expressed at a valency of at least or about 1600 copies on a surface of the extracellular vesicle. In some embodiments, the fusion protein is expressed at a valency of at least or about 1700 copies on a surface of the extracellular vesicle. In some embodiments, the fusion protein is expressed at a valency of at least or about 1800 copies on a surface of the extracellular vesicle. In some embodiments, the fusion protein is expressed at a valency of at least or about 1900 copies on a surface of the extracellular vesicle. In some embodiments, the fusion protein is expressed at a valency of at least or about 2000 copies on a surface of the extracellular vesicle. In some embodiments, the fusion protein is expressed at a valency of at least or about 2100 copies on a surface of the extracellular vesicle. In some embodiments, the fusion protein is expressed at a valency of at least or about 2200 copies on a surface of the extracellular vesicle. In some embodiments, the fusion protein is expressed at a valency of at least or about 2300 copies on a surface of the extracellular vesicle. In some embodiments, the fusion protein is expressed at a valency of at least or about 2400 copies on a surface of the extracellular vesicle. In some embodiments, the fusion protein is expressed at a valency of at least or about 2500 copies on a surface of the extracellular vesicle. In some embodiments, the fusion protein is expressed at a valency of at least or about 2600 copies on a surface of the extracellular vesicle. In some embodiments, the fusion protein is expressed at a valency of at least or about 2700 copies on a surface of the extracellular vesicle. In some embodiments, the fusion protein is expressed at a valency of at least or about 2800 copies on a surface of the extracellular vesicle. In some embodiments, the fusion protein is expressed at a valency of at least or about 2900 copies on a surface of the extracellular vesicle. In some embodiments, the fusion protein is expressed at a valency of at least or about 3000 copies on a surface of the extracellular vesicle.

In some embodiments, the immune checkpoint multivalent particle is an exosome. The exosome as described herein, in some embodiments, comprise a fusion protein, wherein the fusion protein is expressed at multiple copies on a surface of the exosome. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, 3000, 3025, 3050, 3075, 3100, 3125, 3150, 3175, 3200, 3225, 3250, 3275, 3300, 3325, 3350, 3375, 3400, 3425, 3450, 3475, 3500, 3525, 3550, 3575, 3600, 3625, 3650, 3675, 3700, 3725, 3750, 3775, 3800, 3825, 3850, 3875, 3900, 3925, 3950, 3975, 4000, or more than 4000 copies on a surface of the exosome. In some embodiments, the fusion protein is expressed at a valency of at least or about 5 to about 400, about 20 to about 400, about 10 to about 300, about 20 to about 300, about 20 to about 200, about 50 to about 150, about 20 to about 100, about 50 to about 100, or about 10 to about 15 copies on a surface of the exosome. In some embodiments, the fusion protein is expressed at a valency of at least or about 10 copies on a surface of the exosome. In some embodiments, the fusion protein is expressed at a valency of at least or about 15 copies on a surface of the exosome. In some embodiments, the fusion protein is expressed at a valency of at least or about 25 copies on a surface of the exosome. In some embodiments, the fusion protein is expressed at a valency of at least or about 50 copies on a surface of the exosome. In some embodiments, the fusion protein is expressed at a valency of at least or about 75 copies on a surface of the exosome. In some embodiments, the fusion protein is expressed at a valency of at least or about 100 copies on a surface of the exosome. In some embodiments, the fusion protein is expressed at a valency of at least or about 125 copies on a surface of the exosome. In some embodiments, the fusion protein is expressed at a valency of at least or about 150 copies on a surface of the exosome. In some embodiments, the fusion protein is expressed at a valency of at least or about 175 copies on a surface of the exosome. In some embodiments, the fusion protein is expressed at a valency of at least or about 200 copies on a surface of the exosome. In some embodiments, the fusion protein is expressed at a valency of at least or about 225 copies on a surface of the exosome. In some embodiments, the fusion protein is expressed at a valency of at least or about 250 copies on a surface of the exosome. In some embodiments, the fusion protein is expressed at a valency of at least or about 275 copies on a surface of the exosome. In some embodiments, the fusion protein is expressed at a valency of at least or about 300 copies on a surface of the exosome. In some embodiments, the fusion protein is expressed at a valency of at least or about 350 copies on a surface of the exosome. In some embodiments, the fusion protein is expressed at a valency of at least or about 400 copies on a surface of the exosome. In some embodiments, the fusion protein is expressed at a valency of at least or about 450 copies on a surface of the exosome. In some embodiments, the fusion protein is expressed at a valency of at least or about 500 copies on a surface of the exosome. In some embodiments, the fusion protein is expressed at a valency of at least or about 600 copies on a surface of the exosome. In some embodiments, the fusion protein is expressed at a valency of at least or about 700 copies on a surface of the exosome. In some embodiments, the fusion protein is expressed at a valency of at least or about 800 copies on a surface of the exosome. In some embodiments, the fusion protein is expressed at a valency of at least or about 900 copies on a surface of the exosome. In some embodiments, the fusion protein is expressed at a valency of at least or about 1000 copies on a surface of the exosome. In some embodiments, the fusion protein is expressed at a valency of at least or about 1100 copies on a surface of the exosome. In some embodiments, the fusion protein is expressed at a valency of at least or about 1200 copies on a surface of the exosome. In some embodiments, the fusion protein is expressed at a valency of at least or about 1300 copies on a surface of the exosome. In some embodiments, the fusion protein is expressed at a valency of at least or about 1400 copies on a surface of the exosome. In some embodiments, the fusion protein is expressed at a valency of at least or about 1500 copies on a surface of the exosome. In some embodiments, the fusion protein is expressed at a valency of at least or about 1600 copies on a surface of the exosome. In some embodiments, the fusion protein is expressed at a valency of at least or about 1700 copies on a surface of the exosome. In some embodiments, the fusion protein is expressed at a valency of at least or about 1800 copies on a surface of the exosome. In some embodiments, the fusion protein is expressed at a valency of at least or about 1900 copies on a surface of the exosome. In some embodiments, the fusion protein is expressed at a valency of at least or about 2000 copies on a surface of the exosome. In some embodiments, the fusion protein is expressed at a valency of at least or about 2100 copies on a surface of the exosome. In some embodiments, the fusion protein is expressed at a valency of at least or about 2200 copies on a surface of the exosome. In some embodiments, the fusion protein is expressed at a valency of at least or about 2300 copies on a surface of the exosome. In some embodiments, the fusion protein is expressed at a valency of at least or about 2400 copies on a surface of the exosome. In some embodiments, the fusion protein is expressed at a valency of at least or about 2500 copies on a surface of the exosome. In some embodiments, the fusion protein is expressed at a valency of at least or about 2600 copies on a surface of the exosome. In some embodiments, the fusion protein is expressed at a valency of at least or about 2700 copies on a surface of the exosome. In some embodiments, the fusion protein is expressed at a valency of at least or about 2800 copies on a surface of the exosome. In some embodiments, the fusion protein is expressed at a valency of at least or about 2900 copies on a surface of the exosome. In some embodiments, the fusion protein is expressed at a valency of at least or about 3000 copies on a surface of the exosome.

In some embodiments, the immune checkpoint multivalent particle is an ectosome. The ectosome as described herein, in some embodiments, comprise a fusion protein, wherein the fusion protein is expressed at multiple copies on a surface of the ectosome. In some embodiments, the fusion protein is expressed at a valency of at least or about 5, 10, 15, 20, 25, 30, 35, 40, 45, 50, 75, 100, 125, 150, 175, 200, 225, 250, 275, 300, 325, 350, 375, 400, 425, 450, 475, 500, 525, 550, 575, 600, 625, 650, 675, 700, 725, 750, 775, 800, 825, 850, 875, 900, 925, 950, 975, 1000, 1025, 1050, 1075, 1100, 1125, 1150, 1175, 1200, 1225, 1250, 1275, 1300, 1325, 1350, 1375, 1400, 1425, 1450, 1475, 1500, 1525, 1550, 1575, 1600, 1625, 1650, 1675, 1700, 1725, 1750, 1775, 1800, 1825, 1850, 1875, 1900, 1925, 1950, 1975, 2000, 2025, 2050, 2075, 2100, 2125, 2150, 2175, 2200, 2225, 2250, 2275, 2300, 2325, 2350, 2375, 2400, 2425, 2450, 2475, 2500, 2525, 2550, 2575, 2600, 2625, 2650, 2675, 2700, 2725, 2750, 2775, 2800, 2825, 2850, 2875, 2900, 2925, 2950, 2975, 3000, 3025, 3050, 3075, 3100, 3125, 3150, 3175, 3200, 3225, 3250, 3275, 3300, 3325, 3350, 3375, 3400, 3425, 3450, 3475, 3500, 3525, 3550, 3575, 3600, 3625, 3650, 3675, 3700, 3725, 3750, 3775, 3800, 3825, 3850, 3875, 3900, 3925, 3950, 3975, 4000, or more than 4000 copies on a surface of the ectosome. In some embodiments, the fusion protein is expressed at a valency of at least or about 5 to about 400, about 20 to about 400, about 10 to about 300, about 20 to about 300, about 20 to about 200, about 50 to about 150, about 20 to about 100, about 50 to about 100, or about 10 to about 15 copies on a surface of the ectosome. In some embodiments, the fusion protein is expressed at a valency of at least or about 10 copies on a surface of the ectosome. In some embodiments, the fusion protein is expressed at a valency of at least or about 15 copies on a surface of the ectosome. In some embodiments, the fusion protein is expressed at a valency of at least or about 25 copies on a surface of the ectosome. In some embodiments, the fusion protein is expressed at a valency of at least or about 50 copies on a surface of the ectosome. In some embodiments, the fusion protein is expressed at a valency of at least or about 75 copies on a surface of the ectosome. In some embodiments, the fusion protein is expressed at a valency of at least or about 100 copies on a surface of the ectosome. In some embodiments, the fusion protein is expressed at a valency of at least or about 125 copies on a surface of the ectosome. In some embodiments, the fusion protein is expressed at a valency of at least or about 150 copies on a surface of the ectosome. In some embodiments, the fusion protein is expressed at a valency of at least or about 175 copies on a surface of the ectosome. In some embodiments, the fusion protein is expressed at a valency of at least or about 200 copies on a surface of the ectosome. In some embodiments, the fusion protein is expressed at a valency of at least or about 225 copies on a surface of the ectosome. In some embodiments, the fusion protein is expressed at a valency of at least or about 250 copies on a surface of the ectosome. In some embodiments, the fusion protein is expressed at a valency of at least or about 275 copies on a surface of the ectosome. In some embodiments, the fusion protein is expressed at a valency of at least or about 300 copies on a surface of the ectosome. In some embodiments, the fusion protein is expressed at a valency of at least or about 350 copies on a surface of the ectosome. In some embodiments, the fusion protein is expressed at a valency of at least or about 400 copies on a surface of the ectosome. In some embodiments, the fusion protein is expressed at a valency of at least or about 450 copies on a surface of the ectosome. In some embodiments, the fusion protein is expressed at a valency of at least or about 500 copies on a surface of the ectosome. In some embodiments, the fusion protein is expressed at a valency of at least or about 600 copies on a surface of the ectosome. In some embodiments, the fusion protein is expressed at a valency of at least or about 700 copies on a surface of the ectosome. In some embodiments, the fusion protein is expressed at a valency of at least or about 800 copies on a surface of the ectosome. In some embodiments, the fusion protein is expressed at a valency of at least or about 900 copies on a surface of the ectosome. In some embodiments, the fusion protein is expressed at a valency of at least or about 1000 copies on a surface of the ectosome. In some embodiments, the fusion protein is expressed at a valency of at least or about 1100 copies on a surface of the ectosome. In some embodiments, the fusion protein is expressed at a valency of at least or about 1200 copies on a surface of the ectosome. In some embodiments, the fusion protein is expressed at a valency of at least or about 1300 copies on a surface of the ectosome. In some embodiments, the fusion protein is expressed at a valency of at least or about 1400 copies on a surface of the ectosome. In some embodiments, the fusion protein is expressed at a valency of at least or about 1500 copies on a surface of the ectosome. In some embodiments, the fusion protein is expressed at a valency of at least or about 1600 copies on a surface of the ectosome. In some embodiments, the fusion protein is expressed at a valency of at least or about 1700 copies on a surface of the ectosome. In some embodiments, the fusion protein is expressed at a valency of at least or about 1800 copies on a surface of the ectosome. In some embodiments, the fusion protein is expressed at a valency of at least or about 1900 copies on a surface of the ectosome. In some embodiments, the fusion protein is expressed at a valency of at least or about 2000 copies on a surface of the ectosome. In some embodiments, the fusion protein is expressed at a valency of at least or about 2100 copies on a surface of the ectosome. In some embodiments, the fusion protein is expressed at a valency of at least or about 2200 copies on a surface of the ectosome. In some embodiments, the fusion protein is expressed at a valency of at least or about 2300 copies on a surface of the ectosome. In some embodiments, the fusion protein is expressed at a valency of at least or about 2400 copies on a surface of the ectosome. In some embodiments, the fusion protein is expressed at a valency of at least or about 2500 copies on a surface of the ectosome. In some embodiments, the fusion protein is expressed at a valency of at least or about 2600 copies on a surface of the ectosome. In some embodiments, the fusion protein is expressed at a valency of at least or about 2700 copies on a surface of the ectosome. In some embodiments, the fusion protein is expressed at a valency of at least or about 2800 copies on a surface of the ectosome. In some embodiments, the fusion protein is expressed at a valency of at least or about 2900 copies on a surface of the ectosome. In some embodiments, the fusion protein is expressed at a valency of at least or about 3000 copies on a surface of the ectosome.

Described herein, in some embodiments, are immune checkpoint multivalent particles that comprise an oligomerization domain. In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain is a tetramerization domain. In some embodiments, the tetramerization domain comprises an influenza neuraminidase stem domain.

Described herein, in some embodiments, are immune checkpoint multivalent particles that modulates the interaction between an immune checkpoint and its ligand. For example, the immune checkpoint multivalent particles modulate the interaction between PD-1 and its ligand PDL-1 or PDL-2. In some embodiments, the immune checkpoint multivalent particles that modulates the interaction between an immune checkpoint and its ligand result in an inhibitory effect. In some cases, the immune checkpoint multivalent particles inhibit activation. In some cases, the multivalent particles inhibit downstream signaling. In some embodiments, the immune checkpoint multivalent particles that modulates the interaction between an immune checkpoint and its ligand result in a stimulatory effect. In some cases, the immune checkpoint multivalent particles activate downstream signaling.

Described herein, in some embodiments, are immune checkpoint multivalent particles comprising improved binding properties. In some embodiments, the multivalent particle comprises a binding affinity (e.g., K_{D}) to the immune checkpoint of less than 100 pM, less than 200 pM, less than 300 pM, less than 400 pM, less than 500 pM, less than 600 pM, less than 700 pM, less than 800 pM, or less than 900 pM In some embodiments, the multivalent particle comprises a K_{D} of less than 1 nM, less than 1.2 nM, less than 2 nM, less than 5 nM, or less than 10 nM. In some instances, the multivalent particle comprises a K_{D} of less than 1 nM. In some instances, the multivalent particle comprises a K_{D} of less than 1.2 nM. In some instances, the multivalent particle comprises a K_{D} of less than 2 nM. In some instances, the multivalent particle comprises a K_{D} of less than 5 nM. In some instances, the multivalent particle comprises a K_{D} of less than 10 nM.

### Mammalian Immune Checkpoint Polypeptides

Described herein, in some embodiments, are multivalent particles comprises a mammalian immune checkpoint polypeptide and a transmembrane polypeptide. Described herein, in some embodiments, are multivalent particles comprises the extracellular domain of a mammalian immune checkpoint polypeptide and a transmembrane polypeptide. In some embodiments, the mammalian immune checkpoint polypeptide is an immune inhibitory checkpoint polypeptide. In some embodiments, the mammalian immune checkpoint polypeptide is an immune stimulatory checkpoint polypeptide. In some embodiments, the mammalian immune checkpoint polypeptide comprises a polypeptide expressed on T cells. In some embodiments, the mammalian immune checkpoint polypeptide comprises a polypeptide expressed on antigen presenting cells, such as dendritic cells, somatic cells, or tumor cells.

In some embodiments, the mammalian immune checkpoint polypeptide comprises an immune inhibitory checkpoint polypeptide. In some embodiments, the immune inhibitory checkpoint polypeptide is expressed on T cells. In some embodiments, the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9. In some embodiments, the immune inhibitory checkpoint polypeptide is expressed on antigen presenting cells, tumor cells, or normal cells. In some embodiments, the immune inhibitory checkpoint polypeptide comprises PD-L1, PD-L2, CD80, CD86, HVEM, CD48, CD112, CD155, Ceacam1, FGL1, or Galectin-3. In some embodiments, the immune inhibitory checkpoint polypeptide comprises the extracellular domain of PD-1, CTLA4, LAG3, BTLA, CD160, 2B4, CD226, TIGIT, CD96, B7-H3, B7-H4, VISTA, TIM3, SIGLEC7, KLRG1, or SIGLEC9. In some embodiments, the immune inhibitory checkpoint polypeptide comprises the extracellular domain of PD-L1, PD-L2, CD80, CD86, HVEM, CD48, CD112, CD155, Ceacam1, FGL1, or Galectin-3.

In some embodiments, the mammalian immune checkpoint polypeptide comprises an immune stimulatory checkpoint polypeptide. In some embodiments, the immune stimulatory checkpoint polypeptide comprises a polypeptide expressed on T cells. In some embodiments, the immune stimulatory checkpoint polypeptide comprises CD27, CD28, CD40, CD122, 4-1BB, ICOS, OX40, CD2, CD30, or GITR. In some embodiments, the immune stimulatory checkpoint polypeptide is expressed on antigen presenting cells, tumor cells, or normal cells. In some embodiments, the immune stimulatory checkpoint polypeptide comprises CD70, CD80, CD86, CD40L, GITRL, 4-1BBL, OX40L, LIGHT, CD30L, CD48, or ICOSL. In some embodiments, the immune stimulatory checkpoint polypeptide comprises the extracellular domain of CD27, CD28, CD40, CD122, 4-1BB, ICOS, OX40, CD2, CD30, or GITR. In some embodiments, the immune stimulatory checkpoint polypeptide comprises the extracellular domain of CD70, CD80, CD86, CD40L, GITRL, 4-1BBL, OX40L, LIGHT, CD30L, CD48, or ICOSL.

In some embodiments, the mammalian immune checkpoint polypeptide comprises an amino acid sequence of at least 75% sequence identity to an amino acid sequence according to SEQ ID NO: 1-62, 96-115, 153-162. In some embodiments, the mammalian immune checkpoint polypeptide comprises an amino acid sequence of at least 76% sequence identity to an amino acid sequence according to SEQ ID NO: 1-62, 96-115, 153-162. In some embodiments, the mammalian immune checkpoint polypeptide comprises an amino acid sequence of at least 77% sequence identity to an amino acid sequence according to SEQ ID NO: 1-62, 96-115, 153-162. In some embodiments, the mammalian immune checkpoint polypeptide comprises an amino acid sequence of at least 78% sequence identity to an amino acid sequence according to SEQ ID NO: 1-62, 96-115, 153-162. In some embodiments, the mammalian immune checkpoint polypeptide comprises an amino acid sequence of at least 79% sequence identity to an amino acid sequence according to SEQ ID NO: 1-62, 96-115, 153-162. In some embodiments, the mammalian immune checkpoint polypeptide comprises an amino acid sequence of at least 80% sequence identity to an amino acid sequence according to SEQ ID NO: 1-62, 96-115, 153-162. In some embodiments, the mammalian immune checkpoint polypeptide comprises an amino acid sequence of at least 81% sequence identity to an amino acid sequence according to SEQ ID NO: 1-62, 96-115, 153-162. In some embodiments, the mammalian immune checkpoint polypeptide comprises an amino acid sequence of at least 82% sequence identity to an amino acid sequence according to SEQ ID NO: 1-62, 96-115, 153-162. In some embodiments, the mammalian immune checkpoint polypeptide comprises an amino acid sequence of at least 83% sequence identity to an amino acid sequence according to SEQ ID NO: 1-62, 96-115, 153-162. In some embodiments, the mammalian immune checkpoint polypeptide comprises an amino acid sequence of at least 84% sequence identity to an amino acid sequence according to SEQ ID NO: 1-62, 96-115, 153-162. In some embodiments, the mammalian immune checkpoint polypeptide comprises an amino acid sequence of at least 85% sequence identity to an amino acid sequence according to SEQ ID NO: 1-62, 96-115, 153-162. In some embodiments, the mammalian immune checkpoint polypeptide comprises an amino acid sequence of at least 86% sequence identity to an amino acid sequence according to SEQ ID NO: 1-62, 96-115, 153-162. In some embodiments, the mammalian immune checkpoint polypeptide comprises an amino acid sequence of at least 87% sequence identity to an amino acid sequence according to SEQ ID NO: 1-62, 96-115, 153-162. In some embodiments, the mammalian immune checkpoint polypeptide comprises an amino acid sequence of at least 88% sequence identity to an amino acid sequence according to SEQ ID NO: 1-62, 96-115, 153-162. In some embodiments, the mammalian immune checkpoint polypeptide comprises an amino acid sequence of at least 89% sequence identity to an amino acid sequence according to SEQ ID NO: 1-62, 96-115, 153-162. In some embodiments, the mammalian immune checkpoint polypeptide comprises an amino acid sequence of at least 90% sequence identity to an amino acid sequence according to SEQ ID NO: 1-62, 96-115, 153-162. In some embodiments, the mammalian immune checkpoint polypeptide comprises an amino acid sequence of at least 91% sequence identity to an amino acid sequence according to SEQ ID NO: 1-62, 96-115, 153-162. In some embodiments, the mammalian immune checkpoint polypeptide comprises an amino acid sequence of at least 92% sequence identity to an amino acid sequence according to SEQ ID NO: 1-62, 96-115, 153-162. In some embodiments, the mammalian immune checkpoint polypeptide comprises an amino acid sequence of at least 93% sequence identity to an amino acid sequence according to SEQ ID NO: 1-62, 96-115, 153-162. In some embodiments, the mammalian immune checkpoint polypeptide comprises an amino acid sequence of at least 94% sequence identity to an amino acid sequence according to SEQ ID NO: 1-62, 96-115, 153-162. In some embodiments, the mammalian immune checkpoint polypeptide comprises an amino acid sequence of at least 95% sequence identity to an amino acid sequence according to SEQ ID NO: 1-62, 96-115, 153-162. In some embodiments, the mammalian immune checkpoint polypeptide comprises an amino acid sequence of at least 96% sequence identity to an amino acid sequence according to SEQ ID NO: 1-62, 96-115. In some embodiments, the mammalian immune checkpoint polypeptide comprises an amino acid sequence of at least 97% sequence identity to an amino acid sequence according to SEQ ID NO: 1-62, 96-115, 153-162. In some embodiments, the mammalian immune checkpoint polypeptide comprises an amino acid sequence of at least 98% sequence identity to an amino acid sequence according to SEQ ID NO: 1-62, 96-115, 153-162. In some embodiments, the mammalian immune checkpoint polypeptide comprises an amino acid sequence of at least 99% sequence identity to an amino acid sequence according to SEQ ID NO: 1-62, 96-115, 153-162. In some embodiments, the mammalian immune checkpoint polypeptide comprises an amino acid sequence according to SEQ ID NO: 1-62, 96-115, 153-162.

In some embodiments, the mammalian immune checkpoint polypeptide comprises an amino acid sequence of at least 75% sequence homology to an amino acid sequence according to SEQ ID NO: 1-62, 96-115, 153-162. In some embodiments, the mammalian immune checkpoint polypeptide comprises an amino acid sequence of at least 76% sequence homology to an amino acid sequence according to SEQ ID NO: 1-62, 96-115, 153-162. In some embodiments, the mammalian immune checkpoint polypeptide comprises an amino acid sequence of at least 77% sequence homology to an amino acid sequence according to SEQ ID NO: 1-62, 96-115, 153-162. In some embodiments, the mammalian immune checkpoint polypeptide comprises an amino acid sequence of at least 78% sequence homology to an amino acid sequence according to SEQ ID NO: 1-62, 96-115, 153-162. In some embodiments, the mammalian immune checkpoint polypeptide comprises an amino acid sequence of at least 79% sequence homology to an amino acid sequence according to SEQ ID NO: 1-62, 96-115, 153-162. In some embodiments, the mammalian immune checkpoint polypeptide comprises an amino acid sequence of at least 80% sequence homology to an amino acid sequence according to SEQ ID NO: 1-62, 96-115, 153-162. In some embodiments, the mammalian immune checkpoint polypeptide comprises an amino acid sequence of at least 81% sequence homology to an amino acid sequence according to SEQ ID NO: 1-62, 96-115, 153-162. In some embodiments, the mammalian immune checkpoint polypeptide comprises an amino acid sequence of at least 82% sequence homology to an amino acid sequence according to SEQ ID NO: 1-62, 96-115, 153-162. In some embodiments, the mammalian immune checkpoint polypeptide comprises an amino acid sequence of at least 83% sequence homology to an amino acid sequence according to SEQ ID NO: 1-62, 96-115, 153-162. In some embodiments, the mammalian immune checkpoint polypeptide comprises an amino acid sequence of at least 84% sequence homology to an amino acid sequence according to SEQ ID NO: 1-62, 96-115, 153-162. In some embodiments, the mammalian immune checkpoint polypeptide comprises an amino acid sequence of at least 85% sequence homology to an amino acid sequence according to SEQ ID NO: 1-62, 96-115, 153-162. In some embodiments, the mammalian immune checkpoint polypeptide comprises an amino acid sequence of at least 86% sequence homology to an amino acid sequence according to SEQ ID NO: 1-62, 96-115, 153-162. In some embodiments, the mammalian immune checkpoint polypeptide comprises an amino acid sequence of at least 87% sequence homology to an amino acid sequence according to SEQ ID NO: 1-62, 96-115, 153-162. In some embodiments, the mammalian immune checkpoint polypeptide comprises an amino acid sequence of at least 88% sequence homology to an amino acid sequence according to SEQ ID NO: 1-62, 96-115, 153-162. In some embodiments, the mammalian immune checkpoint polypeptide comprises an amino acid sequence of at least 89% sequence homology to an amino acid sequence according to SEQ ID NO: 1-62, 96-115, 153-162. In some embodiments, the mammalian immune checkpoint polypeptide comprises an amino acid sequence of at least 90% sequence homology to an amino acid sequence according to SEQ ID NO: 1-62, 96-115. In some embodiments, the mammalian immune checkpoint polypeptide comprises an amino acid sequence of at least 91% sequence homology to an amino acid sequence according to SEQ ID NO: 1-62, 96-115, 153-162. In some embodiments, the mammalian immune checkpoint polypeptide comprises an amino acid sequence of at least 92% sequence homology to an amino acid sequence according to SEQ ID NO: 1-62, 96-115, 153-162. In some embodiments, the mammalian immune checkpoint polypeptide comprises an amino acid sequence of at least 93% sequence homology to an amino acid sequence according to SEQ ID NO: 1-62, 96-115, 153-162. In some embodiments, the mammalian immune checkpoint polypeptide comprises an amino acid sequence of at least 94% sequence homology to an amino acid sequence according to SEQ ID NO: 1-62, 96-115, 153-162. In some embodiments, the mammalian immune checkpoint polypeptide comprises an amino acid sequence of at least 95% sequence homology to an amino acid sequence according to SEQ ID NO: 1-62, 96-115, 153-162. In some embodiments, the mammalian immune checkpoint polypeptide comprises an amino acid sequence of at least 96% sequence homology to an amino acid sequence according to SEQ ID NO: 1-62, 96-115, 153-162. In some embodiments, the mammalian immune checkpoint polypeptide comprises an amino acid sequence of at least 97% sequence homology to an amino acid sequence according to SEQ ID NO: 1-62, 96-115, 153-162. In some embodiments, the mammalian immune checkpoint polypeptide comprises an amino acid sequence of at least 98% sequence homology to an amino acid sequence according to SEQ ID NO: 1-62, 96-115, 153-162. In some embodiments, the mammalian immune checkpoint polypeptide comprises an amino acid sequence of at least 99% sequence homology to an amino acid sequence according to SEQ ID NO: 1-62, 96-115, 153-162.

In some instances, the mammalian immune checkpoint polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 or more than 800 amino acids of SEQ ID NO: 1-62, 96-115.

The term “sequence identity” means that two polynucleotide sequences are identical (i.e., on a nucleotide-by-nucleotide basis) over the window of comparison. The term “percentage of sequence identity” is calculated by comparing two optimally aligned sequences over the window of comparison, determining the number of positions at which the identical nucleic acid base (e.g., A, T, C, G, U, or I) occurs in both sequences to yield the number of matched positions, dividing the number of matched positions by the total number of positions in the window of comparison (i.e., the window size), and multiplying the result by 100 to yield the percentage of sequence identity. Typically, techniques for determining sequence identity include comparing two nucleotide or amino acid sequences and the determining their percent identity. Sequence comparisons, such as for the purpose of assessing identities, may be performed by any suitable alignment algorithm, including but not limited to the Needleman-Wunsch algorithm (see, e.g., the EMBOSS Needle aligner available at www.ebi.ac.uk/Tools/psa/emboss_needle/, optionally with default settings), the BLAST algorithm (see, e.g., the BLAST alignment tool available at blast.ncbi.nlm.nih.gov/Blast.cgi, optionally with default settings), and the Smith-Waterman algorithm (see, e.g., the EMBOSS Water aligner available at www.ebi.ac.uk/Tools/psa/emboss_water/, optionally with default settings). Optimal alignment may be assessed using any suitable parameters of a chosen algorithm, including default parameters. The “percent identity”, also referred to as “percent homology”, between two sequences may be calculated as the number of exact matches between two optimally aligned sequences divided by the length of the reference sequence and multiplied by 100. Percent identity may also be determined, for example, by comparing sequence information using the advanced BLAST computer program, including version 2.2.9, available from the National Institutes of Health. The BLAST program is based on the alignment method of Karlin and Altschul, Proc. Natl. Acad. Sci. USA 87:2264-2268 (1990) and as discussed in Altschul, et al., J. Mol. Biol. 215:403-410 (1990); Karlin and Altschul, Proc. Natl. Acad. Sci. USA 90:5873-5877 (1993); and Altschul et al., Nucleic Acids Res. 25:3389-3402 (1997). Briefly, the BLAST program defines identity as the number of identical aligned symbols (i.e., nucleotides or amino acids), divided by the total number of symbols in the shorter of the two sequences. The program may be used to determine percent identity over the entire length of the sequences being compared. Default parameters are provided to optimize searches with short query sequences, for example, with the blastp program. The program also allows use of an SEG filter to mask-off segments of the query sequences as determined by the SEG program of Wootton and Federhen, Computers and Chemistry 17: 149-163 (1993). High sequence identity generally includes ranges of sequence identity of approximately 80% to 100% and integer values there between.

### Oligomerization Domains

In some embodiments, the immune checkpoint multivalent particle comprises an oligomerization domain. In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain is a tetramerization domain. In some embodiments, the tetramerization domain comprises an influenza neuraminidase stem domain.

TABLE 1
Exemplary Oligomerization Domain Sequences

| | | | SEQ |
|---|---|---|---|
| Oligomerization | | Amino Acid | ID |
| Domain | Valence | Sequences | No. |
| D4 Variation 1 | Trimer | IGTALQVKMPKSHKA | 65 |
| | | IQADGWMCHASKWVT | |
| | | TCDFRWYGPKYITHS | |
| | | IRSFTPSVEQCKESI | |
| | | EQTKQGTWLNPGFPP | |
| | | QSCGYATVTD7AEAV | |
| | | IVQVTPHHVLVDEYT | |
| | | GEWVDSQFIN8GKCS | |
| | | NYICPTVHNSTTWHS | |
| | | DYKVKGLCDSNLISM | |
| | | DI | |
| D4 Variation 2 | Trimer | IQADGWMCHASKWVT | 66 |
| | | TCDFRWYGPKYITHS | |
| | | IRSFTPSVEQCKESI | |
| | | EQTKQGTWLNPGFPP | |
| | | QSCGYATVTDAEAVI | |
| | | VQVTPHHVLVDEYTG | |
| | | EWVDSQFINGKCSNY | |
| | | ICPTVHNSTTWHSDY | |
| | | KVKGLCDSNL | |
| D4 Variation 3 | Trimer | IQADGWMCHASKWVT | 67 |
| | | TCDFRWYGPKYITHS | |
| | | IRSFTPSVEQCKESI | |
| | | EQTKQGTWLNPGFPP | |
| | | QSCGYATVTDAEAVI | |
| | | VQVTPHHVLVDEYTG | |
| | | EWVDSQFINGKCSNY | |
| | | ICPTVHNSTT | |
| D4 Variation 4 | Trimer | IQADGWMCHASKWVT | 68 |
| | | TCDFRWYGPKYITHS | |
| | | IRSFTPSVEQCKESI | |
| | | EQTKQGTWLNPGFPP | |
| | | QSCGYATVIDAEAVI | |
| | | VQVTPHHVLVDEYTG | |
| | | EWVDSQFING | |
| D4 Variation 5 | Trimer | IQADGWMCHASKWVT | 69 |
| | | TCDFRWYGPKYITHS | |
| | | IRSFTPSVEQCKESI | |
| | | EQTKQGTWLNPGFPP | |
| | | QSCGYATVTDAEAVI | |
| | | VQVTPHHVL | |
| Foldon | Trimer | GYIPEAPRDGQAYVR | 70 |
| | | KDGEWVLLSTFL | |
| Leucine Zipper | Dimer | RMKQLEDKVEELLSK | 71 |
| V1 | | QYHLENEVARLKKLV | |
| | | GER | |
| Leucine Zipper | Dimer | RMKQLEDKVEELLSK | 72 |
| V2 | | NYHLENEVARLKKLV | |
| | | GER | |
| Neuraminidase | Tetramer | MNPNQKIITIGSICL | 73 |
| Stem V1 | | VVGLISLILQIGNII | |
| | | SIWISHSIQT | |
| Neuraminidase | Tetramer | MNPNQKIITIGSICM | 74 |
| Stem V2 | | VTGIVSLMLQIGNMI | |
| | | SIWVSHSIHTGNQHQ | |
| | | SEPISNTNFLTEKAV | |
| | | ASVKLAGNSSLCPIN | |
| Dengue E Fusion | Trimer | KLCIEAKISNTTTDS | 75 |
| V1 | | RCPTQGEATLVEEQD | |
| | | TNFVCRRTFVDRGHG | |
| | | NGCGLFGKGSLITCA | |
| | | KFKCVTKL | |
| Dengue E Fusion | Trimer | IELLKTEVTNPAVLR | 76 |
| V2 | | KLCIEAKISNTTTDS | |
| | | RCPTQGEATLVEEQD | |
| | | TNFVCRRTFVDRGHG | |
| | | NGCGLFGKGSLITCA | |
| | | KFKCVTKL | |
| Dengue E Fusion | Trimer | KLCIEAKISNTTTDS | 77 |
| V3 | | RCPTQGEATLVEEQD | |
| | | TNFVCRRTFVDRGHG | |
| | | NGCGLFGKGSLITCA | |
| | | KFKCVTKLEGKIVQY | |
| | | ENLKYSVI | |
| Dengue E Fusion | Trimer | EAKISNTTTDSRCPT | 78 |
| V4 | | QGEATLVEEQDTNFV | |
| | | CRRTFVDRGHGNGCG | |
| | | LFGKGSLITCAKFK | |

In some embodiments, the oligomerization domain comprises an amino acid sequence disclosed in Table 1, or an amino acid sequence that is substantially identical to an amino acid sequence in Table 1 (e.g. 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity). In some instances, the oligomerization domain comprises an amino acid sequence comprising at least a portion having at least or about 10, 20, 30, 40, 50, 60, 70, 80, 90, 100, 110, 120, 130 amino acid sequences of any sequence according to Table 1. In some embodiments, the oligomerization domain comprises an amino acid sequence that has at least 95% sequence identity to an amino acid sequence according to any one of SEQ ID NOs: 65-78.

### Transmembrane Polypeptides

Described herein, in some embodiments, are multivalent particles comprising mammalian immune checkpoint polypeptide and a transmembrane polypeptide. In some embodiments, the transmembrane polypeptide comprises the transmembrane domain of a Vesicular Stomatitis virus glycoprotein (VSV-G). In some embodiments, the transmembrane polypeptide comprises the transmembrane domain and cytosolic domain of a Vesicular Stomatitis virus glycoprotein (VSV-G). In some embodiments, the transmembrane polypeptide comprises the transmembrane domain of a Dengue E protein. In some embodiments, the transmembrane polypeptide comprises the transmembrane domain and cytosolic domain of a Dengue E protein. In some embodiments, the transmembrane polypeptide comprises the transmembrane domain of influenza Hemagglutinin (HA). In some embodiments, the transmembrane polypeptide comprises the transmembrane domain and cytosolic domain of influenza Hemagglutinin (HA). In some embodiments, the transmembrane polypeptide comprises the transmembrane domain of HIV surface glycoprotein GP120 or GP41. In some embodiments, the transmembrane polypeptide comprises the transmembrane domain and cytosolic domain of HIV surface glycoprotein GP120 or GP41. In some embodiments, the transmembrane domain comprises the transmembrane polypeptide of measles virus surface glycoprotein hemagglutinin (H) protein. In some embodiments, the transmembrane polypeptide comprises the transmembrane domain and cytosolic domain of measles virus surface glycoprotein hemagglutinin (H) protein. In some embodiments, the transmembrane polypeptide comprises the transmembrane domain of influenza Neuraminidase (NA). In some embodiments, the transmembrane polypeptide comprises the transmembrane domain and cytosolic domain of influenza Neuraminidase (NA).

TABLE 2
Exemplary Transmembrane Domain Sequences

| | | SEQ |
|---|---|---|
| | | ID |
| Domain | Amino Acid Sequence | NO: |
| VSV-G | IASFFFIIGLIIGLFLVLRV | 79 |
| Transmembrane | GI | |
| (TM) V1 | | |
| VSV-G | PIELVEGWFSSWKSSIASFF | 80 |
| Transmembrane | FIIGLIIGLFLVLRVGI | |
| (TM) V2 | | |
| VSV-G | DDESLFFGDTGLSKNPIELV | 81 |
| Transmembrane | EGWFSSWKSSIASFFFIIGL | |
| (TM) V3 | IIGLFLVLRVGIH | |
| VSV-G | GMLDSDLHLSSKAQVFEHPH | 82 |
| Transmembrane | IQDAASQLPDDESLFFGDTG | |
| (TM) V4 | LSKNPIELVEGWFSSWKSSI | |
| | ASFFFIIGLIIGLFLVLRVG | |
| | I | |
| VSV-G | HLCIKLKHTKKRQIYTDIEM | 83 |
| Cytosolic Tail | NRLGK | |
| (CT) | | |
| Influenza | IITIGSVCMTIGMANLILQI | 84 |
| Neuraminidase | GNI | |
| TM (N1) | | |
| Influenza | LAIYSTVASSLVLVVSLGAI | 85 |
| Hemagglutinin | SFW | |
| TM (H1) | | |
| Dengue E | AYGVLFSGVSWTMKIGIGIL | 86 |
| Protein TM | LTWLGLNSRSTSLSMTCIAV | |
| | GMVTLYLGVMVQ | |
| HIV GP41 TM | LFIMIVGGLVGLRIVFAVLS | 87 |
| with Cytosolic | IVNRVRQGYSPLSFQTHLPT | |
| Tail | PRG | |
| Spike Protein S1 | WYVWLGFIAGLIAIVMVTIL | 88 |
| with Cytosolic | LCCMTSCCSCLKGACSCGSC | |
| Tail | CKFDEDDSEPVLKGVKLHYT | |
| Spike Protein S2 | WYIWLGFIAGLIAIVMVTIM | 89 |
| with Cytosolic | LCCMTSCCSCLKGCCSCGSC | |
| Tail | CKFDEDDSEPVLKGVKLHYT | |
| Sindbis Virus | WLFALFGGASSLLIIGLMIF | 90 |
| Envelope | ACSMMLTSTRR | |
| (SINDBIS) | | |
| Protein TM with | | |
| Cytosolic Tail | | |
| Hemagglutinin | MSPQRDRINAFYKDNPHPKG | 91 |
| Envelope Protein | SRIVINREHLMIDRPYVLLA | |
| from Measles | VLFVMFLSLIGLLAIAGI | |
| Virus TM with | | |
| Cytosolic Tail | | |
| Envelope | IVYILIAVCLGGLIGIPALI | 92 |
| Glycoprotein of | CCCRGR | |
| Measles Virus | | |
| Fusion (F) | | |
| Protein TM with | | |
| Cytosolic Tail | | |
| RD114 TM with | LLTLLLILTIGPCVFSRLMA | 93 |
| Cytosolic Tail | FINDRLNVVHAMVLAQQYQA | |
| | LKAEEEAQD | |
| BaEV TM with | LLTLLLLLTIGPCIFNRLTA | 94 |
| Cytosolic Tail | FINDKLNIIHAMVLTQQYQV | |
| | LRTDEEAQD | |
| GP120 TM with | IKIFIMIVGGLVGLRIIFAV | 95 |
| Cytosolic Tail | LSIVNRVRQGYSPLSFQIHS | |
| | HHQREPDRPEGIEEGGGEQG | |
| | KDRSVRLVSGFLALAWDDLR | |
| | SLCLFSYHRLRDFILVAART | |
| | VELLGHSSLKGLRLGWEGLK | |
| | YLGNLLIYWSQELKTSAISL | |
| | FDTIAIAVAGWTDRVIEIAQ | |
| | RAGRAIIHIPRRIRQGLERA | |
| | LQ | |

In some embodiments, the transmembrane domain comprises an amino acid sequence disclosed in Table 2, or an amino acid sequence that is substantially identical to an amino acid sequence in Table 2 (e.g. about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% sequence identity). In some instances, the transmembrane domain comprises an amino acid sequence comprising at least a portion having at least or about 10, 20, 30, 40, 50, 60, 70, 80, 90, 100, 110, 120, 130 amino acid sequences of any sequence according to Table 2.

Described herein, in some embodiments, are multivalent particles comprising mammalian immune checkpoint polypeptide and a transmembrane polypeptide. In some embodiments, the transmembrane polypeptide anchors the fusion protein to a lipid bilayer of the multivalent particle. In some embodiments, the transmembrane polypeptide comprises a spike glycoprotein, a mammalian membrane protein, an envelope protein, a nucleocapsid protein, or a cellular transmembrane protein. In some embodiments, the transmembrane polypeptide comprises VSVG, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120. In some embodiments, the transmembrane polypeptide comprises VSVG. In some embodiments, the VSVG comprises full length VSVG or a truncated VSVG. In some embodiments, the VSVG comprises a transmembrane domain and cytoplasmic tail. In some embodiments, the hemagglutinin envelope protein from measles virus is a variant of the hemagglutinin envelope protein from measles virus. In some instances, the variant is HCΔ18.

In some embodiments, the transmembrane polypeptide comprises an amino acid sequence of at least 75% sequence identity to an amino acid sequence according to SEQ ID NO: 63. In some embodiments, the transmembrane polypeptide comprises an amino acid sequence of at least 76% sequence identity to an amino acid sequence according to SEQ ID NO: 63. In some embodiments, the transmembrane polypeptide comprises an amino acid sequence of at least 77% sequence identity to an amino acid sequence according to SEQ ID NO: 63. In some embodiments, the transmembrane polypeptide comprises an amino acid sequence of at least 78% sequence identity to an amino acid sequence according to SEQ ID NO: 63. In some embodiments, the transmembrane polypeptide comprises an amino acid sequence of at least 79% sequence identity to an amino acid sequence according to SEQ ID NO: 63. In some embodiments, the transmembrane polypeptide comprises an amino acid sequence of at least 80% sequence identity to an amino acid sequence according to SEQ ID NO: 63. In some embodiments, the transmembrane polypeptide comprises an amino acid sequence of at least 81% sequence identity to an amino acid sequence according to SEQ ID NO: 63. In some embodiments, the transmembrane polypeptide comprises an amino acid sequence of at least 82% sequence identity to an amino acid sequence according to SEQ ID NO: 63. In some embodiments, the transmembrane polypeptide comprises an amino acid sequence of at least 83% sequence identity to an amino acid sequence according to SEQ ID NO: 63. In some embodiments, the transmembrane polypeptide comprises an amino acid sequence of at least 84% sequence identity to an amino acid sequence according to SEQ ID NO: 63. In some embodiments, the transmembrane polypeptide comprises an amino acid sequence of at least 85% sequence identity to an amino acid sequence according to SEQ ID NO: 63. In some embodiments, the transmembrane polypeptide comprises an amino acid sequence of at least 86% sequence identity to an amino acid sequence according to SEQ ID NO: 63. In some embodiments, the transmembrane polypeptide comprises an amino acid sequence of at least 87% sequence identity to an amino acid sequence according to SEQ ID NO: 63. In some embodiments, the transmembrane polypeptide comprises an amino acid sequence of at least 88% sequence identity to an amino acid sequence according to SEQ ID NO: 63. In some embodiments, the transmembrane polypeptide comprises an amino acid sequence of at least 89% sequence identity to an amino acid sequence according to SEQ ID NO: 63. In some embodiments, the transmembrane polypeptide comprises an amino acid sequence of at least 90% sequence identity to an amino acid sequence according to SEQ ID NO: 63. In some embodiments, the transmembrane polypeptide comprises an amino acid sequence of at least 91% sequence identity to an amino acid sequence according to SEQ ID NO: 63. In some embodiments, the transmembrane polypeptide comprises an amino acid sequence of at least 92% sequence identity to an amino acid sequence according to SEQ ID NO: 63. In some embodiments, the transmembrane polypeptide comprises an amino acid sequence of at least 93% sequence identity to an amino acid sequence according to SEQ ID NO: 63. In some embodiments, the transmembrane polypeptide comprises an amino acid sequence of at least 94% sequence identity to an amino acid sequence according to SEQ ID NO: 63. In some embodiments, the transmembrane polypeptide comprises an amino acid sequence of at least 95% sequence identity to an amino acid sequence according to SEQ ID NO: 63. In some embodiments, the transmembrane polypeptide comprises an amino acid sequence of at least 96% sequence identity to an amino acid sequence according to SEQ ID NO: 63. In some embodiments, the transmembrane polypeptide comprises an amino acid sequence of at least 97% sequence identity to an amino acid sequence according to SEQ ID NO: 63. In some embodiments, the transmembrane polypeptide comprises an amino acid sequence of at least 98% sequence identity to an amino acid sequence according to SEQ ID NO: 63. In some embodiments, the transmembrane polypeptide comprises an amino acid sequence of at least 99% sequence identity to an amino acid sequence according to SEQ ID NO: 63. In some embodiments, the transmembrane polypeptide comprises an amino acid sequence according to SEQ ID NO: 63.

In some embodiments, the transmembrane polypeptide comprises an amino acid sequence of at least 75% sequence homology to an amino acid sequence according to SEQ ID NO: 63. In some embodiments, the transmembrane polypeptide comprises an amino acid sequence of at least 76% sequence homology to an amino acid sequence according to SEQ ID NO: 63. In some embodiments, the transmembrane polypeptide comprises an amino acid sequence of at least 77% sequence homology to an amino acid sequence according to SEQ ID NO: 63. In some embodiments, the transmembrane polypeptide comprises an amino acid sequence of at least 78% sequence homology to an amino acid sequence according to SEQ ID NO: 63. In some embodiments, the transmembrane polypeptide comprises an amino acid sequence of at least 79% sequence homology to an amino acid sequence according to SEQ ID NO: 63. In some embodiments, the transmembrane polypeptide comprises an amino acid sequence of at least 80% sequence homology to an amino acid sequence according to SEQ ID NO: 63. In some embodiments, the transmembrane polypeptide comprises an amino acid sequence of at least 81% sequence homology to an amino acid sequence according to SEQ ID NO: 63. In some embodiments, the transmembrane polypeptide comprises an amino acid sequence of at least 82% sequence homology to an amino acid sequence according to SEQ ID NO: 63. In some embodiments, the transmembrane polypeptide comprises an amino acid sequence of at least 83% sequence homology to an amino acid sequence according to SEQ ID NO: 63. In some embodiments, the transmembrane polypeptide comprises an amino acid sequence of at least 84% sequence homology to an amino acid sequence according to SEQ ID NO: 63. In some embodiments, the transmembrane polypeptide comprises an amino acid sequence of at least 85% sequence homology to an amino acid sequence according to SEQ ID NO: 63. In some embodiments, the transmembrane polypeptide comprises an amino acid sequence of at least 86% sequence homology to an amino acid sequence according to SEQ ID NO: 63. In some embodiments, the transmembrane polypeptide comprises an amino acid sequence of at least 87% sequence homology to an amino acid sequence according to SEQ ID NO: 63. In some embodiments, the transmembrane polypeptide comprises an amino acid sequence of at least 88% sequence homology to an amino acid sequence according to SEQ ID NO: 63. In some embodiments, the transmembrane polypeptide comprises an amino acid sequence of at least 89% sequence homology to an amino acid sequence according to SEQ ID NO: 63. In some embodiments, the transmembrane polypeptide comprises an amino acid sequence of at least 90% sequence homology to an amino acid sequence according to SEQ ID NO: 63. In some embodiments, the transmembrane polypeptide comprises an amino acid sequence of at least 91% sequence homology to an amino acid sequence according to SEQ ID NO: 63. In some embodiments, the transmembrane polypeptide comprises an amino acid sequence of at least 92% sequence homology to an amino acid sequence according to SEQ ID NO: 63. In some embodiments, the transmembrane polypeptide comprises an amino acid sequence of at least 93% sequence homology to an amino acid sequence according to SEQ ID NO: 63. In some embodiments, the transmembrane polypeptide comprises an amino acid sequence of at least 94% sequence homology to an amino acid sequence according to SEQ ID NO: 63. In some embodiments, the transmembrane polypeptide comprises an amino acid sequence of at least 95% sequence homology to an amino acid sequence according to SEQ ID NO: 63. In some embodiments, the transmembrane polypeptide comprises an amino acid sequence of at least 96% sequence homology to an amino acid sequence according to SEQ ID NO: 63. In some embodiments, the transmembrane polypeptide comprises an amino acid sequence of at least 97% sequence homology to an amino acid sequence according to SEQ ID NO: 63. In some embodiments, the transmembrane polypeptide comprises an amino acid sequence of at least 98% sequence homology to an amino acid sequence according to SEQ ID NO: 63. In some embodiments, the transmembrane polypeptide comprises an amino acid sequence of at least 99% sequence homology to an amino acid sequence according to SEQ ID NO: 63.

In some instances, the transmembrane polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or more than 490 amino acids of SEQ ID NO: 63.

In some embodiments, the transmembrane polypeptide comprises an amino acid sequence of at least 75% sequence identity to an amino acid sequence according to SEQ ID NO: 64. In some embodiments, the transmembrane polypeptide comprises an amino acid sequence of at least 76% sequence identity to an amino acid sequence according to SEQ ID NO: 64. In some embodiments, the transmembrane polypeptide comprises an amino acid sequence of at least 77% sequence identity to an amino acid sequence according to SEQ ID NO: 64. In some embodiments, the transmembrane polypeptide comprises an amino acid sequence of at least 78% sequence identity to an amino acid sequence according to SEQ ID NO: 64. In some embodiments, the transmembrane polypeptide comprises an amino acid sequence of at least 79% sequence identity to an amino acid sequence according to SEQ ID NO: 64. In some embodiments, the transmembrane polypeptide comprises an amino acid sequence of at least 80% sequence identity to an amino acid sequence according to SEQ ID NO: 64. In some embodiments, the transmembrane polypeptide comprises an amino acid sequence of at least 81% sequence identity to an amino acid sequence according to SEQ ID NO: 64. In some embodiments, the transmembrane polypeptide comprises an amino acid sequence of at least 82% sequence identity to an amino acid sequence according to SEQ ID NO: 64. In some embodiments, the transmembrane polypeptide comprises an amino acid sequence of at least 83% sequence identity to an amino acid sequence according to SEQ ID NO: 64. In some embodiments, the transmembrane polypeptide comprises an amino acid sequence of at least 84% sequence identity to an amino acid sequence according to SEQ ID NO: 64. In some embodiments, the transmembrane polypeptide comprises an amino acid sequence of at least 85% sequence identity to an amino acid sequence according to SEQ ID NO: 64. In some embodiments, the transmembrane polypeptide comprises an amino acid sequence of at least 86% sequence identity to an amino acid sequence according to SEQ ID NO: 64. In some embodiments, the transmembrane polypeptide comprises an amino acid sequence of at least 87% sequence identity to an amino acid sequence according to SEQ ID NO: 64. In some embodiments, the transmembrane polypeptide comprises an amino acid sequence of at least 88% sequence identity to an amino acid sequence according to SEQ ID NO: 64. In some embodiments, the transmembrane polypeptide comprises an amino acid sequence of at least 89% sequence identity to an amino acid sequence according to SEQ ID NO: 64. In some embodiments, the transmembrane polypeptide comprises an amino acid sequence of at least 90% sequence identity to an amino acid sequence according to SEQ ID NO: 64. In some embodiments, the transmembrane polypeptide comprises an amino acid sequence of at least 91% sequence identity to an amino acid sequence according to SEQ ID NO: 64. In some embodiments, the transmembrane polypeptide comprises an amino acid sequence of at least 92% sequence identity to an amino acid sequence according to SEQ ID NO: 64. In some embodiments, the transmembrane polypeptide comprises an amino acid sequence of at least 93% sequence identity to an amino acid sequence according to SEQ ID NO: 64. In some embodiments, the transmembrane polypeptide comprises an amino acid sequence of at least 94% sequence identity to an amino acid sequence according to SEQ ID NO: 64. In some embodiments, the transmembrane polypeptide comprises an amino acid sequence of at least 95% sequence identity to an amino acid sequence according to SEQ ID NO: 64. In some embodiments, the transmembrane polypeptide comprises an amino acid sequence of at least 96% sequence identity to an amino acid sequence according to SEQ ID NO: 64. In some embodiments, the transmembrane polypeptide comprises an amino acid sequence of at least 97% sequence identity to an amino acid sequence according to SEQ ID NO: 64. In some embodiments, the transmembrane polypeptide comprises an amino acid sequence of at least 98% sequence identity to an amino acid sequence according to SEQ ID NO: 64. In some embodiments, the transmembrane polypeptide comprises an amino acid sequence of at least 99% sequence identity to an amino acid sequence according to SEQ ID NO: 64. In some embodiments, the transmembrane polypeptide comprises an amino acid sequence according to SEQ ID NO: 64.

In some embodiments, the transmembrane polypeptide comprises an amino acid sequence of at least 75% sequence homology to an amino acid sequence according to SEQ ID NO: 64. In some embodiments, the transmembrane polypeptide comprises an amino acid sequence of at least 76% sequence homology to an amino acid sequence according to SEQ ID NO: 64. In some embodiments, the transmembrane polypeptide comprises an amino acid sequence of at least 77% sequence homology to an amino acid sequence according to SEQ ID NO: 64. In some embodiments, the transmembrane polypeptide comprises an amino acid sequence of at least 78% sequence homology to an amino acid sequence according to SEQ ID NO: 64. In some embodiments, the transmembrane polypeptide comprises an amino acid sequence of at least 79% sequence homology to an amino acid sequence according to SEQ ID NO: 64. In some embodiments, the transmembrane polypeptide comprises an amino acid sequence of at least 80% sequence homology to an amino acid sequence according to SEQ ID NO: 64. In some embodiments, the transmembrane polypeptide comprises an amino acid sequence of at least 81% sequence homology to an amino acid sequence according to SEQ ID NO: 64. In some embodiments, the transmembrane polypeptide comprises an amino acid sequence of at least 82% sequence homology to an amino acid sequence according to SEQ ID NO: 64. In some embodiments, the transmembrane polypeptide comprises an amino acid sequence of at least 83% sequence homology to an amino acid sequence according to SEQ ID NO: 64. In some embodiments, the transmembrane polypeptide comprises an amino acid sequence of at least 84% sequence homology to an amino acid sequence according to SEQ ID NO: 64. In some embodiments, the transmembrane polypeptide comprises an amino acid sequence of at least 85% sequence homology to an amino acid sequence according to SEQ ID NO: 64. In some embodiments, the transmembrane polypeptide comprises an amino acid sequence of at least 86% sequence homology to an amino acid sequence according to SEQ ID NO: 64. In some embodiments, the transmembrane polypeptide comprises an amino acid sequence of at least 87% sequence homology to an amino acid sequence according to SEQ ID NO: 64. In some embodiments, the transmembrane polypeptide comprises an amino acid sequence of at least 88% sequence homology to an amino acid sequence according to SEQ ID NO: 64. In some embodiments, the transmembrane polypeptide comprises an amino acid sequence of at least 89% sequence homology to an amino acid sequence according to SEQ ID NO: 64. In some embodiments, the transmembrane polypeptide comprises an amino acid sequence of at least 90% sequence homology to an amino acid sequence according to SEQ ID NO: 64. In some embodiments, the transmembrane polypeptide comprises an amino acid sequence of at least 91% sequence homology to an amino acid sequence according to SEQ ID NO: 64. In some embodiments, the transmembrane polypeptide comprises an amino acid sequence of at least 92% sequence homology to an amino acid sequence according to SEQ ID NO: 64. In some embodiments, the transmembrane polypeptide comprises an amino acid sequence of at least 93% sequence homology to an amino acid sequence according to SEQ ID NO: 64. In some embodiments, the transmembrane polypeptide comprises an amino acid sequence of at least 94% sequence homology to an amino acid sequence according to SEQ ID NO: 64. In some embodiments, the transmembrane polypeptide comprises an amino acid sequence of at least 95% sequence homology to an amino acid sequence according to SEQ ID NO: 64. In some embodiments, the transmembrane polypeptide comprises an amino acid sequence of at least 96% sequence homology to an amino acid sequence according to SEQ ID NO: 64. In some embodiments, the transmembrane polypeptide comprises an amino acid sequence of at least 97% sequence homology to an amino acid sequence according to SEQ ID NO: 64. In some embodiments, the transmembrane polypeptide comprises an amino acid sequence of at least 98% sequence homology to an amino acid sequence according to SEQ ID NO: 64. In some embodiments, the transmembrane polypeptide comprises an amino acid sequence of at least 99% sequence homology to an amino acid sequence according to SEQ ID NO: 64.

In some instances, the transmembrane polypeptide comprises an amino acid sequence comprising at least a portion having at least or about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, or more than 490 amino acids of SEQ ID NO: 64.

### Mammalian Immune Checkpoint Polypeptide and Transmembrane Polypeptide Combinations

Described herein, in some embodiments, are multivalent particles comprising a fusion protein that comprises a mammalian immune checkpoint polypeptide and a transmembrane polypeptide. In some embodiments, the mammalian immune checkpoint polypeptide is an immune stimulatory checkpoint polypeptide. In some embodiments, the mammalian immune checkpoint polypeptide comprises a polypeptide expressed on T cells. In some embodiments, the mammalian immune checkpoint polypeptide comprises a polypeptide expressed on antigen presenting cells, cancer cells, and normal somatic cells.

In some embodiments, the mammalian immune checkpoint polypeptide comprises an immune inhibitory checkpoint polypeptide. In some embodiments, the mammalian immune checkpoint polypeptide comprises the extracellular domain of an immune inhibitory checkpoint polypeptide. In some embodiments, the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9. In some embodiments, the immune inhibitory checkpoint polypeptide comprises PD-L1, PD-L2, CD80, CD86, HVEM, CD48, CD112, CD155, Ceacam1, FGL1, or Galectin-3. In some embodiments, the immune inhibitory checkpoint polypeptide comprises the extracellular domain of PD-1, CTLA4, LAG3, BTLA, CD160, 2B4, CD226, TIGIT, CD96, B7-H3, B7-H4, VISTA, TIM3, SIGLEC7, KLRG1, or SIGLEC9. In some embodiments, the immune inhibitory checkpoint polypeptide comprises the extracellular domain of PD-L1, PD-L2, CD80, CD86, HVEM, CD48, CD112, CD155, Ceacam1, FGL1, or Galectin-3.

In some embodiments, the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises VSVG transmembrane domain. In some embodiments, the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises spike protein S1 transmembrane domain. In some embodiments, the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises spike protein S2 transmembrane domain. In some embodiments, the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises the transmembrane domain of a surface glycoprotein of an enveloped virus. In some embodiments, the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises the transmembrane domain of Sindbis virus envelope (SINDBIS) protein. In some embodiments, the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises BaEV transmembrane domain. In some embodiments, the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises GP41 transmembrane domain. In some embodiments, the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises GP120 transmembrane domain.

In some embodiments, the immune inhibitory checkpoint polypeptide comprises PD-L1, PD-L2, CD80, CD86, HVEM, CD48, CD112, CD155, Ceacam1, FGL1, or Galectin-3 and the transmembrane polypeptide comprises VSVG transmembrane domain. In some embodiments, the immune inhibitory checkpoint polypeptide comprises PD-L1, PD-L2, CD80, CD86, HVEM, CD48, CD112, CD155, Ceacam1, FGL1, or Galectin-3 and the transmembrane polypeptide comprises spike protein S1 transmembrane domain. In some embodiments, the immune inhibitory checkpoint polypeptide comprises PD-L1, PD-L2, CD80, CD86, HVEM, CD48, CD112, CD155, Ceacam1, FGL1, or Galectin-3 and the transmembrane polypeptide comprises spike protein S2 transmembrane domain. In some embodiments, the immune inhibitory checkpoint polypeptide comprises PD-L1, PD-L2, CD80, CD86, HVEM, CD48, CD112, CD155, Ceacam1, FGL1, or Galectin-3 and the transmembrane polypeptide comprises the transmembrane domain of a surface glycoprotein of an enveloped virus. In some embodiments, the immune inhibitory checkpoint polypeptide comprises PD-L1, PD-L2, CD80, CD86, HVEM, CD48, CD112, CD155, Ceacam1, FGL1, or Galectin-3 and the transmembrane polypeptide comprises the transmembrane domain of Sindbis virus envelope (SINDBIS) protein. In some embodiments, the immune inhibitory checkpoint polypeptide comprises PD-L1, PD-L2, CD80, CD86, HVEM, CD48, CD112, CD155, Ceacam1, FGL1, or Galectin-3 and the transmembrane polypeptide comprises BaEV transmembrane domain. In some embodiments, the immune inhibitory checkpoint polypeptide comprises PD-L1, PD-L2, CD80, CD86, HVEM, CD48, CD112, CD155, Ceacam1, FGL1, or Galectin-3 and the transmembrane polypeptide comprises GP41 transmembrane domain. In some embodiments, the immune inhibitory checkpoint polypeptide comprises PD-L1, PD-L2, CD80, CD86, HVEM, CD48, CD112, CD155, Ceacam1, FGL1, or Galectin-3 and the transmembrane polypeptide comprises GP120 transmembrane domain.

In some embodiments, the immune inhibitory checkpoint polypeptide comprises the extracellular domain of PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises VSVG transmembrane domain. In some embodiments, the immune inhibitory checkpoint polypeptide comprises the extracellular domain of PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises spike protein S1 transmembrane domain. In some embodiments, the immune inhibitory checkpoint polypeptide comprises the extracellular domain of PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises spike protein S2 transmembrane domain. In some embodiments, the immune inhibitory checkpoint polypeptide comprises the extracellular domain of PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises the transmembrane domain of a surface glycoprotein of an enveloped virus. In some embodiments, the immune inhibitory checkpoint polypeptide comprises the extracellular domain of PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises the transmembrane domain of Sindbis virus envelope (SINDBIS) protein. In some embodiments, the immune inhibitory checkpoint polypeptide comprises the extracellular domain of PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises BaEV transmembrane domain. In some embodiments, the immune inhibitory checkpoint polypeptide comprises the extracellular domain of PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises GP41 transmembrane domain. In some embodiments, the immune inhibitory checkpoint polypeptide comprises the extracellular domain of PD-1, CTLA4, LAG3, BTLA, CD160, 2B4, CD226, TIGIT, CD96, B7-H3, B7-H4, VISTA, TIM3, SIGLEC7, KLRG1, or SIGLEC9 and the transmembrane polypeptide comprises GP120 transmembrane domain.

In some embodiments, the immune inhibitory checkpoint polypeptide comprises the extracellular domain of PD-L1, PD-L2, CD80, CD86, HVEM, CD48, CD112, CD155, Ceacam1, FGL1, or Galectin-3 and the transmembrane polypeptide comprises VSVG transmembrane domain. In some embodiments, the immune inhibitory checkpoint polypeptide comprises the extracellular domain of PD-L1, PD-L2, CD80, CD86, HVEM, CD48, CD112, CD155, Ceacam1, FGL1, or Galectin-3 and the transmembrane polypeptide comprises spike protein S1 transmembrane domain. In some embodiments, the immune inhibitory checkpoint polypeptide comprises the extracellular domain of PD-L1, PD-L2, CD80, CD86, HVEM, CD48, CD112, CD155, Ceacam1, FGL1, or Galectin-3 and the transmembrane polypeptide comprises spike protein S2 transmembrane domain. In some embodiments, the immune inhibitory checkpoint polypeptide comprises the extracellular domain of PD-L1, PD-L2, CD80, CD86, HVEM, CD48, CD112, CD155, Ceacam1, FGL1, or Galectin-3 and the transmembrane polypeptide comprises the transmembrane domain of a surface glycoprotein of an enveloped virus. In some embodiments, the immune inhibitory checkpoint polypeptide comprises the extracellular domain of PD-L1, PD-L2, CD80, CD86, HVEM, CD48, CD112, CD155, Ceacam1, FGL1, or Galectin-3 and the transmembrane polypeptide comprises the transmembrane domain of Sindbis virus envelope (SINDBIS) protein. In some embodiments, the immune inhibitory checkpoint polypeptide comprises the extracellular domain of PD-L1, PD-L2, CD80, CD86, HVEM, CD48, CD112, CD155, Ceacam1, FGL1, or Galectin-3 and the transmembrane polypeptide comprises BaEV transmembrane domain. In some embodiments, the immune inhibitory checkpoint polypeptide comprises the extracellular domain of PD-L1, PD-L2, CD80, CD86, HVEM, CD48, CD112, CD155, Ceacam1, FGL1, or Galectin-3 and the transmembrane polypeptide comprises GP41 transmembrane domain. In some embodiments, the immune inhibitory checkpoint polypeptide comprises the extracellular domain of PD-L1, PD-L2, CD80, CD86, HVEM, CD48, CD112, CD155, Ceacam1, FGL1, or Galectin-3 and the transmembrane polypeptide comprises GP120 transmembrane domain.

In some embodiments, the mammalian immune checkpoint polypeptide comprises an immune stimulatory checkpoint polypeptide. In some embodiments, the immune stimulatory checkpoint polypeptide comprises a polypeptide expressed on T cells. In some embodiments, the mammalian immune checkpoint polypeptide comprises a polypeptide expressed on antigen presenting cells. In some embodiments, the immune stimulatory checkpoint polypeptide comprises CD27, CD28, CD40, CD122, 4-1BB, ICOS, OX40, CD2, CD30, or GITR. In some embodiments, the immune stimulatory checkpoint polypeptide comprises CD70, CD80, CD86, CD40L, GITRL, 4-1BBL, OX40L, LIGHT, CD30L, CD48, or ICOSL. In some embodiments, the immune stimulatory checkpoint polypeptide comprises the extracellular domain of CD27, CD28, CD40, CD122, 4-1BB, ICOS, OX40, CD2, CD30, or GITR. In some embodiments, the immune stimulatory checkpoint polypeptide comprises the extracellular domain of CD70, CD80, CD86, CD40L, GITRL, 4-1BBL, OX40L, LIGHT, CD30L, CD48, or ICOSL.

In some embodiments, the immune stimulatory checkpoint polypeptide comprises CD27, CD28, CD40, CD122, 4-1BB, ICOS, OX40, CD2, CD30, or GITR and the transmembrane polypeptide comprises VSVG transmembrane domain. In some embodiments, the immune stimulatory checkpoint polypeptide comprises CD27, CD28, CD40, CD122, 4-1BB, ICOS, OX40, CD2, CD30, or GITR and the transmembrane polypeptide comprises spike protein S1 transmembrane domain. In some embodiments, the immune stimulatory checkpoint polypeptide comprises CD27, CD28, CD40, CD122, 4-1BB, ICOS, OX40, CD2, CD30, or GITR and the transmembrane polypeptide comprises spike protein S2 transmembrane domain. In some embodiments, the immune stimulatory checkpoint polypeptide comprises CD27, CD28, CD40, CD122, 4-1BB, ICOS, OX40, CD2, CD30, or GITR and the transmembrane polypeptide comprises the transmembrane domain of a surface glycoprotein of an enveloped virus. In some embodiments, the immune stimulatory checkpoint polypeptide comprises CD27, CD28, CD40, CD122, 4-1BB, ICOS, OX40, CD2, CD30, or GITR and the transmembrane polypeptide comprises the transmembrane domain of Sindbis virus envelope (SINDBIS) protein. In some embodiments, the immune stimulatory checkpoint polypeptide comprises CD27, CD28, CD40, CD122, 4-1BB, ICOS, OX40, CD2, CD30, or GITR and the transmembrane polypeptide comprises BaEV transmembrane domain. In some embodiments, the immune stimulatory checkpoint polypeptide comprises CD27, CD28, CD40, CD122, 4-1BB, ICOS, OX40, CD2, CD30, or GITR and the transmembrane polypeptide comprises GP41 transmembrane domain. In some embodiments, the immune stimulatory checkpoint polypeptide comprises CD27, CD28, CD40, CD122, 4-1BB, ICOS, OX40, CD2, CD30, or GITR and the transmembrane polypeptide comprises GP120 transmembrane domain.

In some embodiments, the immune stimulatory checkpoint polypeptide comprises CD70, CD80, CD86, CD40L, GITRL, 4-1BBL, OX40L, LIGHT, CD30L, CD48, or ICOSL and the transmembrane polypeptide comprises VSVG transmembrane domain. In some embodiments, the immune stimulatory checkpoint polypeptide comprises CD70, CD80, CD86, CD40L, GITRL, 4-1BBL, OX40L, LIGHT, CD30L, CD48, or ICOSL and the transmembrane polypeptide comprises spike protein S1 transmembrane domain. In some embodiments, the immune stimulatory checkpoint polypeptide comprises CD70, CD80, CD86, CD40L, GITRL, 4-1BBL, OX40L, LIGHT, CD30L, CD48, or ICOSL and the transmembrane polypeptide comprises spike protein S2 transmembrane domain. In some embodiments, the immune stimulatory checkpoint polypeptide comprises CD70, CD80, CD86, CD40L, GITRL, 4-1BBL, OX40L, LIGHT, CD30L, CD48, or ICOSL and the transmembrane polypeptide comprises the transmembrane domain of a surface glycoprotein of an enveloped virus. In some embodiments, the immune stimulatory checkpoint polypeptide comprises CD70, CD80, CD86, CD40L, GITRL, 4-1BBL, OX40L, LIGHT, CD30L, CD48, or ICOSL and the transmembrane polypeptide comprises the transmembrane domain of Sindbis virus envelope (SINDBIS) protein. In some embodiments, the immune stimulatory checkpoint polypeptide comprises CD70, CD80, CD86, CD40L, GITRL, 4-1BBL, OX40L, LIGHT, CD30L, CD48, or ICOSL and the transmembrane polypeptide comprises BaEV transmembrane domain. In some embodiments, the immune stimulatory checkpoint polypeptide comprises CD70, CD80, CD86, CD40L, GITRL, 4-1BBL, OX40L, LIGHT, CD30L, CD48, or ICOSL and the transmembrane polypeptide comprises GP41 transmembrane domain. In some embodiments, the immune stimulatory checkpoint polypeptide comprises CD70, CD80, CD86, CD40L, GITRL, 4-1BBL, OX40L, LIGHT, CD30L, CD48, or ICOSL and the transmembrane polypeptide comprises GP120 transmembrane domain.

In some embodiments, the immune stimulatory checkpoint polypeptide comprises the extracellular domain of CD27, CD28, CD40, CD122, 4-1BB, ICOS, OX40, CD2, CD30, or GITR and the transmembrane polypeptide comprises VSVG transmembrane domain. In some embodiments, the immune stimulatory checkpoint polypeptide comprises the extracellular domain of CD27, CD28, CD40, CD122, 4-1BB, ICOS, OX40, CD2, CD30, or GITR and the transmembrane polypeptide comprises spike protein S1 transmembrane domain. In some embodiments, the immune stimulatory checkpoint polypeptide comprises the extracellular domain of CD27, CD28, CD40, CD122, 4-1BB, ICOS, OX40, CD2, CD30, or GITR and the transmembrane polypeptide comprises spike protein S2 transmembrane domain. In some embodiments, the immune stimulatory checkpoint polypeptide comprises the extracellular domain of CD27, CD28, CD40, CD122, 4-1BB, ICOS, OX40, CD2, CD30, or GITR and the transmembrane polypeptide comprises the transmembrane domain of a surface glycoprotein of an enveloped virus. In some embodiments, the immune stimulatory checkpoint polypeptide comprises the extracellular domain of CD27, CD28, CD40, CD122, 4-1BB, ICOS, OX40, CD2, CD30, or GITR and the transmembrane polypeptide comprises the transmembrane domain of Sindbis virus envelope (SINDBIS) protein. In some embodiments, the immune stimulatory checkpoint polypeptide comprises the extracellular domain of CD27, CD28, CD40, CD122, 4-1BB, ICOS, OX40, CD2, CD30, or GITR and the transmembrane polypeptide comprises BaEV transmembrane domain. In some embodiments, the immune stimulatory checkpoint polypeptide comprises the extracellular domain of CD27, CD28, CD40, CD122, 4-1BB, ICOS, OX40, CD2, CD30, or GITR and the transmembrane polypeptide comprises GP41 transmembrane domain. In some embodiments, the immune stimulatory checkpoint polypeptide comprises the extracellular domain of CD27, CD28, CD40, CD122, 4-1BB, ICOS, OX40, CD2, CD30, or GITR and the transmembrane polypeptide comprises GP120 transmembrane domain.

In some embodiments, the immune stimulatory checkpoint polypeptide comprises the extracellular domain of CD70, CD80, CD86, CD40L, GITRL, 4-1BBL, OX40L, LIGHT, CD30L, CD48, or ICOSL and the transmembrane polypeptide comprises VSVG transmembrane domain. In some embodiments, the immune stimulatory checkpoint polypeptide comprises the extracellular domain of CD70, CD80, CD86, CD40L, GITRL, 4-1BBL, OX40L, LIGHT, CD30L, CD48, or ICOSL and the transmembrane polypeptide comprises spike protein S1 transmembrane domain. In some embodiments, the immune stimulatory checkpoint polypeptide comprises the extracellular domain of CD70, CD80, CD86, CD40L, GITRL, 4-1BBL, OX40L, LIGHT, CD30L, CD48, or ICOSL and the transmembrane polypeptide comprises spike protein S2 transmembrane domain. In some embodiments, the immune stimulatory checkpoint polypeptide comprises the extracellular domain of CD70, CD80, CD86, CD40L, GITRL, 4-1BBL, OX40L, LIGHT, CD30L, CD48, or ICOSL and the transmembrane polypeptide comprises the transmembrane domain of a surface glycoprotein of an enveloped virus. In some embodiments, the immune stimulatory checkpoint polypeptide comprises the extracellular domain of CD70, CD80, CD86, CD40L, GITRL, 4-1BBL, OX40L, LIGHT, CD30L, CD48, or ICOSL and the transmembrane polypeptide comprises the transmembrane domain of Sindbis virus envelope (SINDBIS) protein. In some embodiments, the immune stimulatory checkpoint polypeptide comprises the extracellular domain of CD70, CD80, CD86, CD40L, GITRL, 4-1BBL, OX40L, LIGHT, CD30L, CD48, or ICOSL and the transmembrane polypeptide comprises BaEV transmembrane domain. In some embodiments, the immune stimulatory checkpoint polypeptide comprises the extracellular domain of CD70, CD80, CD86, CD40L, GITRL, 4-1BBL, OX40L, LIGHT, CD30L, CD48, or ICOSL and the transmembrane polypeptide comprises GP41 transmembrane domain. In some embodiments, the immune stimulatory checkpoint polypeptide comprises the extracellular domain of CD70, CD80, CD86, CD40L, GITRL, 4-1BBL, OX40L, LIGHT, CD30L, CD48, or ICOSL and the transmembrane polypeptide comprises GP120 transmembrane domain.

Described herein, in some embodiments, are multivalent particles comprising a fusion protein that comprises a mammalian immune checkpoint polypeptide and a transmembrane polypeptide, wherein the multivalent particles further comprise an oligomerization domain.

In some embodiments, the oligomerization domain is a dimerization domain. In some embodiments, the dimerization domain comprises a leucine zipper dimerization domain. In some embodiments, the oligomerization domain is a trimerization domain. In some embodiments, the trimerization domain comprises a post-fusion oligomerization domain of viral surface protein. In some embodiments, the trimerization domain comprises a D4 post-fusion trimerization domain of VSV-G protein. In some embodiments, the trimerization domain comprises a Dengue E protein post-fusion trimerization domain. In some embodiments, the trimerization domain comprises a foldon trimerization domain. In some embodiments, the oligomerization domain is a tetramerization domain. In some embodiments, the tetramerization domain comprises an influenza neuraminidase stem domain.

In some embodiments, when the fusion protein is expressed on the surface of the multivalent particle, the oligomerization domain is outside of the multivalent particle. In some embodiments, when the fusion protein is expressed on the surface of the multivalent particle, the oligomerization domain is outside of the multivalent particle and adjacent to a signal peptide. In some embodiments, when the fusion protein is expressed on the surface of the multivalent particle, the oligomerization domain is inside of the multivalent particle. In some embodiments, when the fusion protein is expressed on the surface of the multivalent particle, the oligomerization domain is inside of the multivalent particle and adjacent to the transmembrane domain.

In some embodiments, the fusion protein comprises a signal peptide.

In some embodiments, domains of the fusion protein are arranged from the N-terminus to the C-terminus in the following orders: (a) signal peptide, mammalian immune checkpoint polypeptide, oligomerization domain, transmembrane domain, and cytosolic domain; (b) signal peptide, mammalian immune checkpoint polypeptide, transmembrane domain, oligomerization domain, and cytosolic domain; or (c) signal peptide, oligomerization domain, mammalian immune checkpoint polypeptide, transmembrane domain, and cytosolic domain. In some embodiments, domains of the fusion protein are arranged from the N-terminus to the C-terminus in the following order: signal peptide, mammalian immune checkpoint polypeptide, oligomerization domain, transmembrane domain, and cytosolic domain. In some embodiments, domains of the fusion protein are arranged from the N-terminus to the C-terminus in the following order: signal peptide, mammalian immune checkpoint polypeptide, transmembrane domain, oligomerization domain, and cytosolic domain. In some embodiments, domains of the fusion protein are arranged from the N-terminus to the C-terminus in the following order: signal peptide, oligomerization domain, mammalian immune checkpoint polypeptide, transmembrane domain, and cytosolic domain.

Disclosed herein are fusion proteins comprising a transmembrane domain, a cytosolic domain, a mammalian immune checkpoint polypeptide, and an oligomerization domain wherein when the fusion protein is expressed on the surface of a multivalent particle, the fusion protein is displayed in an oligomeric format.

In some embodiments, the immune checkpoint polypeptide comprises an amino acid sequence with at least 90% sequence identity to SEQ ID NO: 1 or 2, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 3 or 4, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 5 or 6, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 7 or 8, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 9 or 10, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 11 or 12, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 17 or 18, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 23 or 24, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 25 or 26, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 27 or 28, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 29 or 30, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 31 or 32, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 33 or 34, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 35 or 36, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 37 or 38, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 39 or 40, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 41 or 42, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 43 or 44, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 45 or 46, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 49 or 50, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 51 or 52, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 59 or 60, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 61 or 62, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 102 or 103, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 108 or 109, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 153 or 154, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69. In some embodiments, the immune checkpoint polypeptide comprises an amino acid sequence with at least 90% sequence identity to SEQ ID NO: 161 or 162, the transmembrane polypeptide comprises an amino acid sequence with at least 90% sequence identity to any one of SEQ ID NOs: 63, 79-83, and the oligomerization domain comprises an amino acid sequence with at least 90% sequence identity to any one of SEQ ID NOs: 65-69.

In some embodiments, the immune checkpoint polypeptide comprises an amino acid sequence with at least 90% sequence identity to SEQ ID NO: 47 or 48, the transmembrane polypeptide comprises an amino acid sequence with at least 90% sequence identity to SEQ ID NO: 84, and the oligomerization domain comprises an amino acid sequence with at least 90% sequence identity to SEQ ID NO: 73 or 74. In some embodiments, the immune checkpoint polypeptide comprises an amino acid sequence with at least 90% sequence identity to SEQ ID NO: 53 or 54, the transmembrane polypeptide comprises an amino acid sequence with at least 90% sequence identity to SEQ ID NO: 84, and the oligomerization domain comprises an amino acid sequence with at least 90% sequence identity to SEQ ID NO: 73 or 74. In some embodiments, the immune checkpoint polypeptide comprises an amino acid sequence with at least 90% sequence identity to SEQ ID NO: 110 or 111, the transmembrane polypeptide comprises an amino acid sequence with at least 90% sequence identity to SEQ ID NO: 84, and the oligomerization domain comprises an amino acid sequence with at least 90% sequence identity to SEQ ID NO: 73 or 74. In some embodiments, the immune checkpoint polypeptide comprises an amino acid sequence with at least 90% sequence identity to SEQ ID NO: 114 or 115, the transmembrane polypeptide comprises an amino acid sequence with at least 90% sequence identity to SEQ ID NO: 84, and the oligomerization domain comprises an amino acid sequence with at least 90% sequence identity to SEQ ID NO: 73 or 74. In some embodiments, the immune checkpoint polypeptide comprises an amino acid sequence with at least 90% sequence identity to SEQ ID NO: 157 or 158, the transmembrane polypeptide comprises an amino acid sequence with at least 90% sequence identity to SEQ ID NO: 84, and the oligomerization domain comprises an amino acid sequence with at least 90% sequence identity to SEQ ID NO: 73 or 74. In some embodiments, the immune checkpoint polypeptide comprises an amino acid sequence with at least 90% sequence identity to SEQ ID NO: 159 or 160, the transmembrane polypeptide comprises an amino acid sequence with at least 90% sequence identity to SEQ ID NO: 84, and the oligomerization domain comprises an amino acid sequence with at least 90% sequence identity to SEQ ID NO: 73 or 74.

In some embodiments, the fusion protein comprises an amino acid sequence of at least 75% sequence identity to an amino acid sequence according to SEQ ID NOs: 116-152. In some embodiments, the fusion protein comprises an amino acid sequence of at least 76% sequence identity to an amino acid sequence according to SEQ ID NOs: 116-152. In some embodiments, the fusion protein comprises an amino acid sequence of at least 77% sequence identity to an amino acid sequence according to SEQ ID NOs: 116-152. In some embodiments, the fusion protein comprises an amino acid sequence of at least 78% sequence identity to an amino acid sequence according to SEQ ID NOs: 116-152. In some embodiments, the fusion protein comprises an amino acid sequence of at least 79% sequence identity to an amino acid sequence according to SEQ ID NOs: 116-152. In some embodiments, the fusion protein comprises an amino acid sequence of at least 80% sequence identity to an amino acid sequence according to SEQ ID NOs: 116-152. In some embodiments, the fusion protein comprises an amino acid sequence of at least 81% sequence identity to an amino acid sequence according to SEQ ID NOs: 116-152. In some embodiments, the fusion protein comprises an amino acid sequence of at least 82% sequence identity to an amino acid sequence according to SEQ ID NOs: 116-152. In some embodiments, the fusion protein comprises an amino acid sequence of at least 83% sequence identity to an amino acid sequence according to SEQ ID NOs: 116-152. In some embodiments, the fusion protein comprises an amino acid sequence of at least 84% sequence identity to an amino acid sequence according to SEQ ID NOs: 116-152. In some embodiments, the fusion protein comprises an amino acid sequence of at least 85% sequence identity to an amino acid sequence according to SEQ ID NOs: 116-152. In some embodiments, the fusion protein comprises an amino acid sequence of at least 86% sequence identity to an amino acid sequence according to SEQ ID NOs: 116-152. In some embodiments, the fusion protein comprises an amino acid sequence of at least 87% sequence identity to an amino acid sequence according to SEQ ID NOs: 116-152. In some embodiments, the fusion protein comprises an amino acid sequence of at least 88% sequence identity to an amino acid sequence according to SEQ ID NOs: 116-152. In some embodiments, the fusion protein comprises an amino acid sequence of at least 89% sequence identity to an amino acid sequence according to SEQ ID NOs: 116-152. In some embodiments, the fusion protein comprises an amino acid sequence of at least 90% sequence identity to an amino acid sequence according to SEQ ID NOs: 116-152. In some embodiments, the fusion protein comprises an amino acid sequence of at least 91% sequence identity to an amino acid sequence according to SEQ ID NOs: 116-152. In some embodiments, the fusion protein comprises an amino acid sequence of at least 92% sequence identity to an amino acid sequence according to SEQ ID NOs: 116-152. In some embodiments, the fusion protein comprises an amino acid sequence of at least 93% sequence identity to an amino acid sequence according to SEQ ID NOs: 116-152. In some embodiments, the fusion protein comprises an amino acid sequence of at least 94% sequence identity to an amino acid sequence according to SEQ ID NOs: 116-152. In some embodiments, the fusion protein comprises an amino acid sequence of at least 95% sequence identity to an amino acid sequence according to SEQ ID NOs: 116-152. In some embodiments, the fusion protein comprises an amino acid sequence of at least 96% sequence identity to an amino acid sequence according to SEQ ID NOs: 116-152. In some embodiments, the fusion protein comprises an amino acid sequence of at least 97% sequence identity to an amino acid sequence according to SEQ ID NOs: 116-152. In some embodiments, the fusion protein comprises an amino acid sequence of at least 98% sequence identity to an amino acid sequence according to SEQ ID NOs: 116-152. In some embodiments, the fusion protein comprises an amino acid sequence of at least 99% sequence identity to an amino acid sequence according to SEQ ID NOs: 116-152. In some embodiments, the fusion protein comprises an amino acid sequence according to SEQ ID NOs: 116-152.

### Compositions for Generation of Immune Checkpoint Multivalent Particles

Described herein, in some embodiments, are compositions comprising a multivalent particle comprising a fusion protein that comprises a mammalian immune checkpoint polypeptide and a transmembrane polypeptide. In some embodiments, the compositions comprise a first nucleic acid sequence encoding the immune checkpoint multivalent particle described herein.

Compositions for generating multivalent particles, in some embodiments, further comprise a second nucleic acid sequence that encodes one or more viral proteins. In some embodiments, the one or more viral proteins is a lentiviral protein, a retroviral protein, an adenoviral protein, or combinations thereof. In some embodiments, the one or more viral proteins comprises gag, pol, pre, tat, rev, or combinations thereof.

Compositions for generating multivalent particles, in some embodiments, further comprise a second nucleic acid sequence that encodes an expression construct for specifically targeting the mammalian immune checkpoint polypeptide to the surface of an extracellular vesicle. In some embodiments, the second nucleic acid sequence encodes an expression construct for specifically targeting the mammalian immune checkpoint polypeptide to the surface of an exosome.

Compositions for generating multivalent particles, in some embodiments, further comprise a third nucleic acid sequence that encodes a replication incompetent viral genome, a reporter, a therapeutic molecule, or combinations thereof. In some embodiments, the viral genome is derived from vesicular stomatitis virus, measles virus, Hepatitis virus, influenza virus, or combinations thereof.

In some embodiments, the reporter protein is a fluorescent protein or an enzyme. Exemplary reporter genes include, but are not limited to, acetohydroxyacid synthase (AHAS), alkaline phosphatase (AP), beta galactosidase (LacZ), beta glucuronidase (GUS), chloramphenicol acetyltransferase (CAT), green fluorescent protein (GFP), red fluorescent protein (RFP), yellow fluorescent protein (YFP), cyan fluorescent protein (CFP), cerulean fluorescent protein, citrine fluorescent protein, orange fluorescent protein, cherry fluorescent protein, turquoise fluorescent protein, blue fluorescent protein, horseradish peroxidase (HRP), luciferase (Luc), nopaline synthase (NOS), octopine synthase (OCS), luciferase, and derivatives thereof. Methods to determine modulation of a reporter gene are well known in the art, and include, but are not limited to, fluorometric methods (e.g. fluorescence spectroscopy, Fluorescence Activated Cell Sorting (FACS), fluorescence microscopy), and antibiotic resistance determination. In some embodiments, the reporter is a fluorescent protein. In some embodiments, the fluorescent protein is green fluorescent protein. In some embodiments, the reporter protein emits green fluorescence, yellow fluorescence, or red fluorescence. In some embodiments, the reporter is an enzyme. In some embodiments, the enzyme is β-galactosidase, alkaline phosphatase, β-lactamase, or luciferase.

In some embodiments, the therapeutic molecule is a cellular signal modulating molecule, a proliferation modulating molecule, a cell death modulating molecule, or combinations thereof. In some embodiments, the therapeutic molecule is an inflammatory cytokine. In some embodiments, the inflammatory cytokine comprises IL-1, IL-12, IL-18, TNF-alpha, or TNF-beta. In some embodiments, the therapeutic molecule is a proliferation cytokine. In some embodiments, the proliferation cytokine comprises IL-2, IL-4, IL-7, or IL-15. In some embodiments, the cell death molecule comprises Fas or a death receptor.

In some embodiments, the first nucleic acid sequence, the second nucleic acid sequence, and the third nucleic acid sequence are within a same vector. In some embodiments, the first nucleic acid sequence, the second nucleic acid sequence, and the third nucleic acid sequence are within different vectors.

Various vectors, in some embodiments, are used herein. In some embodiments, the vector is a eukaryotic or prokaryotic vector. In some embodiments, the vector is a viral vector. In some embodiments, the vector is a lentivirus vector, an adenovirus vector, or an adeno-associated virus vector. Exemplary vectors include, without limitation, mammalian expression vectors: pSF-CMV-NEO-NH2-PPT-3×FLAG, pSF-CMV-NEO-COOH-3×FLAG, pSF-CMV-PURO-NH2-GST-TEV, pSF-OXB20-COOH-TEV-FLAG(R)-6His, pCEP4 pDEST27, pSF-CMV-Ub-KrYFP, pSF-CMV-FMDV-daGFP, pEFla-mCherry-N1 Vector, pEFla-tdTomato Vector, pSF-CMV-FMDV-Hygro, pSF-CMV-PGK-Puro, pMCP-tag(m), and pSF-CMV-PURO-NH2-CMYC; bacterial expression vectors: pSF-OXB20-BetaGal, pSF-OXB20-Fluc, pSF-OXB20, and pSF-Tac; plant expression vectors: pRI 101-AN DNA and pCambia2301; and yeast expression vectors: pTYB21 and pKLAC2, and insect vectors: pAc5.1/V5-His A and pDEST8.

### Compositions and Pharmaceutical Compositions

Described herein, in some embodiments, are compositions comprising a multivalent particle comprising a fusion protein that comprises a mammalian immune checkpoint polypeptide and a transmembrane polypeptide. Described herein, in some embodiments, are pharmaceutical compositions comprising a multivalent particle comprising a fusion protein that comprises a mammalian immune checkpoint polypeptide and a transmembrane polypeptide.

For administration to a subject, the immune checkpoint multivalent particles as disclosed herein, may be provided in a pharmaceutical composition together with one or more pharmaceutically acceptable carriers or excipients. In some embodiments, the immune checkpoint multivalent particles as disclosed herein, may be provided in a composition together with one or more carriers or excipients. The term “pharmaceutically acceptable carrier” includes, but is not limited to, any carrier that does not interfere with the effectiveness of the biological activity of the ingredients and that is not toxic to the patient to whom it is administered. Examples of suitable pharmaceutical carriers are well known in the art and include phosphate buffered saline solutions, water, emulsions, such as oil/water emulsions, various types of wetting agents, sterile solutions etc. Such carriers can be formulated by conventional methods and can be administered to the subject at a suitable dose. Preferably, the compositions are sterile. These compositions may also contain adjuvants such as preservative, emulsifying agents and dispersing agents. Prevention of the action of microorganisms may be ensured by the inclusion of various antibacterial and antifungal agents.

The pharmaceutical composition may be in any suitable form, (depending upon the desired method of administration). It may be provided in unit dosage form, may be provided in a sealed container and may be provided as part of a kit. Such a kit may include instructions for use. It may include a plurality of said unit dosage forms.

The pharmaceutical composition may be adapted for administration by any appropriate route, including a parenteral (e.g., subcutaneous, intramuscular, intravenous, or inhalation) route. Such compositions may be prepared by any method known in the art of pharmacy, for example by mixing the active ingredient with the carrier(s) or excipient(s) under sterile conditions.

Dosages of the substances of the present disclosure can vary between wide limits, depending upon the disease or disorder to be treated, the age and condition of the individual to be treated, etc. and a physician will ultimately determine appropriate dosages to be used.

### Methods of Use

Multivalent particles described herein, in some embodiments, immune checkpoint multivalent particles are used to treat cancer. In some embodiments, the cancer is a hematological malignancy. In some embodiments, the cancer is leukemia or lymphoma. In some embodiments, the lymphoma is B-cell lymphoma. In some embodiments, the cancer is a solid tumor. In some embodiments, the solid tumor is sarcoma, melanoma, breast cancer, lung cancer, pancreatic cancer, ovarian cancer, gastric cancer, brain cancer, or carcinoma. In some embodiments, the lung cancer is non-small cell lung cancer.

In some embodiments, administration of the immune checkpoint multivalent particles reduces or eliminates the cancer. In some embodiments, administration of the immune checkpoint multivalent particles increases anti-tumor immunity, increases cancer cell death, decreases tumor size, decreases cancer metastasis, or combinations thereof. In some embodiments, cell death is increased by about 1-fold to about 2.5-fold, about 1-fold to about 5-fold, about 2-fold to about 10-fold. In some embodiments, cell death is increased by at least 5-fold, at least 10-fold, at least 20-fold, at least 30-fold, at least 40-fold, at least 50-fold, at least 60-fold, at least 70-fold, at least 80-fold, at least 90-fold, at least 95-fold, 100-fold, or greater than 100-fold. In some embodiments, tumor size is decreased by about 1-fold to about 2.5-fold, about 1-fold to about 5-fold, about 2-fold to about 10-fold. In some embodiments, tumor size is decreased by at least 5-fold, at least 10-fold, at least 20-fold, at least 30-fold, at least 40-fold, at least 50-fold, at least 60-fold, at least 70-fold, at least 80-fold, at least 90-fold, at least 95-fold, 100-fold, or greater than 100-fold. In some embodiments, cancer metastasis is decreased by about 1-fold to about 2.5-fold, about 1-fold to about 5-fold, about 2-fold to about 10-fold. In some embodiments, cancer metastasis is decreased by at least 5-fold, at least 10-fold, at least 20-fold, at least 30-fold, at least 40-fold, at least 50-fold, at least 60-fold, at least 70-fold, at least 80-fold, at least 90-fold, at least 95-fold, 100-fold, or greater than 100-fold.

In some embodiments, administration of the immune checkpoint multivalent particles reduces or eliminates the cancer as compared to a level prior to administration of the immune checkpoint multivalent particles in the subject. In some embodiments, administration of the immune checkpoint multivalent particles reduces or eliminates the cancer as compared to a level if the subject had not received the immune checkpoint multivalent particles. In some embodiments, administration of the immune checkpoint multivalent particles reduces or eliminates the cancer as compared to a level if the subject had received a different cancer treatment including but not limited to, radiation, surgery, and chemotherapy.

In some embodiments, the immune checkpoint multivalent particles induce T cell mediated cytotoxicity against tumor cells. In some embodiments, the immune checkpoint multivalent particles inhibit T cell mediated cytotoxicity against normal tissues.

Multivalent particles described herein, in some embodiments, are used to treat an autoimmune disease. In some embodiments, the autoimmune disease is rheumatoid arthritis, systemic lupus erythematosus, multiple sclerosis, inflammatory bowel diseases, psoriasis, or aplastic anemia.

In some embodiments, administration of the immune checkpoint multivalent particles dampens or inhibits autoimmune responses as compared to a level prior to administration of the multivalent particles in the subject. In some embodiments, administration of the immune checkpoint multivalent particles dampens or inhibits autoimmune responses as compared to a level if the subject had not received the multivalent particles. In some embodiments, administration of the immune checkpoint multivalent particles dampens or inhibits autoimmune responses as compared to a level if the subject had received a different treatment.

In some instances, the subject is a mammal. In some instances, the subject is a mouse, rabbit, dog, pig, cattle, or human. Subjects treated by methods described herein may be infants, adults, or children. In some embodiments, the multivalent particles are administered by inhalation, injection, ingestion, transfusion, implantation or transplantation. In some embodiments, the multivalent particles are administered transarterially, subcutaneously, intradermally, intratumorally, intranodally, intramedullary, intramuscularly, by intravenous (i.v.) injection, or intraperitoneally. In some embodiments, the multivalent particles are administered intravenously. In some embodiments, the multivalent particles are administered by inhalation. In some embodiments, the multivalent particles are administered by an intraperitoneal injection. In some embodiments, the multivalent particles are administered by a subcutaneous injection.

The following examples are set forth to illustrate more clearly the principle and practice of embodiments disclosed herein to those skilled in the art and are not to be construed as limiting the scope of any claimed embodiments. Unless otherwise stated, all parts and percentages are on a weight basis.

### EXAMPLES

The following examples are given for the purpose of illustrating various embodiments of the disclosure and are not meant to limit the present disclosure in any fashion. The present examples, along with the methods described herein are presently representative of preferred embodiments, are exemplary, and are not intended as limitations on the scope of the disclosure. Changes therein and other uses which are encompassed within the spirit of the disclosure as defined by the scope of the claims will occur to those skilled in the art.

### Example 1: Generation and Characterization of Multivalent Immune Checkpoint Particles (IC-MVPs)

This example describes generation of multivalent immune checkpoint particles (IC-MVPs) that express immune stimulatory molecules or immune inhibitory molecules.

### Design of IC-MVP Display Vectors

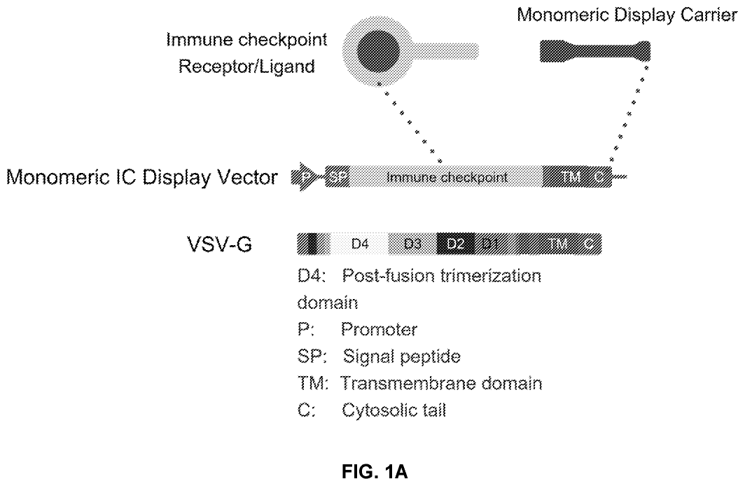

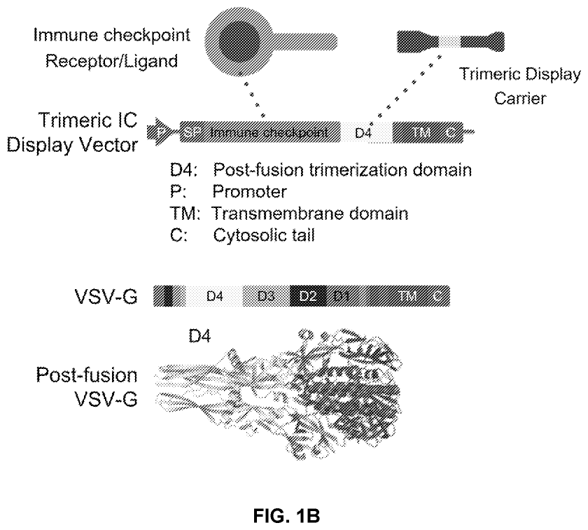

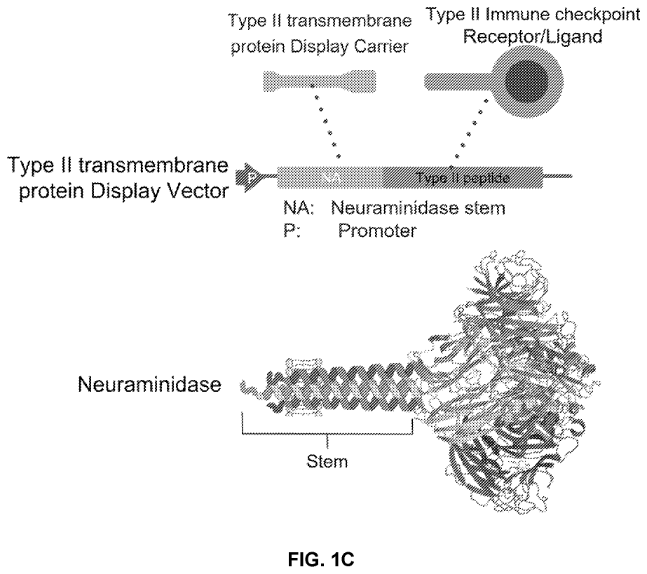

Three different types of IC-MVP display vectors were designed for displaying immune checkpoints on vesicles in various oligomeric forms (FIGS. 1A-1C). For displaying immune checkpoints in monomeric form, the display vector expressed a fusion protein comprising the extracellular domain of the desired immune checkpoint linked to the VSV-G protein transmembrane and intracellular domains (FIG. 1A). For displaying immune checkpoints in trimeric form, the vector expressed a fusion protein comprising the extracellular domain of the desired immune checkpoint linked to the D4 post-fusion trimerization domain, the transmembrane domain and the intracellular domain of VSV-G (FIG. 1B). For immune checkpoints that are type II transmembrane proteins, the vector expressed a fusion protein comprising the Influenza neuraminidase stem and transmembrane domains, followed by the extracellular domain of the type II immune checkpoint, and the fusion protein formed tetramers (FIG. 1C). These vectors can be used to produce monomeric, trimeric or tetrameric IC-MVPs.

### Generation of Monomeric IC-MVPs

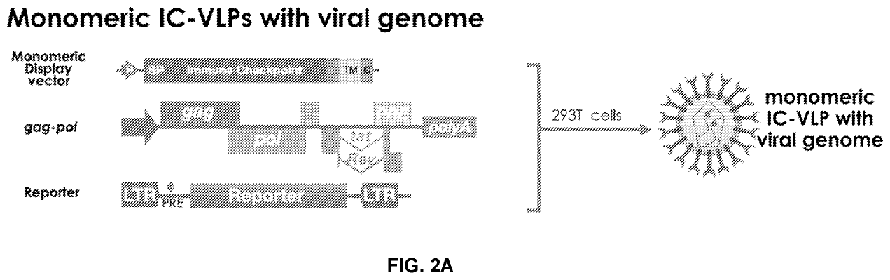

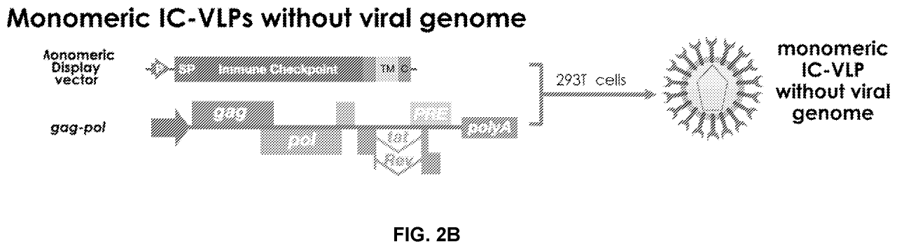

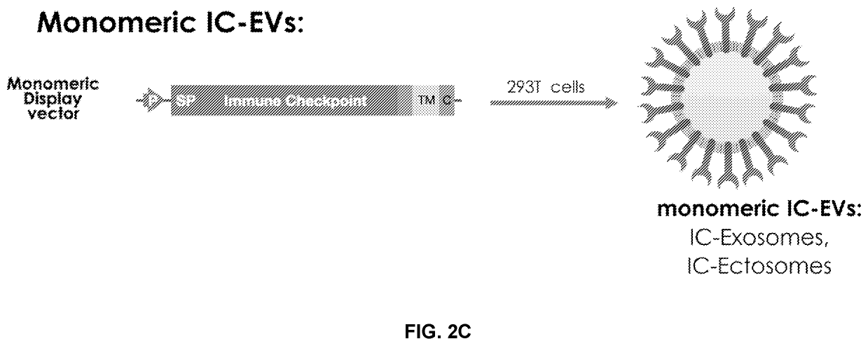

Multivalent immune checkpoints can be displayed as monomers on the surface of viral-like particles (VLP) and extracellular vesicles (EV), such as exosomes and ectosomes, using the monomeric display vector. To produce monomeric immune checkpoint VLPs (IC-VLPs) with viral RNA genomes, the monomeric immune checkpoint fusion construct was co-transfected into the HEK 293T cells with a lentiviral packaging construct expressing essential packaging components, such as Gag-Pol and Rev proteins, and a viral genome transfer vector encoding a GFP/luciferase reporter (FIG. 2A). Alternatively, monomeric IC-VLPs without RNA genome were produced by co-transfecting displaying vector together with only a lentiviral packaging construct but not the viral genome transfer vector (FIG. 2B). Finally, monomeric immune checkpoint extracellular vesicles (IC-EV), including IC-Exosomes and IC-Ectosomes, were produced by transfecting only the monomeric immune checkpoint displaying vector into 293T cells (FIG. 2C).

### Generation of Trimeric IC-MVPs

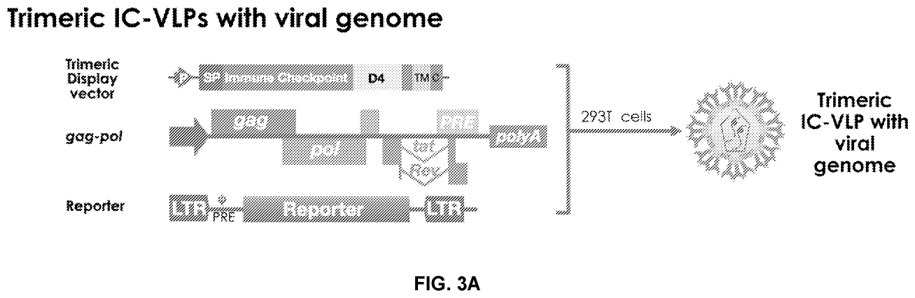

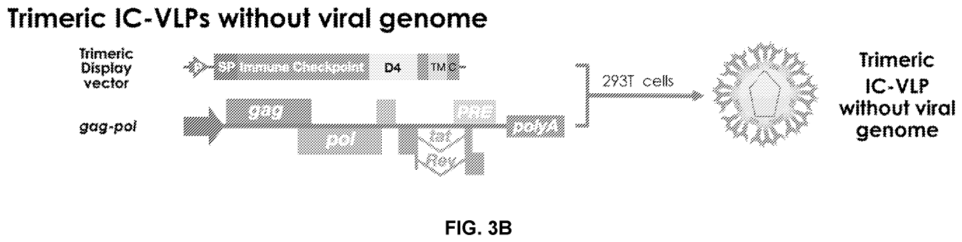

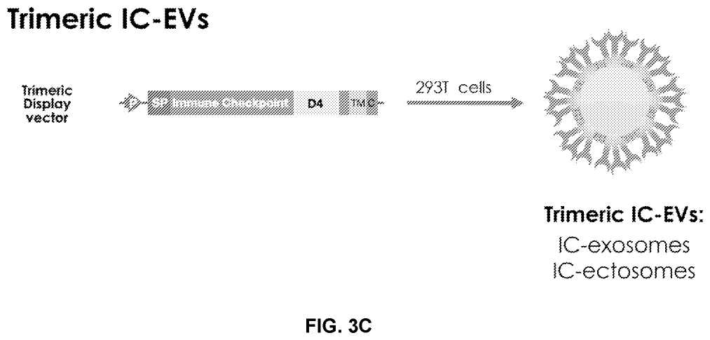

Multivalent immune checkpoints can be displayed as trimers on the surface of viral-like particle (VLP) and extracellular vesicle (EV), such as exosomes and ectosomes, by using the trimeric display vector. To produce trimeric VLP-ICs with viral RNA genomes, the trimeric immune checkpoint fusion construct was co-transfected into the HEK 293T cells with a lentiviral packaging construct expressing essential packaging components, such as Gag-Pol and Rev proteins, and a viral genome transfer vector encoding a GFP/luciferase reporter (FIG. 3A). Alternatively, trimeric VLP-ICs without RNA genome were produced by co-transfecting display vector together with only a lentiviral packaging construct but not the viral genome transfer vector (FIG. 3B). Finally, trimeric IC-EVs, including IC-Exosomes and IC-Ectosomes, were produced by transfecting only the trimeric immune checkpoint display vector into 293T cells (FIG. 3C).

### Generation of Mixed Monomeric and Trimeric IC-MVPs

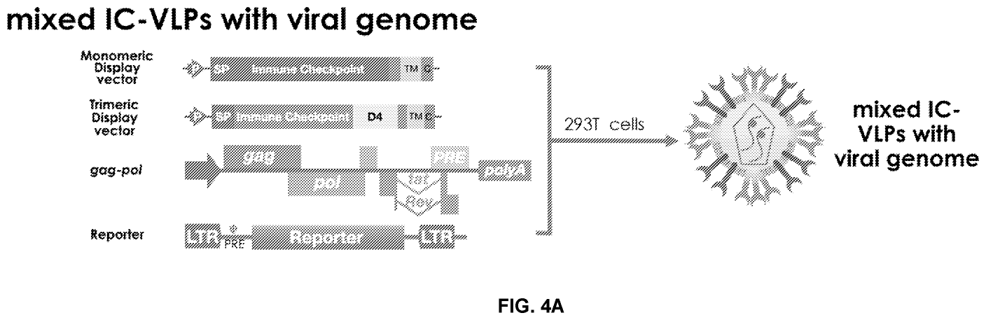

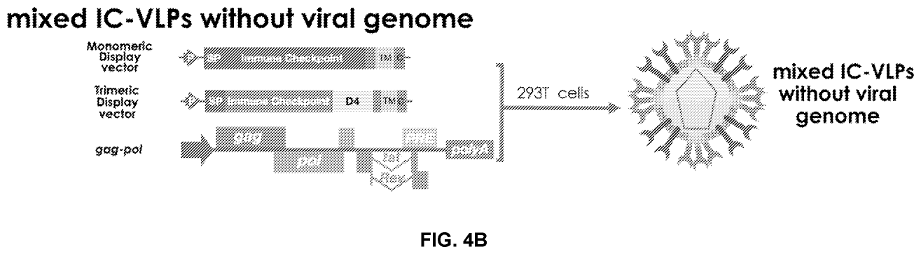

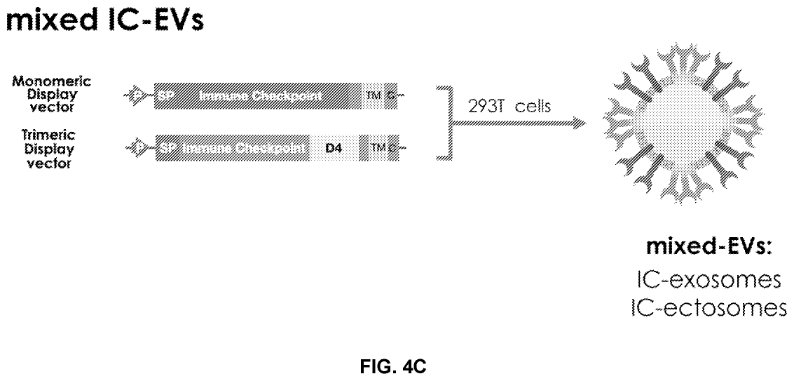

MVPs displaying mixed monomeric and trimeric immune checkpoints were generated by co-transfecting HEK 293T cells with monomeric and trimeric immune checkpoint display constructs. Such design can be used to increase the display density of an immune checkpoint or to create combinatorial displays of distinct immune checkpoint molecules. Mixed monomeric and trimeric IC-MVPs can be built with viral-like particles (VLP) and extracellular vesicles (EV), such as exosomes and ectosomes, by co-transfecting monomeric and trimeric display vectors. To produce mixed IC-VLPs with viral RNA genomes, the mixed monomeric and trimeric immune checkpoint fusion constructs were co-transfected into the 293T cells with a lentiviral packaging construct expressing essential packaging components, such as Gag-Pol and Rev proteins, and a viral genome transfer vector encoding a GFP/luciferase reporter (FIG. 4A). Alternatively, mixed IC-VLPs without RNA genome were produced by co-transfecting the mixed monomeric and trimeric display vector together with only a lentiviral packaging construct but not the viral genome transfer vector (FIG. 4B). Finally, mixed IC-EVs, including mixed IC-Exosomes and IC-Ectosomes, were produced by transfecting the mixed monomeric and trimeric immune checkpoint fusion constructs into 293T cells (FIG. 4C).

### Peptide Display Configurations on IC-MVPs

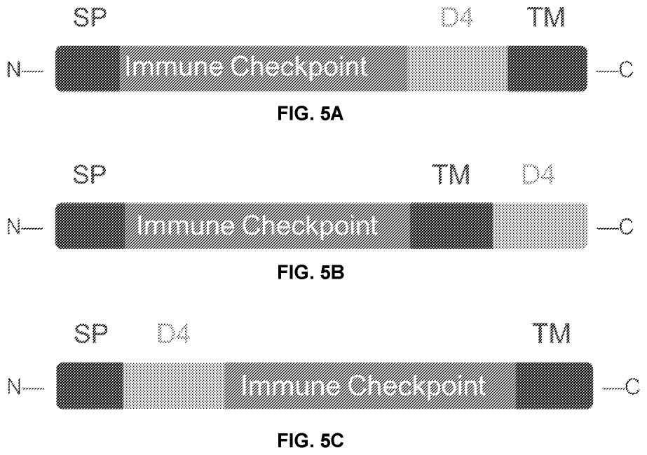

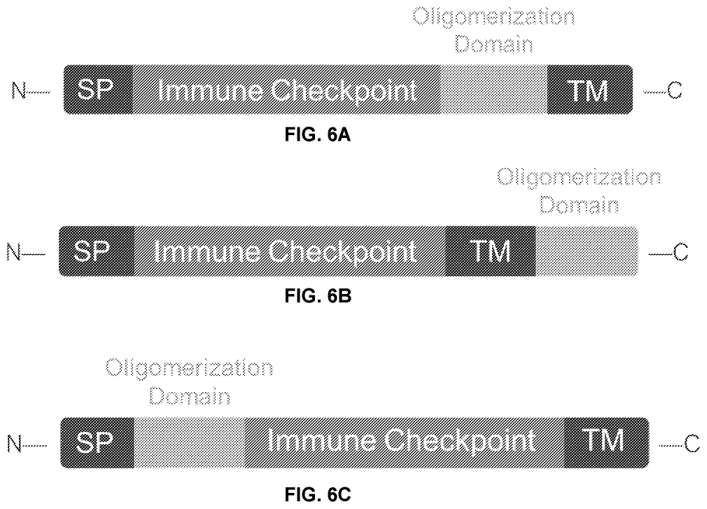

IC-MVPs can be genetically programmed to display immune checkpoints in various configurations by modifying the display vector (FIGS. 5A-5C, 6A-6C, Table 3). The VSV-G D4 trimerization domain can be placed at various positions of the fusion peptide: extracellular and juxtaposed to the transmembrane domain (FIG. 5A); (2) intracellular and juxtaposed to the transmembrane domain (FIG. 5B); (3) extracellular and after the signal peptide (FIG. 5C). Furthermore, various oligomerization domains may be used for distinct surface display patterns that are suitable for the function of immune checkpoint molecules (FIGS. 6A-6C, Table 3). In addition to the VSV-G D4 trimerization domain, the Dengue E protein post-fusion trimerization domain or the T4 phage Foldon domain can also be used to create trimeric display patterns on the surface of VLPs and EVs. Leucine zipper domains and the Influenza neuraminidase stem domain can be used to create dimeric and tetrameric display patterns on the surface of VLPs and EVs, respectively. Exemplary oligomerization domains and valence are summarized in Table 3. With these displaying configurations, combinatorial IC-MVPs can be programmed with mixed monomeric, dimeric, trimeric, and tetrameric immune checkpoint display patterns optimized for the displayed checkpoint's function in T cell regulation.

TABLE 3
Exemplary oligomerization domains and valence

| Oligomerization Domain | Valence |
|---|---|
| VSV-G protein D4 | Trimer |
| Dengue E protein fusion protein | Trimer |
| Foldon | Trimer |
| Leucine Zipper | Dimer |
| Influenza Neuraminidase stem | Tetramer |

### Characterization of Immune Checkpoint Display on IC-MVPs

The concentration of VLP- or EV-based IC-MVPs was measured by P24 ELISA or tunable resistive pulse sensing (TRPS, qNano), respectively. The number of copies of immune checkpoints displayed on MVPs was determined by quantitative Western-blot analysis. The oligomerization patterns of immune checkpoint displayed on the MVPs was discerned by non-reducing PAGE analyses. IC-MVPs displaying at least 10 copies of immune checkpoint molecules on the surface of VLPs and EVs were generated with monomeric or trimeric configurations.

### Binding of IC-MVPs to Target Cells Expressing Cognate Receptor/Ligand

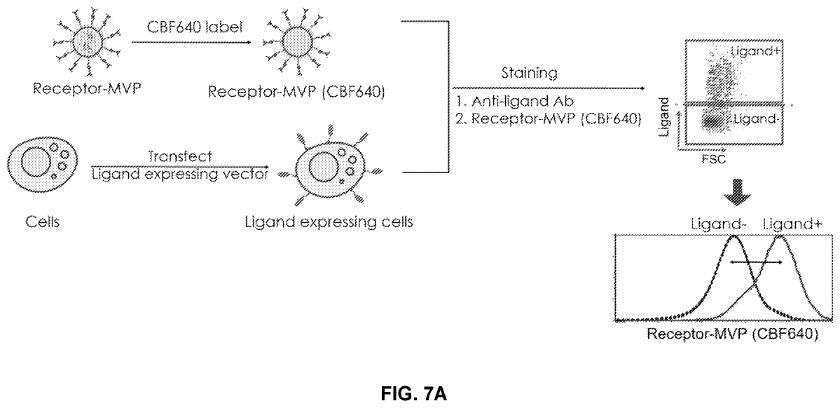

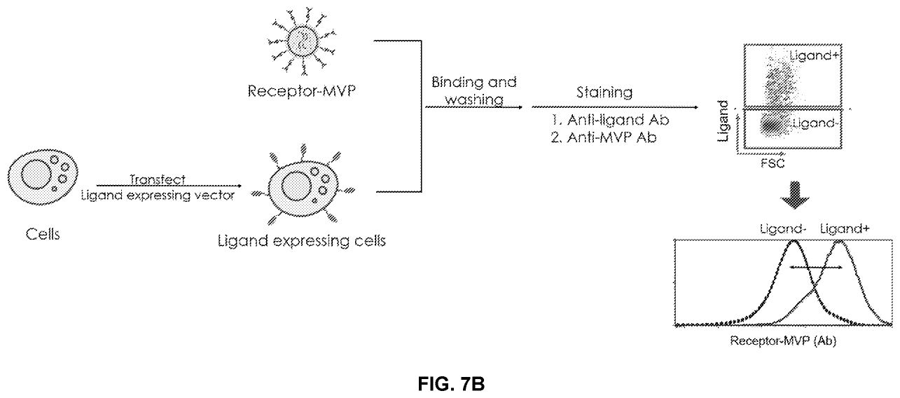

To confirm that IC-MVPs display functional immune checkpoint molecules, it was tested whether IC-MVPs can bind to target cells expressing cognate receptors or ligands using fluorescence-activated cell sorting (FACS)-based analyses (FIGS. 7A, 7B). Two different approaches were used to evaluate the specific interaction between IC-MVPs and target cells. In the first approach (FIG. 7A), target cell lines were established by transfecting 293T cells with constructs expressing the cognate ligand or receptor for the immune checkpoint molecules expressed on IC-MVPs. IC-MVPs were then labelled with CBF640 or other compatible fluorescent dyes. Transfected 293T cells were stained with dye-labelled IC-MVPs and antibodies specific for the ligand. Finally, specific-binding of IC-MVPs to target cells expressing its cognate ligand or receptor was analyzed by FACS. In the second approach (FIG. 7B), transfected target cells were stained with unlabeled IC-MVPs, and then target cells were stained with fluorescent antibodies specific for the immune checkpoint and its ligand. Again, specific-binding of IC-MVPs to target cells expressing its cognate ligand or receptor was analyzed by FACS. In some cases, when the expression of cognate receptor or ligand on T cells was confirmed, T cells were also stained with dye-labelled IC-MVPs and specific-binding of IC-MVPs to T cells was analyzed. These approaches provided confirmation of functional immune checkpoint expression on IC-MVPs and insight into how to optimize copy number and oligomerization patterns of immune checkpoints to enhance IC-MVP interactions with target cells.

### Control of T Cell Activation, Proliferation, Differentiation, and Apoptosis by IC-MVPs

Both stimulatory and inhibitory immune checkpoints play critical roles in regulating T cell activation, proliferation, apoptosis and differentiation. The following assays were designed to interrogate the effects of IC-MVPs on T cells. T cells activated with anti-CD3 antibody were treated with various concentrations of IC-MVPs. The potential activating or inhibitory effects of IC-MVPs on T cell activation can be read out at day 2 post-activation by examining CD69 and CD25—early T cell activation markers—expression on treated T cells. Alternatively, Pmel T cells stimulated with dendritic cells loaded with GP100 peptide antibody were treated with various concentrations of IC-MVPs. The potential activating or inhibitory effects of IC-MVPs on antigen-specific T cell activation can be read out at day 2 post-activation by examining CD69 and CD25—early T cell activation markers—expression on treated T cells. Furthermore, effects of IC-MVPs on T cell proliferation can be determined by monitoring cell counts in treated cell cultures for 8-10 days, and the effects of IC-MVPs on effector and memory T cell differentiation can be determined by FACS analyses of CD62L and CD44 expression in treated cell cultures. Finally, at 8-10 days post-activation, cultured T cells were stained with PI and 7-AAD to determine the effects of IC-MVPs on cultured T cell apoptosis.

### Control of Cytotoxic T Cells (CTL) Activity by IC-MVPs

To interrogate the activity of IC-MVPs in controlling cytotoxic T cells (CTL), how IC-MVPs perturb the cytolytic activity of Pmel T cells against B16F0 melanoma cells was examined. Pmel T cells bear transgenic T cell receptors (TCRs) recognizing the gp100 peptide EGSRNQDWL bound to MHC-I H2-Db presented on B16F0 melanoma cells. Furthermore, whether IC-MVP treatment of T cells enhanced the expression of Granzyme A and Perforin in treated T cells was examined by intracellular staining and FACS analyses. Granzyme A and Perforin are two important proteins in the granule exocytosis pathway for T cell and NK cell-mediated cell killing. Finally, whether IC-MVP treated T cells express elevated levels of inflammatory cytokines, such as IFN-γ and TNF-α, was examined by intracellular staining and FACS analyses. T cells with higher levels of IFN-γ and TNF-α have enhanced inflammatory functions.

### Control of Tumor Development by IC-MVPs

Syngeneic mouse tumor models for lung, breast, pancreatic, and melanoma cancer were used to examine the effects of IC-MVPs on tumor development. Purified IC-MVPs were injected into mice after tumor implantation through tail-vein injection. Mice were repeatedly dosed with IC-MVPs every 3 days for 6 times. Tumors were measured at various time points after treatment to determine whether IC-MVPs can potentiate or inhibit tumor growth in vivo. The effects of IC-MVPs on tumor control were compared to positive control checkpoint blockade antibodies, such as anti-PD-1 or anti-CTLA-4 antibody. The tumor control functions of IC-MVPs displaying individual immune checkpoints was first examined, then IC-MVPs displaying combinations of immune checkpoints were tested which can further enhance the tumor controlling abilities of IC-MVPs.

### Modulating ARDS by IC-MVPs

Acute respiratory distress syndrome (ARDS) was used as an inflammation model. It was examined whether inhibitory IC-MVPs can be used to control and reduce the damage caused by systemic inflammation. The excessive proinflammatory responses that lead to ARDS may be initiated and driven by Toll-like receptors (TLRs), which recognize pathogen-derived constituents such as lipopolysaccharide (LPS), bacteria lipoproteins, and non-methylated CpG DNA, leading to rapid escalation of systemic immune responses. Such conditions can be partially recapitulated in a mouse model of LPS-induced systemic inflammation. In this lethal model, untreated mice reached experimental endpoint within 72 hours. If IC-MVP treatment can save mice from the lethality, it would demonstrate that IC-MVPs can effectively dampen systemic inflammation induced by LPS.

### Materials and Methods

### Immune Checkpoint Displaying Constructs.

Codon-optimized immune checkpoint sequences were synthesized (Twist) and cloned into a display construct to create fusion peptides consisting of the extracellular domain of an immune checkpoint and a display anchoring protein. To generate MVPs displaying monomeric immune checkpoints, the extracellular domains of immune checkpoints were fused to a synthetic VSV-G sequence encoding the transmembrane and cytoplasmic tail domains. To generate MVPs displaying oligomerized immune checkpoints, the extracellular domains of immune checkpoints were fused to a synthetic VSV-G sequence encoding the D4 post-fusion trimerization domain and the transmembrane and cytoplasmic tail domains.

### Production of IC-MVPs Based on VLPs or Extracellular Vesicles

IC-MVPs based on VLPs or extracellular vesicles were produced from transfected 293T cells. To produce lentiviral-VLP based IC-MVPs with viral genomes, immune checkpoint display construct, lentiviral packaging vector (i.e. psPAX2), and lentiviral genome transfer vector were co-transfected into 293T cells. To produce lentiviral-VLP based IC-MVPs without viral genomes, immune checkpoint displaying construct and lentiviral packaging vector (i.e. psPAX2) were co-transfected into 293T cells. Finally, to produce extracellular vesicle-based IC-MVPs, only immune checkpoint displaying construct was transfected into 293T cells.

In preparation for transfection, 7.5×10⁶ HEK293T cells (ATCC CRL-3216) were seeded overnight in 10-cm dishes containing DMEM media with glucose, L-glutamine and sodium pyruvate (Corning) supplemented with 10% fetal bovine serum (Sigma) and 1% Penicillin Streptomycin (Life Technologies), referred to as “293T Growth Media.” Cells should reach about 90% confluence the next day at the time of transfection. The following day, transfection DNA mixture along with polyethylenimine (PEI) in OPTI-MEM reduced serum medium (Gibco) was prepared. Transfection mixture was incubated at room temperature for 15 minutes before being added to cells, which were then incubated at 37° C. in 5% CO2. 6 hours post-transfection, 293T Growth Media was changed to 293T Growth Media supplemented with 0.1% sodium butyrate (referred to as “Transfection Media”) before being returned to incubation. After incubating for 24 hours at 37° C. with 5% CO2 in Transfection Media, supernatant containing pseudovirus was collected, centrifuged at 1680 rpm for 5 minutes to remove cellular debris and mixed with 1× polyethylene glycol 8000 solution (PEG, Hampton Research), before being stored at 4° C. for 24 hours to allow fractionation. Cells were replenished with fresh Transfection Media, and a second pseudovirus supernatant collection was performed at 48 hours. Supernatant collections were then pooled, PEG precipitated and purified by size exclusion chromatography using Sephacryl S-300 High Resolution Beads (Sigma Aldrich).

### Lentiviral Particle Quantification by p24 ELISA and Tunable Resistive Pulse Sensing

P24 concentrations in pseudovirus samples of pseudotyped coronaviruses, influenza viruses and antibody-based antivirus particles were determined using an HIV p24 SimpleStep ELISA kit (Abcam) per the manufacturer's protocol. Concentrations of lentiviral pseudovirus particles were extrapolated from the assumption that each lentiviral particle contains approximately 2000 molecules of p24, or 1.25×10⁴ pseudovirus particles per picogram of p24 protein.

Pseudovirus concentrations determined via p24 ELISA were corroborated by tunable resistive pulse sensing (TRPS, qNano, IZON). Purified pseudovirus collections were diluted in 0.2 μm filtered phosphate buffered saline (PBS) with 0.03% Tween-20 (Thermo Fisher Scientific) prior to qNano analysis. Concentration and size distributions of pseudotyped particles were then determined using an NP200 nanopore at a 45.5 mm stretch, and applied voltages between 0.5 and 0.7V were used to achieve a stable current of 130 nA through the nanopore. Measurements for each pseudovirus sample were taken at pressures of 3, 5 and 8 mbar, and considered valid if at least 500 events were recorded, particle rate was linear and root mean squared signal noise was maintained below 10 pA. Pseudovirus concentrations were then determined by comparison to a standardized multi-pressure calibration using CPC200 (mode diameter: 200 nm) (IZON) carboxylated polystyrene beads diluted 1:200 in 0.2 μM filtered PBS from their original concentration of 7.3×10¹¹ particles per/mL. Measurements were analyzed using IZON Control Suite 3.4 software to determine original sample concentrations.

### Quantification of Lentiviral VLP-Based IC-MVPs

P24 concentrations of the IC-MVP samples were determined by using the Abcam HIV P24 SimpleStep ELISA kit following manufacturer's instruction. The concentrations of lentiviral pseudovirion particles were derived based on the assumption that each lentiviral particle contains about ˜2000 molecules of P24 or 1.25×10⁴ viral particles/picogram of P24 protein.

### Quantification of Extracellular Vesicle-Based IC-MVPs

The sizes and concentrations of extracellular vesicle-based IC-MVPs were determined by tunable resistive pulse sensing (TRPS, qNano, IZON). Purified pseudovirus collections were diluted in 0.2 μm filtered PBS with 0.03% Tween-20 (Thermo Fisher Scientific) prior to qNano analysis. Concentration and size distributions of IC-MVPs were then determined using an NP200 nanopore at a 45.5 mm stretch, and applied voltages between 0.5 and 0.7 V were used to achieve a stable current of 130 nA through the nanopore. Measurements for each pseudovirus sample were taken at pressures of 3, 5 and 8 mbar, and considered valid if at least 500 events were recorded, particle rate was linear and root mean squared signal noise was maintained below 10 pA. IC-MVPs concentrations were then determined by comparison to a standardized multi-pressure calibration using CPC200 (mode diameter: 200 nm) (IZON) carboxylated polystyrene beads diluted 1:200 in 0.2 μM filtered PBS from their original concentration of 7.3×10¹¹ particles per/mL. Measurements were analyzed using IZON Control Suite 3.4 software to determine original sample concentrations.

### Western Blot Analysis of IC-MVPs

Expression of immune checkpoint fusion proteins on MVPs was confirmed via western blot analysis of purified particles. Samples of purified IC-MVPs were lysed at 4° C. for 10 minutes with cell lysis buffer (Cell Signaling) before being mixed with NuPage LDS sample buffer (Thermo Fisher Scientific) and boiled at 95° C. for 5 minutes. Differences in oligomerization were determined by running samples in reducing and non-reducing conditions. Under reducing conditions, 5% 2-Mercaptoethanol (Thermo Fisher Scientific) was added to samples to dissociate oligomerized IC-MVPs. Protein samples were then separated on NuPAGE 4-12% Bis-Tris gels (Thermo Fisher Scientific) and transferred onto a polyvinylidene fluoride (PVDF) membrane (Life Technologies). PVDF membranes were blocked with TRIS-buffered saline with Tween-20 (TBST) and 5% skim milk (Research Products International) for 1 hour, prior to overnight incubation with primary antibody diluted in 5% milk. For immune checkpoint fusion constructs expressing VSVG-tag, an anti-VSV-G epitope tag rabbit polyclonal antibody (BioLegend, Poly29039) was used at a 1:2000 dilution. The following day, the PVDF membrane was washed 3 times with 1×TBST and stained with a goat-anti-rabbit secondary antibody (IRDye 680) at a 1:5000 dilution for 60 minutes in 5% milk. Post-secondary antibody staining, the PVDF membrane was again washed 3 times with TBST before imaging on a Licor Odyssey scanner.

Alternatively, western blot analyses were performed using an automated Simple Western size-based protein assay (Protein Simple) following the manufacturer's protocols. Unless otherwise mentioned, all reagents used here were from Protein Simple. Concentrated samples were lysed as described above, before being diluted 1:10 in 0.1× sample buffer for loading on capillaries. Immune checkpoint fusion protein expression levels were identified using the same primary rabbit polyclonal antibody at a 1:400 dilution and an HRP conjugated anti-rabbit secondary antibody (Protein Simple). Chemiluminescence signal analysis and absolute quantitation were performed using Compass software (Protein Simple).

### Quantitative Western Blot Analyses

Quantitative western blot analyses were performed to determine the copies of immune checkpoint fusion protein displayed per particle. P24 ELISA or TRPS (qNano) assays were used to determine the IC-MVP sample concentrations. Purified IC-MVP samples were processed and analyzed via western blot under reducing conditions as described above. A reference decoy-MVP with a known display copy number was used to generate a standard curve, from which copy numbers of displayed immune checkpoint on respective particles were determined.

### Binding of IC-MVPs to Target Cells

To verify the specific binding between IC-MVPs, purified IC-MVPs were stained with CSFE or other fluorescent dyes and then passed through a size exclusion column to remove unbound dyes. T cells or 293T cells transfected with cognate immune checkpoint ligands or receptors were incubated with dye-labelled IC-MVPs at room temperature for 30 minutes. Stained cells were then washed with FACS buffer and analyzed on flow cytometer to determine specific-binding of IC-MVPs with target cells.

### Effects of IC-MVPs on T Cells Activation, Proliferation, Apoptosis, and Differentiation

Purified mouse spleen T cells or human peripheral blood T cells were used to examine the effects of IC-MVPs on T cell activation, proliferation, apoptosis, and differentiation. T cells stimulated with suboptimal dose of anti-CD3 antibody were treated various concentration of IC-MVPs. Alternatively, Pmel T cells stimulated with dendritic cells loaded with GP100 peptides antibody were treated various concentration of IC-MVPs. At day 2 or 3 posted IC-MVP treatment, cells were analyzed by FACS to determine the expression of early activation markers CD69 and CD25. Cell counts were monitored for 8-10 days to determine the effects of IC-MVPs on T cell proliferation. The composition of effector and memory cells was quantified by FACS analyses of CD62L and CD44 expression to determine the effect of IC-MVPs on T cell differentiation. Finally, at 8-10 days post-activation, cultured T cells were stained with PI and 7-AAD to determine the effects of IC-MVP on cultured T cell apoptosis.

### Effects of IC-MVPs on CTL

To determine the effects of IC-MVPs on the ability of CD8 T cells to kill tumor cells, CD8 T cells were purified from Pmel mice expressing a transgenic T cell receptor (TCR) that specifically recognize gp100 peptide EGSRNQDWL bound to MHC-I H2-Db. Pmel T cells were then activated by incubation with EGSRNQDWL loaded (2 ug/ml) bone marrow-derived dendritic cells (2×10⁵ cells/well). The activated cells were treated with PBS (as a control) or IC-MVPs with or without PD-L1 antibody blocking and then co-cultured with CellTrace™ Violet dye-labelled B16-F0 cells for 48 hours at the effector to target ratio (E:T) of 1:1. Cells were harvested, labelled with 7-aminoactinomycin D (7-AAD, BD Pharmingen), and analyzed by FACS to determine the killing of target cells by T cells. The population of CellTrace™ Violet dye+/7-AAD+ cells represented the target cells that have been killed and CellTrace™ Violet dye+/7-AAD− population represented the remaining viable target cells. Percentage of specific lysis was calculated by using the formula: specific lysis (%)=(CellTrace™ Violet dye+/7-AAD+)/(CellTrace™ Violet dye+/7-AAD+ plus CellTrace™ Violet dye+/7-AAD−)−target/CTV/7AAD background ratio.

### Effects of IC-MVPs on Tumor Development

Syngeneic mouse tumor models for lung, breast, pancreatic, and melanoma cancer were used to examine the effects of IC-MVPs on tumor development. Tumor cells were cultured and expanded before implantation. To generate melanoma models, 1×10⁵ B16F0 cells were injected subcutaneously into 6 to 8 week-old female C57BL/6 mice. To generate lung cancer models, 2×10⁵-2×10⁶ Lewis lung cancer cells (LLC) were delivered directly into the lungs of 6 to 8 week-old female C57BL/6 mice through intratracheal instillation. To generate pancreatic cancer models, 2×10⁵-2×10⁶ KPC cells were delivered directly into the pancreas of 6 to 8 week-old female C57BL/6 mice. After tumor implantation, the mice were observed daily and sacrificed upon signs of morbidity. Mice were checked twice weekly for tumor formation by palpation or caliper measurement. Mice were sacrificed and tumors were harvested once tumor size reached 2.0 cm in diameter or upon skin ulceration. The weights and sizes of tumor were documented. For all tumor treatment studies, mice were randomized pre-experiment to ensure that there were no size biases at the onset of the experiments. To examine the effects of IC-MVPs on tumor development, purified IC-MVPs were injected into mice after tumor implantation through tail-vein injection. Mice were repeatedly dosed with IC-MVPs every 3 days for 6 times. Tumors were measured using a digital caliper and the tumor volume were calculated by the formula: (width)²×length/2.

### Mouse Model of ARDS

Balbc mice of 8-10 weeks were administered with 6 mg/kg LPS intraperitoneally. The mortality of mice was recorded every after the LPS injection for 3 to 4 days. Mice were initially treated 16 hours after LPS challenge and then treated daily with intranasally delivered IC-MVPs. Effects of IC-MVP treatment on mouse survival were recorded. In this lethal model, untreated mice usually reached experimental endpoint within 72 hours. When IC-MVP treatment saved mice from the lethality, it demonstrated that IC-MVPs can effectively dampen systemic inflammation induced by LPS. To facilitate collection of bronchoalveolar lavage (BAL) fluid, a blunt 23-gauge needle was placed within a small opening in the upper trachea and secured in position with Mersilk suture (Ethicon). The lungs were lavaged with a total volume of 700 ml of ice-cold PBS, which was instilled in 350-ml aliquots via the tracheal cannula, followed by gentle aspiration. BAL fluid was centrifuged at 425 g for 10 min at 4° C., and the cell pellets were resuspended in 100 ml of ice-cold PBS. Total viable cell counts were conducted using a hemocytometer under trypan blue exclusion. After collection of BAL fluid, lung lobes were homogenized for 4 min. Samples were centrifuged at 18,000 g for 15 min at 4° C., and supernatant cytokine levels were quantified by ELISA.

### Example 2: Exemplary Sequences

TABLE 4
Sequences

| | | | |
|---|---|---|---|
| | | | SEQ |
| | NCBI | | ID |
| Gene Name | Identifier | Full AA Sequence | NO. |

INHIBITORY IMMUNE CHECKPOINT RECEPTORS

| | | | |
|---|---|---|---|
| murine PD-1 | NP_ | MWVRQVPWSFTWAVLQLSWQ | 1 |
| | 032824.1 | SGWLLEVPNGPWRSLTFYPA | |
| | | WLTVSEGANATFTCSLSNWS | |
| | | EDLMLNWNRLSPSNQTEKQA | |
| | | AFCNGLSQPVQDARFQIIQL | |
| | | PNRHDFHMNILDTRRNDSGI | |
| | | YLCGAISLHPKAKIEESPGA | |
| | | ELVVTERILETSTRYPSPSP | |
| | | KPEGRFQGM | |
| human PD-1 | NP_ | MQIPQAPWPVVWAVLQLGWR | 2 |
| | 005009.2 | PGWFLDSPDRPWNPPTFSPA | |
| | | LLVVTEGDNATFTCSFSNTS | |
| | | ESFVLNWYRMSPSNQTDKLA | |
| | | AFPEDRSQPGQDCRFRVTQL | |
| | | PNGRDFHMSVVRARRNDSGT | |
| | | YLCGAISLAPKAQIKESLRA | |
| | | ELRVTERRAEVPTAHPSPSP | |
| | | RPAGQFQTLVVGVVGGLLGS | |
| | | LVLLVWVLAVICSRAARGTI | |
| | | GARRTGQPLKEDPSAVPVFS | |
| | | VDYGELDFQWREKTPEPPVP | |
| | | CVPEQTEYATIVFPSGMGTS | |
| | | SPARRGSADGPRSAQPLRPE | |
| | | DGHCSWPL | |
| murine CTLA-4 | NP_ | MACLGLRRYKAQLQLPSRTW | 3 |
| | 033973.2 | PFVALLTLLFIPVFSEAIQV | |
| | | TQPSVVLASSHGVASFPCEY | |
| | | SPSHNTDEVRVTVLRQTNDQ | |
| | | MTEVCATTFTEKNTVGFLDY | |
| | | PFCSGTFNESRVNLTIQGLR | |
| | | AVDTGLYLCKVELMYPPPYF | |
| | | VGMGNGTQIYVIDPEPCPDS | |
| | | DFLLWILVAVSLGLFFYSFL | |
| | | VTAVSLSKMLKKRSPLTTGV | |
| | | YVKMPPTEPECEKQFQPYFI | |
| | | PIN | |
| human CTLA-4 | NP_ | MACLGFQRHKAQLNLATRTW | 4 |
| | 005205.2 | PCTLLFFLLFIPVFCKAMHV | |
| | | AQPAVVLASSRGIASFVCEY | |
| | | ASPGKATEVRVTVLRQADSQ | |
| | | VTEVCAATYMMGNELTFLDD | |
| | | SICTGTSSGNQVNLTIQGLR | |
| | | AMDTGLYICKVELMYPPPYY | |
| | | LGIGNGTQIYVIDPEPCPDS | |
| | | DFLLWILAAVSSGLFFYSFL | |
| | | LTAVSLSKMLKKRSPLTTGV | |
| | | YVKMPPTEPECEKQFQPYFI | |
| | | PIN | |
| murine LAG3 | NP_ | MGEDLLLGFLLLGLLWEAPV | 5 |
| | 032505.1 | VSSGPGKELPVVWAQEGAPV | |
| | | HLPCSLKSPNLDPNFLRRGG | |
| | | VIWQHQPDSGQPTPIPALDL | |
| | | HQGMPSPRQPAPGRYTVLSV | |
| | | APGGLRSGRQPLHPHVQLEE | |
| | | RGLQRGDFSLWLRPALRTDA | |
| | | GEYHATVRLPNRALSCSLRL | |
| | | RVGQASMIASPSGVLKLSDW | |
| | | VLLNCSFSRPDRPVSVHWFQ | |
| | | GQNRVPVYNSPRHFLAETFL | |
| | | LLPQVSPLDSGTWGCVLTYR | |
| | | DGFNVSITYNLKVLGLEPVA | |
| | | PLTVYAAEGSRVELPCHLPP | |
| | | GVGTPSLLIAKWTPPGGGPE | |
| | | LPVAGKSGNFTLHLEAVGLA | |
| | | QAGTYTCSIHLQGQQLNATV | |
| | | TLAVITVTPKSFGLPGSRGK | |
| | | LLCEVTPASGKERFVWRPLN | |
| | | NLSRSCPGPVLEIQEARLLA | |
| | | ERWQCQLYEGQRLLGATVYA | |
| | | AESSSGAHSARRISGDLKGG | |
| | | HLVLVLILGALSLFLLVAGA | |
| | | FGFHKRIQ | |
| human LAG3 | NP_ | MWEAQFLGLLFLQPLWVAPV | 6 |
| | 002277.4 | KPLQPGAEVPVVWAQEGAPA | |
| | | QLPCSPTIPLQDLSLLRRAG | |
| | | VTWQHQPDSGPPAAAPGHPL | |
| | | APGPHPAAPSSWGPRPRRYT | |
| | | VLSVGPGGLRSGRLPLQPRV | |
| | | QLDERGRQRGDFSLWLRPAR | |
| | | RADAGEYRAAVHLRDRALSC | |
| | | RLRLRLGQASMTASPPGSLR | |
| | | ASDWVILNCSFSRPDRPASV | |
| | | HWFRNRGQGRVPVRESPHHH | |
| | | LAESFLFLPQVSPMDSGPWG | |
| | | CILTYRDGFNVSIMYNLTVL | |
| | | GLEPPTPLTVYAGAGSRVGL | |
| | | PCRLPAGVGTRSFLTAKWTP | |
| | | PGGGPDLLVTGDNGDFTLRL | |
| | | EDVSQAQAGTYTCHIHLQEQ | |
| | | QLNATVTLAIITVTPKSFGS | |
| | | PGSLGKLLCEVTPVSGQERF | |
| | | VWSSLDTPSQRSFSGPWLEA | |
| | | QEAQLLSQPWQCQLYQGERL | |
| | | LGAAVYFTELSSPGAQRSGR | |
| | | APGALPAGHLLLFLILGVLS | |
| | | LLLLVTGAFGFHLWRRQWRP | |
| | | RRFSALEQGIHPPQAQSKIE | |
| | | ELEQEPEPEPEPEPEPEPEP | |
| | | EPEQL | |
| murine BTLA | NP_ | MKTVPAMLGTPRLFREFFIL | 7 |
| | 001032808.2 | HLGLWSILCEKATKRNDEEC | |
| | | PVQLTITRNSKQSARTGELF | |
| | | KIQCPVKYCVHRPNVTWCKH | |
| | | NGTICVPLEVSPQLYTSWEE | |
| | | NQSVPVFVLHFKPIHLSDNG | |
| | | SYSCSTNFNSQVINSHSVTI | |
| | | HVTERTQNSSEHPLITVSDI | |
| | | PDATNASGPSTMEERPGRTW | |
| | | LLYT | |
| human BTLA | NP_ | MKTLPAMLGTGKLFWVFFLI | 8 |
| | 861445.4 | PYLDIWNIHGKESCDVQLYI | |
| | | KRQSEHSILAGDPFELECPV | |
| | | KYCANRPHVTWCKLNGTTCV | |
| | | KLEDRQTSWKEEKNISFFIL | |
| | | HFEPVLPNDNGSYRCSANFQ | |
| | | SNLIESHSTTLYVTDVKSAS | |
| | | ERPSKDEMASRPWLLYRLLP | |
| | | LGGLPLLITTCFCLFCCLRR | |
| | | HQGKQNELSDTAGREINLVD | |
| | | AHLKSEQTEASTRQNSQVLL | |
| | | SETGIYDNDPDLCFRMQEGS | |
| | | EVYSNPCLEENKPGIVYASL | |
| | | NHSVIGPNSRLARNVKEAPT | |
| | | EYASICVRS | |
| murine CD160 | NP_ | MERILMAPGQSCCALAILLA | 9 |
| | 001156968.1 | IVNFQHGGCIHVTSSASQKG | |
| | | GRLDLTCTLWHKKDEAEGLI | |
| | | LFWCKDNPWNCSPETNLEQL | |
| | | RVKRDPETDGITEKSSQLVF | |
| | | TIEQATPSDSGTYQCCARSQ | |
| | | KPEIYIHGHFLSVLVTGNHT | |
| | | EIRQRQRSHPDFSHINGTLS | |
| | | SGFLQVKAWGMLVTSLVALQ | |
| | | ALYTLAA | |
| Human CD160 | NP_ | MLLEPGRGCCALAILLAIVD | 10 |
| | 008984.1 | IQSGGCINITSSASQEGTRL | |
| | | NLICTVWHKKEEAEGFVVFL | |
| | | CKDRSGDCSPETSLKQLRLK | |
| | | RDPGIDGVGEISSQLMFTIS | |
| | | QVTPLHSGTYQCCARSQKSG | |
| | | IRLQGHFFSILFTETGNYTV | |
| | | TGLKQRQHLEFSHNEGTLSS | |
| | | GFLQEKVWVMLVTSLVALQA | |
| | | L | |
| murine CD244 | XP_ | MIGQAVLFTTFLLLRAHQGQ | 11 |
| (2B4) | 006496758.1 | DCPDSSEEVVGVSGKPVQLR | |
| | | PSNIQTKDVSVQWKKTEQGS | |
| | | HRKIEILNWYNDGPSWSNVS | |
| | | FSDIYGFDYGDFALSIKSAK | |
| | | LQDSGHYLLEITNTGGKVCN | |
| | | KNFQLLILDHVETPNLKAQW | |
| | | KPWTNGTCQLFLSCLVTKDD | |
| | | NVSYALYRGSTLISNQRNST | |
| | | HWENQIDASSLHTYTCNVSN | |
| | | RASWANHTLNFTHGCQSVPS | |
| | | NFRFLPFGVIIVILVTLFLG | |
| | | AIICFCVWTKKRKAA | |
| human CD244 | NM_ | MLGQVVTLILLLLLKVYQGK | 12 |
| (2B4) | 016382.4 | GCQGSADHVVSISGVPLQLQ | |
| | | PNSIQTKVDSIAWKKLLPSQ | |
| | | NGFHHILKWENGSLPSNTSN | |
| | | DRFSFIVKNLSLLIKAAQQQ | |
| | | DSGLYCLEVTSISGKVQTAT | |
| | | FQVFVFDKVEKPRLQGQGKI | |
| | | LDRGRCQVALSCLVSRDGNV | |
| | | SYAWYRGSKLIQTAGNLTYL | |
| | | DEEVDINGTHTYTCNVSNPV | |
| | | SWESHTLNLTQDCQNAHQEF | |
| | | RFWPFLVIIVILSALFLGTL | |
| | | ACFCVWRRKRKEKQSETSPK | |
| | | EFLTIYEDVKDLKTRRNHEQ | |
| | | EQTFPGGGSTIYSMIQSQSS | |
| | | APTSQEPAYTLYSLIQPSRK | |
| | | SGSRKRNHSPSFNSTIYEVI | |
| | | GKSQPKAQNPARLSRKELEN | |
| | | FDVYS | |
| murine | NP_ | MADSSIYSTLELPEAPQVQD | 13 |
| WT KLRG-1 | 058666.1 | ESRWKLKAVLHRPHLSRFAM | |
| | | VALGLLTVILMSLLMYQRIL | |
| | | CCGSKDSTCSHCPSCPILWT | |
| | | RNGSHCYYFSMEKKDWNSSL | |
| | | KFCADKGSHLLTFPDNQGVK | |
| | | LFGEYLGQDFYWIGLRNIDG | |
| | | WRWEGGPALSLRILTNSLIQ | |
| | | RCGAIHRNGLQASSCEVALQ | |
| | | WICKKVLY | |
| human wt | NP_ | MTDSVIYSMLELPTATQAQN | 14 |
| KLRG-1 | 001316028.1 | DYGPQQKSSSSRPSCSCLVA | |
| | | IALGLLTAVLLSVLLYQWIL | |
| | | CQGSNYSTCASCPSCPDRWM | |
| | | KYGNHCYYFSVEEKDWNSSL | |
| | | EFCLARDSHLLVITDNQEMS | |
| | | LLQVFLSEAFCWIGLRNNSG | |
| | | WRWEDGSPLNFSRISSNSFV | |
| | | QTCGAINKNGLQASSCEVPL | |
| | | HWVCKKCPFADQALF | |
| murine CD226 | NP_ | MAYVTWLLAILHVHKALCEE | 15 |
| | 848802 | TLWDTTVRLSETMTLECVYP | |
| | | LTHNLTQVEWTKNTGTKTVS | |
| | | IAVYNPNHNMHIESNYLHRV | |
| | | HFLNSTVGFRNMSLSFYNAS | |
| | | EADIGIYSCLFHAFPNGPWE | |
| | | KKIKVVWSDSFEIAAPSDSY | |
| | | LSAEPGQDVTLTCQLPRTWP | |
| | | VQQVIWEKVQPHQVDILASC | |
| | | NLSQETRYTSKYLRQTRSNC | |
| | | SQGSMKSILIIPNAMAADSG | |
| | | LYRCRSEAITGKNKSFVIRL | |
| | | IITDGGTNKHFILPIVGGLV | |
| | | SLLLVILIIIIFILYNRKRR | |
| | | RQVRIPLKEPRDKQSKVATN | |
| | | CRSPTSPIQSTDDEKEDIYV | |
| | | NYPTFSRRPKPRL | |
| human CD226 | NP_ | MDYPTLLLALLHVYRALCEE | 16 |
| | 001290547 | VLWHTSVPFAENMSLECVYP | |
| | | SMGILTQVEWFKIGTQQDSI | |
| | | AIFSPTHGMVIRKPYAERVY | |
| | | FLNSTMASNNMTLFFRNASE | |
| | | DDVGYYSCSLYTYPQGTWQK | |
| | | VIQVVQSDSFEAAVPSNSHI | |
| | | VSEPGKNVTLTCQPQMTWPV | |
| | | QAVRWEKIQPRQIDLLTYCN | |
| | | LVHGRNFTSKFPRQIVSNCS | |
| | | HGRWSVIVIPDVTVSDSGLY | |
| | | RCYLQASAGENETFVMRLTV | |
| | | AEGKTDNQYTLFVAGGTVLL | |
| | | LLFVISITTIIVIFLNRRRR | |
| | | RERRDLFTESWDTQKAPNNY | |
| | | RSPISTSQPTNQSMDDTRED | |
| | | IYVNYPTFSRRPKTRV | |
| murine TIGIT | NP_ | MKGWLLLVWVQGLIQAAFLA | 17 |
| | 001139797.1 | TGATAGTIDTKRNISAEEGG | |
| | | SVILQCHFSSDTAEVTQVDW | |
| | | KQQDQLLAIYSVDLGWHVAS | |
| | | VFSDRVVPGPSLGLTFQSLT | |
| | | MNDTGEYFCTYHTYPGGIYK | |
| | | GRIFLKVQESSVAQFQTAPL | |
| | | GGTMAAVLGLICLMVTGVTV | |
| | | LARKK | |
| human TIGIT | NP_ | MRWCLLLIWAQGLRQAPLAS | 18 |
| | 776160.2 | GMMTGTIETTGNISAEKGGS | |
| | | IILQCHLSSTTAQVTQVNWE | |
| | | QQDQLLAICNADLGWHISPS | |
| | | FKDRVAPGPGLGLTLQSLTV | |
| | | NDTGEYFCIYHTYPDGTYTG | |
| | | RIFLEVLESSVAEHGARFQI | |
| | | PLLGAMAATLVVICTAVIVV | |
| | | VALTRKKKALRIHSVEGDLR | |
| | | RKSAGQEEWSPSAPSPPGSC | |
| | | VQAEAAPAGLCGEORGEDCA | |
| | | ELHDYFNVLSYRSLGNCSFF | |
| | | TETG | |
| murine CD276 | NP_ | MLRGWGGPSVGVCVRTALGV | 19 |
| (B7-H3) | 598744 | LCLCLTGAVEVQVSEDPVVA | |
| | | LVDTDATLRCSFSPEPGFSL | |
| | | AQLNLIWQLTDTKQLVHSFT | |
| | | EGRDQGSAYSNRTALFPDLL | |
| | | VQGNASLRLQRVRVTDEGSY | |
| | | TCFVSIQDFDSAAVSLQVAA | |
| | | PYSKPSMTLEPNKDLRPGNM | |
| | | VTITCSSYQGYPEAEVFWKD | |
| | | GQGVPLTGNVTTSQMANERG | |
| | | LFDVHSVLRVVLGANGTYSC | |
| | | LVRNPVLQQDAHGSVTITGQ | |
| | | PLTFPPEALWVTVGLSVCLV | |
| | | VLLVALAFVCWRKIKQSCEE | |
| | | ENAGAEDQDGDGEGSKTALR | |
| | | PLKPSENKEDDGQEIA | |
| Human CD276 | NP_ | MLRRRGSPGMGVHVGAALGA | 20 |
| (B7-H3) | 001019907 | LWFCLTGALEVQVPEDPVVA | |
| | | LVGTDATLCCSFSPEPGFSL | |
| | | AQLNLIWQLTDTKQLVHSFA | |
| | | EGQDQGSAYANRTALFPDLL | |
| | | AQGNASLRLQRVRVADEGSF | |
| | | TCFVSIRDFGSAAVSLQVAA | |
| | | PYSKPSMTLEPNKDLRPGDT | |
| | | VTITCSSYQGYPEAEVFWQD | |
| | | GQGVPLTGNVTTSQMANEQG | |
| | | LFDVHSILRVVLGANGTYSC | |
| | | LVRNPVLQQDAHSSVTITPQ | |
| | | RSPTGAVEVQVPEDPVVALV | |
| | | GTDATLRCSFSPEPGFSLAQ | |
| | | LNLIWQLTDTKQLVHSFTEG | |
| | | RDQGSAYANRTALFPDLLAQ | |
| | | GNASLRLQRVRVADEGSFTC | |
| | | FVSIRDFGSAAVSLQVAAPY | |
| | | SKPSMTLEPNKDLRPGDTVT | |
| | | ITCSSYRGYPEAEVFWQDGQ | |
| | | GVPLTGNVTTSQMANEQGLF | |
| | | DVHSVLRVVLGANGTYSCLV | |
| | | RNPVLQQDAHGSVTITGQPM | |
| | | TFPPEALWVTVGLSVCLIAL | |
| | | LVALAFVCWRKIKQSCEEEN | |
| | | AGAEDQDGEGEGSKTALQPL | |
| | | KHSDSKEDDGQEIA | |
| murine VISTA | NP_ | MGVPAVPEASSPRWGTLLLA | 21 |
| | 001153044 | IFLAASRGLVAAFKVTTPYS | |
| | | LYVCPEGQNATLTCRILGPV | |
| | | SKGHDVTIYKTWYLSSRGEV | |
| | | QMCKEHRPIRNFTLQHLQHH | |
| | | GSHLKANASHDQPQKHGLEL | |
| | | ASDHHGNFSITLRNVTPRDS | |
| | | GLYCCLVIELKNHHPEQRFY | |
| | | GSMELQVQAGKGSGSTCMAS | |
| | | NEQDSDSITAAALATGACIV | |
| | | GILCLPLILLLVYKQRQVAS | |
| | | HRRAQELVRMDSNTQGIENP | |
| | | GFETTPPFQGMPEAKTRPPL | |
| | | SYVAQRQPSESGRYLLSDPS | |
| | | TPLSPPGPGDVFFPSLDPVP | |
| | | DSPNSEAI | |
| human VISTA | NP_ | MGVPTALEAGSWRWGSLLFA | 22 |
| | 071436.1 | LFLAASLGPVAAFKVATPYS | |
| | | LYVCPEGQNVTLTCRLLGPV | |
| | | DKGHDVTFYKTWYRSSRGEV | |
| | | QTCSERRPIRNLTFQDLHLH | |
| | | HGGHQAANTSHDLAQRHGLE | |
| | | SASDHHGNFSITMRNLTLLD | |
| | | SGLYCCLVVEIRHHHSEHRV | |
| | | HGAMELQVQTGKDAPSNCVV | |
| | | YPSSSQDSENITAAALATGA | |
| | | CIVGILCLPLILLLVYKQRQ | |
| | | AASNRRAQELVRMDSNIQGI | |
| | | ENPGFEASPPAQGIPEAKVR | |
| | | HPLSYVAQRQPSESGRHLLS | |
| | | EPSTPLSPPGPGDVFFPSLD | |
| | | PVPDSPNFEVI | |
| Murine | NP_ | MFSGLTLNCVLLLLQLLLAR | 23 |
| HAVCR2 | 599011 | SLENAYVFEVGKNAYLPCSY | |
| (TIM-3) | | TLSTPGALVPMCWGKGFCPW | |
| | | SQCTNELLRTDERNVTYQKS | |
| | | SRYQLKGDLNKGDVSLIIKN | |
| | | VTLDDHGTYCCRIQFPGLMN | |
| | | DKKLELKLDIKAAKVTPAQT | |
| | | AHGDSTTASPRTLTTERNGS | |
| | | ETQTLVTLHNNNGTKISTWA | |
| | | DEIKDSGETIRTAIHIGVGV | |
| | | SAGLTLALIIGVLILKWYSC | |
| | | KKKKLSSLSLITLANLPPGG | |
| | | LANAGAVRIRSEENIYTIEE | |
| | | NVYEVENSNEYYCYVNSQQP | |
| | | S | |
| human | NP_ | MFSHLPFDCVLLLLLLLLTR | 24 |
| HAVCR2 | 116171.3 | SSEVEYRAEVGQNAYLPCFY | |
| (TIM-3) | | TPAAPGNLVPVCWGKGACPV | |
| | | FECGNVVLRTDERDVNYWTS | |
| | | RYWLNGDFRKGDVSLTIENV | |
| | | TLADSGIYCCRIQIPGIMND | |
| | | EKFNLKLVIKPAKVTPAPTR | |
| | | QRDFTAAFPRMLTTRGHGPA | |
| | | ETQTLGSLPDINLTQISTLA | |
| | | NELRDSRLANDLRDSGATIR | |
| | | IGIYIGAGICAGLALALIFG | |
| | | ALIFKWYSHSKEKIQNLSLI | |
| | | SLANLPPSGLANAVAEGIRS | |
| | | EENIYTIEENVYEVEEPNEY | |
| | | YCYVSSRQQPSQPLGCRFAM | |
| | | P | |

INHIBITORY IMMUNE CHECKPOINT LIGANDS

| | | | |
|---|---|---|---|
| murine PD-L1 | NM_ | MRIFAGIIFTACCHLLRAFT | 25 |
| | 021893.3 | ITAPKDLYVVEYGSNVTMEC | |
| | | RFPVERELDLLALVVYWEKE | |
| | | DEQVIQFVAGEEDLKPQHSN | |
| | | FRGRASLPKDQLLKGNAALQ | |
| | | ITDVKLQDAGVYCCIISYGG | |
| | | ADYKRITLKVNAPYRKINQR | |
| | | ISVDPATSEHELICQAEGYP | |
| | | EAEVIWTNSDHQPVSGKRSV | |
| | | TTSRTEGMLLNVTSSLRVNA | |
| | | TANDVFYCTFWRSQPGQNHT | |
| | | AELIIPELPATHPPQNRTHW | |
| | | VLLGSILLFLIVVSTVLLFL | |
| | | RKQVRMLDVEKCGVEDTSSK | |
| | | NRNDTQFEET | |
| human PD-L1 | NM_ | MRIFAVFIFMTYWHLLNAFT | 26 |
| | 014143.4 | VTVPKDLYVVEYGSNMTIEC | |
| | | KFPVEKQLDLAALIVYWEME | |
| | | DKNIIQFVHGEEDLKVQHSS | |
| | | YRQRARLLKDQLSLGNAALQ | |
| | | ITDVKLQDAGVYRCMISYGG | |
| | | ADYKRITVKVNAPYNKINQR | |
| | | ILVVDPVTSEHELTCQAEGY | |
| | | PKAEVIWTSSDHQVLSGKTT | |
| | | TTNSKREEKLFNVTSTLRIN | |
| | | TTTNEIFYCTFRRLDPEENH | |
| | | TAELVIPELPLAHPPNERTH | |
| | | LVILGAILLCLGVALTFIFR | |
| | | LRKGRMMDVKKCGIQDTNSK | |
| | | KQSDTHLEET | |
| murine PD-L2 | Q9WUL5 | MLLLLPILNLSLQLHPVAAL | 27 |
| | | FTVTAPKEVYTVDVGSSVSL | |
| | | ECDFDRRECTELEGIRASLQ | |
| | | KVENDTSLQSERATLLEEQL | |
| | | PLGKALFHIPSVQVRDSGQY | |
| | | RCLVICGAAWDYKYLTVKVK | |
| | | ASYMRIDTRILEVPGTGEVO | |
| | | LTCQARGYPLAEVSWQNVSV | |
| | | PANTSHIRTPEGLYQVTSVL | |
| | | RLKPQPSRNFSCMFWNAHMK | |
| | | ELTSAIIDPLSRMEPKVPRT | |
| | | WPLHVFIPACTIALIFLAIV | |
| | | IIQRKRI | |
| human PD-L2 | NM_ | MIFLLLMLSLELQLHQIAAL | 28 |
| | 025239.4 | FTVTVPKELYIIEHGSNVTL | |
| | | ECNFDTGSHVNLGAITASLQ | |
| | | KVENDTSPHRERATLLEEQL | |
| | | PLGKASFHIPQVQVRDEGQY | |
| | | QCIIIYGVAWDYKYLTLKVK | |
| | | ASYRKINTHILKVPETDEVE | |
| | | LTCQATGYPLAEVSWPNVSV | |
| | | PANTSHSRTPEGLYQVTSVL | |
| | | RLKPPPGRNFSCVFWNTHVR | |
| | | ELTLASIDLQSQMEPRTHPT | |
| | | WLLHIFIPFCIIAFIFIATV | |
| | | IALRKQLCQKLYSSKDTTKR | |
| | | PVTTTKREVNSAI | |
| murine HVEM | Q71F55 | MEPLPGWGSAPWSQAPTDNT | 29 |
| | | FRLVPCVFLLNLLQRISAQP | |
| | | SCRQEEFLVGDECCPMCNPG | |
| | | YHVKQVCSEHTGTVCAPCPP | |
| | | QTYTAHANGLSKCLPCGVCD | |
| | | PDMGLLTWQECSSWKDTVCR | |
| | | CIPGYFCENQDGSHCSTCLQ | |
| | | HTTCPPGQRVEKRGTHDQDT | |
| | | VCADCLTGTFSLGGTQEECL | |
| | | PWTNCSAFQQEVRRGTNSTD | |
| | | TTCSSQVVYYVVSILLPLVI | |
| | | VGVGIAGFLICTRRHLHTSS | |
| | | VAKELEPFQQEQQENTIRFP | |
| | | VTEVGFAETEEETASN | |
| human HVEM | NM_ | MEPPGDWGPPPWRSTPKTDV | 30 |
| | 003820.4 | LRLVLYLTFLGAPCYAPALP | |
| | | SCKEDEYPVGSECCPKCSPG | |
| | | YRVKEACGELTGTVCEPCPP | |
| | | GTYIAHLNGLSKCLQCQMCD | |
| | | PAMGLRASRNCSRTENAVCG | |
| | | CSPGHFCIVQDGDHCAACRA | |
| | | YATSSPGQRVQKGGTESQDT | |
| | | LCQNCPPGTFSPNGTLEECQ | |
| | | HQTKCSWLVTKAGAGTSSSH | |
| | | WVWWFLSGSLVIVIVCSTVG | |
| | | LIICVKRRKPRGDVVKVIVS | |
| | | VQRKRQEAEGEATVIEALQA | |
| | | PPDVTTVAVEETIPSFTGRS | |
| | | PNH | |
| murine CD48 | P18181 | MCFIKQGWCLVLELLLLPLG | 31 |
| | | TGFQGHSIPDINATTGSNVT | |
| | | LKIHKDPLGPYKRITWLHTK | |
| | | NQKILEYNYNSTKTIFESEF | |
| | | KGRVYLEENNGALHISNVRK | |
| | | EDKGTYYMRVLRETENELKI | |
| | | TLEVFDPVPKPSIEINKTEA | |
| | | STDSCHLRLSCEVKDQHVDY | |
| | | TWYESSGPFPKKSPGYVLDL | |
| | | IVTPQNKSTFYTCQVSNPVS | |
| | | SKNDTVYFTLPCDLARSSGV | |
| | | CWTATWLVVTTLIIHRILLT | |
| human CD48 | P09326 | MCSRGWDSCLALELLLLPLS | 32 |
| | | LLVTSIQGHLVHMTVVSGSN | |
| | | VTLNISESLPENYKQLTWFY | |
| | | TFDQKIVEWDSRKSKYFESK | |
| | | FKGRVRLDPQSGALYISKVQ | |
| | | KEDNSTYIMRVLKKTGNEQE | |
| | | WKIKLQVLDPVPKPVIKIEK | |
| | | IEDMDDNCYLKLSCVIPGES | |
| | | VNYTWYGDKRPFPKELQNSV | |
| | | LETTLMPHNYSRCYTCQVSN | |
| | | SVSSKNGTVCLSPPCTLARS | |
| | | FGVEWIASWLVVTVPTILGL | |
| | | LLT | |
| murine CD112 | P32507 | MARAAVLPPSRLSPTLPLLP | 33 |
| | | LLLLLLQETGAQDVRVRVLP | |
| | | EVRGRLGGTVELPCHLLPPT | |
| | | TERVSQVTWORLDGTVVAAF | |
| | | HPSFGVDFPNSQFSKDRLSF | |
| | | VRARPETNADLRDATLAFRG | |
| | | LRVEDEGNYTCEFATFPNGT | |
| | | RRGVTWLRVIAQPENHAEAQ | |
| | | EVTIGPQSVAVARCVSTGGR | |
| | | PPARITWISSLGGEAKDTQE | |
| | | PGIQAGTVTIISRYSLVPVG | |
| | | RADGVKVTCRVEHESFEEPI | |
| | | LLPVTLSVRYPPEVSISGYD | |
| | | DNWYLGRSEAILTCDVRSNP | |
| | | EPTDYDWSTTSGVFPASAVA | |
| | | QGSQLLVHSVDRMVNTTFIC | |
| | | TATNAVGTGRAEQVILVRES | |
| | | PSTAGAGATGGIIGGIIAAI | |
| | | IATAVAGTGILICRQQRKEQ | |
| | | RLQAADEEEELEGPPSYKPP | |
| | | TPKAKLEEPEMPSQLFTLGA | |
| | | SEHSPVKTPYFDAGVSCADQ | |
| | | EMPRYHELPTLEERSGPLLL | |
| | | GATGLGPSLLVPPGPNVVEG | |
| | | VSLSLEDEEEDDEEEDFLDK | |
| | | INPIYDALSYPSPSDSYQSK | |
| | | DFFVSRAMYV | |
| human CD112 | Q92692 | MARAAALLPSRSPPTPLLWP | 34 |
| | | LLLLLLLETGAQDVRVQVLP | |
| | | EVRGQLGGTVELPCHLLPPV | |
| | | PGLYISLVTWQRPDAPANHQ | |
| | | NVAAFHPKMGPSFPSPKPGS | |
| | | ERLSFVSAKQSTGQDTEAEL | |
| | | QDATLALHGLTVEDEGNYTC | |
| | | EFATFPKGSVRGMTWLRVIA | |
| | | KPKNQAEAQKVTFSQDPTTV | |
| | | ALCISKEGRPPARISWLSSL | |
| | | DWEAKETQVSGTLAGTVTVT | |
| | | SRFTLVPSGRADGVTVTCKV | |
| | | EHESFEEPALIPVTLSVRYP | |
| | | PEVSISGYDDNWYLGRTDAT | |
| | | LSCDVRSNPEPTGYDWSTTS | |
| | | GTFPTSAVAQGSQLVIHAVD | |
| | | SLFNTTFVCTVTNAVGMGRA | |
| | | EQVIFVRETPNTAGAGATGG | |
| | | IIGGIIAAIIATAVAATGIL | |
| | | ICRQQRKEQTLQGAEEDEDL | |
| | | EGPPSYKPPTPKAKLEAQEM | |
| | | PSQLFTLGASEHSPLKTPYF | |
| | | DAGASCTEQEMPRYHELPTL | |
| | | EERSGPLHPGATSLGSPIPV | |
| | | PPGPPAVEDVSLDLEDEEGE | |
| | | EEEEYLDKINPIYDALSYSS | |
| | | PSDSYQGKGFVMSRAMYV | |
| murine CD155 | Q91WP1 | MAQLARATRSPLSWLLLLFC | 35 |
| | | YALRKAGGDIRVLVPYNSTG | |
| | | VLGGSTTLHCSLTSNENVTI | |
| | | TQITWMKKDSGGSHALVAVF | |
| | | HPKKGPNIKEPERVKFLAAQ | |
| | | QDLRNASLAISNLSVEDEGI | |
| | | YECQIATFPRGSRSTNAWLK | |
| | | VQARPKNTAEALEPSPTLIL | |
| | | QDVAKCISANGHPPGRISWP | |
| | | SNVNGSHREMKEPGSQPGTT | |
| | | TVTSYLSMVPSRQADGKNIT | |
| | | CTVEHESLQELDQLLVTLSQ | |
| | | PYPPENVSISGYDGNWYVGL | |
| | | TNLTLTCEAHSKPAPDMAGY | |
| | | NWSTTTGDFPNSVKRQGNML | |
| | | LISTVEDGLNNTVIVCEVTN | |
| | | ALGSGQGQVHIIVKEKPENM | |
| | | QQNTRLHLGYIFLIVFVLAV | |
| | | VIIIAALYTIRRCRHGRALQ | |
| | | SNPSERENVQYSSVNGDCRL | |
| | | NMEPNSTR | |
| human CD155 | P15151 | MARAMAAAWPLLLVALLVLS | 36 |
| | | WPPPGTGDVVVQAPTQVPGF | |
| | | LGDSVTLPCYLQVPNMEVTH | |
| | | VSQLTWARHGESGSMAVFHQ | |
| | | TQGPSYSESKRLEFVAARLG | |
| | | AELRNASLRMFGLRVEDEGN | |
| | | YTCLFVTFPQGSRSVDIWLR | |
| | | VLAKPQNTAEVQKVQLTGEP | |
| | | VPMARCVSTGGRPPAQITWH | |
| | | SDLGGMPNTSQVPGFLSGTV | |
| | | TVTSLWILVPSSQVDGKNVT | |
| | | CKVEHESFEKPQLLTVNLTV | |
| | | YYPPEVSISGYDNNWYLGQN | |
| | | EATLTCDARSNPEPTGYNWS | |
| | | TTMGPLPPFAVAQGAQLLIR | |
| | | PVDKPINTTLICNVTNALGA | |
| | | RQAELTVQVKEGPPSEHSGI | |
| | | SRNAIIFLVLGILVFLILLG | |
| | | IGIYFYWSKCSREVLWHCHL | |
| | | CPSSTEHASASANGHVSYSA | |
| | | VSRENSSSQDPQTEGTR | |
| murine | P31809 | MELASAHLHKGQVPWGGLLL | 37 |
| Ceacam-1 | | TASLLASWSPATTAEVTIEA | |
| | | VPPQVAEDNNVLLLVHNLPL | |
| | | ALGAFAWYKGNTTAIDKEIA | |
| | | RFVPNSNMNFTGQAYSGREI | |
| | | IYSNGSLLFQMITMKDMGVY | |
| | | TLDMTDENYRRTQATVRFHV | |
| | | HPILLKPNITSNNSNPVEGD | |
| | | DSVSLTCDSYTDPDNINYLW | |
| | | SRNGESLSEGDRLKLSEGNR | |
| | | TLTLLNVTRNDTGPYVCETR | |
| | | NPVSVNRSDPFSLNIIYGPD | |
| | | TPIISPSDIYLHPGSNLNLS | |
| | | CHAASNPPAQYFWLINEKPH | |
| | | ASSQELFIPNITTNNSGTYT | |
| | | CFVNNSVTGLSRTTVKNITV | |
| | | LEPVTQPFLQVTNTTVKELD | |
| | | SVTLTCLSNDIGANIQWLFN | |
| | | SQSLQLTERMTLSQNNSILR | |
| | | IDPIKREDAGEYQCEISNPV | |
| | | SVRRSNSIKLDIIFDPTQGG | |
| | | LSDGAIAGIVIGVVAGVALI | |
| | | AGLAYFLYSRKSGGGSDQRD | |
| | | LTEHKPSTSNHNLAPSDNSP | |
| | | NKVDDVAYTVLNFNSQQPNR | |
| | | PTSAPSSPRATETVYSEVKK | |
| | | K | |
| human Ceacam- | P13688 | MGHLSAPLHRVRVPWQGLLL | 38 |
| 1 | | TASLLTFWNPPTTAQLTTES | |
| | | MPFNVAEGKEVLLLVHNLPQ | |
| | | QLFGYSWYKGERVDGNRQIV | |
| | | GYAIGTQQATPGPANSGRET | |
| | | IYPNASLLIQNVTQNDTGFY | |
| | | TLQVIKSDLVNEEATGQFHV | |
| | | YPELPKPSISSNNSNPVEDK | |
| | | DAVAFTCEPETQDTTYLWWI | |
| | | NNQSLPVSPRLQLSNGNRTL | |
| | | TLLSVTRNDTGPYECEIQNP | |
| | | VSANRSDPVTLNVTYGPDTP | |
| | | TISPSDTYYRPGANLSLSCY | |
| | | AASNPPAQYSWLINGTFQQS | |
| | | TQELFIPNITVNNSGSYTCH | |
| | | ANNSVTGCNRTTVKTIIVTE | |
| | | LSPVVAKPQIKASKTTVTGD | |
| | | KDSVNLTCSTNDTGISIRWF | |
| | | FKNQSLPSSERMKLSQGNTT | |
| | | LSINPVKREDAGTYWCEVFN | |
| | | PISKNQSDPIMLNVNYNALP | |
| | | QENGLSPGAIAGIVIGVVAL | |
| | | VALIAVALACFLHFGKTGRA | |
| | | SDQRDLTEHKPSVSNHTQDH | |
| | | SNDPPNKMNEVTYSTLNFEA | |
| | | QQPTQPTSASPSLTATEIIY | |
| | | SEVKKQ | |
| murine | P16110 | MADSFSLNDALAGSGNPNPQ | 39 |
| Galectin-3 | | GYPGAWGNQPGAGGYPGAAY | |
| | | PGAYPGQAPPGAYPGQAPPG | |
| | | AYPGQAPPSAYPGPTAPGAY | |
| | | PGPTAPGAYPGQPAPGAFPG | |
| | | QPGAPGAYPQCSGGYPAAGP | |
| | | YGVPAGPLTVPYDLPLPGGV | |
| | | MPRMLITIMGTVKPNANRIV | |
| | | LDFRRGNDVAFHFNPRFNEN | |
| | | NRRVIVCNTKQDNNWGKEER | |
| | | QSAFPFESGKPFKIQVLVEA | |
| | | DHFKVAVNDAHLLQYNHRMK | |
| | | NLREISQLGISGDITLTSAN | |
| | | HAMI | |
| human | P17931 | MADNFSLHDALSGSGNPNPQ | 40 |
| Galectin-3 | | GWPGAWGNQPAGAGGYPGAS | |
| | | YPGAYPGQAPPGAYPGQAPP | |
| | | GAYPGAPGAYPGAPAPGVYP | |
| | | GPPSGPGAYPSSGQPSATGA | |
| | | YPATGPYGAPAGPLIVPYNL | |
| | | PLPGGVVPRMLITILGTVKP | |
| | | NANRIALDFQRGNDVAFHFN | |
| | | PRFNENNRRVIVCNTKLDNN | |
| | | WGREERQSVFPFESGKPFKI | |
| | | QVLVEPDHFKVAVNDAHLLQ | |
| | | YNHRVKKLNEISKLGISGDI | |
| | | DLTSASYTMI | |
| murine FGL-1 | Q71KU9 | MGKIYSFVLVAIALMMGREG | 41 |
| | | WALESESCLREQVRLRAQVH | |
| | | QLETRVKQQQTMIAQLLHEK | |
| | | EVQFLDKGSENSFIDLGGKR | |
| | | QYADCSEIYNDGFKQSGFYK | |
| | | IKPLQSLAEFSVYCDMSDGG | |
| | | GWTVIQRRSDGSENFNRGWN | |
| | | DYENGFGNFVQNNGEYWLGN | |
| | | KNINLLTIQGDYTLKIDLTD | |
| | | FEKNSSFAQYQSFKVGDKKS | |
| | | FYELNIGEYSGTAGDSLSGT | |
| | | FHPEVQWWASHQRMKFSTWD | |
| | | RDNDNYQGNCAEEEQSGWWF | |
| | | NRCHSANLNGVYYRGSYRAE | |
| | | TDNGVVWYTWHGWWYSLKSV | |
| | | VMKIRPSDFIPNII | |
| human FGL-1 | Q08830 | MAKVFSFILVTTALTMGREI | 42 |
| | | SALEDCAQEQMRLRAQVRLL | |
| | | ETRVKQQQVKIKQLLQENEV | |
| | | QFLDKGDENTVIDLGSKRQY | |
| | | ADCSEIFNDGYKLSGFYKIK | |
| | | PLQSPAEFSVYCDMSDGGGW | |
| | | TVIQRRSDGSENFNRGWKDY | |
| | | ENGFGNFVQKHGEYWLGNKN | |
| | | LHFLTTQEDYTLKIDLADFE | |
| | | KNSRYAQYKNFKVGDEKNFY | |
| | | ELNIGEYSGTAGDSLAGNFH | |
| | | PEVQWWASHQRMKFSTWDRD | |
| | | HDNYEGNCAEEDQSGWWFNR | |
| | | CHSANLNGVYYSGPYTAKTD | |
| | | NGIVWYTWHGWWYSLKSVVM | |
| | | KIRPNDFIPNVI | |

T cell activating immune checkpoint

| | | | |
|---|---|---|---|
| human CD80 | NC_ | MGHTRRQGTSPSKCPYLNFF | 43 |
| | 000003.12 | QLLVLAGLSHFCSGVIHVTK | |
| | | EVKEVATLSCGHNVSVEELA | |
| | | QTRIYWQKEKKMVLTMMSGD | |
| | | MNIWPEYKNRTIFDITNNLS | |
| | | IVILALRPSDEGTYECVVLK | |
| | | YEKDAFKREHLAEVTLSVKA | |
| | | DFPTPSISDFEIPTSNIRRI | |
| | | ICSTSGGFPEPHLSWLENGE | |
| | | ELNAINTTVSQDPETELYAV | |
| | | SSKLDFNMTTNHSFMCLIKY | |
| | | GHLRVNQTFNWNTTKQEHFP | |
| | | DNLLPSWAITLISVNGIFVI | |
| | | CCLTYCFAPRCRERRRNERL | |
| | | RRESVRPV | |
| murine CD80 | NM_ | MACNCQLMQDTPLLKFPCPR | 44 |
| | 001359898.1 | LILLFVLLIRLSQVSSDVDE | |
| | | QLSKSVKDKVLLPCRYNSPH | |
| | | EDESEDRIYWQKHDKVVLSV | |
| | | IAGKLKVWPEYKNRTLYDNT | |
| | | TYSLIILGLVLSDRGTYSCV | |
| | | VQKKERGTYEVKHLALVKLS | |
| | | IKADFSTPNITESGNPSADT | |
| | | KRITCFASGGFPKPRFSWLE | |
| | | NGRELPGINTTISQDPESEL | |
| | | YTISSQLDFNTTRNHTIKCL | |
| | | IKYGDAHVSEDFTWEKPPED | |
| | | PPDSKNTLVLFGAGFGAVIT | |
| | | VVVIVVIIKCFCKHRSCFRR | |
| | | NEASRETNNSLTFGPEEALA | |
| | | EQTVFL | |
| human CD86 | CR541844.1 | MGLSNILFVMAFLLSGAAPL | 45 |
| | | KIQAYFNETADLPCQFANSQ | |
| | | NQSLSELVVFWQDQENLVLN | |
| | | EVYLGKEKFDSVHSKYMGRT | |
| | | SFDSDSWTLRLHNLQIKDKG | |
| | | LYQCIIHHKKPTGMIRIHQM | |
| | | NSELSVLANFSQPEIVPISN | |
| | | ITENVYINLTCSSIHGYPEP | |
| | | KKMSVLLRTKNSTIEYDGIM | |
| | | QKSQDNVTELYDVSISLSVS | |
| | | FPDVTSNMTIFCILETDKTR | |
| | | LLSSPFSIELEDPQPPPDHI | |
| | | PWITAVLPTVIICVMVFCLI | |
| | | LWKWKKKKRPRNSYKCGTNT | |
| | | MEREESEQTKKREKIHIPER | |
| | | SDEAQRVFKSSKTSSCDKSD | |
| | | TCF | |
| murine CD86 | NM_ | MDPRCTMGLAILIFVTVLLI | 46 |
| | 019388.3 | SDAVSVETQAYFNGTAYLPC | |
| | | PFTKAQNISLSELVVFWQDQ | |
| | | QKLVLYEHYLGTEKLDSVNA | |
| | | KYLGRTSFDRNNWTLRLHNV | |
| | | QIKDMGSYDCFIQKKPPTGS | |
| | | IILQQTLTELSVIANFSEPE | |
| | | IKLAQNVTGNSGINLTCTSK | |
| | | QGHPKPKKMYFLITNSTNEY | |
| | | GDNMQISQDNVTELFSISNS | |
| | | LSLSFPDGVWHMTVVCVLET | |
| | | ESMKISSKPLNFTQEFPSPQ | |
| | | TYWKEITASVTVALLLVMLL | |
| | | IIVCHKKPNQPSRPSNTASK | |
| | | LERDSNADRETINLKELEPQ | |
| | | IASAKPNAE | |
| human 4-1BBL | NM 003811.4 | MEYASDASLDPEAPWPPAPR | 47 |
| | | ARACRVLPWALVAGLLLLLL | |
| | | LAAACAVFLACPWAVSGARA | |
| | | SPGSAASPRLREGPELSPDD | |
| | | PAGLLDLRQGMFAQLVAQNV | |
| | | LLIDGPLSWYSDPGLAGVSL | |
| | | TGGLSYKEDTKELVVAKAGV | |
| | | YYVFFQLELRRVVAGEGSGS | |
| | | VSLALHLQPLRSAAGAAALA | |
| | | LTVDLPPASSEARNSAFGFQ | |
| | | GRLLHLSAGQRLGVHLHTEA | |
| | | RARHAWQLTQGATVLGLFRV | |
| | | TPEIPAGLPSPRSE | |
| murine 4-1BBL | NM_ | MDQHTLDVEDTADARHPAGT | 48 |
| | 009404.3 | SCPSDAALLRDTGLLADAAL | |
| | | LSDTVRPTNAALPTDAAYPA | |
| | | VNVRDREAAWPPALNFCSRH | |
| | | PKLYGLVALVLLLLIAACVP | |
| | | IFTRTEPRPALTITTSPNLG | |
| | | TRENNADQVTPVSHIGCPNT | |
| | | TQQGSPVFAKLLAKNQASLC | |
| | | NTTLNWHSQDGAGSSYLSQG | |
| | | LRYEEDKKELVVDSPGLYYV | |
| | | FLELKLSPTFTNTGHKVQGW | |
| | | VSLVLQAKPQVDDFDNLALT | |
| | | VELFPCSMENKLVDRSWSQL | |
| | | LLLKAGHRLSVGLRAYLHGA | |
| | | QDAYRDWELSYPNTTSFGLF | |
| | | LVKPDNPWE | |
| human 4-1BB | U03397.1 | MGNSCYNIVATLLLVLNFER | 49 |
| | | TRSLQDPCSNCPAGTFCDNN | |
| | | RNQICSPCPPNSFSSAGGQR | |
| | | TCDICRQCKGVFRTRKECSS | |
| | | TSNAECDCTPGFHCLGAGCS | |
| | | MCEQDCKQGQELTKKGCKDC | |
| | | CFGTFNDQKRGICRPWTNCS | |
| | | LDGKSVLVNGTKERDVVCGP | |
| | | SPADLSPGASSVTPPAPARE | |
| | | PGHSPQIISFFLALTSTALL | |
| | | FLLFFLTLRFSVVKRGRKKL | |
| | | LYIFKQPFMRPVQTTQEEDG | |
| | | CSCRFPEEEEGGCEL | |
| murine 4-1BB | NM_ | MGNNCYNVVVIVLLLVGCEK | 50 |
| | 011612.2 | VGAVQNSCDNCQPGTFCRKY | |
| | | NPVCKSCPPSTFSSIGGQPN | |
| | | CNICRVCAGYFRFKKFCSST | |
| | | HNAECECIEGFHCLGPQCTR | |
| | | CEKDCRPGQELTKQGCKTCS | |
| | | LGTENDQNGTGVCRPWTNCS | |
| | | LDGRSVLKTGTTEKDVVCGP | |
| | | PVVSFSPSTTISVTPEGGPG | |
| | | GHSLQVLTLFLALTSALLLA | |
| | | LIFITLLFSVLKWIRKKFPH | |
| | | IFKQPFKKTTGAAQEEDACS | |
| | | CRCPQEEEGGGGGYEL | |
| human OX40 | AJ277151.1 | MCVGARRLGRGPCAALLLLG | 51 |
| | | LGLSTVTGLHCVGDTYPSND | |
| | | RCCHECRPGNGMVSRCSRSQ | |
| | | NTVCRPCGPGFYNDVVSSKP | |
| | | CKPCTWCNLRSGSERKQLCT | |
| | | ATQDTVCRCRAGTQPLDSYK | |
| | | PGVDCAPCPPGHFSPGDNQA | |
| | | CKPWTNCTLAGKHTLQPASN | |
| | | SSDAICEDRDPPATQPQETQ | |
| | | GPPARPITVQPTEAWPRTSQ | |
| | | GPSTRPVEVPGGRAVAAILG | |
| | | LGLVLGLLGPLAILLALYLL | |
| | | RRDQRLPPDAHKPPGGGSFR | |
| | | TPIQEEQADAHSTLAKI | |
| murine OX40 | X85214.1 | MYVWVQQPTALLLLGLTLGV | 52 |
| | | TARRLNCVKHTYPSGHKCCR | |
| | | ECQPGHGMVSRCDHTRDTLC | |
| | | HPCETGFYNEAVNYDTCKQC | |
| | | TQCNHRSGSELKQNCTPTQD | |
| | | TVCRCRPGTQPRQDSGYKLG | |
| | | VDCVPCPPGHFSPGNNQACK | |
| | | PWTNCTLSGKQTRHPASDSL | |
| | | DAVCEDRSLLATLLWETQRP | |
| | | TFRPTTVQSTTVWPRTSELP | |
| | | SPPTLVTPEGPAFAVLLGLG | |
| | | LGLLAPLTVLLALYLLRKAW | |
| | | RLPNTPKPCWGNSFRTPIQE | |
| | | EHTDAHFTLAKI | |
| human OX40L | NM_ | MVSHRYPRIQSIKVQFTEYK | 53 |
| | 001297562.2 | KEKGFILTSQKEDEIMKVQN | |
| | | NSVIINCDGFYLISLKGYFS | |
| | | QEVNISLHYQKDEEPLFQLK | |
| | | KVRSVNSLMVASLTYKDKVY | |
| | | LNVTTDNTSLDDFHVNGGEL | |
| | | ILIHQNPGEFCVL | |
| murine OX40L | U12763.1 | MEGEGVQPLDENLENGSRPR | 54 |
| | | FKWKKTLRLVVSGIKGAGML | |
| | | LCFIYVCLQLSSSPAKDPPI | |
| | | QRLRGAVTRCEDGQLFISSY | |
| | | KNEYQTMEVQNNSVVIKCDG | |
| | | LYIIYLKGSFFQEVKIDLHF | |
| | | REDHNPISIPMLNDGRRIVF | |
| | | TVVASLAFKDKVYLTVNAPD | |
| | | TLCEHLQINDGELIVVQLTP | |
| | | GYCAPEGSYHSTVNQVPL | |
| human CD40 | P25942 | MVRLPLQCVLWGCLLTAVHP | 55 |
| | | EPPTACREKQYLINSQCCSL | |
| | | CQPGQKLVSDCTEFTETECL | |
| | | PCGESEFLDTWNRETHCHQH | |
| | | KYCDPNLGLRVQQKGTSETD | |
| | | TICTCEEGWHCTSEACESCV | |
| | | LHRSCSPGFGVKQIATGVSD | |
| | | TICEPCPVGFFSNVSSAFEK | |
| | | CHPWTSCETKDLVVQQAGTN | |
| | | KTDVVCGPQDRLRALVVIPI | |
| | | IFGILFAILLVLVFIKKVAK | |
| | | KPTNKAPHPKQEPQEINFPD | |
| | | DLPGSNTAAPVQETLHGCQP | |
| | | VTQEDGKESRISVQERQ | |
| murine CD40 | P27512 | MVSLPRLCALWGCLLTAVHL | 56 |
| | | GQCVTCSDKQYLHDGQCCDL | |
| | | CQPGSRLTSHCTALEKTQCH | |
| | | PCDSGEFSAQWNREIRCHQH | |
| | | RHCEPNQGLRVKKEGTAESD | |
| | | TVCTCKEGQHCTSKDCEACA | |
| | | QHTPCIPGFGVMEMATETTD | |
| | | TVCHPCPVGFFSNQSSLFEK | |
| | | CYPWTSCEDKNLEVLQKGTS | |
| | | QTNVICGLKSRMRALLVIPV | |
| | | VMGILITIFGVFLYIKKVVK | |
| | | KPKDNEILPPAARRQDPQEM | |
| | | EDYPGHNTAAPVQETLHGCQ | |
| | | PVTQEDGKESRISVQERQVT | |
| | | DSIALRPLV | |
| human CD40 L | P29965 | MIETYNQTSPRSAATGLPIS | 57 |
| | | MKIFMYLLTVFLITQMIGSA | |
| | | LFAVYLHRRLDKIEDERNLH | |
| | | EDFVFMKTIQRCNTGERSLS | |
| | | LLNCEEIKSQFEGFVKDIML | |
| | | NKEETKKENSFEMQKGDQNP | |
| | | QIAAHVISEASSKTTSVLQW | |
| | | AEKGYYTMSNNLVTLENGKQ | |
| | | LTVKRQGLYYIYAQVTFCSN | |
| | | REASSQAPFIASLCLKSPGR | |
| | | FERILLRAANTHSSAKPCGQ | |
| | | QSIHLGGVFELQPGASVFVN | |
| | | VTDPSQVSHGTGFTSFGLLK | |
| | | L | |
| murine CD40 L | P27548 | MIETYSQPSPRSVATGLPAS | 58 |
| | | MKIFMYLLTVFLITQMIGSV | |
| | | LFAVYLHRRLDKVEEEVNLH | |
| | | EDFVFIKKLKRCNKGEGSLS | |
| | | LLNCEEMRRQFEDLVKDITL | |
| | | NKEEKKENSFEMQRGDEDPQ | |
| | | IAAHVVSEANSNAASVLQWA | |
| | | KKGYYTMKSNLVMLENGKQL | |
| | | TVKREGLYYVYTQVTFCSNR | |
| | | EPSSQRPFIVGLWLKPSSGS | |
| | | ERILLKAANTHSSSQLCEQQ | |
| | | SVHLGGVFELQAGASVFVNV | |
| | | TEASQVIHRVGFSSFGLLKL | |
| human ICOS L | NM_ | MRLGSPGLLFLLFSSLRADT | 59 |
| | 015259.6 | QEKEVRAMVGSDVELSCACP | |
| | | EGSRFDLNDVYVYWQTSESK | |
| | | TVVTYHIPQNSSLENVDSRY | |
| | | RNRALMSPAGMLRGDFSLRL | |
| | | FNVTPQDEQKFHCLVLSQSL | |
| | | GFQEVLSVEVTLHVAANFSV | |
| | | PVVSAPHSPSQDELTFTCTS | |
| | | INGYPRPNVYWINKTDNSLL | |
| | | DQALQNDTVFLNMRGLYDVV | |
| | | SVLRIARTPSVNIGCCIENV | |
| | | LLQQNLTVGSQTGNDIGERD | |
| | | KITENPVSTGEKNAATWSIL | |
| | | AVLCLLVVVAVAIGWVCRDR | |
| | | CLQHSYAGAWAVSPETELTG | |
| | | HV | |
| murine ICOS L | NM_ | MQLKCPCFVSLGTRQPVWKK | 60 |
| | 015790.3 | LHVSSGFFSGLGLFLLLLSS | |
| | | LCAASAETEVGAMVGSNVVL | |
| | | SCIDPHRRHFNLSGLYVYWQ | |
| | | IENPEVSVTYYLPYKSPGIN | |
| | | VDSSYKNRGHLSLDSMKQGN | |
| | | FSLYLKNVTPQDTQEFTCRV | |
| | | FMNTATELVKILEEVVRLRV | |
| | | AANFSTPVISTSDSSNPGQE | |
| | | RTYTCMSKNGYPEPNLYWIN | |
| | | TTDNSLIDTALQNNTVYLNK | |
| | | LGLYDVISTLRLPWTSRGDV | |
| | | LCCVENVALHQNITSISQAE | |
| | | SFTGNNTKNPQETHNNELKV | |
| | | LVPVLAVLAAAAFVSFIIYR | |
| | | RTRPHRSYTGPKTVQLELTD | |
| | | HA | |
| human ICOS | NM_ | MKSGLWYFFLFCLRIKVLTG | 61 |
| | 012092.4 | EINGSANYEMFIFHNGGVQI | |
| | | LCKYPDIVQQFKMQLLKGGQ | |
| | | ILCDLTKTKGSGNTVSIKSL | |
| | | KFCHSQLSNNSVSFFLYNLD | |
| | | HSHANYYFCNLSIFDPPPFK | |
| | | VTLTGGYLHIYESQLCCQLK | |
| | | FWLPIGCAAFVVVCILGCIL | |
| | | ICWLTKKKYSSSVHDPNGEY | |
| | | MFMRAVNTAKKSRLTDVTL | |
| murine ICOS | NM_ | MKPYFCRVFVFCFLIRLLTG | 62 |
| | 017480.2 | EINGSADHRMFSFHNGGVQI | |
| | | SCKYPETVQQLKMRLFRERE | |
| | | VLCELTKTKGSGNAVSIKNP | |
| | | MLCLYHLSNNSVSFFLNNPD | |
| | | SSQGSYYFCSLSIFDPPPFQ | |
| | | ERNLSGGYLHIYESQLCCQL | |
| | | KLWLPVGCAAFVVVLLFGCI | |
| | | LIIWFSKKKYGSSVHDPNSE | |
| | | YMFMAAVNTNKKSRLAGVTS | |
| Transmembrane | NP_ | KFTIVFPHNQKGNWKNVPSN | 63 |
| Domain 1 | 955548 | YHYCPSSSDLNWHNDLIGTA | |
| | | LQVKMPKSHKAIQADGWMCH | |
| | | ASKWVTTCDFRWYGPKYITH | |
| | | SIRSFTPSVEQCKESIEQTK | |
| | | QGTWLNPGFPPQSCGYATVT | |
| | | DAEAVIVQVTPHHVLVDEYT | |
| | | GEWVDSQFINGKCSNYICPT | |
| | | VHNSTTWHSDYKVKGLCDSN | |
| | | LISMDITFFSEDGELSSLGK | |
| | | EGTGFRSNYFAYETGGKACK | |
| | | MQYCKHWGVRLPSGVWFEMA | |
| | | DKDLFAAARFPECPEGSSIS | |
| | | APSQTSVDVSLIQDVERILD | |
| | | YSLCQETWSKIRAGLPISPV | |
| | | DLSYLAPKNPGTGPAFTIIN | |
| | | GTLKYFETRYIRVDIAAPIL | |
| | | SRMVGMISGTTTERELWDDW | |
| | | APYEDVEIGPNGVLRTSSGY | |
| | | KFPLYMIGHGMLDSDLHLSS | |
| | | KAQVFEHPHIQDAASQLPDD | |
| | | ESLFFGDTGLSKNPIELVEG | |
| | | WFSSWKSSIASFFFIIGLII | |
| | | GLFLVLRVGIHLCIKLKHTK | |
| | | KRQIYTDIEMNRLGK | |
| Transmembrane | QJF75467 | MFVFLVLLPLVSSQCVNLTT | 64 |
| Domain 2 | | RTQLPPAYTNSFTRGVYYPD | |
| | | KVFRSSVLHSTQDLFLPFFS | |
| | | NVTWFHAIHVSGTNGTKRFD | |
| | | NPVLPFNDGVYFASTEKSNI | |
| | | IRGWIFGTTLDSKTQSLLIV | |
| | | NNATNVVIKVCEFQFCNDPF | |
| | | LGVYYHKNNKSWMESEFRVY | |
| | | SSANNCTFEYVSQPFLMDLE | |
| | | GKQGNFKNLREFVFKNIDGY | |
| | | FKIYSKHTPINLVRDLPQGF | |
| | | SALEPLVDLPIGINITRFQT | |
| | | LLALHRSYLTPGDSSSGWTA | |
| | | GAAAYYVGYLQPRTFLLKYN | |
| | | ENGTITDAVDCALDPLSETK | |
| | | CTLKSFTVEKGIYQTSNFRV | |
| | | QPTESIVRFPNITNLCPFGE | |
| | | VFNATRFASVYAWNRKRISN | |
| | | CVADYSVLYNSASFSTFKCY | |
| | | GVSPTKLNDLCFTNVYADSF | |
| | | VIRGDEVRQIAPGQTGKIAD | |
| | | YNYKLPDDFTGCVIAWNSNN | |
| | | LDSKVGGNYNYLYRLFRKSN | |
| | | LKPFERDISTEIYQAGSTPC | |
| | | NGVEGFNCYFPLQSYGFQPT | |
| | | NGVGYQPYRVVVLSFELLHA | |
| | | PATVCGPKKSTNLVKNKCVN | |
| | | FNFNGLTGTGVLTESNKKFL | |
| | | PFQQFGRDIADTTDAVRDPQ | |
| | | TLEILDITPCSFGGVSVITP | |
| | | GTNTSNQVAVLYQDVNCTEV | |
| | | PVAIHADQLTPTWRVYSTGS | |
| | | NVFQTRAGCLIGAEHVNNSY | |
| | | ECDIPIGAGICASYQTQTNS | |
| | | PRRARSVASQSIIAYTMSLG | |
| | | AENSVAYSNNSIAIPTNFTI | |
| | | SVTTEILPVSMTKTSVDCTM | |
| | | YICGDSTECSNLLLQYGSFC | |
| | | TQLNRALTGIAVEQDKNTQE | |
| | | VFAQVKQIYKTPPIKDFGGF | |
| | | NFSQILPDPSKPSKRSFIED | |
| | | LLFNKVTLADAGFIKQYGDC | |
| | | LGDIAARDLICAQKFNGLTV | |
| | | LPPLLTDEMIAQYTSALLAG | |
| | | TITSGWTFGAGAALQIPFAM | |
| | | QMAYRFNGIGVTQNVLYENQ | |
| | | KLIANQFNSAIGKIQDSLSS | |
| | | TASALGKLQDVVNQNAQALN | |
| | | TLVKQLSSNFGAISSVLNDI | |
| | | LSRLDKVEAEVQIDRLITGR | |
| | | LQSLQTYVTQQLIRAAEIRA | |
| | | SANLAATKMSECVLGQSKRV | |
| | | DFCGKGYHLMSFPQSAPHGV | |
| | | VFLHVTYVPAQEKNFTTAPA | |
| | | ICHDGKAHFPREGVFVSNGT | |
| | | HWFVTQRNFYEPQIITTDNT | |
| | | FVSGNCDVVIGIVNNTVYDP | |
| | | LQPELDSFKEELDKYFKNHT | |
| | | SPDVDLGDISGINASVVNIQ | |
| | | KEIDRLNEVAKNLNESLIDL | |
| | | QELGKYEQYIKWPWYIWLGF | |
| | | IAGLIAIVMVTIMLCCMTSC | |
| | | CSCLKGCCSCGSCCKFDEDD | |
| | | SEPVLKGVKLHYT | |

TABLE 5
Additional Exemplary Immune Checkpoint Sequences

| | | |
|---|---|---|
| | | SEQ |
| Gene | | ID |
| Name | Amino Acid Sequences | Nos. |

Inhibitory Immune Checkpoints

| | | |
|---|---|---|
| Human | MEKKWKYCAVYYIIQIHFVKGVWEKTVNTE | 96 |
| CD96 | ENVYATLGSDVNLTCQTQTVGFFVQMQWSK | |
| | VTNKIDLIAVYHPQYGFYCAYGRPCESLVT | |
| | FTETPENGSKWTLHLRNMSCSVSGRYECML | |
| | VLYPEGIQTKIYNLLIQTHVTADEWNSNHT | |
| | IEIEINQTLEIPCFQNSSSKISSEFTYAWS | |
| | VENSSTDSWVLLSKGIKEDNGTQETLISQN | |
| | HLISNSTLLKDRVKLGTDYRLHLSPVQIFD | |
| | DGRKFSCHIRVGPNKILRSSTTVKVFAKPE | |
| | IPVIVENNSTDVLVERRFTCLLKNVFPKAN | |
| | ITWFIDGSFLHDEKEGIYITNEERKGKDGF | |
| | LELKSVLTRVHSNKPAQSDNLTIWCMALSP | |
| | VPGNKVWNISSEKITFLLGSEISSTDPPLS | |
| | VTESTLDTQPSPASSVSPARYPATSSVTLV | |
| | DVSALRPNTTPQPSNSSMTTRGFNYPWTSS | |
| | GTDTKKSVSRIPSETYSSSPSGAGSTLHDN | |
| | VFTSTARAFSEVPTTANGSTKTNHVHITGI | |
| | VVNKPKDGMSWPVIVAALLFCCMILFGLGV | |
| | RKWCQYQKEIMERPPPFKPPPPPIKYTCIQ | |
| | EPNESDLPYHEMETL | |
| Murine | MGRKWTYCVVYTIIQIQFFRGVWEELFNVG | 97 |
| CD96 | DDVYALPGSDINLTCQTKEKNFLVQMQWSK | |
| | VTDKNDMIALYHPQYGLYCGQEHACESQVA | |
| | ATETEKGVTNWTLYLRNISSALGGKYECIF | |
| | TLYPEGIKTTVYNLIVEPYTQDEHNYTIEI | |
| | ETNRTLEIPCFQNTSSEIPPRFTFSWLVEK | |
| | DGVEEVLFTHHHHVNNSTSFKGRIRLGGDY | |
| | RLHLSPVQIQDDGRTFSCHLTVNPLKAWKM | |
| | STTVKVFAKPEILMTVENSTMDVLGERVFT | |
| | CLLKNVFPKANITWFIDGRFLQGNEEGIYI | |
| | TNEEKNCSSGFWELKSVLTRMHSGPSQSNN | |
| | MTAWCMALSPGPRNKMWNTSSQPITVSFDS | |
| | VIAPTKHLPTVTGSTLGTQPFSDAGVSPTG | |
| | YLATPSVTIVDENGLTPDATPQTSNSSMTT | |
| | KDGNYLEASSGTDAKNSSRAAASSKSGSWP | |
| | FPFTSPPEWHSLPGTSTGPQEPDSPVSWIP | |
| | SEVHTSAPLDASLAPHDTIISTTTEFPNVL | |
| | TTANGTTKIDHGPITSIIVNQPSDGMSWPV | |
| | LVAALLFFCTLLFGLGVRKWYRYQNEIMER | |
| | PPPFKPPPPPIKYTYIQEPIGCDLCCHEME | |
| | VL | |
| Human | MASLGQILFWSIISIIIILAGAIALIIGFG | 98 |
| B7-H4 | ISGRHSITVTTVASAGNIGEDGILSCTFEP | |
| | DIKLSDIVIQWLKEGVLGLVHEFKEGKDEL | |
| | SEQDEMFRGRTAVFADQVIVGNASLRLKNV | |
| | QLTDAGTYKCYIITSKGKGNANLEYKTGAF | |
| | SMPEVNVDYNASSETLRCEAPRWFPQPTVV | |
| | WASQVDQGANFSEVSNTSFELNSENVTMKV | |
| | VSVLYNVTINNTYSCMIENDIAKATGDIKV | |
| | TESEIKRRSHLQLLNSKASLCVSSFFAISW | |
| | ALLPLSPYLMLK | |
| Murine | MASLGQIIFWSIINIIIILAGAIALIIGFG | 99 |
| B7-H4 | ISGKHFITVTTFTSAGNIGEDGTLSCTFEP | |
| | DIKLNGIVIQWLKEGIKGLVHEFKEGKDDL | |
| | SQQHEMFRGRTAVFADQVVVGNASLRLKNV | |
| | QLTDAGTYTCYIRTSKGKGNANLEYKTGAF | |
| | SMPEINVDYNASSESLRCEAPRWFPQPTVA | |
| | WASQVDQGANFSEVSNTSFELNSENVTMKV | |
| | VSVLYNVTINNTYSCMIENDIAKATGDIKV | |
| | TDSEVKRRSQLQLLNSGPSPCVFSSAFVAG | |
| | WALLSLSCCLMLR | |
| Human | MLLLLLLPLLWGRERVEGQKSNRKDYSLTM | 100 |
| SIGLE | QSSVTVQEGMCVHVRCSFSYPVDSQTDSDP | |
| C7 | VHGYWFRAGNDISWKAPVATNNPAWAVQEE | |
| | TRDRFHLLGDPQTKNCTLSIRDARMSDAGR | |
| | YFFRMEKGNIKWNYKYDQLSVNVTALTHRP | |
| | NILIPGTLESGCFQNLTCSVPWACEQGTPP | |
| | MISWMGTSVSPLHPSTTRSSVLTLIPQPQH | |
| | HGTSLTCQVTLPGAGVTTNRTIQLNVSYPP | |
| | QNLTVTVFQGEGTASTALGNSSSLSVLEGQ | |
| | SLRLVCAVDSNPPARLSWTWRSLTLYPSQP | |
| | SNPLVLELQVHLGDEGEFTCRAQNSLGSQH | |
| | VSLNLSLQQEYTGKMRPVSGVLLGAVGGAG | |
| | ATALVFLSFCVIFIVVRSCRKKSARPAADV | |
| | GDIGMKDANTIRGSASQGNLTESWADDNPR | |
| | HHGLAAHSSGEEREIQYAPLSFHKGEPQDL | |
| | SGQEATNNEYSEIKIPK | |
| Human | MLLLLLPLLWGRERAEGQTSKLLTMQSSVT | 101 |
| SIGLE | VQEGLCVHVPCSFSYPSHGWIYPGPVVHGY | |
| C9 | WFREGANTDQDAPVATNNPARAVWEETRDR | |
| | FHLLGDPHTKNCTLSIRDARRSDAGRYFFR | |
| | MEKGSIKWNYKHHRLSVNVTALTHRPNILI | |
| | PGTLESGCPQNLTCSVPWACEQGTPPMISW | |
| | IGTSVSPLDPSTTRSSVLTLIPQPQDHGTS | |
| | LTCQVTFPGASVTTNKTVHLNVSYPPQNLT | |
| | MTVFQGDGTVSTVLGNGSSLSLPEGQSLRL | |
| | VCAVDAVDSNPPARLSLSWRGLTLCPSQPS | |
| | NPGVLELPWVHLRDAAEFTCRAQNPLGSQQ | |
| | VYLNVSLQSKATSGVTQGVVGGAGATALVF | |
| | LSFCVIFVVVRSCRKKSARPAAGVGDTGIE | |
| | DANAVRGSASQGPLTEPWAEDSPPDQPPPA | |
| | SARSSVGEGELQYASLSFQMVKPWDSRGQE | |
| | ATDTEYSEIKIHR | |

Stimulatory Immune Checkpoints

| | | |
|---|---|---|
| Human | MARPHPWWLCVLGTLVGLSATPAPKSCPER | 102 |
| CD27 | HYWAQGKLCCQMCEPGTFLVKDCDQHRKAA | |
| | QCDPCIPGVSFSPDHHTRPHCESCRHCNSG | |
| | LLVRNCTITANAECACRNGWQCRDKECTEC | |
| | DPLPNPSLTARSSQALSPHPQPTHLPYVSE | |
| | MLEARTAGHMQTLADFRQLPARTLSTHWPP | |
| | QRSLCSSDFIRILVIFSGMFLVFTLAGALF | |
| | LHQRRKYRSNKGESPVEPAEPCHYSCPREE | |
| | EGSTIPIQEDYRKPEPACSP | |
| Murine | MAWPPPYWLCMLGTLVGLSATLAPNSCPDK | 103 |
| CD27 | HYWTGGGLCCRMCEPGTFFVKDCEQDRTAA | |
| | QCDPCIPGTSFSPDYHTRPHCESCRHCNSG | |
| | FLIRNCTVTANAECSCSKNWQCRDQECTEC | |
| | DPPLNPALTRQPSETPSPQPPPTHLPHGTE | |
| | KPSWPLHRQLPNSTVYSQRSSHRPLCSSDC | |
| | IRIFVTFSSMFLIFVLGAILFFHQRRNHGP | |
| | NEDRQAVPEEPCPYSCPREEEGSAIPIQED | |
| | YRKPEPAFYP | |
| Human | MLRLLLALNLFPSIQVTGNKILVKQSPMLV | 104 |
| CD28 | AYDNAVNLSCKYSYNLFSREFRASLHKGLD | |
| | SAVEVCVVYGNYSQQLQVYSKTGFNCDGKL | |
| | GNESVTFYLQNLYVNQTDIYFCKIEVMYPP | |
| | PYLDNEKSNGTIIHVKGKHLCPSPLFPGPS | |
| | KPFWVLVVVGGVLACYSLLVTVAFIIFWVR | |
| | SKRSRLLHSDYMNMTPRRPGPTRKHYQPYA | |
| | PPRDFAAYRS | |
| Murine | MTLRLLFLALNFFSVQVTENKILVKQSPLL | 105 |
| CD28 | VVDSNEVSLSCRYSYNLLAKEFRASLYKGV | |
| | NSDVEVCVGNGNFTYQPQFRSNAEFNCDGD | |
| | FDNETVTFRLWNLHVNHTDIYFCKIEFMYP | |
| | PPYLDNERSNGTIIHIKEKHLCHTQSSPKL | |
| | FWALVVVAGVLFCYGLLVTVALCVIWTNSR | |
| | RNRLLQSDYMNMTPRRPGLTRKPYQPYAPA | |
| | RDFAAYRP | |
| Human | MAAPALSWRLPLLILLLPLATSWASAAVNG | 106 |
| CD122 | TSQFTCFYNSRANISCVWSQDGALQDTSCQ | |
| | VHAWPDRRRWNQTCELLPVSQASWACNLIL | |
| | GAPDSQKLTTVDIVTLRVLCREGVRWRVMA | |
| | IQDFKPFENLRLMAPISLaQVVHVETHRCN | |
| | ISWEISQASHYFERHLEFE | |
| | ARTLSPGHTWEEAPLLTLKQKQEWICLETL | |
| | TPDTQYEFQVRVKPLQGEFTTWSPWSQPLA | |
| | FRTKPAALGKDTIPWLGHLLVGLSGAFGFI | |
| | ILVYLLINCRNTGPWLKKVLKCNTPDPSKF | |
| | FSQLSSEHGGDVQKWLSSPFPSSSFSPGGL | |
| | APEISPLEVLERDKVTQLLLQQDKVPEPAS | |
| | LSSNHSLTSCFTNQGYFFFHLPDALEIEAC | |
| | QVYFTYDPYSEEDPDEGVAGAPTGSSPQPL | |
| | QPLSGEDDAYCTFPSRDDLLLFSPSLLGGP | |
| | SPPSTAPGGSGAGEERMPPSLQERVPRDWD | |
| | PQPLGPPTPGVPDLVDFQPPPELVLREAGE | |
| | EVPDAGPREGVSFPWSRPPGQGEFRALNAR | |
| | LPLNTDAYLSLQELQGQDPTHLV | |
| Murine | MATIALPWSLSLYVFLLLLATPWASAAVKN | 107 |
| CD122 | CSHLECFYNSRANVSCMWSHEEALNVTTCH | |
| | VHAKSNLRHWNKTCELTLVRQASWACNLIL | |
| | GSFPESQSLTSVDLLDINVVCWEEKGWRRV | |
| | KTCDFHPFDNLRLVAPHSLQVLHIDTQRCN | |
| | ISWKVSQVSHYIEPYLEFEARRRLLGHSWE | |
| | DASVLSLKQRQQWLFLEMLIPSTSYEVQVR | |
| | VKAQRNNTGTWSPWSQPLTFRTRPADPMKE | |
| | ILPMSWLRYLLLVLGCFSGFFSCVYILVKC | |
| | RYLGPWLKTVLKCHIPDPSEFFSQLSSQHG | |
| | GDLQKWLSSPVPLSFFSPSGPAPEISPLEV | |
| | LDGDSKAVQLLLLQKDSAPLPSPSGHSQAS | |
| | CFTNQGYFFFHLPNALEIESCQVYFTYDPC | |
| | VEEEVEEDGSRLPEGSPHPPLLPLAGEQDD | |
| | YCAFPPRDDLLLFSPSLSTPNTAYGGSRAP | |
| | EERSPLSLHEGLPSLASRDLMGLQRPLERM | |
| | PEGDGEGLSANSSGEQASVPEGNLHGQDQD | |
| | RGQGPILTLNTDAYLSLQELQAQDSVHLI | |
| Human | MAQHGAMGAFRALCGLALLCALSLGQRPTG | 108 |
| GITR | GPGCGPGRLLLGTGTDARCCRVHTTRCCRD | |
| | YPGEECCSEWDCMCVQPEFHCGDPCCTTCR | |
| | HHPCPPGQGVQSQGKFSFGFQCIDCASGTF | |
| | SGGHEGHCKPWTDCTQFGFLTVFPGNKTHN | |
| | AVCVPGSPPAEPLGWLTVVLLAVAACVLLL | |
| | TSAQLGLHIWQLRSQCMWPRETQLLLEVPP | |
| | STEDARSCQFPEEERGERSAEEKGRLGDLW | |
| | V | |
| Murine | MGAWAMLYGVSMLCVLDLGQPSVVEEPGCG | 109 |
| GITR | PGKVQNGSGNNTRCCSLYAPGKEDCPKERC | |
| | ICVTPEYHCGDPQCKICKHYPCQPGQRVES | |
| | QGDIVFGFRCVACAMGTFSAGRDGHCRLWT | |
| | NCSQFGFLTMFPGNKTHNAVCIPEPLPTEQ | |
| | YGHLTVIFLVMAACIFFLTTVQLGLHIWQL | |
| | RRQHMCPRETQPFAEVQLSAEDACSFQFPE | |
| | EERGEQTEEKCHLGGRWP | |
| Human | MPEEGSGCSVRRRPYGCVLRAALVPLVAGL | 110 |
| CD70 | VICLVVCIQRFAQAQQQLPLESLGWDVAEL | |
| | QLNHTGPQQDPRLYWQGGPALGRSFLHGPE | |
| | LDKGQLRIHRDGIYMVHIQVTLAICSSTTA | |
| | SRHHPTTLAVGICSPASRSISLLRLSFHQG | |
| | CTIASQRLTPLARGDTLCTNLTGTLLPSRN | |
| | TDETFFGVQWVRP | |
| Murine | MPEEGRPCPWVRWSGTAFQRQWPWLLLVVF | 111 |
| CD70 | ITVFCCWFHCSGLLSKQQQRLLEHPEPHTA | |
| | ELQLNLTVPRKDPTLRWGAGPALGRSFTHG | |
| | PELEEGHLRIHQDGLYRLHIQVTLANCSSP | |
| | GSTLQHRATLAVGICSPAAHGISLLRGRFG | |
| | QDCTVALQRLTYLVHGDVLCTNLTLPLLPS | |
| | RNADETFFGVQWICP | |
| Human | MYRMQLLSCIALSLALVTNSAPTSSSTKKT | 112 |
| IL-2 | QLQLEHLLLDLQMILNGINNYKNPKLTRML | |
| | TFKFYMPKKATELKHLQCLEEELKPLEEVL | |
| | NLAQSKNFHLRPRDLISNINVIVLELKGSE | |
| | TTFMCEYADETATIVEFLNRWITFCQSIIS | |
| | TLT | |
| Murine | MYSMQLASCVTLTLVLLVNSAPTSSSTSSS | 113 |
| IL-2 | TAEAQQQQQQQQQQQQHLEQLLMDLQELLS | |
| | RMENYRNLKLPRMLTFKFYLPKQATELKDL | |
| | QCLEDELGPLRHVLDLTQSKSFQLEDAENF | |
| | ISNIRVTVVKLKGSDNTFECQFDDESATVV | |
| | DFLRRWIAFCQSIISTSPQ | |
| Human | MTLHPSPITCEFLFSTALISPKMCLSHLEN | 114 |
| GITRL | MPLSHSRTQGAQRSSWKLWLFCSIVMLLFL | |
| | CSFSWLIFIFLQLETAKEPCMAKFGPLPSK | |
| | WQMASSEPPCVNKVSDWKLEILQNGLYLIY | |
| | GQVAPNANYNDVAPFEVRLYKNKDMIQTLT | |
| | NKSKIQNVGGTYELHVGDTIDLIFNSEHQV | |
| | LKNNTYWGIILLANPQFIS | |
| Murine | MEEMPLRESSPQRAERCKKSWLLCIVALLL | 115 |
| GITRL | MLLCSLGTLIYTSLKPTAIESCMVKFELSS | |
| | SKWHMTSPKPHCVNTTSDGKLKILQSGTYL | |
| | IYGQVIPVDKKYIKDNAPFVVQIYKKNDVL | |
| | QTLMNDFQILPIGGVYELHAGDNIYLKFNS | |
| | KDHIQKTNTYWGIILMPDLPFIS | |
| Human | MRVLLAALGLLFLGALRAFPQDRPFEDTCH | 153 |
| CD30 | GNPSHYYDKAVRRCCYRCPMGLFPTQQCPQ | |
| | RPTDCRKQCEPDYYLDEADRCTACVTCSRD | |
| | DLVEKTPCAWNSSRVCECRPGMFCSTSAVN | |
| | SCARCFFHSVCPAGMIVKFPGTAQKNTVCE | |
| | PASPGVSPACASPENCKEPSSGTIPQAKPT | |
| | PVSPATSSASTMPVRGGTRLAQEAASKLTR | |
| | APDSPSSVGRPSSDPGLSPTQPCPEGSGDC | |
| | RKQCEPDYYLDEAGRCTACVSCSRDDLVEK | |
| | TPCAWNSSRTCECRPGMICATSATNSCARC | |
| | VPYPICAAETVTKPQDMAEKDTTFEAPPLG | |
| | TQPDCNPTPENGEAPASTSPTQSLLVDSQA | |
| | SKTLPIPTSAPVALSSTGKPVLDAGPVLFW | |
| | VILVLVVVVGSSAFLLCHRRACRKRIRQKL | |
| | HLCYPVQTSQPKLELVDSRPRRSSTQLRSG | |
| | ASVTEPVAEERGLMSQPLMETCHSVGAAYL | |
| | ESLPLQDASPAGGPSSPRDLPEPRVSTEHT | |
| | NNKIEKIYIMKADTVIVGTVKAELPEGRGL | |
| | AGPAEPELEEELEADHTPHYPEQETEPPLG | |
| | SCSDVMLSVEEEGKEDPLPTAASGK | |
| Murine | MSALLTAAGLLFLGMLQAFPTDRPLKTTCA | 154 |
| CD30 | GDLSHYPGEAARNCCYQCPSGLSPTQPCPR | |
| | GPAHCRKQCAPDYYVNEDGKCTACVTCLPG | |
| | LVEKAPCSGNSPRICECQPGMHCCTPAVNS | |
| | CARCKLHCSGEEVVKSPGTAKKDTICELPS | |
| | SGSGPNCSNPGDRKTLTSHATPQAMPTLES | |
| | PANDSARSLLPMRVTNLVQEDATELVKVPE | |
| | SSSSKAREPSPDPGNAEKNMTLELPSPGTL | |
| | PDISTSENSKEPASTASTLSLVVDAWTSSR | |
| | MQPTSPLSTGTPFLDPGPVLFWVAMVVLLV | |
| | GSGSFLLCYWKACRRRFQQKFHLDYLVQTF | |
| | QPKMEQTDSCPTEKLTQPQRSGSVTDPSTG | |
| | HKLSPVSPPPAVETCASVGATYLENLPLLD | |
| | DSPAGNPFSPREPPEPRVSTEHTNNRIEKI | |
| | YIMKADTVIVGSVKTEVPEGRAPAGSTESE | |
| | LEAELEVDHAPHYPEQETEPPLGSCTEVMF | |
| | SVEEGGKEDHGPTTVSEK | |
| Human | MIETYNQTSPRSAATGLPISMKIFMYLLTV | 155 |
| CD40L | FLITQMIGSALFAVYLHRRLDKIEDERNLH | |
| | EDFVFMKTIQRCNTGERSLSLLNCEEIKSQ | |
| | FEGFVKDIMLNKEETKKENSFEMQKGDQNP | |
| | QIAAHVISEASSKTTSVLQWAEKGYYTMSN | |
| | NLVTLENGKQLTVKRQGLYYIYAQVTFCSN | |
| | REASSQAPFIASLCLKSPGRFERILLRAAN | |
| | THSSAKPCGQQSIHLGGVFELQPGASVFVN | |
| | VTDPSQVSHGTGFTSFGLLKL | |
| Murine | MIETYSQPSPRSVATGLPASMKIFMYLLTV | 156 |
| CD40L | FLITQMIGSVLFAVYLHRRLDKVEEEVNLH | |
| | EDFVFIKKLKRCNKGEGSLSLLNCEEMRRQ | |
| | FEDLVKDITLNKEEKKENSFEMQRGDEDPQ | |
| | IAAHVVSEANSNAASVLQWAKKGYYTMKSN | |
| | LVMLENGKQLTVKREGLYYVYTQVTFCSNR | |
| | EPSSQRPFIVGLWLKPSSGSERILLKAANT | |
| | HSSSQLCEQQSVHLGGVFELQAGASVFVNV | |
| | TEASQVIHRVGFSSFGLLKL | |
| Human | MEESVVRPSVFVVDGQTDIPFTRLGRSHRR | 157 |
| LIGHT | QSCSVARVGLGLLLLLMGAGLAVQGWFLLQ | |
| | LHWRLGEMVTRLPDGPAGSWEQLIQERRSH | |
| | EVNPAAHLTGANSSLTGSGGPLLWETQLGL | |
| | AFLRGLSYHDGALVVTKAGYYYIYSKVQLG | |
| | GVGCPLGLASTITHGLYKRTPRYPEELELL | |
| | VSQQSPCGRATSSSRVWWDSSFLGGVVHLE | |
| | AGEKVVVRVLDERLVRLRDGTRSYFGAFMV | |
| Murine | MESVVQPSVFVVDGQTDIPFRRLEQNHRRR | 158 |
| LIGHT | RCGTVQVSLALVLLLGAGLATQGWFLLRLH | |
| | QRLGDIVAHLPDGGKGSWEKLIQDQRSHQA | |
| | NPAAHLTGANASLIGIGGPLLWETRLGLAF | |
| | LRGLTYHDGALVTMEPGYYYVYSKVQLSGV | |
| | GCPQGLANGLPITHGLYKRTSRYPKELELL | |
| | VSRRSPCGRANSSRVWWDSSFLGGVVHLEA | |
| | GEEVVVRVPGNRLVRPRDGTRSYFGAFMV | |
| Human | MDPGLQQALNGMAPPGDTAMHVPAGSVASH | 159 |
| CD30L | LGTTSRSYFYLTTATLALCLVFTVATIMVL | |
| | VVQRTDSIPNSPDNVPLKGGNCSEDLLCIL | |
| | KRAPFKKSWAYLQVAKHLNKTKLSWNKDGI | |
| | LHGVRYQDGNLVIQFPGLYFIICQLQFLVQ | |
| | CPNNSVDLKLELLINKHIKKQALVTVCESG | |
| | MQTKHVYQNLSQFLLDYLQVNTTISVNVDT | |
| | FQYIDTSTFPLENVLSIFLYSNSD | |
| Murine | MEPGLQQAGSCGAPSPDPAMQVQPGSVASP | 160 |
| CD30L | WRSTRPWRSTSRSYFYLSTTALVCLVVAVA | |
| | IILVLVVQKKDSTPNTTEKAPLKGGNCSED | |
| | LFCTLKSTPSKKSWAYLQVSKHLNNTKLSW | |
| | NEDGTIHGLIYQDGNLIVQFPGLYFIVCQL | |
| | QFLVQCSNHSVDLTLQLLINSKIKKQTLVT | |
| | VCESGVQSKNIYQNLSQFLLHYLQVNSTIS | |
| | VRVDNFQYVDTNTFPLDNVLSVFLYSSSD | |
| Human | MSFPCKFVASFLLIFNVSSKGAVSKEITNA | 161 |
| CD2 | LETWGALGQDINLDIPSFQMSDDIDDIKWE | |
| | KTSDKKKIAQFRKEKETFKEKDTYKLFKNG | |
| | TLKIKHLKTDDQDIYKVSIYDTKGKNVLEK | |
| | IFDLKIQERVSKPKISWTCINTTLTCEVMN | |
| | GTDPELNLYQDGKHLKLSQRVITHKWTTSL | |
| | SAKFKCTAGNKVSKESSVEPVSCPEKGLDI | |
| | YLIIGICGGGSLLMVFVALLVFYITKRKKQ | |
| | RSRRNDEELETRAHRVATEERGRKPHQIPA | |
| | STPQNPATSQHPPPPPGHRSQAPSHRPPPP | |
| | GHRVQHQPQKRPPAPSGTQVHQQKGPPLPR | |
| | PRVQPKPPHGAAENSLSPSSN | |
| Murine | MKCKFLGSFFLLFSLSGKGADCRDNETIWG | 162 |
| CD2 | VLGHGITLNIPNFQMTDDIDEVRWVRRGTL | |
| | VAEFKRKKPPFLISETYEVLANGSLKIKKP | |
| | MMRNDSGTYNVMVYGTNGMTRLEKDLDVRI | |
| | LERVSKPMIHWECPNTTLTCAVLQGTDFEL | |
| | KLYQGETLLNSLPQKNMSYQWTNLNAPFKC | |
| | EAINPVSKESKMEVVNCPEKGLSFYVTVGV | |
| | GAGGLLLVLLVALFIFCICKRRKRNRRRKD | |
| | EELEIKASRTSTVERGPKPHSTPAAAAQNS | |
| | VALQAPPPPGHHLQTPGHRPLPPGHRTREH | |
| | QQKKRPPPSGTQIHQQKGPPLPRPRVQPKP | |
| | PCGSGDGVSLPPPN | |

TABLE 6
Exemplary Immune Checkpoint Fusion
Protein Sequences

| | | | | SEQ |
|---|---|---|---|---|
| IC- | | | Amino Acid | ID |
| MVPs | TM¹ | OD² | Sequences | No. |
| Murine | VGTM | D4 | MGVRQVPWSFTWAVLQLSWQ | 116 |
| PD-1- | | | SGWLLEVPNGPWRSLTFYPA | |
| MVP | | | WLTVSEGANATFTCSLSNWS | |
| | | | EDLMLNWNRLSPSNQTEKQA | |
| | | | AFCNGLSQPVQDARFQIIQL | |
| | | | PNRHDFHMNILDTRRNDSGI | |
| | | | YLCGAISLHPKAKIEESPGA | |
| | | | ELVVTERILETSTRYPSPSP | |
| | | | KPEGRFQGMVIGIMSALVGI | |
| | | | PVLLLLAWALDIIQADGWMC | |
| | | | HASKWVTTCDFRWYGPKYIT | |
| | | | HSIRSFTPSVEQCKESIEQT | |
| | | | KQGTWLNPGFPPQSCGYATV | |
| | | | TDAEAVIVQVTPHHVLVDEY | |
| | | | TGEWVDSQFINGKCSNYICP | |
| | | | TVHNSTTWHSDYKVKGLCDS | |
| | | | NLGMLDSDLHLSSKAQVFEH | |
| | | | PHIQDAASQLPDDESLFFGD | |
| | | | TGLSKNPIELVEGWFSSWKS | |
| | | | SIASFFFIIGLIIGLFLVLR | |
| | | | VGIHLCIKLKHTKKRQIYTD | |
| | | | IEMNRLGK | |
| Murine | VGTM | D4 | MRIFAGIIFTACCHLLRAFT | 117 |
| PD-L1- | | | ITAPKDLYVVEYGSNVTMEC | |
| MVP | | | RFPVERELDLLALVVYWEKE | |
| | | | DEQVIQFVAGEEDLKPQHSN | |
| | | | FRGRASLPKDQLLKGNAALQ | |
| | | | ITDVKLQDAGVYCCIISYGG | |
| | | | ADYKRITLKVNAPYRKINQR | |
| | | | ISVDPATSEHELICQAEGYP | |
| | | | EAEVIWTNSDHQPVSGKRSV | |
| | | | TTSRTEGMLLNVTSSLRVNA | |
| | | | TANDVFYCTFWRSQPGQNHT | |
| | | | AELIIPELPATHPPQNRDII | |
| | | | QADGWMCHASKWVTTCDFRW | |
| | | | YGPKYITHSIRSFTPSVEQC | |
| | | | KESIEQTKQGTWLNPGFPPQ | |
| | | | SCGYATVIDAEAVIVQVTPH | |
| | | | HVLVDEYTGEWVDSQFINGK | |
| | | | CSNYICPTVHNSTTWHSDYK | |
| | | | VKGLCDSNLGMLDSDLHLSS | |
| | | | KAQVFEHPHIQDAASQLPDD | |
| | | | ESLFFGDTGLSKNPIELVEG | |
| | | | WFSSWKSSIASFFFIIGLII | |
| | | | GLFLVLRVGIHLCIKLKHTK | |
| | | | KRQIYTDIEMNRLGK | |
| Murine | VGTM | D4 | MIGQAVLFTTFLLLRAHQGQ | 118 |
| 2B4- | | | DCPDSSEEVVGVSGKPVQLR | |
| MVP | | | PSNIQTKDVSVQWKKTEQGS | |
| | | | HRKIEILNWYNDGPSWSNVS | |
| | | | FSDIYGFDYGDFALSIKSAK | |
| | | | LQDSGHYLLEITNTGGKVCN | |
| | | | KNFQLLILDHVETPNLKAQW | |
| | | | KPWTNGTCQLFLSCLVTKDD | |
| | | | NVSYALYRGSTLISNQRNST | |
| | | | HWENQIDASSLHTYTCNVSN | |
| | | | RASWANHTLNFTHGCQSVPS | |
| | | | NFRFLPDIIQADGWMCHASK | |
| | | | WVTTCDFRWYGPKYITHSIR | |
| | | | SFTPSVEQCKESIEQTKQGT | |
| | | | WLNPGFPPQSCGYATVTDAE | |
| | | | AVIVQVTPHHVLVDEYTGEW | |
| | | | VDSQFINGKCSNYICPTVHN | |
| | | | STTWHSDYKVKGLCDSNLGM | |
| | | | LDSDLHLSSKAQVFEHPHIQ | |
| | | | DAASQLPDDESLFFGDTGLS | |
| | | | KNPIELVEGWFSSWKSSIAS | |
| | | | FFFIIGLIIGLFLVLRVGIH | |
| | | | LCIKLKHTKKRQIYTDIEMN | |
| | | | RLGK | |
| Murine | VGTM | D4 | MLLLLPILNLSLQLHPVAAL | 119 |
| PD-L2- | | | FTVTAPKEVYTVDVGSSVSL | |
| MVP | | | ECDFDRRECTELEGIRASLQ | |
| | | | KVENDTSLQSERATLLEEQL | |
| | | | PLGKALFHIPSVQVRDSGQY | |
| | | | RCLVICGAAWDYKYLTVKVK | |
| | | | ASYMRIDTRILEVPGTGEVQ | |
| | | | LTCQARGYPLAEVSWQNVSV | |
| | | | PANTSHIRTPEGLYQVTSVL | |
| | | | RLKPQPSRNFSCMFWNAHMK | |
| | | | ELTSAIIDPLSRMEPKVPRD | |
| | | | IIQADGWMCHASKWVTTCDF | |
| | | | RWYGPKYITHSIRSFTPSVE | |
| | | | QCKESIEQTKQGTWLNPGFP | |
| | | | PQSCGYATVTDAEAVIVQVT | |
| | | | PHHVLVDEYTGEWVDSQFIN | |
| | | | GKCSNYICPTVHNSTTWHSD | |
| | | | YKVKGLCDSNLGMLDSDLHL | |
| | | | SSKAQVFEHPHIQDAASQLP | |
| | | | DDESLFFGDTGLSKNPIELV | |
| | | | EGWFSSWKSSIASFFFIIGL | |
| | | | IIGLFLVLRVGIHLCIKLKH | |
| | | | TKKRQIYTDIEMNRLGK | |
| Murine | VGTM | D4 | MTCLGLRRYKAQLQLPSRTW | 120 |
| CTLA4 | | | PFVALLTLLFIPVFSEAIQV | |
| -MVP | | | TQPSVVLASSHGVASFPCEY | |
| | | | SPSHNTDEVRVTVLRQTNDQ | |
| | | | MTEVCATTFTEKNTVGFLDY | |
| | | | PFCSGTFNESRVNLTIQGLR | |
| | | | AVDTGLYLCKVELMYPPPYF | |
| | | | VGMGNGTQIYVIDPEPCPDS | |
| | | | DFLLWILVAVSLGLFFYSFL | |
| | | | VTAVSLSKRIQDIIQADGWM | |
| | | | CHASKWVTTCDFRWYGPKYI | |
| | | | THSIRSFTPSVEQCKESIEQ | |
| | | | TKQGTWLNPGFPPQSCGYAT | |
| | | | VTDAEAVIVQVTPHHVLVDE | |
| | | | YTGEWVDSQFINGKCSNYIC | |
| | | | PTVHNSTTWHSDYKVKGLCD | |
| | | | SNLGMLDSDLHLSSKAQVFE | |
| | | | HPHIQDAASQLPDDESLFFG | |
| | | | DTGLSKNPIELVEGWFSSWK | |
| | | | SSIASFFFIIGLIIGLFLVL | |
| | | | RVGIHLCIKLKHTKKRQIYT | |
| | | | DIEMNRLGK | |
| Murine | VGTM | D4 | MADSFSLNDALAGSGNPNPQ | 121 |
| Galecti | | | GYPGAWGNQPGAGGYPGAAY | |
| n-3- | | | PGAYPGQAPPGAYPGQAPPG | |
| MVP | | | AYPGQAPPSAYPGPTAPGAY | |
| | | | PGPTAPGAYPGQPAPGAFPG | |
| | | | QPGAPGAYPQCSGGYPAAGP | |
| | | | YGVPAGPLTVPYDLPLPGGV | |
| | | | MPRMLITIMGTVKPNANRIV | |
| | | | LDFRRGNDVAFHFNPRFNEN | |
| | | | NRRVIVCNTKQDNNWGKEER | |
| | | | QSAFPFESGKPFKIQVLVEA | |
| | | | DHFKVAVNDAHLLQYNHRMK | |
| | | | NLREISQLGISGDITLTSAN | |
| | | | HAMIIIQADGWMCHASKWVT | |
| | | | TCDFRWYGPKYITHSIRSFT | |
| | | | PSVEQCKESIEQTKQGTWLN | |
| | | | PGFPPQSCGYATVTDAEAVI | |
| | | | VQVTPHHVLVDEYTGEWVDS | |
| | | | QFINGKCSNYICPTVHNSTT | |
| | | | WHSDYKVKGLCDSNLGMLDS | |
| | | | DLHLSSKAQVFEHPHIQDAA | |
| | | | SQLPDDESLFFGDTGLSKNP | |
| | | | IELVEGWFSSWKSSIASFFF | |
| | | | IIGLIIGLFLVLRVGIHLCI | |
| | | | KLKHTKKRQIYTDIEMNRLG | |
| | | | K | |
| Murine | VGTM | D4 | MGEDLLLGFLLLGLLWEAPV | 122 |
| LAG- | | | VSSGPGKELPVVWAQEGAPV | |
| 3- | | | HLPCSLKSPNLDPNFLRRGG | |
| MVP | | | VIWQHQPDSGQPTPIPALDL | |
| | | | HQGMPSPRQPAPGRYTVLSV | |
| | | | APGGLRSGRQPLHPHVQLEE | |
| | | | RGLQRGDFSLWLRPALRTDA | |
| | | | GEYHATVRLPNRALSCSLRL | |
| | | | RVGQASMIASPSGVLKLSDW | |
| | | | VLLNCSFSRPDRPVSVHWFQ | |
| | | | GQNRVPVYNSPRHFLAETFL | |
| | | | LLPQVSPLDSGTWGCVLTYR | |
| | | | DGFNVSITYNLKVLGLEPVA | |
| | | | PLTVYAAEGSRVELPCHLPP | |
| | | | GVGTPSLLIAKWTPPGGGPE | |
| | | | LPVAGKSGNFTLHLEAVGLA | |
| | | | QAGTYTCSIHLQGQQLNATV | |
| | | | TLAVITVTPKSFGLPGSRGK | |
| | | | LLCEVTPASGKERFVWRPLN | |
| | | | NLSRSCPGPVLEIQEARLLA | |
| | | | ERWQCQLYEGQRLLGATVYA | |
| | | | AESSSGAHSARRISGDLKGG | |
| | | | DIIQADGWMCHASKWVTTCD | |
| | | | FRWYGPKYITHSIRSFTPSV | |
| | | | EQCKESIEQTKQGTWLNPGF | |
| | | | PPQSCGYATVTDAEAVIVQV | |
| | | | TPHHVLVDEYTGEWVDSQFI | |
| | | | NGKCSNYICPTVHNSTTWHS | |
| | | | DYKVKGLCDSNLGMLDSDLH | |
| | | | LSSKAQVFEHPHIQDAASQL | |
| | | | PDDESLFFGDTGLSKNPIEL | |
| | | | VEGWFSSWKSSIASFFFIIG | |
| | | | LIIGLFLVLRVGIHLCIKLK | |
| | | | HTKKRQIYTDIEMNRLGK | |
| Murine | VGTM | D4 | MGKIYSFVLVAIALMMGREG | 123 |
| FGL1- | | | WALESESCLREQVRLRAQVH | |
| MVP | | | QLETRVKQQQTMIAQLLHEK | |
| | | | EVQFLDKGSENSFIDLGGKR | |
| | | | QYADCSEIYNDGFKQSGFYK | |
| | | | IKPLQSLAEFSVYCDMSDGG | |
| | | | GWTVIQRRSDGSENFNRGWN | |
| | | | D | |
| | | | YENGFGNFVQNNGEYWLGNK | |
| | | | NINLLTIQGDYTLKIDLTDF | |
| | | | EKNSSFAQYQSFKVGDKKSF | |
| | | | YELNIGEYSGTAGDSLSGTF | |
| | | | HPEVQWWASHQRMKFSTWDR | |
| | | | DNDNYQGNCAEEEQSGWWFN | |
| | | | RCHSANLNGVYYRGSYRAET | |
| | | | DNGVVWYTWHGWWYSLKSVV | |
| | | | MKIRPSDFIPNIIIIQADGW | |
| | | | MCHASKWVTTCDFRWYGPKY | |
| | | | ITHSIRSFTPSVEQCKESIE | |
| | | | QTKQGTWLNPGFPPQSCGYA | |
| | | | TVTDAEAVIVQVTPHHVLVD | |
| | | | EYTGEWVDSQFINGKCSNYI | |
| | | | CPTVHNSTTWHSDYKVKGLC | |
| | | | DSNLGMLDSDLHLSSKAQVF | |
| | | | EHPHIQDAASQLPDDESLFF | |
| | | | GDTGLSKNPIELVEGWFSSW | |
| | | | KSSIASFFFIIGLIIGLFLV | |
| | | | LRVGIHLCIKLKHTKKRQIY | |
| | | | TDIEMNRLGK | |
| Murine | VGTM | D4 | MEPLPGWGSAPWSQAPTDNT | 124 |
| HVEM | | | FRLVPCVFLLNLLQRISAQP | |
| -MVP | | | SCRQEEFLVGDECCPMCNPG | |
| | | | YHVKQVCSEHTGTVCAPCPP | |
| | | | QTYTAHANGLSKCLPCGVCD | |
| | | | PDMGLLTWQECSSWKDTVCR | |
| | | | CIPGYFCENQDGSHCSTCLQ | |
| | | | HTTCPPGQRVEKRGTHDQDT | |
| | | | VCADCLTGTFSLGGTQEECL | |
| | | | PWTNCSAFQQEVRRGTNSTD | |
| | | | TTCSSQVDIIQADGWMCHAS | |
| | | | KWVTTCDFRWYGPKYITHSI | |
| | | | RSFTPSVEQCKESIEQTKQG | |
| | | | TWLNPGFPPQSCGYATVTDA | |
| | | | EAVIVQVTPHHVLVDEYTGE | |
| | | | WVDSQFINGKCSNYICPTVH | |
| | | | NSTTWHSDYKVKGLCDSNLG | |
| | | | MLDSDLHLSSKAQVFEHPHI | |
| | | | QDAASQLPDDESLFFGDTGL | |
| | | | SKNPIELVEGWFSSWKSSIA | |
| | | | SFFFIIGLIIGLFLVLRVGI | |
| | | | HLCIKLKHTKKRQIYTDIEM | |
| | | | NRLGK | |
| Murine | VGTM | D4 | MKTVPAMLGTPRLFREFFIL | 125 |
| BTLA- | | | HLGLWSILCEKATKRNDEEC | |
| MVP | | | PVQLTITRNSKQSARTGELF | |
| | | | KIQCPVKYCVHRPNVTWCKH | |
| | | | NGTICVPLEVSPQLYTSWEE | |
| | | | NQSVPVFVLHFKPIHLSDNG | |
| | | | SYSCSTNFNSQVINSHSVTI | |
| | | | HVTERTQNSSEHPLITVSDI | |
| | | | PDATNASGPSTMEERPGRTW | |
| | | | LDIIQADGWMCHASKWVTTC | |
| | | | DFRWYGPKYITHSIRSFTPS | |
| | | | VEQCKESIEQTKQGTWLNPG | |
| | | | FPPQSCGYATVTDAEAVIVQ | |
| | | | VTPHHVLVDEYTGEWVDSQF | |
| | | | INGKCSNYICPTVHNSTTWH | |
| | | | SDYKVKGLCDSNLGMLDSDL | |
| | | | HLSSKAQVFEHPHIQDAASQ | |
| | | | LPDDESLFFGDTGLSKNPIE | |
| | | | LVEGWFSSWKSSIASFFFII | |
| | | | GLIIGLFLVLRVGIHLCIKL | |
| | | | KHTKKRQIYTDIEMNRLGK | |
| Murine | VGTM | D4 | MERILMAPGQSCCALAILLA | 126 |
| CD160 | | | IVNFQHGGCIHVTSSASQKG | |
| -MVP | | | GRLDLTCTLWHKKDEAEGLI | |
| | | | LFWCKDNPWNCSPETNLEQL | |
| | | | RVKRDPETDGITEKSSQLVF | |
| | | | TIEQATPSDSGTYQCCARSQ | |
| | | | KPEIYIHGHFLSVLVTGNHT | |
| | | | EIRQRQRSHPDFSHINGTLS | |
| | | | SGFLQVKAWGMLVTSLVALQ | |
| | | | ALYTLAADIIQADGWMCHAS | |
| | | | KWVTTCDFRWYGPKYITHSI | |
| | | | RSFTPSVEQCKESIEQTKQG | |
| | | | TWLNPGFPPQSCGYATVTDA | |
| | | | EAVIVQVTPHHVLVDEYTGE | |
| | | | WVDSQFINGKCSNYICPTVH | |
| | | | NSTTWHSDYKVKGLCDSNLG | |
| | | | MLDSDLHLSSKAQVFEHPHI | |
| | | | QDAASQLPDDESLFFGDTGL | |
| | | | SKNPIELVEGWFSSWKSSIA | |
| | | | SFFFIIGLIIGLFLVLRVGI | |
| | | | HLCIKLKHTKKRQIYTDIEM | |
| | | | NRLGK | |
| Murine | VGTM | D4 | MCFIKQGWCLVLELLLLPLG | 127 |
| CD48- | | | TGFQGHSIPDINATTGSNVT | |
| MVP | | | LKIHKDPLGPYKRITWLHTK | |
| | | | NQKILEYNYNSTKTIFESEF | |
| | | | KGRVYLEENNGALHISNVRK | |
| | | | EDKGTYYMRVLRETENELKI | |
| | | | TLEVFDPVPKPSIEINKTEA | |
| | | | STDSCHLRLSCEVKDQHVDY | |
| | | | TWYESSGPFPKKSPGYVLDL | |
| | | | IVTPQNKSTFYTCQVSNPVS | |
| | | | SKNDTVYFTLPCDLARSSGV | |
| | | | CWTATWLVVTTLIIHRILLT | |
| | | | IIQADGWMCHASKWVTTCDF | |
| | | | RWYGPKYITHSIRSFTPSVE | |
| | | | QCKESIEQTKQGTWLNPGFP | |
| | | | PQSCGYATVTDAEAVIVQVT | |
| | | | PHHVLVDEYTGEWVDSQFIN | |
| | | | GKCSNYICPTVHNSTTWHSD | |
| | | | YKVKGLCDSNLGMLDSDLHL | |
| | | | SSKAQVFEHPHIQDAASQLP | |
| | | | DDESLFFGDTGLSKNPIELV | |
| | | | EGWFSSWKSSIASFFFIIGL | |
| | | | IIGLFLVLRVGIHLCIKLKH | |
| | | | TKKRQIYTDIEMNRLGK | |
| Murine | VGTM | D4 | MARAAVLPPSRLSPTLPLLP | 128 |
| CD112 | | | LLLLLLQETGAQDVRVRVLP | |
| -MVP | | | EVRGRLGGTVELPCHLLPPT | |
| | | | TERVSQVTWQRLDGTVVAAF | |
| | | | HPSFGVDFPNSQFSKDRLSF | |
| | | | VRARPETNADLRDATLAFRG | |
| | | | LRVEDEGNYTCEFATFPNGT | |
| | | | RRGVTWLRVIAQPENHAEAQ | |
| | | | EVTIGPQSVAVARCVSTGGR | |
| | | | PPARITWISSLGGEAKDTQE | |
| | | | PGIQAGTVTIISRYSLVPVG | |
| | | | RADGVKVTCRVEHESFEEPI | |
| | | | LLPVTLSVRYPPEVSISGYD | |
| | | | DNWYLGRSEAILTCDVRSNP | |
| | | | EPTDYDWSTTSGVFPASAVA | |
| | | | QGSQLLVHSVDRMVNTTFIC | |
| | | | TATNAVGTGRAEQVILVRES | |
| | | | PSTAGAGATGGIIQADGWMC | |
| | | | HASKWVTTCDFRWYGPKYIT | |
| | | | HSIRSFTPSVEQCKESIEQT | |
| | | | KQGTWLNPGFPPQSCGYATV | |
| | | | TDAEAVIVQVTPHHVLVDEY | |
| | | | TGEWVDSQFINGKCSNYICP | |
| | | | TVHNSTTWHSDYKVKGLCDS | |
| | | | NLGMLDSDLHLSSKAQVFEH | |
| | | | PHIQDAASQLPDDESLFFGD | |
| | | | TGLSKNPIELVEGWFSSWKS | |
| | | | SIASFFFIIGLIIGLFLVLR | |
| | | | VGIHLCIKLKHTKKRQIYTD | |
| | | | IEMNRLGK | |
| Murine | VGTM | D4 | MKGWLLLVWVQGLIQAAFLA | 129 |
| TIGIT- | | | TGATAGTIDTKRNISAEEGG | |
| MVP | | | SVILQCHFSSDTAEVTQVDW | |
| | | | KQQDQLLAIYSVDLGWHVAS | |
| | | | VFSDRVVPGPSLGLTFQSLT | |
| | | | MNDTGEYFCTYHTYPGGIYK | |
| | | | GRIFLKVQESSVAQFQTAPD | |
| | | | IIQADGWMCHASKWVTTCDF | |
| | | | RWYGPKYITHSIRSFTPSVE | |
| | | | QCKESIEQTKQGTWLNPGFP | |
| | | | PQSCGYATVTDAEAVIVQVT | |
| | | | PHHVLVDEYTGEWVDSQFIN | |
| | | | GKCSNYICPTVHNSTTWHSD | |
| | | | YKVKGLCDSNLGMLDSDLHL | |
| | | | SSKAQVFEHPHIQDAASQLP | |
| | | | DDESLFFGDTGLSKNPIELV | |
| | | | EGWFSSWKSSIASFFFIIGL | |
| | | | IIGLFLVLRVGIHLCIKLKH | |
| | | | TKKRQIYTDIEMNRLGK | |
| Murine | VGTM | D4 | MAQLARATRSPLSWLLLLFC | 130 |
| CD155 | | | YALRKAGGDIRVLVPYNSTG | |
| -MVP | | | VLGGSTTLHCSLTSNENVTI | |
| | | | TQITWMKKDSGGSHALVAVF | |
| | | | HPKKGPNIKEPERVKFLAAQ | |
| | | | QDLRNASLAISNLSVEDEGI | |
| | | | YECQIATFPRGSRSTNAWLK | |
| | | | VQARPKNTAEALEPSPTLIL | |
| | | | QDVAKCISANGHPPGRISWP | |
| | | | SNVNGSHREMKEPGSQPGTT | |
| | | | TVTSYLSMVPSRQADGKNIT | |
| | | | CTVEHESLQELDQLLVTLSQ | |
| | | | PYPPENVSISGYDGNWYVGL | |
| | | | TNLTLTCEAHSKPAPDMAGY | |
| | | | NWSTTTGDFPNSVKRQGNML | |
| | | | LISTVEDGLNNTVIVCEVTN | |
| | | | ALGSGQGQVHIIVKEKPENM | |
| | | | QQNTRLHLIIQADGWMCHAS | |
| | | | KWVTTCDFRWYGPKYITHSI | |
| | | | RSFTPSVEQCKESIEQTKQG | |
| | | | TWLNPGFPPQSCGYATVIDA | |
| | | | EAVIVQVTPHHVLVDEYTGE | |
| | | | WVDSQFINGKCSNYICPTVH | |
| | | | NSTTWHSDYKVKGLCDSNLG | |
| | | | MLDSDLHLSSKAQVFEHPHI | |
| | | | QDAASQLPDDESLFFGDTGL | |
| | | | SKNPIELVEGWFSSWKSSIA | |
| | | | SFFFIIGLIIGLFLVLRVGI | |
| | | | HLCIKLKHTKKRQIYTDIEM | |
| | | | NRLGK | |
| Human | VGTM | D4 | MFSHLPFDCVLLLLLLLLTR | 131 |
| TIM3- | | | SSEVEYRAEVGQNAYLPCFY | |
| MVP | | | TPAAPGNLVPVCWGKGACPV | |
| | | | FECGNVVLRTDERDVNYWTS | |
| | | | RYWLNGDFRKGDVSLTIENV | |
| | | | TLADSGIYCCRIQIPGIMND | |
| | | | EKFNLKLVIKPAKVTPAPTR | |
| | | | QRDFTAAFPRMLTTRGHGPA | |
| | | | ETQTLGSLPDINLTQISTLA | |
| | | | NELRDSRLANDLRDSGATIR | |
| | | | IDIIQADGWMCHASKWVTTC | |
| | | | DFRWYGPKYITHSIRSFTPS | |
| | | | VEQCKESIEQTKQGTWLNPG | |
| | | | FPPQSCGYATVTDAEAVIVQ | |
| | | | VTPHHVLVDEYTGEWVDSQF | |
| | | | INGKCSNYICPTVHNSTTWH | |
| | | | SDYKVKGLCDSNLGMLDSDL | |
| | | | HLSSKAQVFEHPHIQDAASQ | |
| | | | LPDDESLFFGDTGLSKNPIE | |
| | | | LVEGWFSSWKSSIASFFFII | |
| | | | GLIIGLFLVLRVGIHLCIKL | |
| | | | KHTKKRQIYTDIEMNRLGK | |
| Human | VGTM | D4 | MGHLSAPLHRVRVPWQGLLL | 132 |
| Ceaca | | | TASLLTFWNPPTTAQLTTES | |
| ml- | | | MPFNVAEGKEVLLLVHNLPQ | |
| MVP | | | QLFGYSWYKGERVDGNRQIV | |
| | | | GYAIGTQQATPGPANSGRET | |
| | | | IYPNASLLIQNVTQNDTGFY | |
| | | | TLQVIKSDLVNEEATGQFHV | |
| | | | YPELPKPSISSNNSNPVEDK | |
| | | | DAVAFTCEPETQDTTYLWWI | |
| | | | NNQSLPVSPRLQLSNGNRTL | |
| | | | TLLSVTRNDTGPYECEIQNP | |
| | | | VSANRSDPVTLNVTYGPDTP | |
| | | | TISPSDTYYRPGANLSLSCY | |
| | | | AASNPPAQYSWLINGTFQQS | |
| | | | TQELFIPNITVNNSGSYTCH | |
| | | | ANNSVTGCNRTTVKTIIVTE | |
| | | | LSPVVAKPQIKASKTTVTGD | |
| | | | KDSVNLTCSTNDTGISIRWF | |
| | | | FKNQSLPSSERMKLSQGNTT | |
| | | | LSINPVKREDAGTYWCEVFN | |
| | | | PISKNQSDPIMLNVNYNALP | |
| | | | QENGLSPGIIQADGWMCHAS | |
| | | | KWVTTCDFRWYGPKYITHSI | |
| | | | RSFTPSVEQCKESIEQTKQG | |
| | | | TWLNPGFPPQSCGYATVTDA | |
| | | | EAVIVQVTPHHVLVDEYTGE | |
| | | | WVDSQFINGKCSNYICPTVH | |
| | | | NSTTWHSDYKVKGLCDSNLG | |
| | | | MLDSDLHLSSKAQVFEHPHI | |
| | | | QDAASQLPDDESLFFGDTGL | |
| | | | SKNPIELVEGWFSSWKSSIA | |
| | | | SFFFIIGLIIGLFLVLRVGI | |
| | | | HLCIKLKHTKKRQIYTDIEM | |
| | | | NRLGK | |
| Murine | VGTM | D4 | MDPRCTMGLAILIFVTVLLI | 133 |
| CD86- | | | SDAVSVETQAYFNGTAYLPC | |
| MVP | | | PFTKAQNISLSELVVFWQDQ | |
| | | | QKLVLYEHYLGTEKLDSVNA | |
| | | | KYLGRTSFDRNNWTLRLHNV | |
| | | | QIKDMGSYDCFIQKKPPTGS | |
| | | | IILQQTLTELSVIANFSEPE | |
| | | | IKLAQNVTGNSGINLTCTSK | |
| | | | QGHPKPKKMYFLITNSTNEY | |
| | | | GDNMQISQDNVTELFSISNS | |
| | | | LSLSFPDGVWHMTVVCVLET | |
| | | | ESMKISSKPLNFTQEFPSPQ | |
| | | | TYWKEDIIQADGWMCHASKW | |
| | | | VTTCDFRWYGPKYITHSIRS | |
| | | | FTPSVEQCKESIEQTKQGTW | |
| | | | LNPGFPPQSCGYATVIDAEA | |
| | | | VIVQVTPHHVLVDEYTGEWV | |
| | | | DSQFINGKCSNYICPTVHNS | |
| | | | TTWHSDYKVKGLCDSNLGML | |
| | | | DSDLHLSSKAQVFEHPHIQD | |
| | | | AASQLPDDESLFFGDTGLSK | |
| | | | NPIELVEGWFSSWKSSIASF | |
| | | | FFIIGLIIGLFLVLRVGIHL | |
| | | | CIKLKHTKKRQIYTDIEMNR | |
| | | | LGK | |
| Human | VGTM | D4 | MGLSNILFVMAFLLSGAAPL | 134 |
| CD86- | | | KIQAYFNETADLPCQFANSQ | |
| MVP | | | NQSLSELVVFWQDQENLVLN | |
| | | | EVYLGKEKFDSVHSKYMGRT | |
| | | | SFDSDSWTLRLHNLQIKDKG | |
| | | | LYQCIIHHKKPTGMIRIHQM | |
| | | | NSELSVLANFSQPEIVPISN | |
| | | | ITENVYINLTCSSIHGYPEP | |
| | | | KKMSVLLRTKNSTIEYDGIM | |
| | | | QKSQDNVTELYDVSISLSVS | |
| | | | FPDVTSNMTIFCILETDKTR | |
| | | | LLSSPFSIELEDPQPPPDHI | |
| | | | PWITAVLDIIQADGWMCHAS | |
| | | | KWVTTCDFRWYGPKYITHSI | |
| | | | RSFTPSVEQCKESIEQTKQG | |
| | | | TWLNPGFPPQSCGYATVIDA | |
| | | | EAVIVQVTPHHVLVDEYTGE | |
| | | | WVDSQFINGKCSNYICPTVH | |
| | | | NSTTWHSDYKVKGLCDSNLG | |
| | | | MLDSDLHLSSKAQVFEHPHI | |
| | | | QDAASQLPDDESLFFGDTGL | |
| | | | SKNPIELVEGWFSSWKSSIA | |
| | | | SFFFIIGLIIGLFLVLRVGI | |
| | | | HLCIKLKHTKKRQIYTDIEM | |
| | | | NRLGK | |
| Murine | VGTM | D4 | MACNCQLMQDTPLLKFPCPR | 135 |
| CD80- | | | LILLFVLLIRLSQVSSDVDE | |
| MVP | | | QLSKSVKDKVLLPCRYNSPH | |
| | | | EDESEDRIYWQKHDKVVLSV | |
| | | | IAGKLKVWPEYKNRTLYDNT | |
| | | | TYSLIILGLVLSDRGTYSCV | |
| | | | VQKKERGTYEVKHLALVKLS | |
| | | | IKADFSTPNITESGNPSADT | |
| | | | KRITCFASGGFPKPRFSWLE | |
| | | | NGRELPGINTTISQDPESEL | |
| | | | YTISSQLDFNTTRNHTIKCL | |
| | | | IKYGDAHVSEDFTWEKPPED | |
| | | | PPDSKNTLDIIQADGWMCHA | |
| | | | SKWVTTCDFRWYGPKYITHS | |
| | | | IRSFTPSVEQCKESIEQTKQ | |
| | | | GTWLNPGFPPQSCGYATVTD | |
| | | | AEAVIVQVTPHHVLVDEYTG | |
| | | | EWVDSQFINGKCSNYICPTV | |
| | | | HNSTTWHSDYKVKGLCDSNL | |
| | | | GMLDSDLHLSSKAQVFEHPH | |
| | | | IQDAASQLPDDESLFFGDTG | |
| | | | LSKNPIELVEGWFSSWKSSI | |
| | | | ASFFFIIGLIIGLFLVLRVG | |
| | | | IHLCIKLKHTKKRQIYTDIE | |
| | | | MNRLGK | |
| Human | VGTM | D4 | MGHTRRQGTSPSKCPYLNFF | 136 |
| CD80- | | | QLLVLAGLSHFCSGVIHVTK | |
| MVP | | | EVKEVATLSCGHNVSVEELA | |
| | | | QTRIYWQKEKKMVLTMMSGD | |
| | | | MNIWPEYKNRTIFDITNNLS | |
| | | | IVILALRPSDEGTYECVVLK | |
| | | | YEKDAFKREHLAEVTLSVKA | |
| | | | DFPTPSISDFEIPTSNIRRI | |
| | | | ICSTSGGFPEPHLSWLENGE | |
| | | | ELNAINTTVSQDPETELYAV | |
| | | | SSKLDFNMTTNHSFMCLIKY | |
| | | | GHLRVNQTFNWNTTKQEHFP | |
| | | | DNLLPSDIIQADGWMCHASK | |
| | | | WVTTCDFRWYGPKYITHSIR | |
| | | | SFTPSVEQCKESIEQTKQGT | |
| | | | WLNPGFPPQSCGYATVIDAE | |
| | | | AVIVQVTPHHVLVDEYTGEW | |
| | | | VDSQFINGKCSNYICPTVHN | |
| | | | STTWHSDYKVKGLCDSNLGM | |
| | | | LDSDLHLSSKAQVFEHPHIQ | |
| | | | DAASQLPDDESLFFGDTGLS | |
| | | | KNPIELVEGWFSSWKSSIAS | |
| | | | FFFIIGLIIGLFLVLRVGIH | |
| | | | LCIKLKHTKKRQIYTDIEMN | |
| | | | RLGK | |
| Murine | NA³ | NA⁴ | MYTDIEMNRLGKNPNQKIIT | 137 |
| 4- | | | IGSICMVTGIVSLMLQIGNM | |
| 1BBL- | | | ISIWVSHSIHTGNQHQSEPI | |
| MVP | | | SNTNFLTEKAVASVKLAGNS | |
| | | | SLCPINDRTEPRPALTITTS | |
| | | | PNLGTRENNADQVTPVSHIG | |
| | | | CPNTTQQGSPVFAKLLAKNQ | |
| | | | ASLCNTTLNWHSQDGAGSSY | |
| | | | LSQGLRYEEDKKELVVDSPG | |
| | | | LYYVFLELKLSPTFTNTGHK | |
| | | | VQGWVSLVLQAKPQVDDFDN | |
| | | | LALTVELFPCSMENKLVDRS | |
| | | | WSQLLLLKAGHRLSVGLRAY | |
| | | | LHGAQDAYRDWELSYPNTTS | |
| | | | FGLFLVKPDNPWE | |
| Human | NA³ | NA⁴ | MYTDIEMNRLGKNPNQKIIT | 138 |
| 4- | | | IGSICMVTGIVSLMLQIGNM | |
| 1BBL- | | | ISIWVSHSIHTGNQHQSEPI | |
| MVP | | | SNTNFLTEKAVASVKLAGNS | |
| | | | SLCPINDACPWAVSGARASP | |
| | | | GSAASPRLREGPELSPDDPA | |
| | | | GLLDLRQGMFAQLVAQNVLL | |
| | | | IDGPLSWYSDPGLAGVSLTG | |
| | | | GLSYKEDTKELVVAKAGVYY | |
| | | | VFFQLELRRVVAGEGSGSVS | |
| | | | LALHLQPLRSAAGAAALALT | |
| | | | VDLPPASSEARNSAFGFQGR | |
| | | | LLHLSAGQRLGVHLHTEARA | |
| | | | RHAWQLTQGATVLGLFRVTP | |
| | | | EIPAGLPSPRSE | |
| Murine | NA³ | NA⁴ | MYTDIEMNRLGKNPNQKIIT | 139 |
| OX40L | | | IGSICMVTGIVSLMLQIGNM | |
| -MVP | | | ISIWVSHSIHTGNQHQSEPI | |
| | | | SNTNFLTEKAVASVKLAGNS | |
| | | | SLCPINDSSSPAKDPPIQRL | |
| | | | RGAVTRCEDGQLFISSYKNE | |
| | | | YQTMEVQNNSVVIKCDGLYI | |
| | | | IYLKGSFFQEVKIDLHFRED | |
| | | | HNPISIPMLNDGRRIVFTVV | |
| | | | ASLAFKDKVYLTVNAPDTLC | |
| | | | EHLQINDGELIVVQLTPGYC | |
| | | | APEGSYHSTVNQVPL | |
| Human | NA³ | NA⁴ | MYTDIEMNRLGKNPNQKIIT | 140 |
| OX40L | | | IGSICMVTGIVSLMLQIGNM | |
| -MVP | | | ISIWVSHSIHTGNQHQSEPI | |
| | | | SNTNFLTEKAVASVKLAGNS | |
| | | | SLCPINDMVSHRYPRIQSIK | |
| | | | VQFTEYKKEKGFILTSQKED | |
| | | | EIMKVQNNSVIINCDGFYLI | |
| | | | SLKGYFSQEVNISLHYQKDE | |
| | | | EPLFQLKKVRSVNSLMVASL | |
| | | | TYKDKVYLNVTTDNTSLDDF | |
| | | | HVNGGELILIHQNPGEFCVL | |
| Murine | NA³ | NA⁴ | MYTDIEMNRLGKNPNQKIIT | 141 |
| LIGHT | | | IGSICMVTGIVSLMLQIGNM | |
| -MVP | | | ISIWVSHSIHTGNQHQSEPI | |
| | | | SNTNFLTEKAVASVKLAGNS | |
| | | | SLCPINDLHQRLGDIVAHLP | |
| | | | DGGKGSWEKLIQDQRSHQAN | |
| | | | PAAHLTGANASLIGIGGPLL | |
| | | | WETRLGLAFLRGLTYHDGAL | |
| | | | VTMEPGYYYVYSKVQLSGVG | |
| | | | CPQGLANGLPITHGLYKRTS | |
| | | | RYPKELELLVSRRSPCGRAN | |
| | | | SSRVWWDSSFLGGVVHLEAG | |
| | | | EEVVVRVPGNRLVRPRDGTR | |
| | | | SYFGAFMV | |
| Murine | VGTM | D4 | MSALLTAAGLLFLGMLQAFP | 142 |
| CD30- | | | TDRPLKTTCAGDLSHYPGEA | |
| MVP | | | ARNCCYQCPSGLSPTQPCPR | |
| | | | GPAHCRKQCAPDYYVNEDGK | |
| | | | CTACVTCLPGLVEKAPCSGN | |
| | | | SPRICECQPGMHCCTPAVNS | |
| | | | CARCKLHCSGEEVVKSPGTA | |
| | | | KKDTICELPSSGSGPNCSNP | |
| | | | GDRKTLTSHATPQAMPTLES | |
| | | | PANDSARSLLPMRVTNLVQE | |
| | | | DATELVKVPESSSSKAREPS | |
| | | | PDPGNAEKNMTLELPSPGTL | |
| | | | PDISTSENSKEPASTASTLS | |
| | | | LVVDAWTSSRMQPTSPLSTG | |
| | | | TPFLDPGIIQADGWMCHASK | |
| | | | WVTTCDFRWYGPKYITHSIR | |
| | | | SFTPSVEQCKESIEQTKQGT | |
| | | | WLNPGFPPQSCGYATVIDAE | |
| | | | AVIVQVTPHHVLVDEYTGEW | |
| | | | VDSQFINGKCSNYICPTVHN | |
| | | | STTWHSDYKVKGLCDSNLGM | |
| | | | LDSDLHLSSKAQVFEHPHIQ | |
| | | | DAASQLPDDESLFFGDTGLS | |
| | | | KNPIELVEGWFSSWKSSIAS | |
| | | | FFFIIGLIIGLFLVLRVGIH | |
| | | | LCIKLKHTKKRQIYTDIEMN | |
| | | | RLGK | |
| Murine | NA³ | NA⁴ | MYTDIEMNRLGKNPNQKIIT | 143 |
| CD30L | | | IGSICMVTGIVSLMLQIGNM | |
| -MVP | | | ISIWVSHSIHTGNQHQSEPI | |
| | | | SNTNFLTEKAVASVKLAGNS | |
| | | | SLCPINDQKKDSTPNTTEKA | |
| | | | PLKGGNCSEDLFCTLKSTPS | |
| | | | KKSWAYLQVSKHLNNTKLSW | |
| | | | NEDGTIHGLIYQDGNLIVQF | |
| | | | PGLYFIVCQLQFLVQCSNHS | |
| | | | VDLTLQLLINSKIKKQTLVT | |
| | | | VCESGVQSKNIYQNLSQFLL | |
| | | | HYLQVNSTISVRVDNFQYVD | |
| | | | TNTFPLDNVLSVFLYSSSD | |
| Murine | VGTM | D4 | MKCKFLGSFFLLFSLSGKGA | 144 |
| CD2- | | | DCRDNETIWGVLGHGITLNI | |
| MVP | | | PNFQMTDDIDEVRWVRRGTL | |
| | | | VAEFKRKKPPFLISETYEVL | |
| | | | ANGSLKIKKPMMRNDSGTYN | |
| | | | VMVYGTNGMTRLEKDLDVRI | |
| | | | LERVSKPMIHWECPNTTLTC | |
| | | | AVLQGTDFELKLYQGETLLN | |
| | | | SLPQKNMSYQWTNLNAPFKC | |
| | | | EAINPVSKESKMEVVNCPEK | |
| | | | GLSFYDIIQADGWMCHASKW | |
| | | | VTTCDFRWYGPKYITHSIRS | |
| | | | FTPSVEQCKESIEQTKQGTW | |
| | | | LNPGFPPQSCGYATVTDAEA | |
| | | | VIVQVTPHHVLVDEYTGEWV | |
| | | | DSQFINGKCSNYICPTVHNS | |
| | | | TTWHSDYKVKGLCDSNLGML | |
| | | | DSDLHLSSKAQVFEHPHIQD | |
| | | | AASQLPDDESLFFGDTGLSK | |
| | | | NPIELVEGWFSSWKSSIASF | |
| | | | FFIIGLIIGLFLVLRVGIHL | |
| | | | CIKLKHTKKRQIYTDIEMNR | |
| | | | LGK | |
| Murine | VGTM | D4 | MAWPPPYWLCMLGTLVGLSA | 145 |
| CD27- | | | TLAPNSCPDKHYWTGGGLCC | |
| MVP | | | RMCEPGTFFVKDCEQDRTAA | |
| | | | QCDPCIPGTSFSPDYHTRPH | |
| | | | CESCRHCNSGFLIRNCTVTA | |
| | | | NAECSCSKNWQCRDQECTEC | |
| | | | DPPLNPALTRQPSETPSPQP | |
| | | | PPTHLPHGTEKPSWPLHRQL | |
| | | | PNSTVYSQRSSHRPLCSSDC | |
| | | | IRIIQADGWMCHASKWVTTC | |
| | | | DFRWYGPKYITHSIRSFTPS | |
| | | | VEQCKESIEQTKQGTWLNPG | |
| | | | FPPQSCGYATVTDAEAVIVQ | |
| | | | VTPHHVLVDEYTGEWVDSQF | |
| | | | INGKCSNYICPTVHNSTTWH | |
| | | | SDYKVKGLCDSNLGMLDSDL | |
| | | | HLSSKAQVFEHPHIQDAASQ | |
| | | | LPDDESLFFGDTGLSKNPIE | |
| | | | LVEGWFSSWKSSIASFFFII | |
| | | | GLIIGLFLVLRVGIHLCIKL | |
| | | | KHTKKRQIYTDIEMNRLGK | |
| Murine | NA³ | NA⁴ | MYTDIEMNRLGKNPNQKIIT | 146 |
| CD70- | | | IGSICMVTGIVSLMLQIGNM | |
| MVP | | | ISIWVSHSIHTGNQHQSEPI | |
| | | | SNTNFLTEKAVASVKLAGNS | |
| | | | SLCPINDSKQQQRLLEHPEP | |
| | | | HTAELQLNLTVPRKDPTLRW | |
| | | | GAGPALGRSFTHGPELEEGH | |
| | | | LRIHQDGLYRLHIQVTLANC | |
| | | | SSPGSTLQHRATLAVGICSP | |
| | | | AAHGISLLRGRFGQDCTVAL | |
| | | | QRLTYLVHGDVLCTNLTLPL | |
| | | | LPSRNADETFFGVQWICP | |
| Murine | VGTM | D4 | MQLKCPCFVSLGTRQPVWKK | 147 |
| ICOSL | | | LHVSSGFFSGLGLFLLLLSS | |
| -MVP | | | LCAASAETEVGAMVGSNVVL | |
| | | | SCIDPHRRHFNLSGLYVYWQ | |
| | | | IENPEVSVTYYLPYKSPGIN | |
| | | | VDSSYKNRGHLSLDSMKQGN | |
| | | | FSLYLKNVTPQDTQEFTCRV | |
| | | | FMNTATELVKILEEVVRLRV | |
| | | | AANFSTPVISTSDSSNPGQE | |
| | | | RTYTCMSKNGYPEPNLYWIN | |
| | | | TTDNSLIDTALQNNTVYLNK | |
| | | | LGLYDVISTLRLPWTSRGDV | |
| | | | LCCVENVALHQNITSISQAE | |
| | | | SFTGNNTKNPQETHNNELKD | |
| | | | IIQADGWMCHASKWVTTCDF | |
| | | | RWYGPKYITHSIRSFTPSVE | |
| | | | QCKESIEQTKQGTWLNPGFP | |
| | | | PQSCGYATVTDAEAVIVQVT | |
| | | | PHHVLVDEYTGEWVDSQFIN | |
| | | | GKCSNYICPTVHNSTTWHSD | |
| | | | YKVKGLCDSNLGMLDSDLHL | |
| | | | SSKAQVFEHPHIQDAASQLP | |
| | | | DDESLFFGDTGLSKNPIELV | |
| | | | EGWFSSWKSSIASFFFIIGL | |
| | | | IIGLFLVLRVGIHLCIKLKH | |
| | | | TKKRQIYTDIEMNRLGK | |
| Murine | VGTM | D4 | MKPYFCRVFVFCFLIRLLTG | 148 |
| ICOS- | | | EINGSADHRMFSFHNGGVQI | |
| MVP | | | SCKYPETVQQLKMRLFRERE | |
| | | | VLCELTKTKGSGNAVSIKNP | |
| | | | MLCLYHLSNNSVSFFLNNPD | |
| | | | SSQGSYYFCSLSIFDPPPFQ | |
| | | | ERNLSGGYLHIYESQLCCQL | |
| | | | KLDIIQADGWMCHASKWVTT | |
| | | | CDFRWYGPKYITHSIRSFTP | |
| | | | SVEQCKESIEQTKQGTWLNP | |
| | | | GFPPQSCGYATVTDAEAVIV | |
| | | | QVTPHHVLVDEYTGEWVDSQ | |
| | | | FINGKCSNYICPTVHNSTTW | |
| | | | HSDYKVKGLCDSNLGMLDSD | |
| | | | LHLSSKAQVFEHPHIQDAAS | |
| | | | QLPDDESLFFGDTGLSKNPI | |
| | | | ELVEGWFSSWKSSIASFFFI | |
| | | | IGLIIGLFLVLRVGIHLCIK | |
| | | | LKHTKKRQIYTDIEMNRLGK | |
| Murine | NA³ | NA⁴ | MYTDIEMNRLGKNPNQKIIT | 149 |
| GITRL | | | IGSICMVTGIVSLMLQIGNM | |
| -MVP | | | ISIWVSHSIHTGNQHQSEPI | |
| | | | SNTNFLTEKAVASVKLAGNS | |
| | | | SLCPINDTSLKPTAIESCMV | |
| | | | KFELSSSKWHMTSPKPHCVN | |
| | | | TTSDGKLKILQSGTYLIYGQ | |
| | | | VIPVDKKYIKDNAPFVVQIY | |
| | | | KKNDVLQTLMNDFQILPIGG | |
| | | | VYELHAGDNIYLKFNSKDHI | |
| | | | QKTNTYWGIILMPDLPFIS | |
| Murine | VGTM | D4 | MGAWAMLYGVSMLCVLDLGQ | 150 |
| GITR- | | | PSVVEEPGCGPGKVQNGSGN | |
| MVP | | | NTRCCSLYAPGKEDCPKERC | |
| | | | ICVTPEYHCGDPQCKICKHY | |
| | | | PCQPGQRVESQGDIVFGFRC | |
| | | | VACAMGTFSAGRDGHCRLWT | |
| | | | NCSQFGFLTMFPGNKTHNAV | |
| | | | CIPEPLPTEQYGHIIQADGW | |
| | | | MCHASKWVTTCDFRWYGPKY | |
| | | | ITHSIRSFTPSVEQCKESIE | |
| | | | QTKQGTWLNPGFPPQSCGYA | |
| | | | TVIDAEAVIVQVTPHHVLVD | |
| | | | EYTGEWVDSQFINGKCSNYI | |
| | | | CPTVHNSTTWHSDYKVKGLC | |
| | | | DSNLGMLDSDLHLSSKAQVF | |
| | | | EHPHIQDAASQLPDDESLFF | |
| | | | GDTGLSKNPIELVEGWFSSW | |
| | | | KSSIASFFFIIGLIIGLFLV | |
| | | | LRVGIHLCIKLKHTKKRQIY | |
| | | | TDIEMNRLGK* | |
| Murine | VGTM | D4 | MGNNCYNVVVIVLLLVGCEK | 151 |
| 4-1BB- | | | VGAVQNSCDNCQPGTFCRKY | |
| MVP | | | NPVCKSCPPSTFSSIGGQPN | |
| | | | CNICRVCAGYFRFKKFCSST | |
| | | | HNAECECIEGFHCLGPQCTR | |
| | | | CEKDCRPGQELTKQGCKTCS | |
| | | | LGTFNDQNGTGVCRPWTNCS | |
| | | | LDGRSVLKTGTTEKDVVCGP | |
| | | | PVVSFSPSTTISVTPEGGPG | |
| | | | GHSLQVLTDIIQADGWMCHA | |
| | | | SKWVTTCDFRWYGPKYITHS | |
| | | | IRSFTPSVEQCKESIEQTKQ | |
| | | | GTWLNPGFPPQSCGYATVTD | |
| | | | AEAVIVQVTPHHVLVDEYTG | |
| | | | EWVDSQFINGKCSNYICPTV | |
| | | | HNSTTWHSDYKVKGLCDSNL | |
| | | | GMLDSDLHLSSKAQVFEHPH | |
| | | | IQDAASQLPDDESLFFGDTG | |
| | | | LSKNPIELVEGWFSSWKSSI | |
| | | | ASFFFIIGLIIGLFLVLRVG | |
| | | | IHLCIKLKHTKKRQIYTDIE | |
| | | | MNRLGK | |
| Murine | VGTM | D4 | MYVWVQQPTALLLLGLTLGV | 152 |
| OX40- | | | TARRLNCVKHTYPSGHKCCR | |
| MVP | | | ECQPGHGMVSRCDHTRDTLC | |
| | | | HPCETGFYNEAVNYDTCKQC | |
| | | | TQCNHRSGSELKQNCTPTQD | |
| | | | TVCRCRPGTQPRQDSGYKLG | |
| | | | VDCVPCPPGHFSPGNNQACK | |
| | | | PWTNCTLSGKQTRHPASDSL | |
| | | | DAVCEDRSLLATLLWETQRP | |
| | | | TFRPTTVQSTTVWPRTSELP | |
| | | | SPPTLVTPEGPADIIQADGW | |
| | | | MCHASKWVTTCDFRWYGPKY | |
| | | | ITHSIRSFTPSVEQCKESIE | |
| | | | QTKQGTWLNPGFPPQSCGYA | |
| | | | TVTDAEAVIVQVTPHHVLVD | |
| | | | EYTGEWVDSQFINGKCSNYI | |
| | | | CPTVHNSTTWHSDYKVKGLC | |
| | | | DSNLGMLDSDLHLSSKAQVF | |
| | | | EHPHIQDAASQLPDDESLFF | |
| | | | GDTGLSKNPIELVEGWFSSW | |
| | | | KSSIASFFFIIGLIIGLFLV | |
| | | | LRVGIHLCIKLKHTKKRQIY | |
| | | | TDIEMNRLGK | |

TM¹: Transmembrane Domain
OD²: Oligomerization Domain
NA³: Neuraminidase
NA⁴: Neuraminidase stem

### Example 3. Characterization of Anti-Tumor Immunity by Inhibitory IC-MVPs

This example illustrates characterization of PD-1-MVP and its function in engaging target cells and tumor-control mouse models.

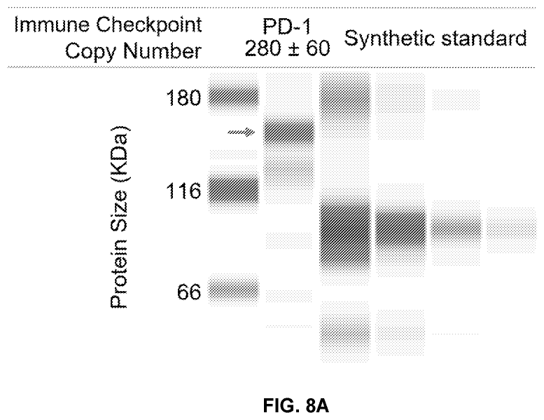

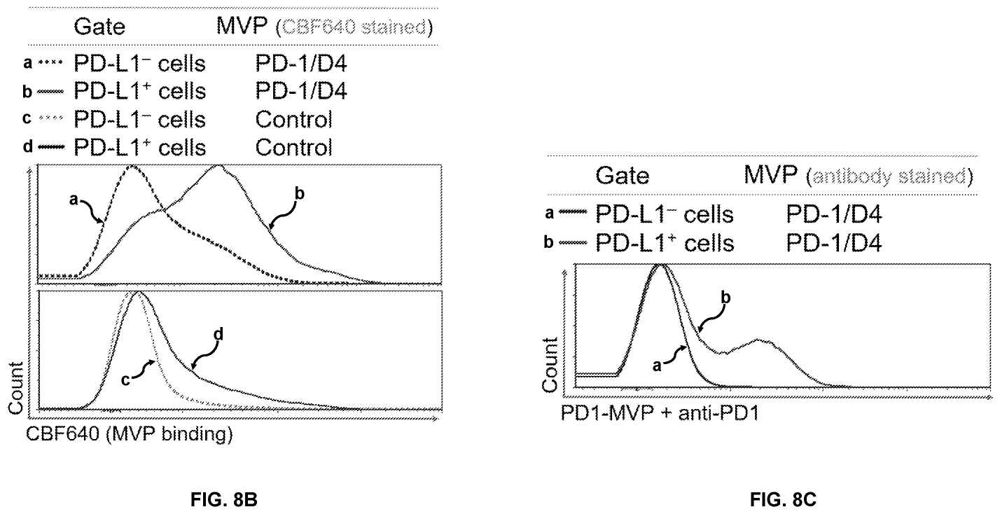

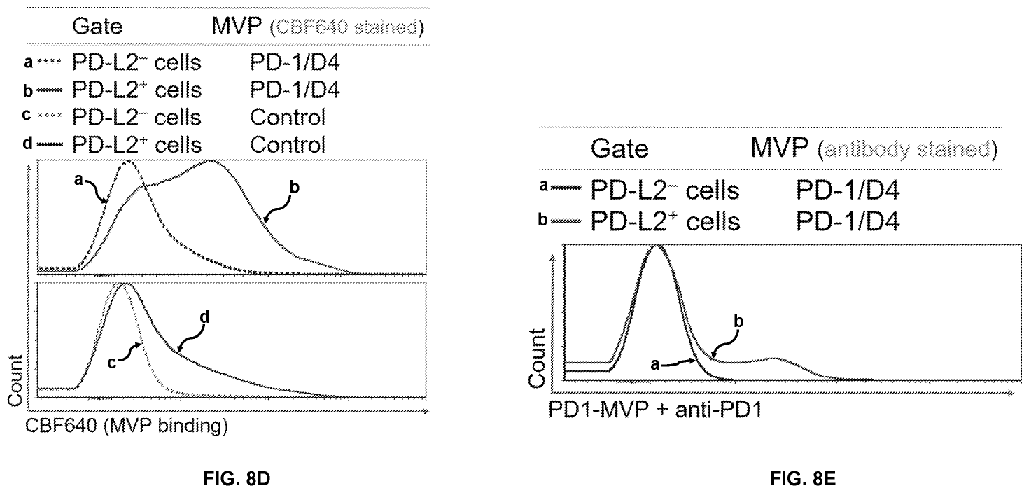

It was examined whether PD-1-MVPs can selectively bind to target cells expressing its cognate ligand PD-L1/PD-L2. PD-1-MVPs were generated by pseudotyping lentiviral VLPs with trimeric PD-1 fusion peptides. Specifically, HEK 293T cells were co-transfected with a trimeric PD-1 display construct, along with a lentiviral packaging construct expressing essential packaging components, including Gag-Pol and Rev proteins, and a viral genome transfer vector encoding a GFP/luciferase reporter (FIG. 3A). The concentration of purified PD-1-MVPs was quantified via P24 ELISA. PD-1-MVPs displayed 280±60 copies of PD-1 per MVP in various oligomerized forms, as determined by quantitative western blot analysis (FIG. 8A). Hence, the D4 display construct can effectively present hundreds of copies of PD-1 in oligomerized form on IC-MVPs.

To confirm that PD-1-MVPs displayed functional PD-1, it was tested whether PD-1-MVPs can selectively bind to target cells expressing PD-L1 or PD-L2, the cognate ligands of PD-1 (FIGS. 8B-8E). First, target cell lines were established by transfecting S293 cells with a construct expressing PD-L1. Transfected cells were then stained with anti-PD-L1 antibody to differentiate PD-L1 positive cells (PD-L1+) from PD-L1 negative cells (PD-L1−). Subsequently, PD-1-MVPs were labeled with a fluorescent dye, transfected cells were stained with labeled PD-1-MVPs, and selective MVP-cell binding was analyzed via FACS (FIG. 8B). The results showed that fluorescently-labeled PD-1-MVP binding caused significantly higher fluorescence shift in PD-L1+ cells as compared to PD-L1− cells (FIG. 8B, upper panel). Moreover, this shift was at least one log higher than the fluorescence shift caused by staining the same cells with control MVPs displaying non-specific ligand (FIG. 8B, lower panel). This demonstrated that PD-1-MVPs displayed functional PD-1 which can selectively bound to PD-L1 on target cells.

This result was further validated through an alternative staining method (FIG. 8C). In this case, PD-L1-transfected S293 cells were first incubated with unlabeled PD-1-MVPs to allow PD-1-MVPs to bind to the target cells. The MVP-cell mixture was then co-stained with fluorescently-labeled anti-PD-1 and anti-PD-L1 antibodies. The PD-1 staining pattern was then examined on PD-L1+ cells and PD-L1− cells via FACS analysis. It was observed that a significant fraction of PD-L1+ cells were also PD-1 positive, as exemplified by a 1-log PD-1 staining shift in PD-L1+ cells from PD-1− background cells (FIG. 8C). Single staining with anti-PD-1 antibody did not compete with PD-L1-MVP binding to target cells, and PD-L1-transfected S293 cells were PD-L negative. These results indicated that PD-1-MVPs displayed functional PD-1. Using similar methods, it was demonstrated that PD-1-MVPs can selectively bound to target cells expressing PD-L2 in both type of binding assays with or without dye labeling (FIGS. 8D, 8E). Collectively, PD-1-MVPs were generated displaying high copy numbers of functional PD-1 proteins, and these MVPs can selectively bound target cells expressing their cognate ligands, PD-L1 or PD-L2.

Checkpoint Blockades with PD-1-MVPs and Other IC-MVPs

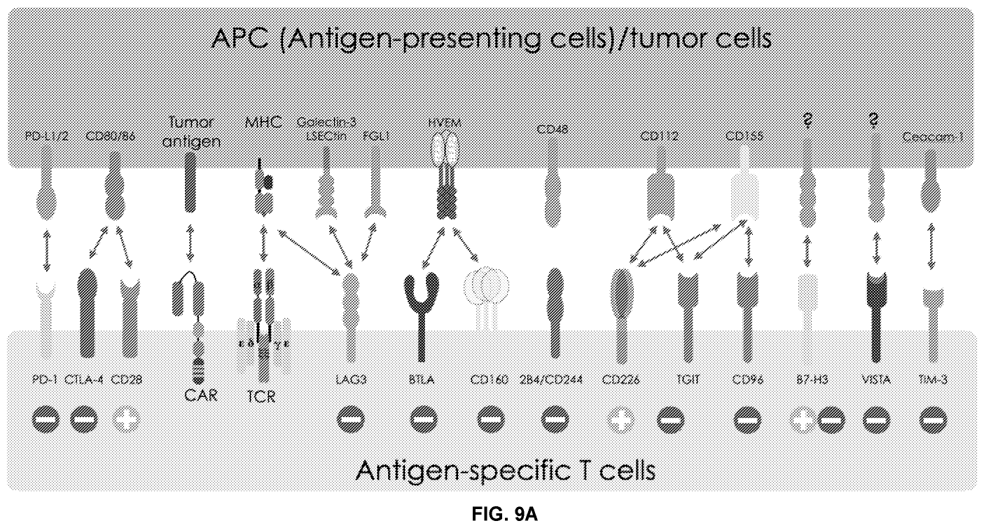

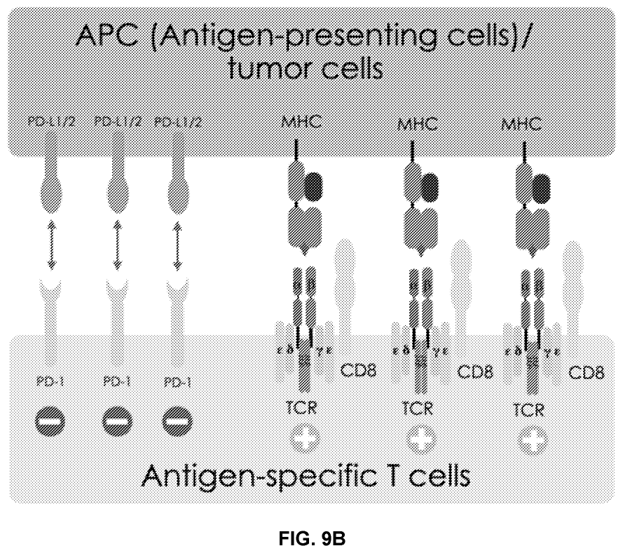

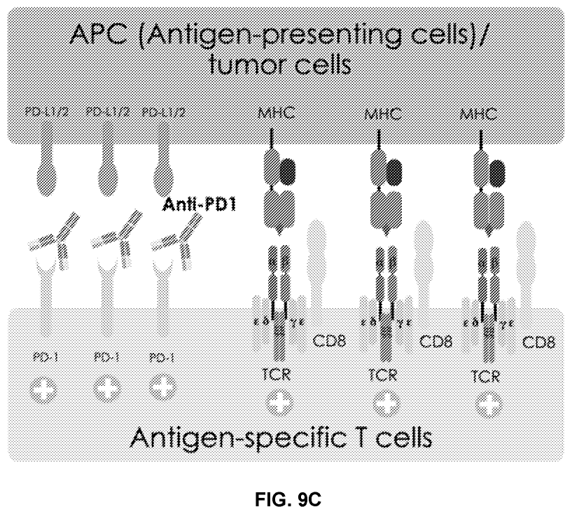

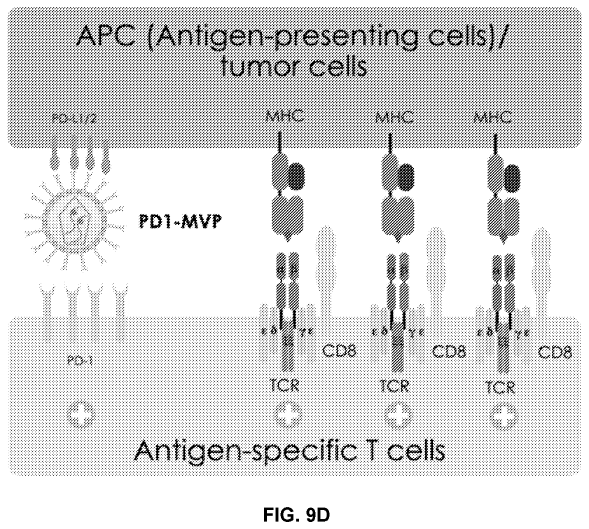

T cell activation in vivo is regulated by diverse group of inhibitory immune checkpoints, including PD-1, CTLA-4, LAG-3, TIM-3, and many others, as illustrated by a schematic depicting the inhibitory immune checkpoints on T cells and their ligands on antigen presenting cells including tumor cells (FIG. 9A). Such regulation is important to maintain effector T cells under a suppressive state and to prevent unintended activation. Among these pathways, many have been shown to be exploited by cancer cells to suppress the function of tumor-targeting T cells (FIG. 9B). Antibodies targeting PD-1 or CTLA-4 can effectively block these inhibitory checkpoint signals mediated by cancer cells and activate anti-tumor T cells for cancer therapy (FIG. 9C). Since it was demonstrated that PD1-MVPs can selectively bind to target cells expressing PD-L1 and PD-L2 (FIGS. 8A-8E), PD1-MVPs can be used to block PD-L1/PDL-2 on cancer cells and prevent them from interacting with the PD-1 molecules on tumor targeting T cells (FIG. 9D). Thus, PD-1-MVPs can be used to therapeutically block the inhibitory checkpoint signals for cancer therapy.

### Inhibition of Tumor Growth by PD-1-MVPs in Mice

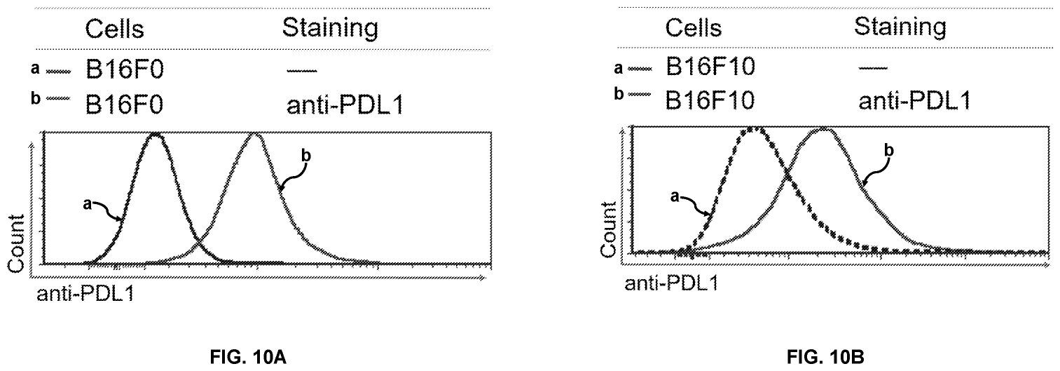

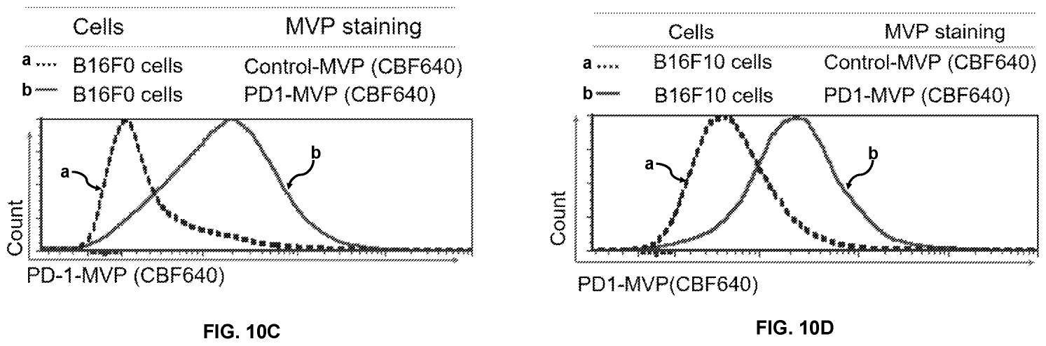

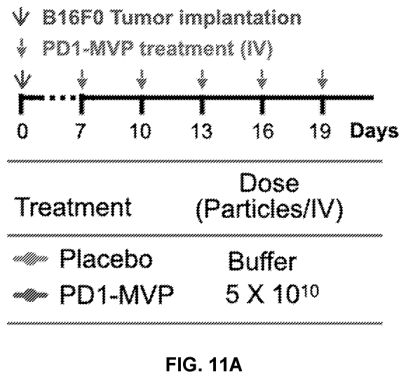

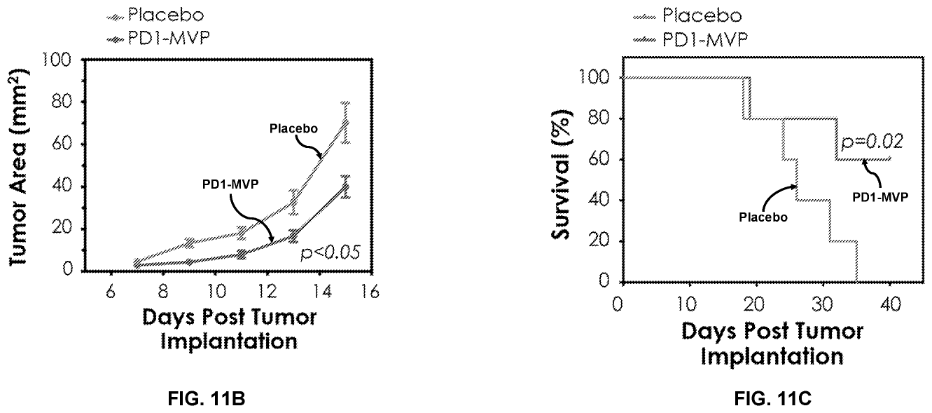

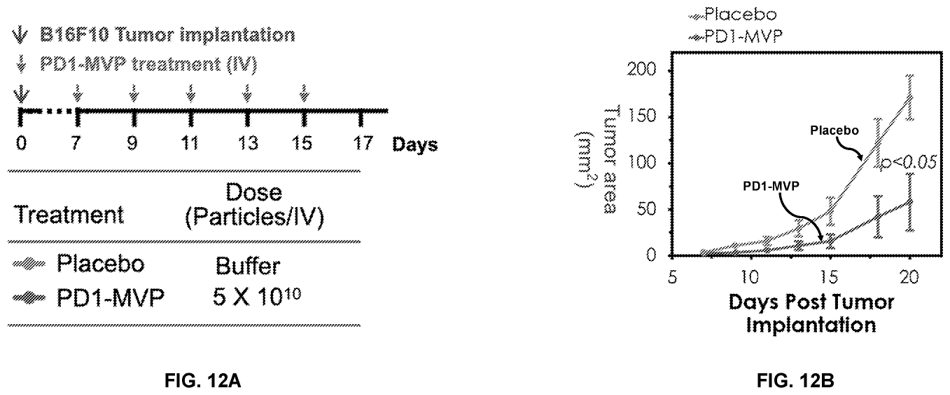

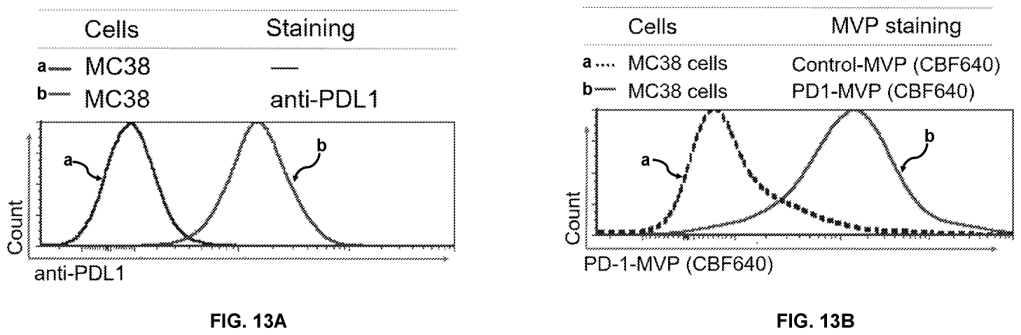

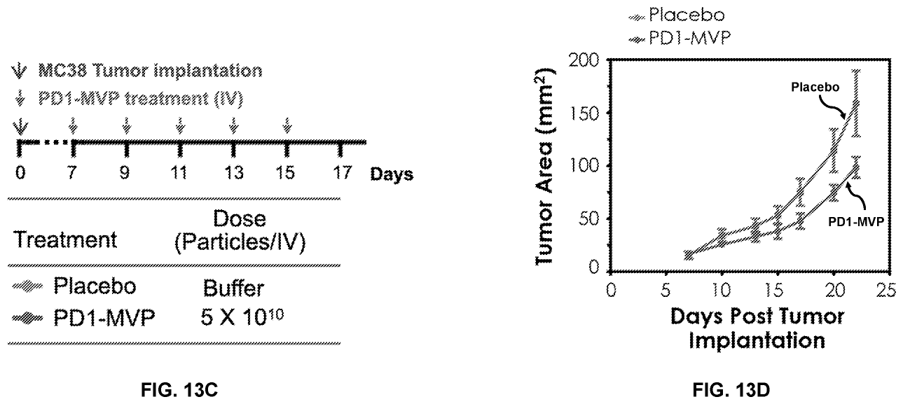

To determine whether PD1-MVPs can control melanoma cancer, it was examined whether PD1-MVPs can bind to PD-L1 expressed on cancel cells. As determined by FACS analyses, both mouse B16F0 (non-metastatic) and mouse B16F10 (metastatic) melanoma cells expressed high levels of PD-L1 (FIGS. 10A, 10B) and can effectively bind to fluorescent dye-labeled PD1-MVPs (FIGS. 10C, 10D), respectively. These results demonstrated that PD-1-MVPs can effectively bind to PD-L1 positive B16F0 and B16F10 melanoma cells. Mice bearing B16F0 melanoma tumors were treated with intravenously delivered PD1-MVPs. These mice received one treatment of 5×10¹⁰ PD-1-MVPs every three days starting at day-7 post tumor-implantation, for a total of 5 doses (FIG. 11A). PD1-MVPs significantly reduced tumor growth (FIG. 11B) and extended the survival (FIG. 11C) of mice bearing B16F0 melanoma tumors. Similarly, mice bearing B16F10 melanoma tumors were treated with 5×10¹⁰ PD1-MVPs every two days starting at Day-7 post tumor-implantation for 5 total treatments (FIG. 12A) and the results indicated that PD1-MVPs significantly reduced the growth of B16F10 tumors in mice (FIG. 12B). Finally, MC38 cells, a mouse colon cancer cell line, was shown to express high levels of PD-L1 (FIG. 13A) and bind to fluorescent dye-labelled PD1-MVPs efficiently (FIG. 13B) as indicated by FACS staining and analyses. Similarly, mice bearing MC38 colon adenocarcinoma tumors were treated with 5×10¹⁰ PD-1-MVPs every two days starting at Day-7 post tumor-implantation for 5 total treatments (FIG. 13C) and the results showed that PD-1-MVPs significantly reduced the growth of MC38 tumors in mice (FIG. 13D). Together, these results demonstrated that PD-1-MVPs can specifically bind to cancer cells expressing cognate ligands PD-L1 and PD-L2 and inhibit tumor development in multiple mouse tumor models. Thus, PD1-MVPs were shown to represent a novel multivalent checkpoint blockade therapeutic for cancer that can block or dampen the inhibitory signals mediated by the inhibitory checkpoints expressed on cancer cells and tumor-targeting T cells.

### Example 4. Control of Inflammation by Inhibitory IC-MVPs

This example illustrates analysis of PD-L1-MVP and 2B4-MVP and their functions in engaging target cells and controlling inflammatory responses in mouse.

### Use of IC-MVPs to Mimic Inhibitory Checkpoint Signaling

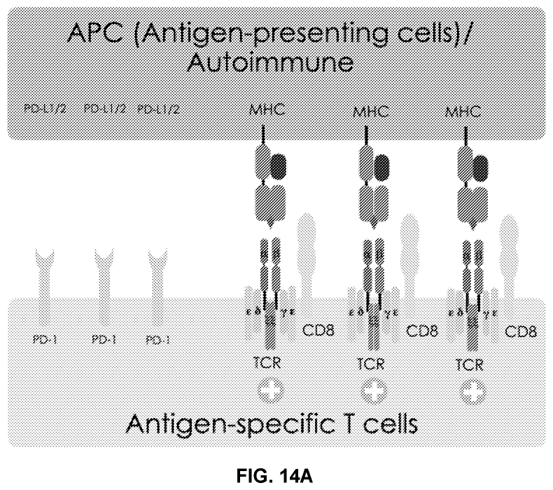

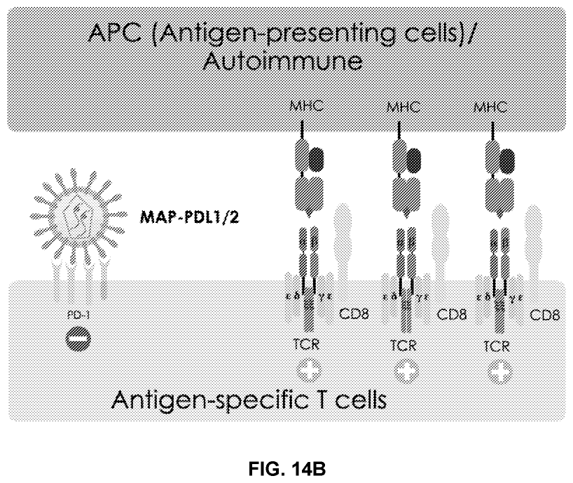

During various inflammatory scenarios, the immune system routinely engages inhibitory immune checkpoints to safeguard against auto-reactive immune cells. Uncontrolled inflammatory responses can lead to acute or chronic damage in the body. For example, during autoimmune, acute and chronic inflammatory conditions, T cells may be activated to damage the body's own tissues or organs in the absence of required inhibitory checkpoint signals, such as PD-L1/PD-1 signaling (FIG. 14A). IC-MVPs can be used to mimic these missing inhibitory immune checkpoint signals. For example, PD-L1-MVPs can be used to engage PD-1 molecules on autoreactive T cells and inactivate such T cells (FIG. 14B). Thus, IC-MVPs, such as PD-L1-MVPs, may be used to inactivate T cells and other immune cells during acute and chronic inflammatory conditions.

### Generation and Characterization of PD-L1-MVPs

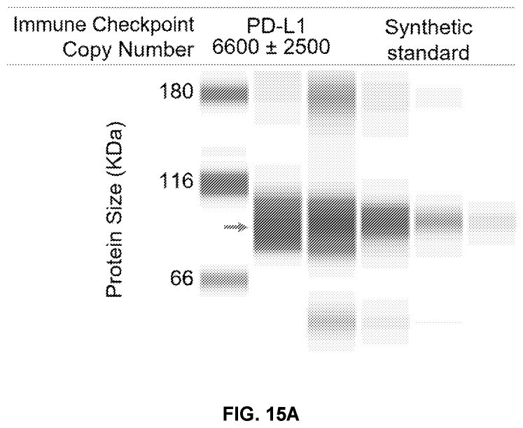

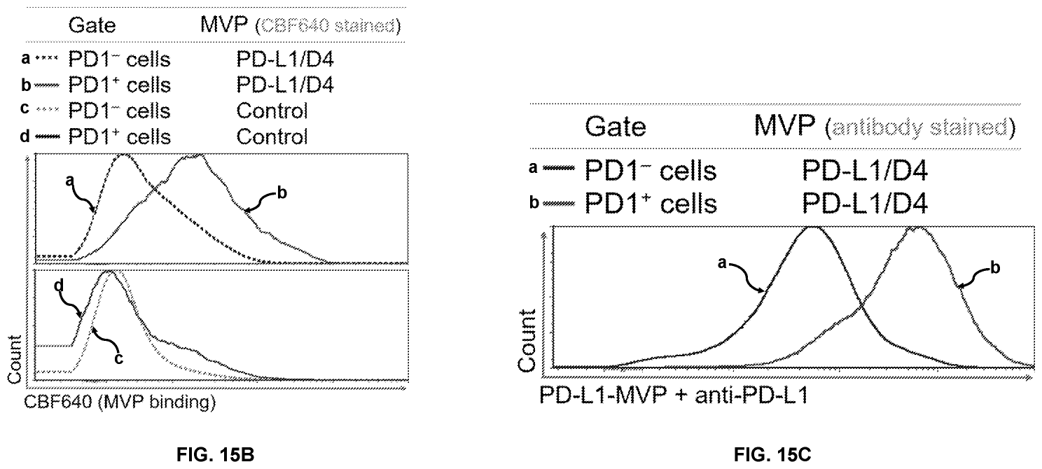

PD-L1-MVPs were generated by pseudotyping lentiviral VLPs with trimeric PD-L1 fusion peptides. Specifically, HEK 293T cells were co-transfected with a trimeric PD-L1 display construct, along with a lentiviral packaging construct expressing essential packaging components, including Gag-Pol and Rev proteins, and a viral genome transfer vector encoding a GFP/luciferase reporter (FIG. 3A). The concentration of purified PDL1-MVPs was quantified via P24 ELISA. PDL1-MVPs displayed 6600±2500 copies of PD-L1 per particle in various oligomerized forms, as determined by quantitative western blot analysis (FIG. 15A). Hence, the D4 display construct can effectively present thousands of copies of PD-L1 in oligomerized form on MVPs.

To confirm that PDL1-MVPs display functional PD-L1, it was tested whether PDL1-MVPs can selectively bind to target cells expressing PD-1, its cognate receptor (FIG. 15B). First, target cell lines were established by transfecting S293 cells with a construct expressing PD-1. Transfected cells were then stained with anti-PD-1 antibody to differentiate PD-1+ from PD-1-cells. Subsequently, PD-L1-MVPs were labeled with fluorescent dye, transfected cells were stained with labeled MVPs, and selective MVP-cell binding was analyzed via FACS (FIG. 15B). The results showed that labeled PDL1-MVP binding caused significantly higher fluorescence shift in PD-1+ cells as compared to PD-1− cells (FIG. 15B, upper panel). Moreover, this shift was at least one log higher than the fluorescence shift caused by staining the same cells with control MVPs displaying non-specific ligand (FIG. 15B, lower panel). This result demonstrated that PD-L1-MVPs displayed functional PD-L1 and can selectively bind to PD-1 positive target cells.

This result was further validated through an alternative staining method (FIG. 15C). In this case, PD-1-transfected cells were first incubated with unlabeled PD-L1-MVPs to allow MVPs to bind to target cells. The cell-MVP mixture was then co-stained with fluorescently-labeled anti-PD-1 and anti-PD-L1 antibodies. PD-L1 staining pattern was then examined on PD-1+ and PD-1− cells via FACS analysis. It was observed that significant fraction of PD-1+ cells were also PD-L1 positive, as exemplified by a 1-log PD-1 staining shift in PD-1+ cells from PD-1− background cells (FIG. 15C). These results demonstrated that PD-L1-MVPs displayed functional PD-L1. Collectively, PDL1-MVPs were generated displaying high copy numbers of functional PD-L1 protein, and the MVPs can selectively bind to target cells expressing their cognate receptor, PD-1.

### Inhibition of ARDS by PD-L1-MVPs in Mice

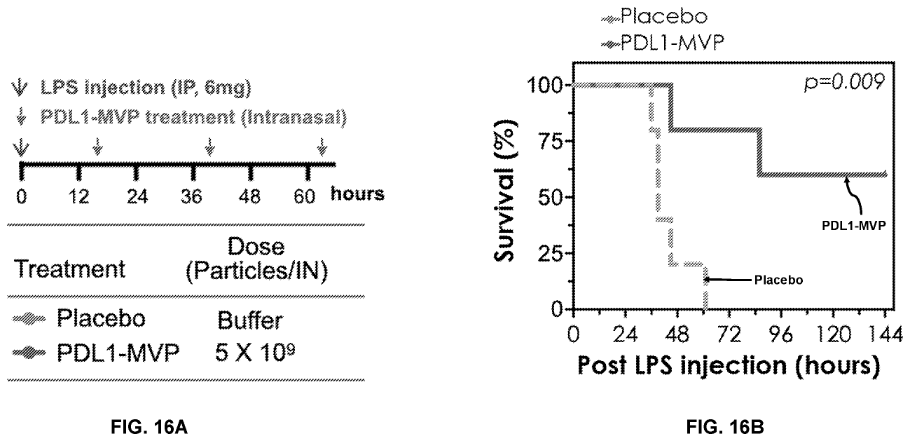

To test the inhibitory checkpoint function of PD-L1-MVPs, acute respiratory distress syndrome (ARDS) was used as an inflammation model to examine whether PDL1-MVPs can be used to control and reduce the damage caused by such systemic inflammation. The excessive proinflammatory responses that lead to ARDS may be initiated and driven by Toll-like receptors (TLRs), which recognize pathogen-derived constituents such as lipopolysaccharide (LPS), bacteria lipoproteins, and non-methylated CpG DNA, resulting in rapid escalation of systemic immune responses. Such conditions can be partially recapitulated in a mouse model of LPS-induced systemic inflammation. Mice were challenged with an intraperitoneal injection of a lethal dose of LPS (6 mg/kg) and treated with intranasally delivered IC-MVPs. Mice were initially treated 16 hours after LPS challenge and then treated daily with intranasally delivered PD-L1-MVPs (FIG. 16A). In this lethal model, untreated mice reached experimental endpoint within 72 hours (FIG. 16B). If IC-MVP treatment can rescue mice from lethality, it demonstrated that IC-MVPs can effectively dampen systemic inflammation induced by LPS. Indeed, it was observed that PD-L1-MVP treatment rescued 3 out 5 mice from lethality (FIG. 16B), a 60% rescue rate, demonstrating that PD-L1-MVPs can effectively inhibit the systemic immune response induced by LPS.

### Generation and Characterization of 2B4-MVP

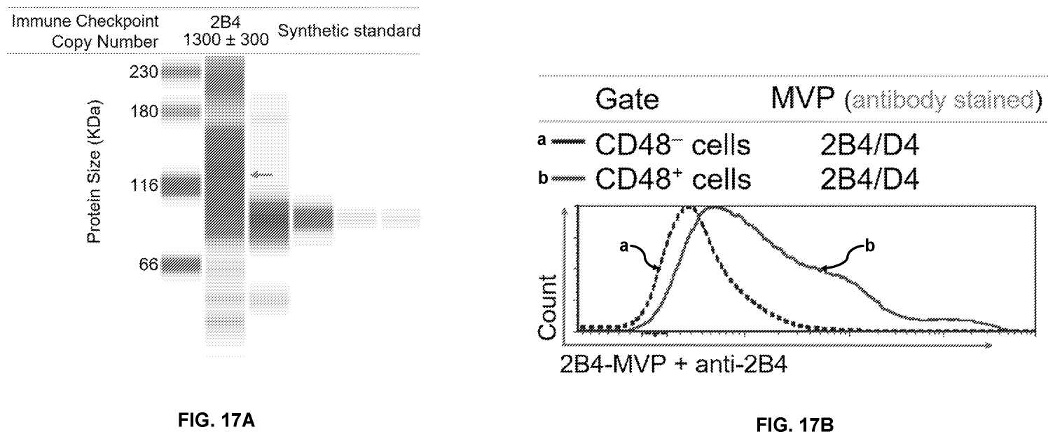

2B4-MVPs were generated by pseudotyping lentiviral VLPs with trimeric PD-L1 fusion peptides. Specifically, HEK 293T cells were co-transfected with a trimeric 2B4 display construct, along with a lentiviral packaging construct expressing essential packaging components, including Gag-Pol and Rev proteins, and a viral genome transfer vector encoding a GFP/luciferase reporter (FIG. 3A). The concentration of purified 2B4-MVPs was quantified via P24 ELISA. PD-L1-MVPs displayed 1300±300 copies of 2B4 per particle in various oligomerized forms, as determined by quantitative western blot analysis (FIG. 17A). Hence, the D4 display construct can effectively present thousands of copies of 2B4 in oligomerized form on MVPs.

To confirm that 2B4-MVPs displayed functional 2B4, it was tested whether 2B4-MVPs can selectively bind to target cells expressing CD48, its cognate receptor (FIG. 17B). CD48 transfected cells were first incubated with unlabeled 2B4-MVPs to allow MVPs to bind to target cells. The cell-MVP mixture was then co-stained with fluorescently-labeled anti-CD48 and anti-2B4 antibodies. 2B4 staining pattern on CD48+ and CD48− cells was then examined via FACS analysis. Significant fraction of CD48+ cells were also 2B4 positive, as exemplified by a 1-log 2B4 staining shift in CD48+ cells from CD48− background cells (FIG. 17B). These results indicated that 2B4-MVPs displayed functional 2B4. Collectively, 2B4-MVPs were generated displaying high copy numbers of functional 2B4 protein, and the MVPs can selectively bind target cells expressing their cognate receptor, CD48.

### Inhibition of ARDS by 2B4-MVPs in Mice

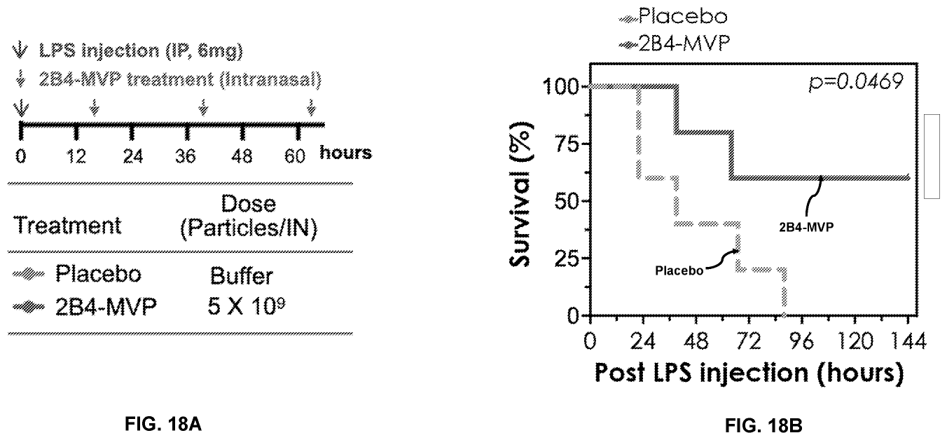

To test the inhibitory checkpoint function of 2B4-MVPs, acute respiratory distress syndrome (ARDS) was used as a model for systemic inflammation to examine whether 2B4-MVPs can be used to control and reduce the damage caused by such systemic inflammation. Mice were challenged with an intraperitoneal injection of a lethal dose of LPS (6 mg/kg) and treated with intranasally delivered 2B4-MVPs. Mice were initially treated 16 hours after LPS challenge and then treated daily with intranasally delivered 2B4-MVPs (FIG. 18A). In this lethal model, untreated mice reached experimental endpoint within 96 hours (FIG. 18B). If 2B4-MVP treatment can rescue mice from ARDS lethality, it would demonstrate that 2B4-MVPs can effectively dampen systemic inflammation induced by LPS. Indeed, the results indicated that 2B4-MVP treatment rescued 3 out 5 mice from lethality (FIG. 18B), a 60% rescue rate, demonstrating that 2B4-MVP can effectively inhibit the systemic immune response induced by LPS.

### Example 5. IC-MVPs Displaying Various Inhibitory Immune Checkpoints

A list of IC-MVPs displaying various inhibitory immune checkpoints were generated and their compositions were characterized by determining the copies of immune checkpoint molecules displayed on each of the VLPs. This example also demonstrates specific binding of IC-MVPs to their target cells expressing cognate ligands or receptors. The list of IC-MVPs include PDL2-MVP, CTLA4-MVP, CD80-MVP, CD86-MVP, GALECTIN3-MVP, LAG3-MVP, FGL1-MVP, HVEM-MVP, BTLA-MVP, CD160-MVP, CD48-MVP, CD112-MVP, TIGIT-MVP, CD155-MVP, TIM3-MVP, and Ceacam1-MVP.

### PD-L2-MVP Composition and Selective Binding to Target Cells Expressing PD-1

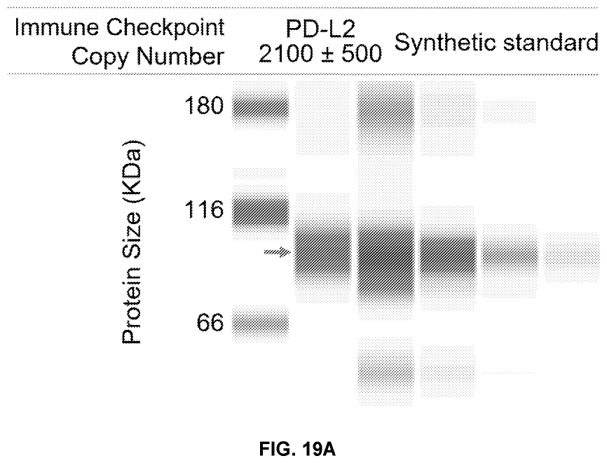

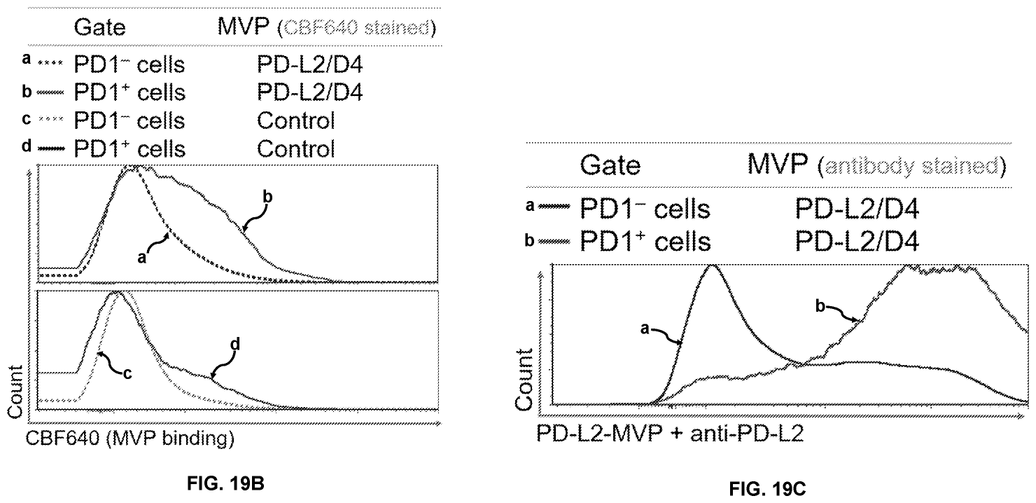

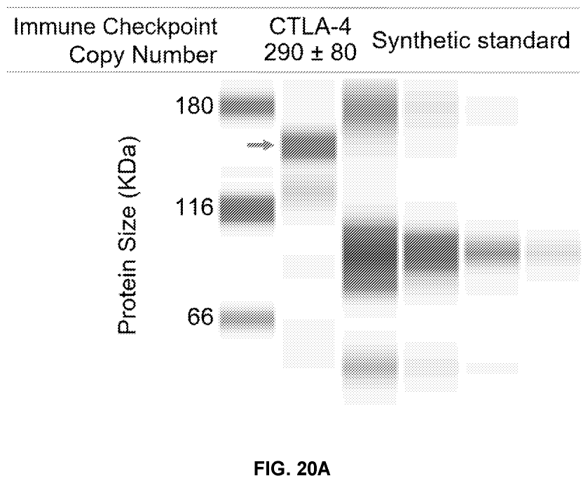

PDL2-MVPs were generated by pseudotyping lentiviral VLPs with trimeric PD-L2 fusion peptides. Specifically, HEK 293T cells were co-transfected with a trimeric PD-L2 display construct, along with a lentiviral packaging construct expressing essential packaging components, including Gag-Pol and Rev proteins, and a viral genome transfer vector encoding a GFP/luciferase reporter (FIG. 3A). The concentration of purified PDL2-MVPs was quantified via P24 ELISA. PDL2-MVPs displayed 2100±500 copies of PD-L2 per MVP in oligomerized forms, as determined by quantitative western blot analysis (FIG. 19A). Hence, the D4 display construct (FIG. 1B) can effectively present thousands of copies of PD-L2 in oligomerized form on MVPs.

To confirm that PD-L2-MVPs display functional PD-L2, it was tested whether PDL2-MVPs can selectively bind to target cells expressing PD-1, its cognate receptor. First, target cell lines were established by transfecting S293 cells with a construct expressing PD-1. Transfected cells were then stained with anti-PD-1 antibody to differentiate PD-1+ from PD-1− cells. Subsequently, PD-L2-MVPs were labeled with fluorescent dye, transfected cells were stained with labeled MVPs, and selective MVP-cell binding was analyzed via FACS (FIG. 19B). The results showed that labeled PD-L2-MVP binding caused significantly higher fluorescence shift in PD-1+ cells as compared to PD-1− cells (FIG. 19B, upper panel). Moreover, this shift was at least one log higher than the fluorescence shift caused by staining the same cells with control MVPs displaying non-specific ligand (FIG. 19B, lower panel). The result demonstrated that PD-L2-MVPs displayed functional PD-L2 and can selectively bind to PD-1 on target cells.

This result was further validated through an alternative staining method (FIG. 19C). In this case, PD-1-transfected cells were first incubated with unlabeled PD-L2-MVPs to allow MVPs to bind to target cells. The cell-MVP mixture was then co-stained with fluorescently-labeled anti-PD-1 and anti-PD-L2 antibodies. PD-L2 staining pattern was then examined on PD-1+ and PD-1-cells via FACS analysis. It was observed that PD-1+ cells were also PD-L2 positive, as exemplified by a 2-log PD-L2 staining shift in PD-1+ cells from PD-1− background cells (FIG. 19C). Single staining with anti-PD-1 antibody did not compete with PDL2-MVP binding to target cells, and PD-1 transfected S293 cells were PD-L2 negative. These results demonstrated that PD-L2-MVPs displayed functional PD-L2. Collectively, PDL2-MVPs were generated displaying high copy numbers of functional protein, and these MVPs can selectively bind target cells expressing their cognate receptor, PD-1.

### CTLA4-MVP Composition and Selective Binding to Target Cells Expressing CD80 CD86

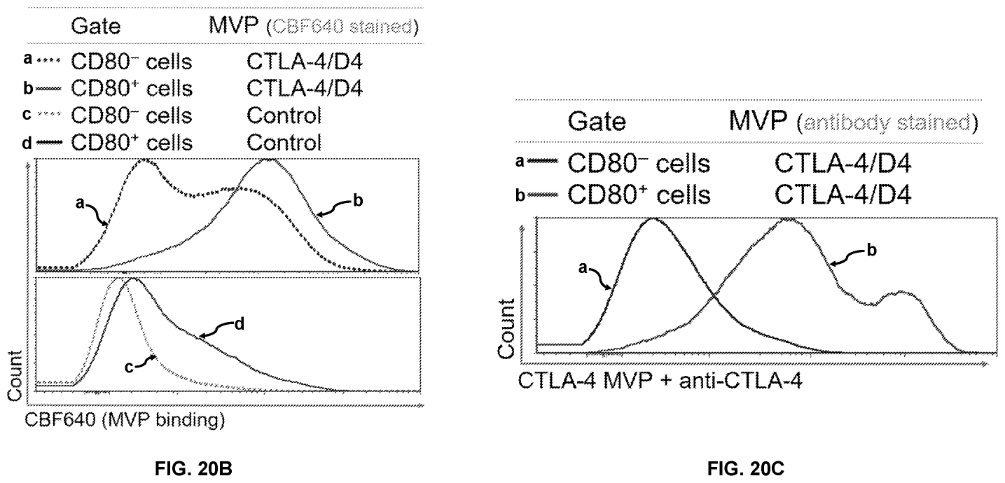

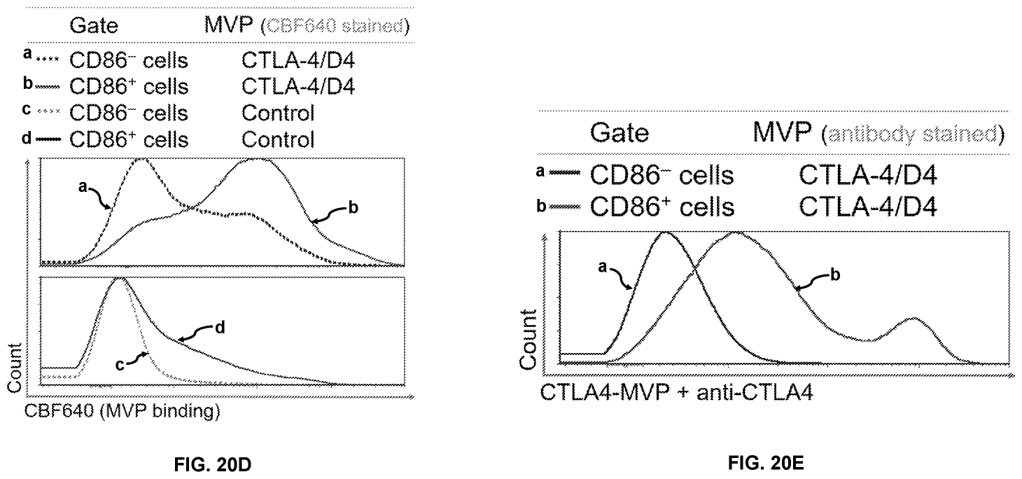

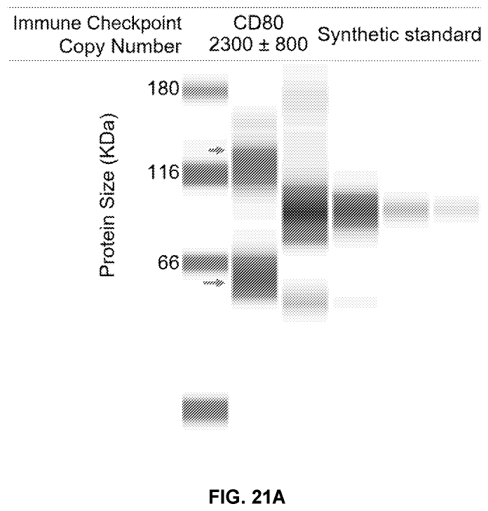

CTLA4-MVPs were generated by pseudotyping lentiviral VLPs with trimeric CTLA-4 fusion peptides. Specifically, HEK 293T cells were co-transfected with a trimeric CTLA-4 display construct, along with a lentiviral packaging construct expressing essential packaging components, including Gag-Pol and Rev proteins, and a viral genome transfer vector encoding a GFP/luciferase reporter (FIG. 3A). The concentration of purified CTLA-4-MVPs was quantified via P24 ELISA. CTLA-4-MVPs displayed 290±80 copies of CTLA-4 per MVP in various oligomerized forms, as determined by quantitative western blot analysis (FIG. 20A). Hence, the D4 display construct (FIG. 1B) can effectively present hundreds of copies of CTLA-4 in oligomerized form on MVPs.

To confirm that CTLA4-MVPs display functional CTLA-4, it was tested whether CTLA-4-MVPs can selectively bind to target cells expressing CD80, its cognate receptor. First, target cell lines were established by transfecting S293 cells with a construct expressing CD80. Transfected cells were then stained with anti-CD80 antibody to differentiate CD80+ from CD80-cells. Subsequently, CTLA4-MVPs were labeled with fluorescent dye, transfected cells were stained with labeled MVPs, and selective MVP-cell binding was analyzed via FACS (FIG. 20B). The results showed that labeled CTLA-4-MVP binding caused significantly higher fluorescence shift in CD80+ cells as compared to CD80− cells (FIG. 20B, upper panel). Moreover, this shift was at least one log higher than the fluorescence shift caused by staining the same cells with control MVPs displaying a non-specific ligand (FIG. 20B, lower panel). The results demonstrated that CTLA4-MVPs displayed functional CTLA-4 and can selectively bind to CD80 on target cells.

This result was further validated through an alternative staining method. In this case, CD80-transfected cells were first incubated with unlabeled CTLA4-MVPs to allow MVPs to bind to target cells. The cell-MVP mixture was then co-stained with fluorescently-labeled anti-CD80 and anti-CTLA-4 antibodies. CTLA-4 staining pattern was then examined on CD80+ and CD80− cells via FACS analysis. The results showed that CD80+ cells were also CTLA4 positive, as exemplified by a 2-log CTLA-4 staining shift in CD80+ cells from CD80− background cells (FIG. 20C). Single staining with anti-CD80 antibody did not compete with CTLA4-MVP binding to target cells, and CD80-transfected S293 cells were CTLA4 negative. These results demonstrated that CTLA4-MVPs displayed functional CTLA-4.

It was also tested whether CTLA-4-MVPs can selectively bind to target cells expressing CD86, another cognate receptor of CTLA-4. First, target cell lines were established by transfecting S293 cells with a construct expressing CD86. Transfected cells were then stained with anti-CD86 antibody to differentiate CD86+ from CD86− cells. Subsequently, CTLA-4-MVPs were labeled with fluorescent dye, transfected cells were stained with labeled MVPs, and selective MVP-cell binding was analyzed via FACS (FIG. 20D). The results showed that labeled CTLA-4-MVP binding caused significantly higher fluorescence shift in CD86+ cells as compared to CD86− cells (FIG. 20D, upper panel). Moreover, this shift is at least one log higher than the fluorescence shift caused by staining the same cells with control MVPs displaying non-specific ligand (FIG. 20D, lower panel). The results demonstrated that CTLA4-MVPs displayed functional CTLA-4 and can selectively bind to CD86 on target cells.

This result was further validated through an alternative staining method. In this case, CD86-transfected cells were first incubated with unlabeled CTLA4-MVPs to allow MVPs to bind to target cells. The cell-MVP mixture was then co-stained with fluorescently-labeled anti-CD86 and anti-CTLA-4 antibodies. CTLA-4 staining pattern on CD86+ and CD86− cells was then examined via FACS analysis. The results showed that CD86+ cells are also CTLA-4 positive, as exemplified by a 2-log CTLA-4 staining shift in CD86+ cells from CD86− background cells (FIG. 20E). Single staining with anti-CD86 antibody did not compete with CTLA-4-MVP binding to target cells, and CD86-transfected S293 cells were CTLA-4 negative. These results demonstrated that CTLA4-MVPs displayed functional CTLA-4. Collectively, CTLA4-MVPs were generated displaying high copy numbers of functional protein, and these MVPs can selectively bind to target cells expressing their cognate receptors CD80 or CD86.

### CD80-MVP Composition and Selective Binding to Target Cells Expressing CTLA-4

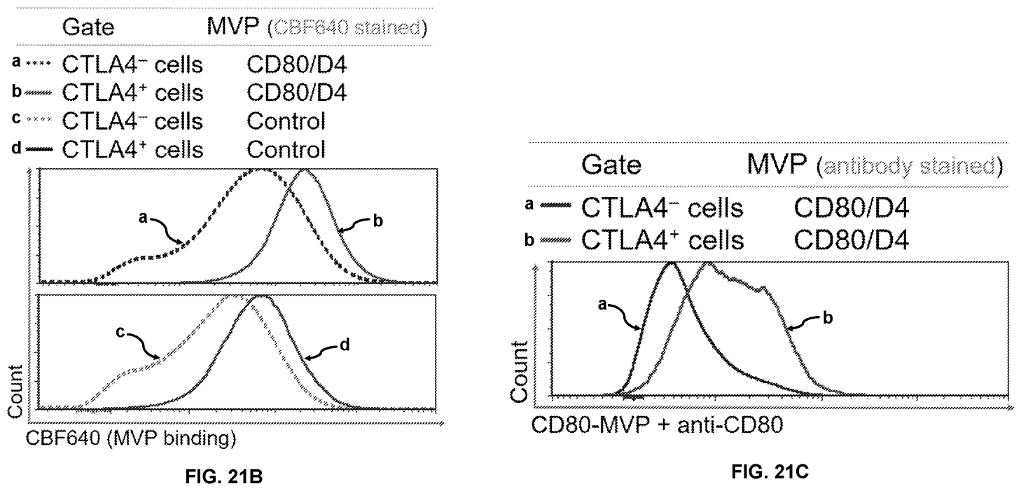

CD80-MVPs were generated by pseudotyping lentiviral VLPs with trimeric CD80 fusion peptides. Specifically, HEK 293T cells were co-transfected with a trimeric CD80 display construct, along with a lentiviral packaging construct expressing essential packaging components, including Gag-Pol and Rev proteins, and a viral genome transfer vector encoding a GFP/luciferase reporter (FIG. 3A). The concentration of purified CD80-MVPs was quantified via P24 ELISA. CD80-MVPs displayed 2300±800 copies of CD80 per MVP in various oligomerized forms, as determined by quantitative western blot analysis (FIG. 21A). Hence, the D4 display construct (FIG. 1B) can effectively present hundreds of copies of CD80 in oligomerized form on MVPs.

To confirm that CD80-MVPs display functional CD80, it was tested whether CD80-MVPs can selectively bind to target cells expressing CTLA-4, a cognate receptor of CD80. First, target cell lines were established by transfecting S293 cells with a construct expressing CTLA-4. Transfected cells were then stained with anti-CTLA-4 antibody to differentiate CTLA-4+ from CTLA-4− cells. Subsequently, CD80-MVPs were labeled with fluorescent dye, transfected cells were stained with labeled MVPs, and selective MVP-cell binding was analyzed via FACS (FIG. 21B). The results showed that labeled CD80-MVP binding caused significantly higher fluorescence shift in CTLA-4+ cells as compared to CTLA-4− cells (FIG. 21B, upper panel). Moreover, this shift was at least one log higher than the fluorescence shift caused by staining the same cells with control MVPs displaying non-specific ligand (FIG. 21B, lower panel). This result demonstrated that CD80-MVPs displayed functional CD80 and can selectively bind CTLA-4 on target cells.

This result was further validated through an alternative staining method (FIG. 21C). In this case, CTLA-4 transfected cells were first incubated with unlabeled CD80-MVPs to allow MVPs to bind to target cells. The cell-MVP mixture was then co-stained with fluorescently-labeled anti-CTLA-4 and anti-CD80 antibodies. CD80 staining pattern was then examined on CTLA-4+ and CTLA-4− cells via FACS analysis. The results showed that CTLA-4+ cells are also CD80 positive, as exemplified by a 0.5-log CD80 staining shift in CTLA-4+ cells from CTLA-4-background cells (FIG. 21C). Single staining with anti-CTLA-4 antibody did not compete with CD80-MVP binding to target cells, and CTLA-4-transfected S293 cells were CD80 negative. These results demonstrated that CD80-MVPs displayed functional CD80. Collectively, CD80-MVPs were generated displaying high copy numbers of functional protein, and these MVPs can selectively bind target cells expressing their cognate receptor CTLA-4.

### CD86-MVP Composition and Selective Binding to Target Cells Expressing CTLA-4

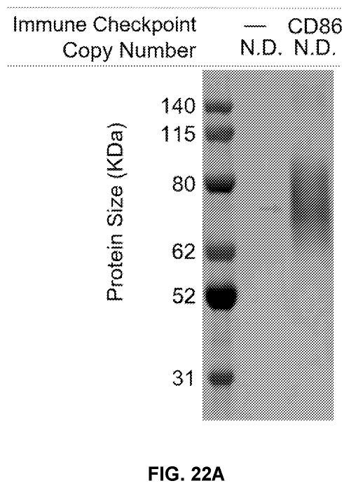

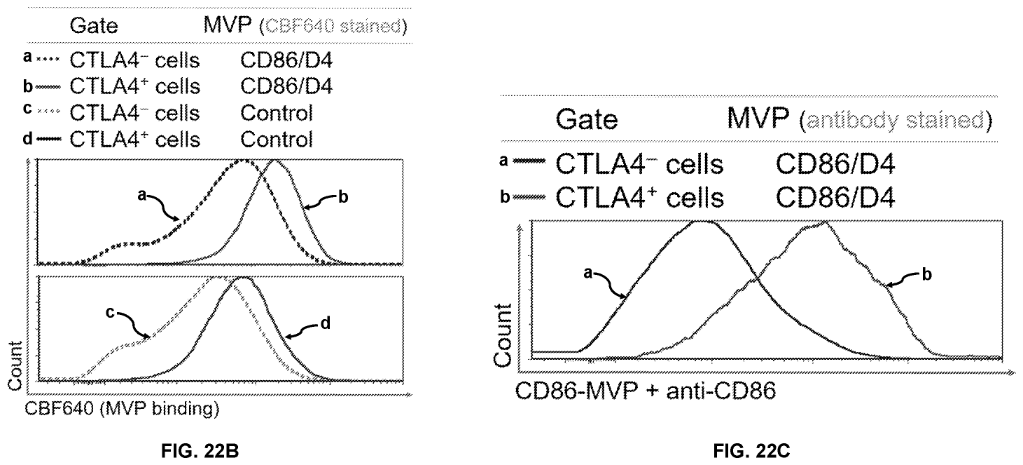

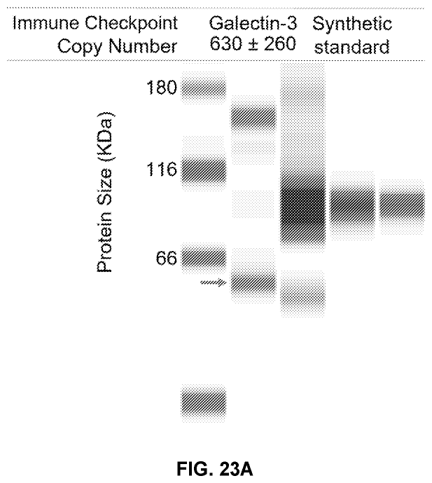

CD86-MVPs were generated by pseudotyping lentiviral VLPs with trimeric CD86 fusion peptides. Specifically, HEK 293T cells were co-transfected with a trimeric CD86 display construct, along with a lentiviral packaging construct expressing essential packaging components, including Gag-Pol and Rev proteins, and a viral genome transfer vector encoding a GFP/luciferase reporter (FIG. 3A). The concentration of purified CD86-MVPs was quantified via P24 ELISA. CD86-MVPs displayed numerous copies of CD86 per MVP in various oligomerized forms, as demonstrated by western blot analysis (FIG. 22A). Hence, the D4 display construct (FIG. 1B) can effectively present CD86 in oligomerized form on MVPs.

To confirm that CD86-MVPs display functional CD86, it was tested whether CD86-MVPs can selectively bind to target cells expressing CTLA-4, a cognate receptor of CD86. First, target cell lines were established by transfecting S293 cells with a construct expressing CTLA-4. Transfected cells were then stained with anti-CTLA-4 antibody to differentiate CTLA-4+ from CTLA-4− cells. Subsequently, CD86-MVPs were labeled with fluorescent dye, transfected cells were stained with labeled MVPs, and selective MVP-cell binding was analyzed via FACS (FIG. 22B). The results showed that labeled CD86-MVP binding caused significantly higher fluorescence shift in CTLA-4+ cells as compared to CTLA-4− cells (FIG. 22B, upper panel). Moreover, this shift was at least one log higher than the fluorescence shift caused by staining the same cells with control MVPs displaying non-specific ligand (FIG. 22B, lower panel). This result demonstrated that CD86-MVPs displayed functional CD86 and can selectively bind CTLA-4 on target cells.

This result was further validated through an alternative staining method (FIG. 22C). In this case, CTLA-4-transfected cells were first incubated with unlabeled CD86-MVPs to allow MVPs to bind to target cells. The cell-MVP mixture was then co-stained with fluorescently-labeled anti-CTLA-4 and anti-CD86 antibodies. CD86 staining pattern on CTLA-4+ and CTLA-4− cells was then examined via FACS analysis. The results showed that CTLA-4+ cells were also CD86 positive, as exemplified by a 1-log CD86 staining shift in CTLA-4+ cells from CTLA-4-background cells (FIG. 22C). Single staining with anti-CTLA-4 antibody did not compete with CD86-MVP binding to target cells, and CTLA-4-transfected S293 cells were CD86 negative. These results demonstrated that CD86-MVPs displayed functional CD86. Collectively, CD86-MVPs were generated displaying high copy numbers of functional protein, and these MVPs can selectively bind to target cells expressing their cognate receptor, CTLA-4.

### GALECTIN3-MVP Composition and Selective Binding to Target Cells Expressing LAG-3

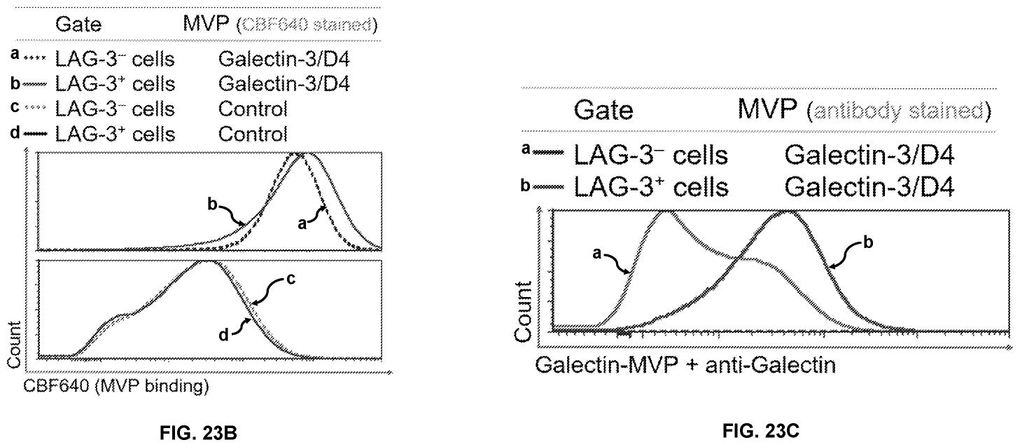

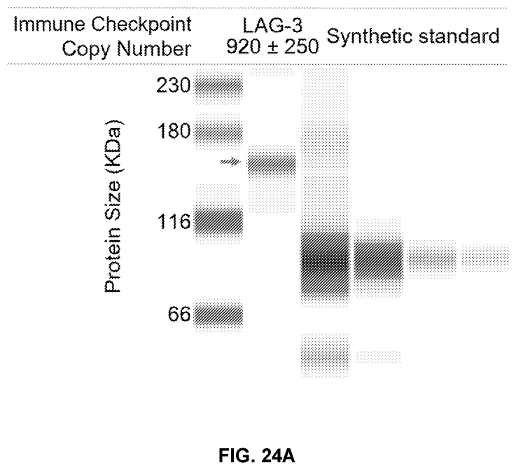

GALECTIN3-MVPs were generated by pseudotyping lentiviral VLPs with trimeric GALECTIN-3 fusion peptides. Specifically, HEK 293T cells were co-transfected with a trimeric GALECTIN-3 display construct, along with a lentiviral packaging construct expressing essential packaging components, including Gag-Pol and Rev proteins, and a viral genome transfer vector encoding a GFP/luciferase reporter (FIG. 3A). The concentration of purified GALECTIN-3-MVPs was quantified via P24 ELISA. GALECTIN3-MVPs displayed 630±260 copies of GALECTIN-3 per MVP in various oligomerized forms, as determined by quantitative western blot analysis (FIG. 23A). Hence, the D4 display construct (FIG. 1B) can effectively present hundreds of copies of GALECTIN-3 in oligomerized form on MVPs.

To confirm that GALECTIN-3-MVPs display functional GALECTIN-3, it was tested whether GALECTIN3-MVPs can selectively bind to target cells expressing LAG-3, a cognate receptor of GALECTIN-3. First, target cell lines were established by transfecting S293 cells with a construct expressing LAG-3. Transfected cells were then stained with anti-LAG-3 antibody to differentiate LAG-3+ from LAG-3− cells. Subsequently, GALECTIN-3-MVPs were labeled with fluorescent dye, transfected cells were stained with labeled MVPs, and selective MVP-cell binding was analyzed via FACS (FIG. 23B). The results showed that labeled GALECTIN3-MVP binding caused significantly higher fluorescence shift in LAG-3+ cells as compared to LAG-3− cells (FIG. 23B, upper panel). Moreover, this shift was at least one log higher than the fluorescence shift caused by staining the same cells with control MVPs displaying non-specific ligand (FIG. 23B, lower panel). This result demonstrated that GALECTIN3-MVPs displayed functional GALECTIN-3 and can selectively bind LAG-3 on target cells.

This result was further validated through an alternative staining method (FIG. 23C). In this case, LAG-3-transfected cells were first incubated with unlabeled GALECTIN3-MVPs to allow MVPs to bind to target cells. The cell-MVP mixture was then co-stained with fluorescently-labeled anti-LAG-3 and anti-GALECTIN-3 antibodies. GALECTIN-3 staining pattern was then examined on LAG-3+ and LAG-3− cells via FACS analysis. The results showed that LAG-3+ cells were also GALECTIN-3 positive, as exemplified by a 1-log GALECTIN-3 staining shift in LAG-3+ cells from LAG-3− background cells (FIG. 23C). Single staining with anti-LAG-3 antibody did not compete with GALECTIN3-MVP binding to target cells, and LAG-3-transfected S293 cells were GALECTIN-3 negative. These results demonstrated that GALECTIN3-MVPs displayed functional GALECTIN-3. Collectively, GALECTIN3-MVPs were generated displaying high copy numbers of functional protein, and these MVPs can selectively bind target cells expressing the GALECTIN-3 cognate receptor, LAG-3.

### LAG3-MVP Composition and Selective Binding to Target Cells Expressing GALECTIN-3

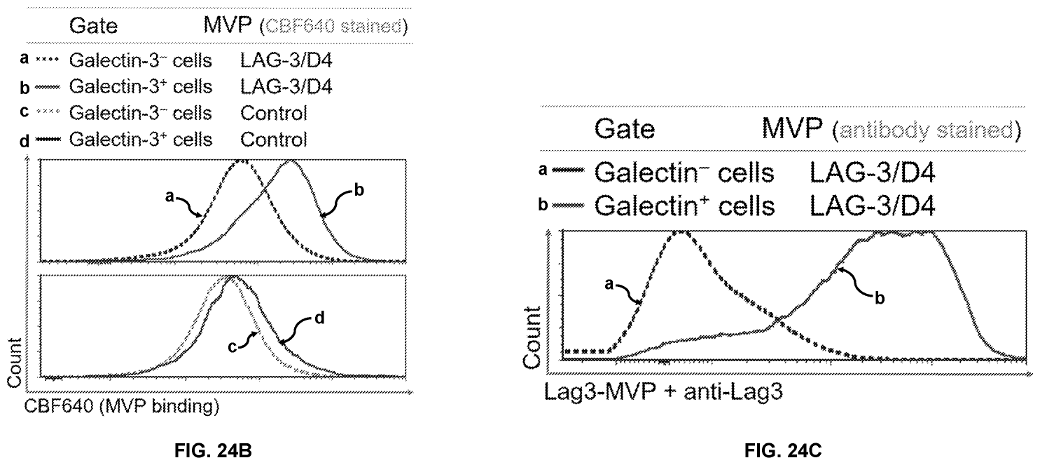

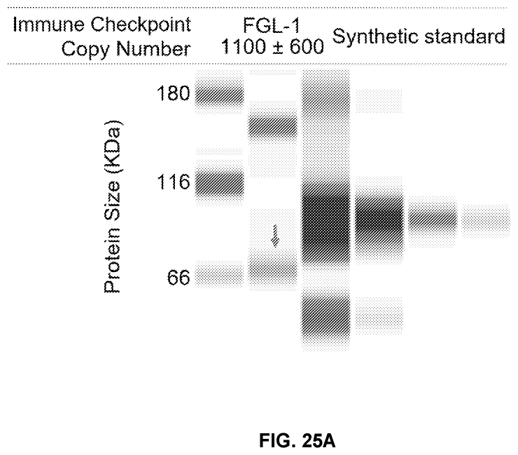

LAG3-MVPs were generated by pseudotyping lentiviral VLPs with trimeric LAG-3 fusion peptides. Specifically, HEK 293T cells were co-transfected with a trimeric LAG-3 display construct, along with a lentiviral packaging construct expressing essential packaging components, including Gag-Pol and Rev proteins, and a viral genome transfer vector encoding a GFP/luciferase reporter (FIG. 3A). The concentration of purified LAG3-MVPs was quantified via P24 ELISA. LAG-3-MVPs displayed 920±250 copies of LAG-3 per MVP in various oligomerized forms, as determined by quantitative western blot analysis (FIG. 24A). Hence, the D4 display construct (FIG. 1B) can effectively present hundreds of copies of LAG-3 in oligomerized form on MVPs.

To confirm that LAG3-MVPs display functional LAG-3, it was tested whether LAG3-MVPs can selectively bind to target cells expressing GALECTIN-3, a cognate receptor of LAG-3. First, target cell lines were established by transfecting S293 cells with a construct expressing GALECTIN-3. Transfected cells were then stained with anti-GALECTIN-3 antibody to differentiate GALECTIN-3+ from GALECTIN-3− cells. Subsequently, LAG3-MVPs were labeled with fluorescent dye, transfected cells were stained with labeled MVPs, and selective MVP-cell binding was analyzed via FACS (FIG. 24B). The results showed that labeled LAG3-MVP binding caused significantly higher fluorescence shift in GALECTIN-3+ cells as compared to GALECTIN-3− cells (FIG. 24B, upper panel). Moreover, this shift was at least one log higher than the fluorescence shift caused by staining the same cells with control MVPs displaying non-specific ligand (FIG. 24B, lower panel). This result demonstrated that LAG3-MVPs displayed functional LAG-3 and can selectively bind GALECTIN-3 on target cells.

This result was further validated through an alternative staining method (FIG. 24C). In this case, GALECTIN-3-transfected cells were first incubated with unlabeled LAG3-MVPs to allow MVPs to bind to target cells. The cell-MVP mixture was then co-stained with fluorescent-labeled anti-GALECTIN-3 and anti-LAG-3 antibodies. LAG-3 staining pattern on GALECTIN-3+ and GALECTIN-3− cells was then examined via FACS analysis. The results showed that GALECTIN-3+ cells are also LAG-3 positive, as exemplified by a 3-log LAG-3 staining shift in GALECTIN-3+ cells from GALECTIN-3− background cells (FIG. 24C). Single staining with anti-LAG-3 antibody did not compete with LAG3-MVP binding to target cells, and GALECTIN-3-transfected S293 cells were LAG-3 negative. These results demonstrated that LAG3-MVPs displayed functional LAG-3. Collectively, LAG3-MVPs were generated displaying high copy numbers of functional protein, and these MVPs can selectively bind target cells expressing their cognate receptor, GALECTIN-3.

### FGL1-MVP Composition and Selective Binding to Target Cells Expressing LAG-3

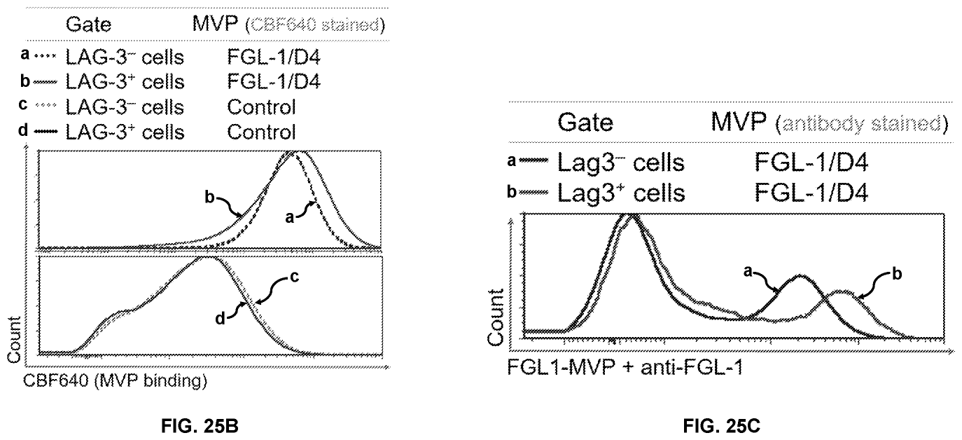

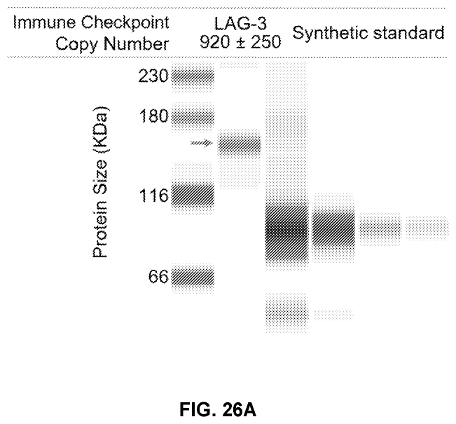

FGL1-MVPs were generated by pseudotyping lentiviral VLPs with trimeric FGL-1 fusion peptides. Specifically, HEK 293T cells were co-transfected with a trimeric FGL-1 display construct, along with a lentiviral packaging construct expressing essential packaging components, including Gag-Pol and Rev proteins, and a viral genome transfer vector encoding a GFP/luciferase reporter (FIG. 3A). The concentration of purified FGL-1-MVPs was quantified via P24 ELISA. FGL-1-MVPs displayed 1100±600 copies of FGL-1 per MVP in various oligomerized forms, as determined by quantitative western blot analysis (FIG. 25A). Hence, the D4 display construct (FIG. 1B) can effectively present hundreds of copies of FGL-1 in oligomerized form on MVPs.

To confirm that FGL1-MVPs display functional FGL-1, it was tested whether FGL1-MVPs can selectively bind to target cells expressing LAG-3, a cognate receptor of FGL-1. First, target cell lines were established by transfecting S293 cells with a construct expressing LAG-3. Transfected cells were then stained with anti-LAG-3 antibody to differentiate LAG-3+ from LAG-3− cells. Subsequently, FGL1-MVPs were labeled with fluorescent dye, transfected cells were stained with labeled MVPs, and selective MVP-cell binding was analyzed via FACS (FIG. 25B). The results showed that labeled FGL1-MVP binding caused significantly higher fluorescence shift in LAG-3+ cells as compared to LAG-3− cells (FIG. 25B, upper panel). Moreover, this shift was at least one log higher than the fluorescence shift caused by staining the same cells with control MVPs displaying non-specific ligand (FIG. 25B, lower panel). This result demonstrated that FGL1-MVPs displayed functional FGL-1 and can selectively bind to LAG-3 on target cells.

This result was further validated through an alternative staining method (FIG. 25C). In this case, LAG-3-transfected cells were first incubated with unlabeled FGL1-MVPs to allow MVPs to bind to target cells. The cell-MVP mixture was then co-stained with fluorescently-labeled anti-LAG-3 and anti-FGL-1 antibodies. FGL-1 staining pattern was then examined on LAG-3+ and LAG-3− cells via FACS analysis. The results showed that LAG-3+ cells were also FGL-1 positive, as exemplified by a 0.5-log FGL-1 staining shift in LAG-3+ cells from LAG-3− background cells (FIG. 25C). Single staining with anti-LAG-3 antibody did not compete with FGL-1-MVP binding to target cells, and LAG-3-transfected S293 cells were FGL-1 negative. These results demonstrated that FGL1-MVPs displayed functional FGL-1. Collectively, FGL1-MVPs were generated displaying high copy numbers of functional protein, and these MVPs can selectively bind to target cells expressing their cognate receptor, LAG-3.

### LAG3-MVP Composition and Selective Binding to Target Cells Expressing FGL-1

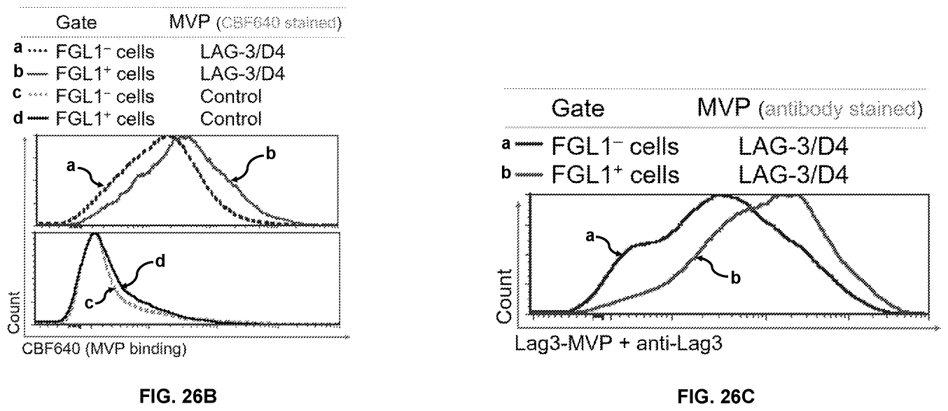

LAG3-MVPs were generated by pseudotyping lentiviral VLPs with trimeric LAG-3 fusion peptides. Specifically, HEK 293T cells were co-transfected with a trimeric LAG-3 display construct, along with a lentiviral packaging construct expressing essential packaging components, including Gag-Pol and Rev proteins, and a viral genome transfer vector encoding a GFP/luciferase reporter (FIG. 3A). The concentration of purified LAG-3-MVPs was quantified via P24 ELISA. LAG-3-MVPs displayed 920±250 copies of LAG-3 per MVP in various oligomerized forms, as determined by quantitative western blot analysis (FIG. 26A). Hence, the D4 display construct (FIG. 1B) can effectively present hundreds of copies of LAG-3 in oligomerized form on MVPs.

To confirm that LAG3-MVPs display functional LAG-3, it was tested whether LAG-3-MVPs can selectively bind to target cells expressing FGL-1, a cognate receptor of LAG-3. First, target cell lines were established by transfecting S293 cells with a construct expressing FGL-1. Transfected cells were then stained with anti-FGL-1 antibody to differentiate FGL-1+ from FGL-1− cells. Subsequently, LAG-3-MVPs were labeled with fluorescent dye, transfected cells were stained with labeled MVPs, and selective MVP-cell binding was analyzed via FACS (FIG. 26B). The results showed that labeled LAG3-MVP binding caused slightly higher fluorescence shift in FGL-1+ cells as compared to FGL-1− cells (FIG. 26B, upper panel). Moreover, this shift was significantly higher than the fluorescence shift caused by staining the same cells with control MVPs displaying non-specific ligand (FIG. 26B, lower panel). This result demonstrated that LAG3-MVPs displayed functional LAG-3 and can selectively bind to FGL-1 on target cells.

This result was further validated through an alternative staining method (FIG. 26C). In this case, FGL-1-transfected cells were first incubated with unlabeled LAG-3-MVPs to allow MVPs to bind to target cells. The cell-MVP mixture was then co-stained with fluorescent-labeled anti-FGL-1 and anti-LAG-3 antibodies. LAG-3 staining pattern was then examined on FGL-1+ and FGL-1− cells via FACS analysis. The results showed that FGL-1+ cells are also LAG-3 positive, as exemplified by a 0.5-log LAG-3 staining shift in FGL-1+ cells from FGL-1− background cells (FIG. 26C). Single staining with anti-FGL-1 antibody did not compete with LAG3-MVP binding to target cells, and FGL-1-transfected S293 cells were LAG-3 negative. These results demonstrated that LAG3-MVPs displayed functional LAG-3. Collectively, LAG3-MVPs were generated displaying high copy numbers of functional protein, and these MVPs can selectively bind to target cells expressing their cognate receptor, FGL-1.

### HVEM-MVP Composition and Selective Binding to Target Cells Expressing BTLA

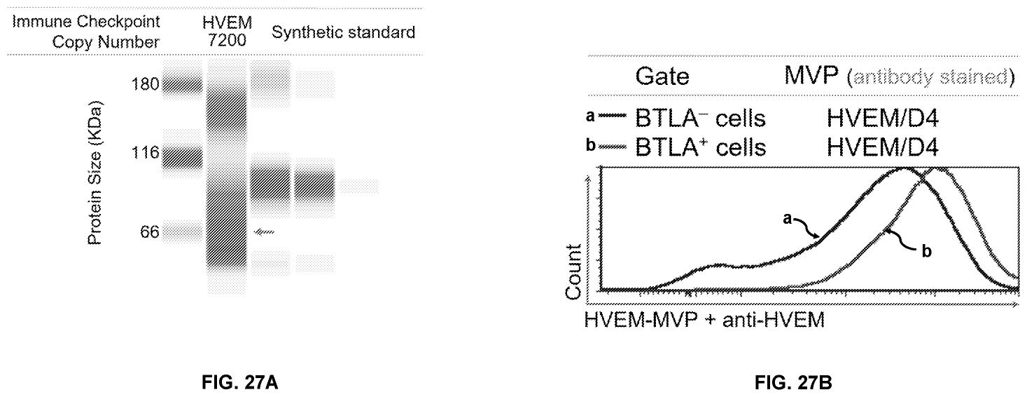

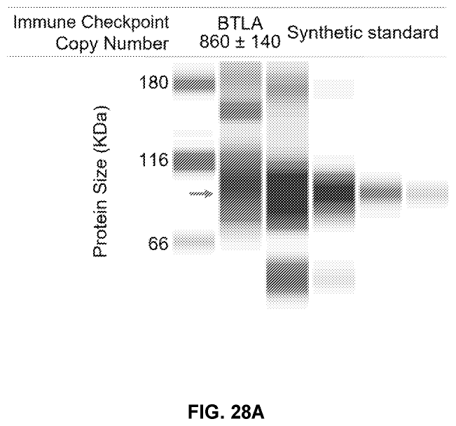

HVEM-MVPs were generated by pseudotyping lentiviral VLPs with trimeric HVEM fusion peptides. Specifically, HEK 293T cells were co-transfected with a trimeric HVEM display construct, along with a lentiviral packaging construct expressing essential packaging components, including Gag-Pol and Rev proteins, and a viral genome transfer vector encoding a GFP/luciferase reporter (FIG. 3A). The concentration of purified HVEM-MVPs was quantified via P24 ELISA. HVEM-MVPs displayed 7200 copies of HVEM per MVP in various oligomerized forms, as determined by quantitative western blot analysis (FIG. 27A). Hence, the D4 display construct (FIG. 1B) can effectively present hundreds of copies of HVEM in oligomerized form on MVPs.

To confirm that HVEM-MVPs display functional HVEM, it was tested whether HVEM-MVPs can selectively bind to target cells expressing BTLA, a cognate receptor of HVEM. First, target cell lines were established by transfecting S293 cells with a construct expressing BTLA. BTLA-transfected cells were then incubated with unlabeled HVEM-MVPs to allow MVPs to bind to target cells. The cell-MVP mixture was then co-stained with fluorescently-labeled anti-BTLA and anti-HVEM antibodies. HVEM staining pattern was then examined on BTLA+ and BTLA-cells via FACS analysis. The results showed that BTLA+ cells were also HVEM positive, as exemplified by a 0.5-log HVEM staining shift in BTLA+ cells from BTLA− background cells (FIG. 27B). Single staining with anti-BTLA antibody did not compete with HVEM-MVP binding to target cells, and BTLA-transfected S293 cells were HVEM negative. These results demonstrated that HVEM-MVPs displayed functional HVEM. Collectively, HVEM-MVPs were generated displaying high copy numbers of functional protein, and these MVPs can selectively bind to target cells expressing their cognate receptor, BTLA.

### BTLA-MVP Composition and Selective Binding to Target Cells Expressing HVEM

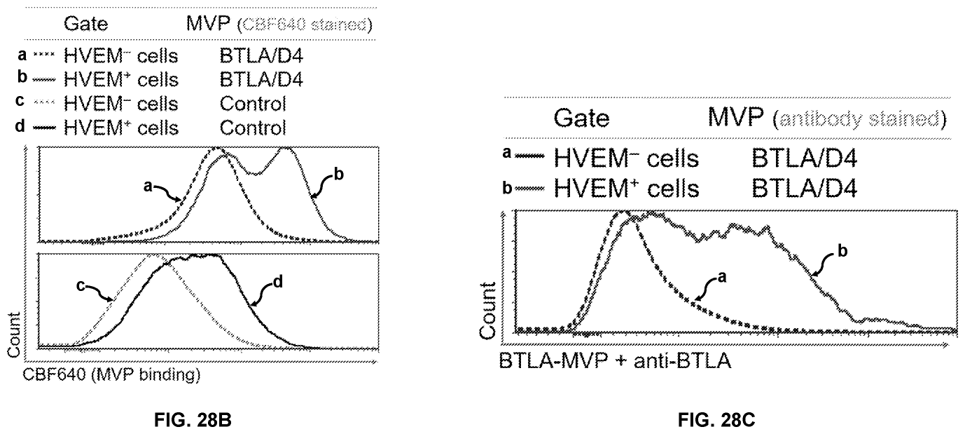

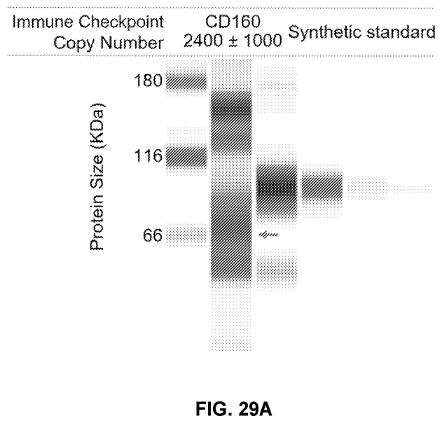

BTLA-MVPs were generated by pseudotyping lentiviral VLPs with trimeric BTLA fusion peptides. Specifically, HEK 293T cells were co-transfected with a trimeric BTLA display construct, along with a lentiviral packaging construct expressing essential packaging components, including Gag-Pol and Rev proteins, and a viral genome transfer vector encoding a GFP/luciferase reporter (FIG. 3A). The concentration of purified BTLA-MVPs was quantified via P24 ELISA. BTLA-MVPs displayed 860±140 copies of BTLA per MVP in various oligomerized forms, as determined by quantitative western blot analysis (FIG. 28A). Hence, the D4 display construct (FIG. 1B) can effectively present hundreds of copies of BTLA in oligomerized form on MVPs.

To confirm that BTLA-MVPs display functional BTLA, it was tested whether BTLA-MVPs can selectively bind to target cells expressing HVEM, a cognate receptor of BTLA. First, target cell lines were established by transfecting S293 cells with a construct expressing HVEM. Transfected cells were then stained with anti-HVEM antibody to differentiate HVEM+ from HVEM− cells. Subsequently, BTLA-MVPs were labeled with fluorescent dye, transfected cells were stained with labeled MVPs, and selective MVP-cell binding was analyzed via FACS (FIG. 28B). The results showed that labeled BTLA-MVP binding caused significantly higher fluorescence shift in HVEM+ cells as compared to HVEM− cells (FIG. 28B, upper panel). Moreover, this shift was at least one log higher than the fluorescence shift caused by staining the same cells with control MVPs displaying non-specific ligand (FIG. 28B, lower panel). This result demonstrated that BTLA-MVPs displayed functional BTLA and can selectively bind to HVEM on target cells.

This result was further validated through an alternative staining method (FIG. 28C). In this case, HVEM-transfected cells were first incubated with unlabeled BTLA-MVPs to allow MVPs to bind to target cells. The cell-MVP mixture was then co-stained with fluorescent-labeled anti-HVEM and anti-BTLA antibodies. BTLA staining pattern was then examined on HVEM+ and HVEM− cells via FACS analysis. The results showed that HVEM+ cells were also BTLA positive, as exemplified by a 1-log BTLA staining shift in HVEM+ cells from HVEM− background cells (FIG. 28C). Single staining with anti-HVEM antibody did not compete with BTLA-MVP binding to target cells, and HVEM-transfected S293 cells were BTLA negative. These results demonstrated that BTLA-MVPs displayed functional BTLA. Collectively, BTLA-MVPs were generated displaying high copy numbers of functional protein, and these MVPs can selectively bind target cells expressing their cognate receptor, HVEM.

### CD160-MVP Composition and Selective Binding to Target Cells Expressing HVEM

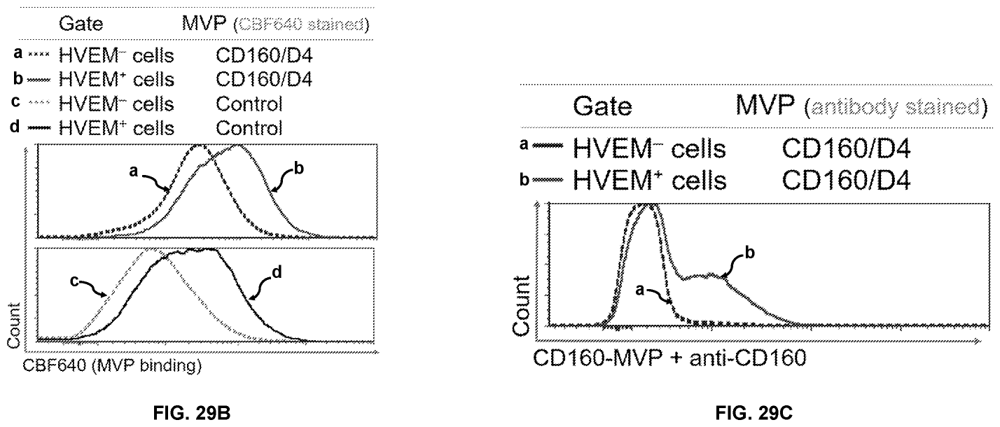

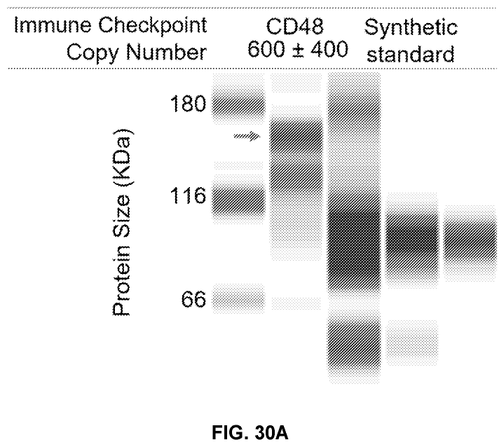

CD160-MVPs were generated by pseudotyping lentiviral VLPs with trimeric CD160 fusion peptides. Specifically, HEK 293T cells were co-transfected with a trimeric CD160 display construct, along with a lentiviral packaging construct expressing essential packaging components, including Gag-Pol and Rev proteins, and a viral genome transfer vector encoding a GFP/luciferase reporter (FIG. 3A). The concentration of purified CD160-MVPs was quantified via P24 ELISA. CD160-MVPs displayed 2400±1000 copies of CD160 per MVP in various oligomerized forms, as determined by quantitative western blot analysis (FIG. 29A). Hence, the D4 display construct (FIG. 1B) can effectively present hundreds of copies of CD160 in oligomerized form on MVPs.

To confirm that CD160-MVPs display functional CD160, it was tested whether CD160-MVPs can selectively bind to target cells expressing HVEM, a cognate receptor of CD160. First, target cell lines were established by transfecting S293 cells with a construct expressing HVEM. Transfected cells were then stained with anti-HVEM antibody to differentiate HVEM+ from HVEM− cells. Subsequently, CD160-MVPs were labeled with fluorescent dye, transfected cells were stained with labeled MVPs, and selective MVP-cell binding was analyzed via FACS (FIG. 29B). The results showed that labeled CD160-MVP binding caused significantly higher fluorescence shift in HVEM+ cells as compared to HVEM− cells (FIG. 29B, upper panel). Moreover, this shift was at least one log higher than the fluorescence shift caused by staining the same cells with control MVPs displaying non-specific ligand (FIG. 29B, lower panel). This result demonstrated that CD160-MVPs displayed functional CD160 and can selectively bind HVEM on target cells.

This result was further validated through an alternative staining method (FIG. 29C). In this case, HVEM-transfected cells were first incubated with unlabeled CD160-MVPs to allow MVPs to bind to target cells. The cell-MVP mixture was then co-stained with fluorescently-labeled anti-HVEM and anti-CD160 antibodies. CD160 staining pattern was then examined on HVEM+ and HVEM− cells via FACS analysis. The results showed that HVEM+ cells were also CD160 positive, as exemplified by a 0.5-log CD160 staining shift in HVEM+ cells from HVEM-background cells (FIG. 29C). Single staining with anti-HVEM antibody did not compete with CD160-MVP binding to target cells, and HVEM-transfected S293 cells were CD160 negative. These results demonstrated that CD160-MVPs displayed functional CD160. Collectively, generated CD160-MVPs were displaying high copy numbers of functional protein, and these MVPs can selectively bind to target cells expressing their cognate receptor, HVEM.

### CD48-MVP Composition and Selective Binding to Target Cells Expressing 2B4

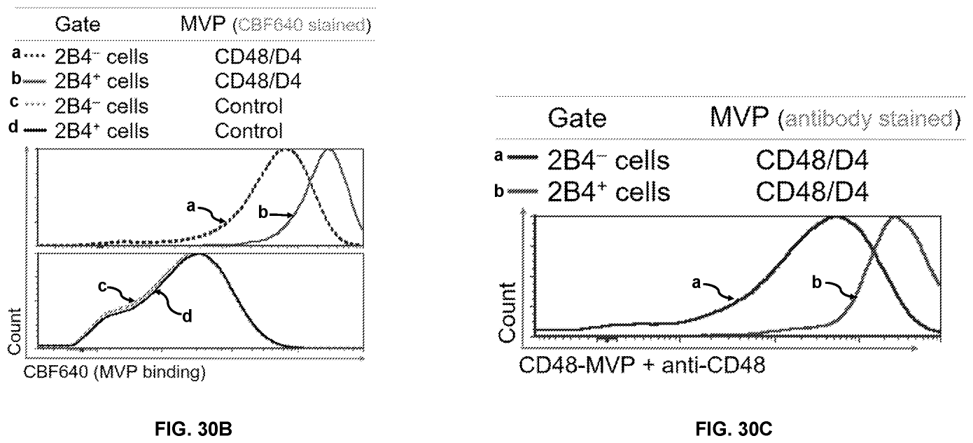

CD48-MVPs were generated by pseudotyping lentiviral VLPs with trimeric CD48 fusion peptides. Specifically, HEK 293T cells were co-transfected with a trimeric CD48 display construct, along with a lentiviral packaging construct expressing essential packaging components, including Gag-Pol and Rev proteins, and a viral genome transfer vector encoding a GFP/luciferase reporter (FIG. 3A). The concentration of purified CD48-MVPs was quantified via P24 ELISA. CD48-MVPs displayed 600±400 copies of CD48 per MVP in various oligomerized forms, as determined by quantitative western blot analysis (FIG. 30A). Hence, the D4 display construct (FIG. 1B) can effectively present hundreds of copies of CD48 in oligomerized form on MVPs.

To confirm that CD48-MVPs display functional CD48, it was tested whether CD48-MVPs can selectively bind to target cells expressing 2B4, a cognate receptor of CD48. First, target cell lines were established by transfecting S293 cells with a construct expressing 2B4. Transfected cells were then stained with anti-2B4 antibody to differentiate 2B4+ from 2B4− cells. Subsequently, CD48-MVPs were labeled with fluorescent dye, transfected cells were stained with labeled MVPs, and selective MVP-cell binding was analyzed via FACS (FIG. 30B). The results showed that labeled CD48-MVP binding caused significantly higher fluorescence shift in 2B4+ cells as compared to 2B4− cells (FIG. 30B, upper panel). Moreover, this shift was at least one log higher than the fluorescence shift caused by staining the same cells with control MVPs displaying non-specific ligand (FIG. 30B, lower panel). This result demonstrated that CD48-MVPs displayed functional CD48 and can selectively bind 2B4 on target cells.

This result was further validated through an alternative staining method (FIG. 30C). In this case, 2B4-transfected cells were first incubated with unlabeled CD48-MVPs to allow MVPs to bind to target cells. The cell-MVP mixture was then co-stained with fluorescent-labeled anti-2B4 and anti-CD48 antibodies. CD48 staining pattern was then examined on 2B4+ and 2B4− cells via FACS analysis. The results showed that 2B4+ cells were also CD48 positive, as exemplified by a 0.5-log CD48 staining shift in 2B4+ cells from 2B4− background cells (FIG. 30C). Single staining with anti-2B4 antibody did not compete with CD48-MVP binding to target cells, and 2B4-transfected S293 cells were CD48 negative. These results demonstrated that CD48-MVPs displayed functional CD48. Collectively, CD48-MVPs were generated displaying high copy numbers of functional protein, and these MVPs can selectively bind target cells expressing their cognate receptor, 2B4.

### CD112-MVP Composition and Selective Binding to Target Cells Expressing TIGIT

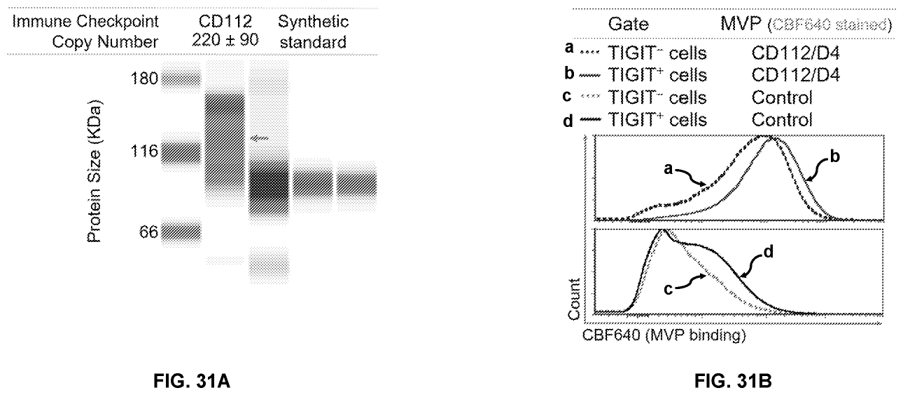

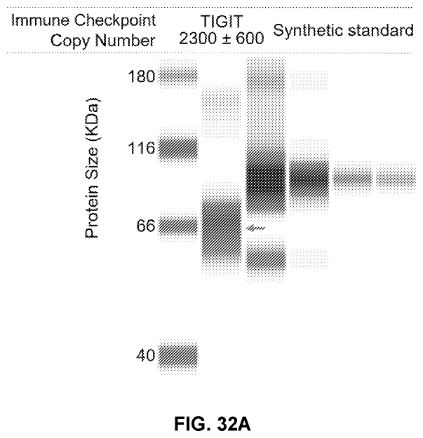

CD112-MVPs were generated by pseudotyping lentiviral VLPs with trimeric CD112 fusion peptides. Specifically, HEK 293T cells were co-transfected with a trimeric CD112 display construct, along with a lentiviral packaging construct expressing essential packaging components, including Gag-Pol and Rev proteins, and a viral genome transfer vector encoding a GFP/luciferase reporter (FIG. 3A). The concentration of purified CD112-MVPs was quantified via P24 ELISA. CD112-MVPs displayed 220±90 copies of CD112 per MVP in various oligomerized forms, as determined by quantitative western blot analysis (FIG. 31A). Hence, the D4 display construct (FIG. 1B) can effectively present hundreds of copies of CD112 in oligomerized form on MVPs.

To confirm that CD112-MVPs display functional CD112, it was tested whether CD112-MVPs can selectively bind to target cells expressing TIGIT, a cognate receptor of CD112. First, target cell lines were established by transfecting S293 cells with a construct expressing TIGIT. Transfected cells were then stained with anti-TIGIT antibody to differentiate TIGIT+ from TIGIT-cells. Subsequently, CD112-MVPs were labeled with fluorescent dye, transfected cells were stained with labeled MVPs, and selective MVP-cell binding was analyzed via FACS (FIG. 31B). The results showed that labeled CD112-MVP binding caused significantly higher fluorescence shift in TIGIT+ cells as compared to TIGIT− cells (FIG. 31B, upper panel). Moreover, this shift was at least one log higher than the fluorescence shift caused by staining the same cells with control MVPs displaying non-specific ligand (FIG. 31B, lower panel). This result demonstrated that CD112-MVPs displayed functional CD112 and can selectively bind to TIGIT on target cells. Collectively, CD112-MVPs were generated displaying high copy numbers of functional protein, and these MVPs can selectively bind target cells expressing their cognate receptor, TIGIT.

### TIGIT-MVP Composition and Selective Binding to Target Cells Expressing CD112

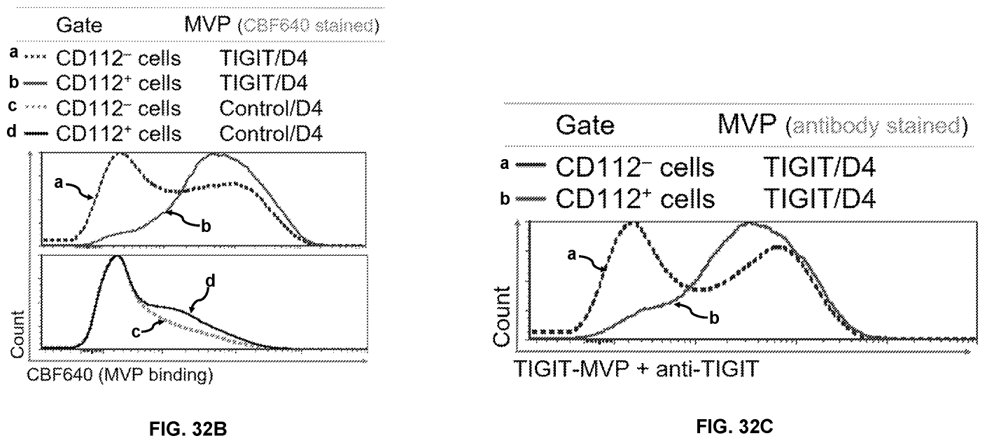

TIGIT-MVPs were generated by pseudotyping lentiviral VLPs with trimeric TIGIT fusion peptides. Specifically, HEK 293T cells were co-transfected with a trimeric TIGIT display construct, along with a lentiviral packaging construct expressing essential packaging components, including Gag-Pol and Rev proteins, and a viral genome transfer vector encoding a GFP/luciferase reporter (FIG. 3A). The concentration of purified TIGIT-MVPs was quantified via P24 ELISA. TIGIT-MVPs displayed 2300±600 copies of TIGIT per MVP in various oligomerized forms, as determined by quantitative western blot analysis (FIG. 32A). Hence, the D4 display construct (FIG. 1B) can effectively present hundreds of copies of TIGIT in oligomerized form on MVPs.

To confirm that TIGIT-MVPs display functional TIGIT, it was tested whether TIGIT-MVPs can selectively bind to target cells expressing CD112, one of TIGIT's cognate receptors. First, target cell lines were established by transfecting S293 cells with a construct expressing CD112. Transfected cells were then stained with anti-CD155 antibody to differentiate CD112+ from CD112− cells. Subsequently, TIGIT-MVPs were labeled with fluorescent dye, transfected cells were stained with labeled MVPs, and selective MVP-cell binding was analyzed via FACS. The results showed that labeled TIGIT-MVP binding caused significantly higher fluorescence shift in CD112+ cells as compared to CD112− cells (FIG. 32B, upper panel). Moreover, this shift was at least three times higher than the fluorescence shift caused by staining the same cells with control MVPs displaying non-specific ligand (FIG. 32B, lower panel). This result demonstrated that TIGIT-MVPs displayed functional TIGIT and can selectively bind to CD112 on target cells.

This result was further validated through an alternative staining method (FIG. 32C). In this case, CD112-transfected cells were first incubated with unlabeled TIGIT-MVPs to allow MVPs to bind to target cells. The cell-MVP mixture was then co-stained with fluorescently-labeled anti-CD112 and anti-TIGIT antibodies. TIGIT staining pattern on CD112+ and CD112− cells was then examined via FACS analysis. The results showed that CD112+ cells were also TIGIT positive, as exemplified by two-log TIGIT staining shift in CD112+ cells from CD112− background cells (FIG. 32C). Single staining with anti-CD112 antibody did not compete with TIGIT-MVP binding to target cells, and CD112-transfected S293 cells were TIGIT negative. These results demonstrated that TIGIT-MVPs displayed functional TIGIT. Collectively, TIGIT-MVPs were generated displaying high copy numbers of functional protein, and these MVPs can selectively bind target cells expressing their cognate receptor, CD112.

### CD155-MVP Composition and Selective Binding to Target Cells Expressing TIGIT

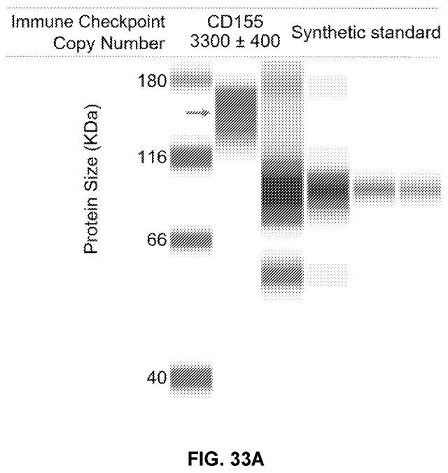

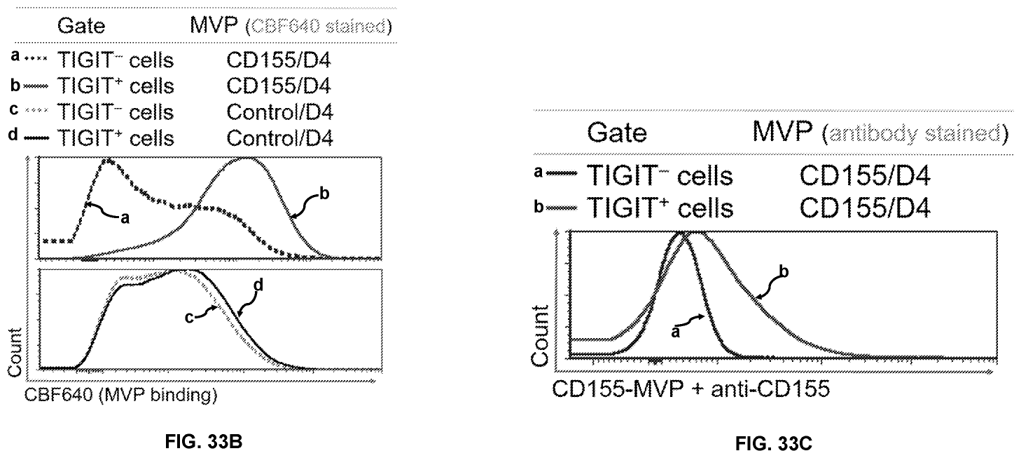

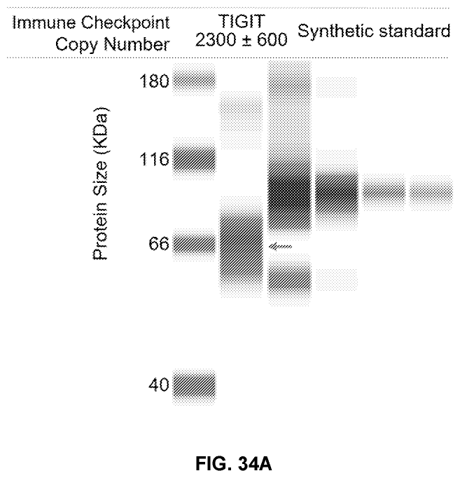

CD155-MVPs were generated by pseudotyping lentiviral VLPs with trimeric CD155 fusion peptides. Specifically, HEK 293T cells were co-transfected with a trimeric CD155 display construct, along with a lentiviral packaging construct expressing essential packaging components, including Gag-Pol and Rev proteins, and a viral genome transfer vector encoding a GFP/luciferase reporter (FIG. 3A). The concentration of purified CD155-MVPs was quantified via P24 ELISA. CD155-MVPs displayed 3300±400 copies of TIGIT per MVP in various oligomerized forms, as determined by quantitative western blot analysis (FIG. 33A). Hence, the D4 display construct (FIG. 1B) can effectively present thousands of copies of CD155 in oligomerized form on MVPs.

To confirm that CD155-MVPs display functional CD155, it was tested whether CD155-MVPs can selectively bind to target cells expressing TIGIT, a cognate receptor of CD155. First, target cell lines were established by transfecting S293 cells with a construct expressing TIGIT. Transfected cells were then stained with anti-CD155 antibody to differentiate CD155+ from CD155− cells. Subsequently, CD155-MVPs were labeled with fluorescent dye, transfected cells were stained with labeled MVPs, and selective MVP-cell binding was analyzed via FACS (FIG. 33B). The results showed that labeled CD155-MVP binding caused significantly higher fluorescence shift in TIGIT+ cells as compared to TIGIT− cells (FIG. 33B, upper panel). Moreover, this shift was at least three times higher than the fluorescence shift caused by staining the same cells with control MVPs displaying non-specific ligand (FIG. 33B, lower panel). This result demonstrated that CD155-MVPs displayed functional CD155 and can selectively bind TIGIT on target cells.

This result was further validated through an alternative staining method (FIG. 33C). In this case, TIGIT-transfected cells were first incubated with unlabeled CD155-MVPs to allow MVPs to bind to target cells. The cell-MVP mixture was then co-stained with fluorescently-labeled anti-CD155 and anti-TIGIT antibodies. CD155 staining pattern on TIGIT+ and TIGIT− cells was then examined via FACS analysis. The results showed that TIGIT+ cells were also CD155 positive, as exemplified by two-log CD155 staining shift in TIGIT+ cells from TIGIT-background cells (FIG. 33C). Single staining with anti-TIGIT antibody did not compete with CD155-MVP binding to target cells, and TIGIT-transfected S293 cells were CD155 negative. These results demonstrated that CD155-MVPs displayed functional CD155. Collectively, CD155-MVPs were generated displaying high copy numbers of functional protein, and these MVPs can selectively bind target cells expressing their cognate receptor, TIGIT.

### TIGIT-MVP Composition and Selectively Bind to Target Cells Expressing CD155

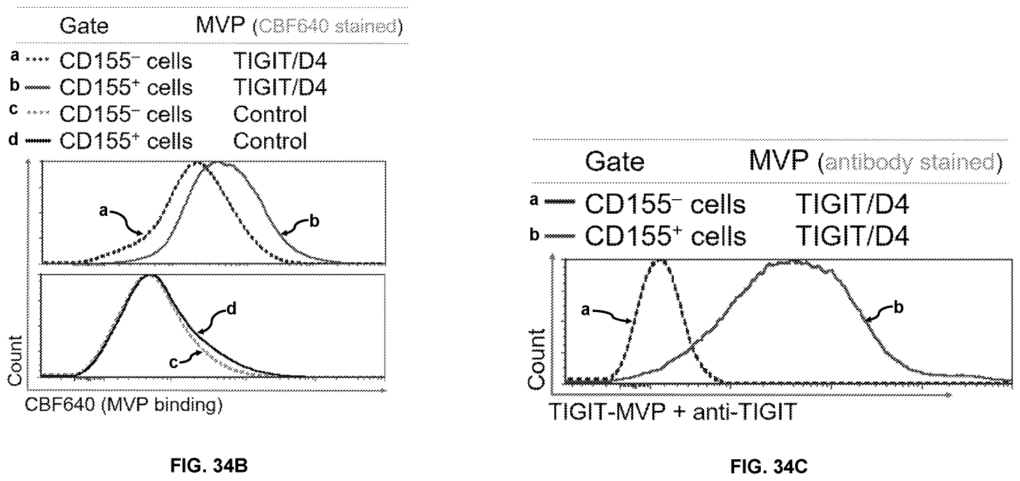

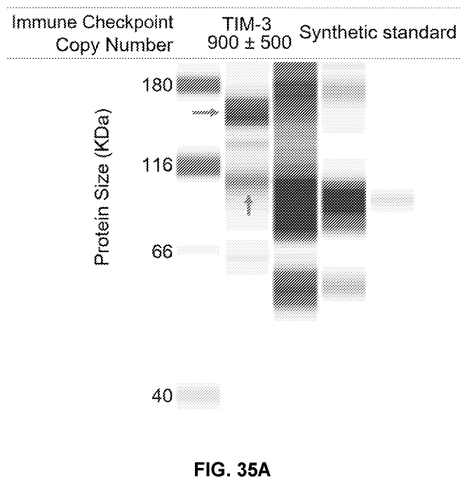

TIGIT-MVPs were generated by pseudotyping lentiviral VLPs with trimeric TIGIT fusion peptides. Specifically, HEK 293T cells were co-transfected with a trimeric TIGIT display construct, along with a lentiviral packaging construct expressing essential packaging components, including Gag-Pol and Rev proteins, and a viral genome transfer vector encoding a GFP/luciferase reporter (FIG. 3A). The concentration of purified TIGIT-MVPs was quantified via P24 ELISA. TIGIT-MVPs displayed 2300±600 copies of TIGIT per MVP in various oligomerized forms, as determined by quantitative western blot analysis (FIG. 34A). Hence, the D4 display construct (FIG. 1B) can effectively present hundreds of copies of TIGIT in oligomerized form on MVPs.

To confirm that TIGIT-MVPs display functional TIGIT, it was tested whether TIGIT-MVPs can selectively bind to target cells expressing CD155, one of TIGIT's cognate receptors. First, target cell lines were established by transfecting S293 cells with a construct expressing CD155. Transfected cells were then stained with anti-CD155 antibody to differentiate CD155+ from CD155− cells. Subsequently, TIGIT-MVPs were labeled with fluorescent dye, transfected cells were stained with labeled MVPs, and selective MVP-cell binding was analyzed via FACS (FIG. 34B). The results showed that labeled TIGIT-MVP binding caused significantly higher fluorescence shift in CD155+ cells as compared to CD155− cells (FIG. 34B, upper panel). Moreover, this shift was at least three times higher than the fluorescence shift caused by staining the same cells with control MVPs displaying non-specific ligand (FIG. 34B, lower panel). This result demonstrated that TIGIT-MVPs displayed functional TIGIT and can selectively bind CD155 on target cells.

This result was further validated through an alternative staining method (FIG. 34C). In this case, CD155-transfected cells were first incubated with unlabeled TIGIT-MVPs to allow MVPs to bind to target cells. The cell-MVP mixture was then co-stained with fluorescently-labeled anti-CD155 and anti-TIGIT antibodies. TIGIT staining pattern on CD155+ and CD155− cells was then examined via FACS analysis. The results showed that CD155+ cells were also TIGIT positive, as exemplified by two-log TIGIT staining shift in CD155+ cells from CD155− background cells (FIG. 34C). Single staining with anti-CD155 antibody did not compete with TIGIT-MVP binding to target cells, and CD155-transfected S293 cells were TIGIT negative. These results demonstrated that TIGIT-MVPs displayed functional TIGIT. Collectively, TIGIT-MVPs were generated displaying high copy numbers of functional protein, and these MVPs can selectively bind target cells expressing their cognate receptor, CD155.

### TIM3-MVP Composition and Selective Binding to Target Cells Expressing Ceacam-1

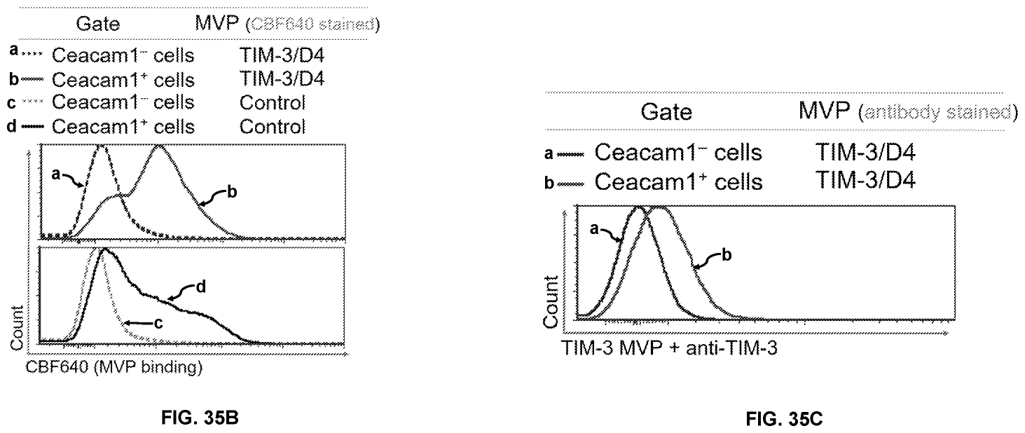

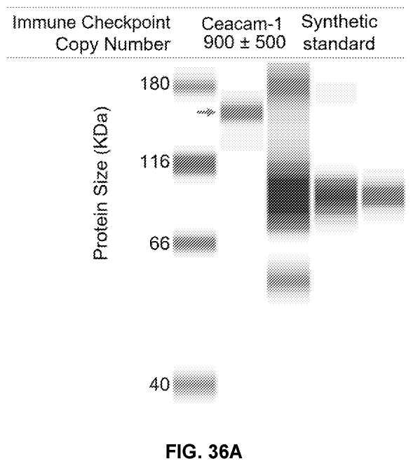

TIM3-MVPs were generated by pseudotyping lentiviral VLPs with trimeric TIM-3 fusion peptides. Specifically, HEK 293T cells were co-transfected with a trimeric TIM-3 display construct, along with a lentiviral packaging construct expressing essential packaging components, including Gag-Pol and Rev proteins, and a viral genome transfer vector encoding a GFP/luciferase reporter (FIG. 3A). The concentration of purified TIM3-MVPs was quantified via P24 ELISA. TIM3-MVPs displayed 900±500 copies of TIM3 per MVP in various oligomerized forms, as determined by quantitative western blot analysis (FIG. 35A). Hence, the D4 display construct (FIG. 1B) can effectively present hundreds of copies of TIM3 in oligomerized form on MVPs.

To confirm that TIM3-MVPs display functional TIM3, it was tested whether TIM3-MVPs can selectively bind to target cells expressing Ceacam1, a cognate receptor of TIM-3. First, target cell lines were established by transfecting S293 cells with a construct expressing Ceacam1. Transfected cells were then stained with anti-Ceacam1 antibody to differentiate Ceacam1+ from Ceacam1− cells. Subsequently, TIM3-MVPs were labeled with fluorescent dye, transfected cells were stained with labeled MVPs, and selective MVP-cell binding was analyzed via FACS (FIG. 35B). The results showed that labeled TIM3-MVP binding caused significantly higher fluorescence shift in Ceacam1+ cells as compared to Ceacam1− cells (FIG. 35B, upper panel). Moreover, this shift was at least four to five times higher than the fluorescence shift caused by staining the same cells with control MVPs displaying non-specific ligand (FIG. 35B, lower panel). This result demonstrated that TIM3-MVPs displayed functional TIM-3 and can selectively bind Ceacam1 on target cells.

This result was further validated through an alternative staining method (FIG. 35C). In this case, Ceacam1-transfected cells were first incubated with unlabeled TIM-3-MVPs to allow MVPs to bind to target cells. The cell-MVP mixture was then co-stained with fluorescently-labeled anti-Ceacam1 and anti-TIM3 antibodies. TIM-3 staining pattern on Ceacam1+ and Ceacam1− cells was then examined via FACS analysis. The results showed that Ceacam1+ cells were also TIM-3 positive, as exemplified by two to three times higher TIM-3 staining shift in Ceacam1+ cells from Ceacam1− background cells (FIG. 35C). Single staining with anti-Ceacam1 antibody did not compete with TIM3-MVP binding to target cells, and Ceacam1-transfected S293 cells were TIM-3 negative. These results demonstrated that TIM3-MVPs displayed functional TIM-3. Collectively, TIM3-MVPs were generated displaying high copy numbers of functional protein, and these MVPs can selectively bind target cells expressing their cognate receptor, Ceacam-1.

### Ceacam1-MVP Composition and Selective Binding to Target Cells Expressing TIM-3

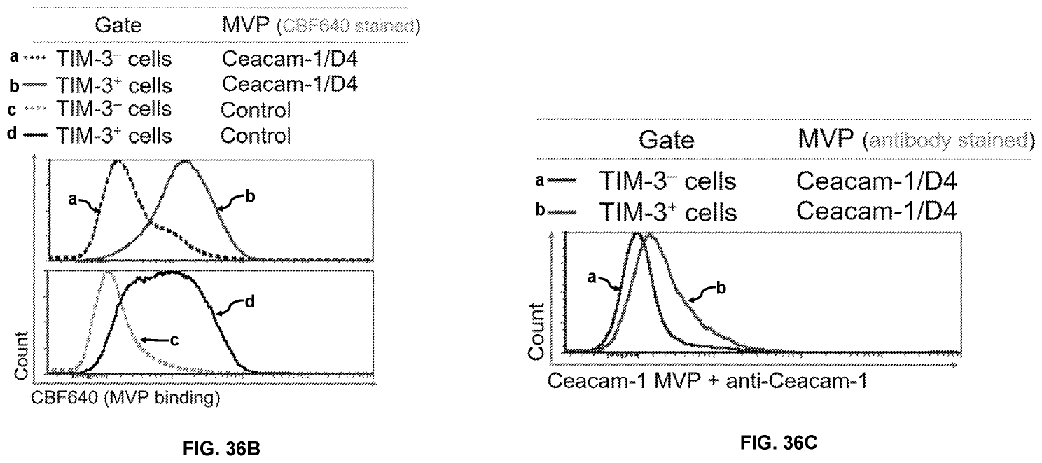

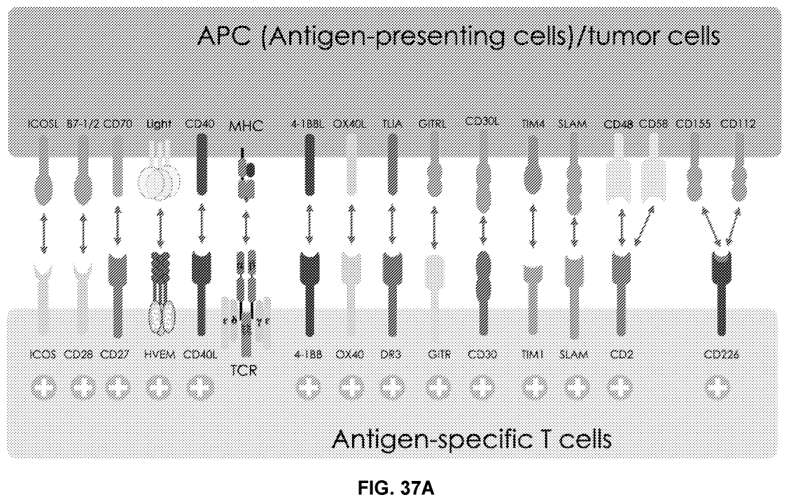

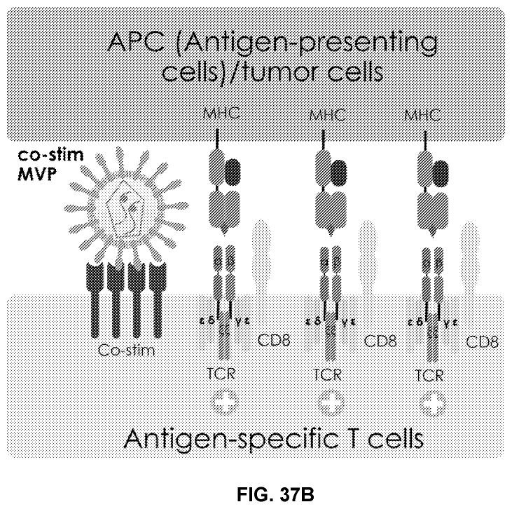

Ceacam1-MVPs were generated by pseudotyping lentiviral VLPs with trimeric Ceacam1 fusion peptides. Specifically, HEK 293T cells were co-transfected with a trimeric Ceacam1 display construct, along with a lentiviral packaging construct expressing essential packaging components, including Gag-Pol and Rev proteins, and a viral genome transfer vector encoding a GFP/luciferase reporter (FIG. 3A). The concentration of purified Ceacam1-MVPs was quantified via P24 ELISA. Ceacam1-MVPs displayed 900±500 copies of Ceacam1 per MVP in various oligomerized forms, as determined by quantitative western blot analysis (FIG. 36A). Hence, the D4 display construct (FIG. 1B) can effectively present hundreds of copies of Ceacam1 in oligomerized form on MVPs.

To confirm that Ceacam1-MVPs display functional Ceacam1, it was tested whether Ceacam1-MVPs can selectively bind to target cells expressing TIM-3, a cognate receptor of Ceacam1. First, target cell lines were established by transfecting S293 cells with a construct expressing TIM-3. Transfected cells were then stained with anti-TIM-3 antibody to differentiate TIM-3+ from TIM-3− cells. Subsequently, Ceacam1-MVPs were labeled with fluorescent dye, transfected cells were stained with labeled MVPs, and selective MVP-cell binding was analyzed via FACS (FIG. 36B). The results showed that labeled Ceacam1-MVP binding caused slightly higher fluorescence shift in TIM-3+ cells as compared to TIM-3− cells (FIG. 36B, upper panel). Moreover, this shift was around two times higher than the fluorescence shift caused by staining the same cells with control MVPs displaying non-specific ligand (FIG. 36B, lower panel). This result demonstrated that Ceacam1-MVPs displayed functional Ceacam1 and can selectively bind to TIM-3 on target cells.

This result was further validated through an alternative staining method (FIG. 36C). In this case, TIM-3-transfected cells were first incubated with unlabeled Ceacam1-MVPs to allow MVPs to bind to target cells. The cell-MVP mixture was then co-stained with fluorescently-labeled anti-TIM-3 and anti-Ceacam1 antibodies. Ceacam1 staining pattern on TIM-3+ and TIM-3− cells was then examined via FACS analysis. The results showed that TIM-3+ cells were also Ceacam1 positive, as exemplified by about two times higher Ceacam1 staining shift in TIM-3+ cells from TIM-3− background cells (FIG. 36C). Single staining with anti-TIM-3 antibody did not compete with Ceacam1-MVP binding to target cells, and TIM-3-transfected S293 cells were Ceacam1 negative. These results demonstrated that Ceacam1-MVPs displayed functional Ceacam1. Collectively, Ceacam1-MVPs were generated displaying high copy numbers of functional protein, and these MVPs can selectively bind target cells expressing their cognate receptor, TIM-3.

### Example 6. T Cell Stimulation with Activating IC-MVPs

This example illustrates a list of IC-MVPs displaying activating immune checkpoints which were generated and their compositions were characterized by determining the copy number of immune checkpoint molecules displayed on each of the VLPs. The results demonstrated specific binding of these IC-MVPs to target cells expressing cognate ligands or receptors and their co-stimulatory function in T cell activation, proliferation, and differentiation. The list of IC-MVPs illustrated in this example including, CD80-MVP, CD86-MVP, 41BBL-MVP, and OX40L-MVP.

### Use of Activating IC-MVPs to Provide Co-Stimulatory Signals for T Cells

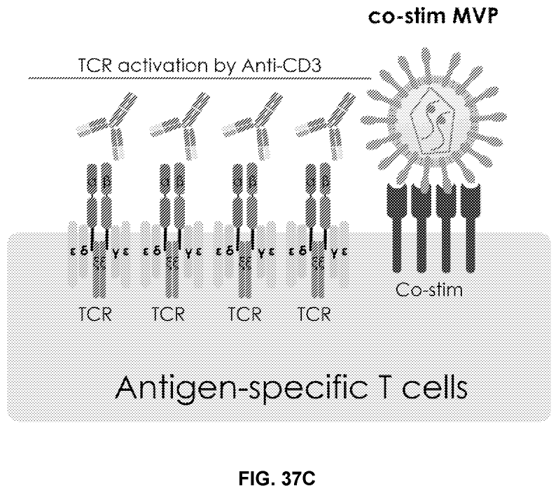

During T cell activation, two stimuli are usually required to fully activate the immune response. The first signal is antigen-specific, which is provided through T cell receptor (TCR) interactions with peptide-MHC molecules on the membrane of antigen presenting cells (APC). The second signal is non-antigen specific and is provided through the interaction of co-stimulatory molecules expressed on the membranes of the APC and T cell (FIG. 37A). Both helper T cells and cytotoxic T cells require these secondary co-stimulatory signals to become fully activated and programmed to function and differentiate. Interruption of the co-stimulatory pathway inhibits T cell immune responses in vitro and in vivo. In the case of helper T cells, the first co-stimulatory signal is provided by CD28. This molecule expressed on T cells binds to one of two molecules on the APC: B7.1 (CD80) or B7.2 (CD86) to initiate T-cell proliferation. Cytotoxic T cells rely less on CD28 for activation but still require signals from other co-stimulatory molecules such as OX-40 and 4-1BB (CD137). T cells activated by different co-stimulatory molecules may lead to distinct proliferation capabilities and fates during differentiation. Notably, multivalent engagement is critical for TCR engagement with peptide:MHC peptide complexes and the engagement of co-stimulatory molecules on T cells and APCs. Activating IC-MVPs may be used to provide multivalent co-stimulatory signals for T cells and help APCs including cancer cells to properly activate tumor-targeting T cells in culture or in animals (FIG. 37B). Furthermore, activating IC-MVPs may serve as co-stimulatory signals during T cell activation in vitro with anti-CD3 antibody (FIG. 37C). Various combination of co-stimulatory IC-MVPs may be used to program T cell activation and differentiation to generate therapeutic T cells with desirable functional properties. This example illustrates the function of CD80-MVP, CD86-MVP, 4-1BB-MVP, and OX40-L-MVP in T cell activation, proliferation, and differentiation.

### CD86-MVPs as Co-Stimulatory Signals for T Cells

CD86-MVPs were produced displaying either mouse or human CD86 to test their function in T cell activation, proliferation, and differentiation. CD86 provides costimulatory signals for T cell activation and survival. CD86 also belongs to the B7 family of immunoglobulin superfamily. Both CD80 and CD86 bind as ligands to costimulatory molecule CD28 on the surface of all naïve T cells and to the inhibitory receptor cytotoxic T-lymphocyte antigen-4 (CTLA4). The interaction between CD86 expressed on the surface of an antigen-presenting cell with CD28 on the surface of T cell is important for T cell activation. This interaction is essential for T lymphocytes to receive the full activation signal, which in turn leads to T cell differentiation and division, production of interleukin 2 and cell expansion.

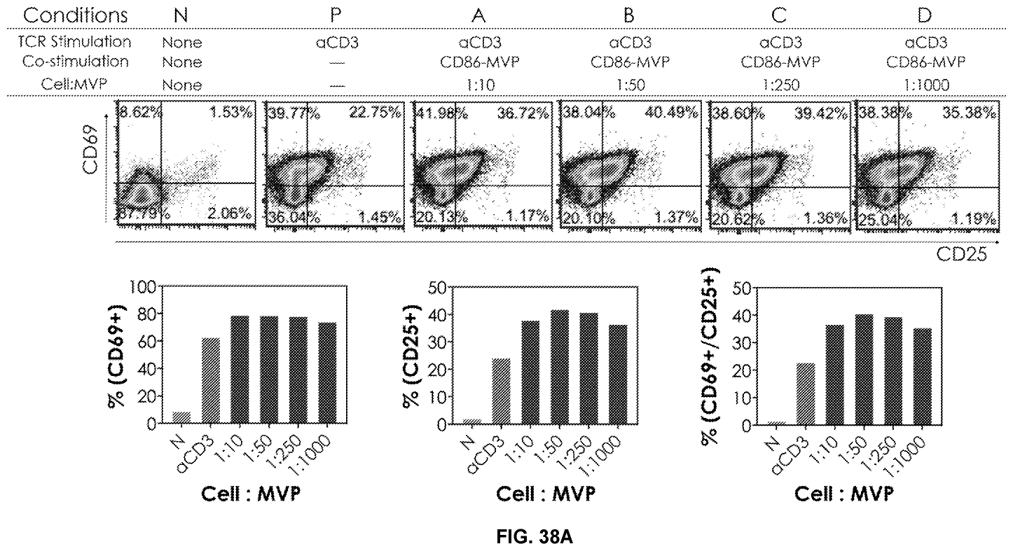

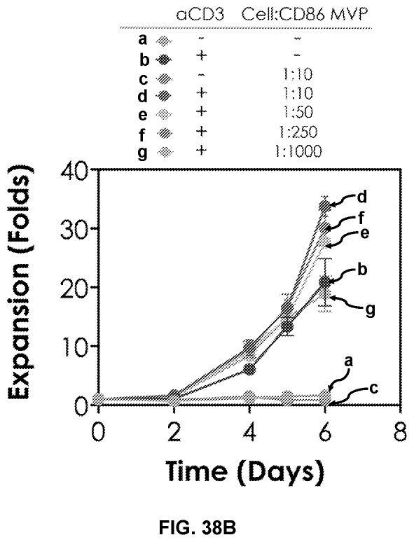

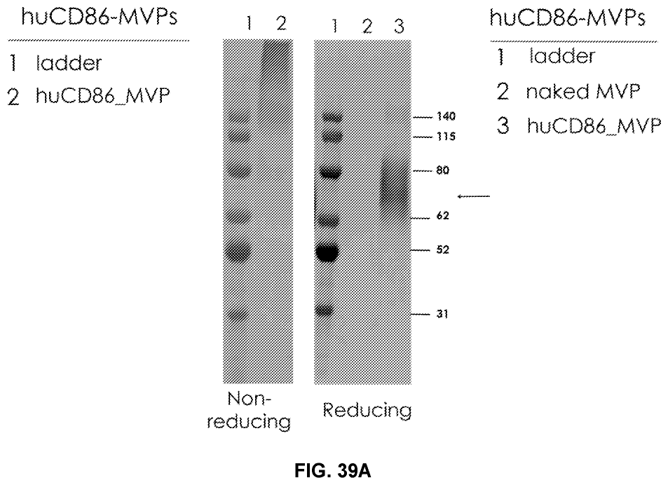

To this end, mouse spleen T cells were activated with plate coated with anti-CD3 antibody to provide TCR activation signals and murine CD86-MVPs were supplemented as co-stimulatory signals at varied cell to CD86-MVP ratios (FIG. 38A). At day-2 post T cell activation, FACS analyses were carried out to determine the expression of early T cell activation markers CD69 and CD25 on the activated T cells. The results showed that addition of murine CD86-MVPs further increased the proportion of T cells with CD69+CD25+ phenotype from ˜22.75% to over 40%, demonstrating that CD86-MVPs provided co-stimulatory signals and boosted T cell activation (FIG. 38A). Notably, optimal cell to CD86-MVP was at 1:50 and further increase of this ratio would reduce the amount of T cells with CD69+CD25+ phenotype (FIG. 38A). Furthermore, enhanced T cell activation with the addition of murine CD86-MVPs was translated into increased T cell proliferation as indicated by the fold of expansion (FIG. 38B). In the control group of T cells that were activated with only primary or secondary co-stimulatory signals, it was not enough to induce full T cell activation and proliferation (FIG. 38B).

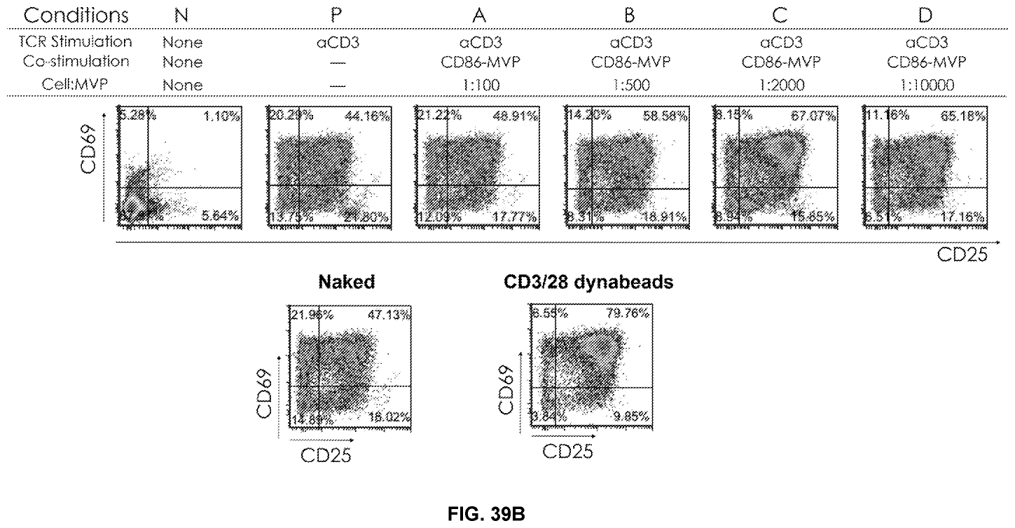

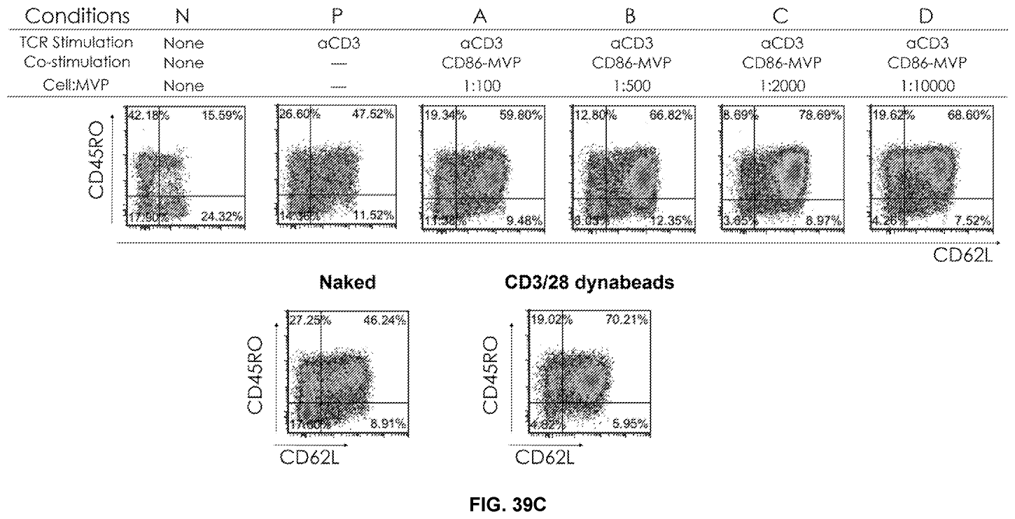

It was further examined whether human CD86-MVPs had similar effects on T cell activation, proliferation, and differentiation. Human CD86-MVPs were generated by pseudotyping lentiviral VLPs with trimeric CD86 fusion peptides. Specifically, HEK 293T cells were co-transfected with a trimeric CD86 display construct, along with a lentiviral packaging construct expressing essential packaging components, including Gag-Pol and Rev proteins, and a viral genome transfer vector encoding a GFP/luciferase reporter (FIG. 3A). Western-blot analyses demonstrated the expression of CD86 on purified human CD86-MVPs and high molecular weight oligomers were observed under non-reducing conditions (FIG. 39A). Hence, the D4 display construct (FIG. 1B) can effectively present many copies of CD86 in oligomerized form on MVPs. Human peripheral blood T cells were activated with plate coated anti-human CD3 antibody to provide TCR activation signals and human CD86-MVPs were supplemented as co-stimulatory signals at varied cell to CD86-MVP ratios (FIG. 39B). At day-2 post T cell activation, FACS analyses were carried out to determine the expression of early T cell activation markers CD69 and CD25 on the activated T cells. The results showed that addition of human CD86-MVPs further increased proportion of T cells with CD69+CD25+ phenotype from ˜44% to over 67% (FIG. 39B), demonstrating that CD86-MVPs provided co-stimulatory signals and boosted T cell activation. Further, the effect of co-stimulatory MVPs on T cell differentiation from naïve T cells (CD62L+CD45RO−) into central memory (CD62L+CD45RO+) and effector memory (CD62L-CD45RO−) T cells was analyzed by FACS (FIG. 39C). At day-8 post activation, cells were analyzed by FACS to determine the effects of CD86-MVPs on differentiation status based on the expression of CD45RO and CD62L markers. The results showed that addition of human CD86-MVPs to T cell activation also boosted the percent of T cells produced with CD62L+CD45RO+ central memory phenotype (FIG. 39C) from ˜47% to 78% (FIG. 39C). Collectively, these results demonstrated that CD86-MVPs provided important co-stimulatory signals for T cell activation, proliferation, and differentiation

### CD80-MVPs as Co-Stimulatory Signals for T Cells

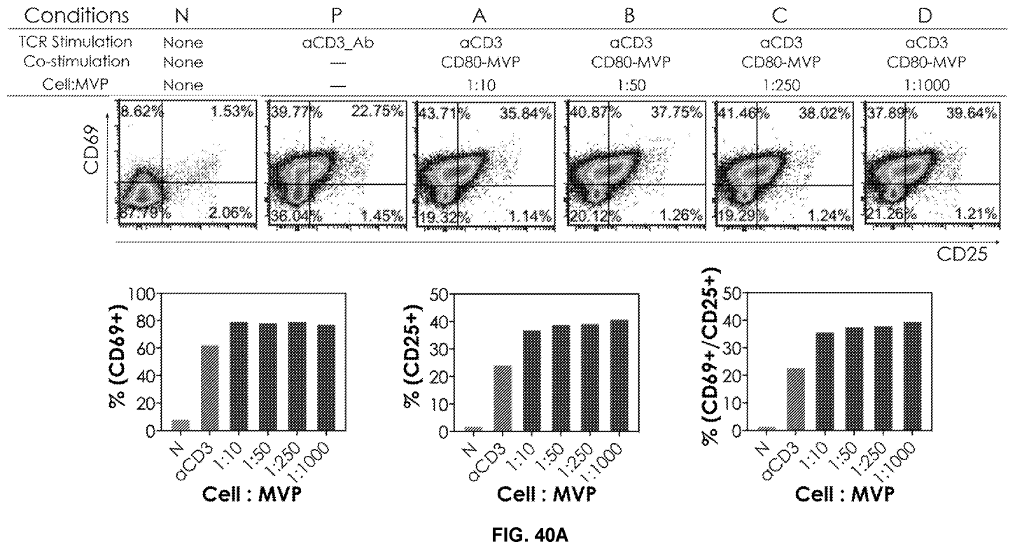

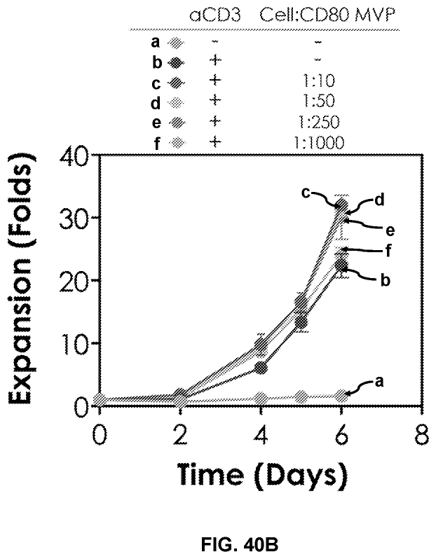

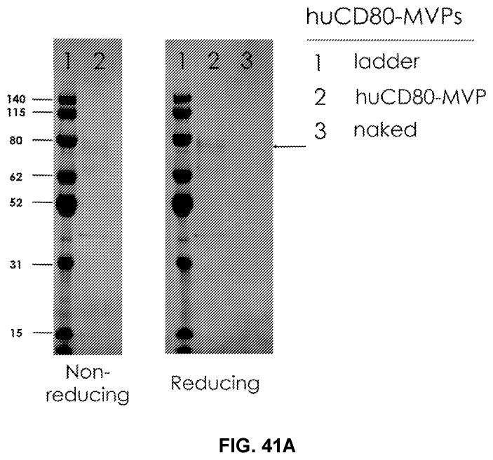

CD80-MVPs were produced displaying either mouse or human CD80 to test their function in T cell activation, proliferation, and differentiation. Mouse spleen T cells were activated with plate coated anti-CD3 antibody to provide TCR activation signals and murine CD80-MVPs were supplemented as co-stimulatory signals at varied cell to murine CD80-MVP ratios (FIG. 40A). At day-2 post T cell activation, FACS analyses were carried out to determine the expression of early T cell activation markers CD69 and CD25 on the activated T cells. The results showed that addition of murine CD80-MVPs further increased proportion of T cells with CD69+CD25+ phenotype from ˜22.75% to over 39% (FIG. 40A), demonstrating that murine CD80-MVPs provided co-stimulatory signals and boosted T cell activation. Notably, optimal cell to murine CD86-MVP was at 1:1000 and further increase of this ratio would reduce the amount of T cells with CD69+CD25+ phenotype (FIG. 40A). Furthermore, enhanced T cell activation with the addition of murine CD86-MVPs was translated into increased T cell proliferation as indicated by the fold of expansion (FIG. 40B). The control T cells activated with only primary or secondary co-stimulatory signals were not enough to induce full T cell activation and proliferation (FIG. 40B).

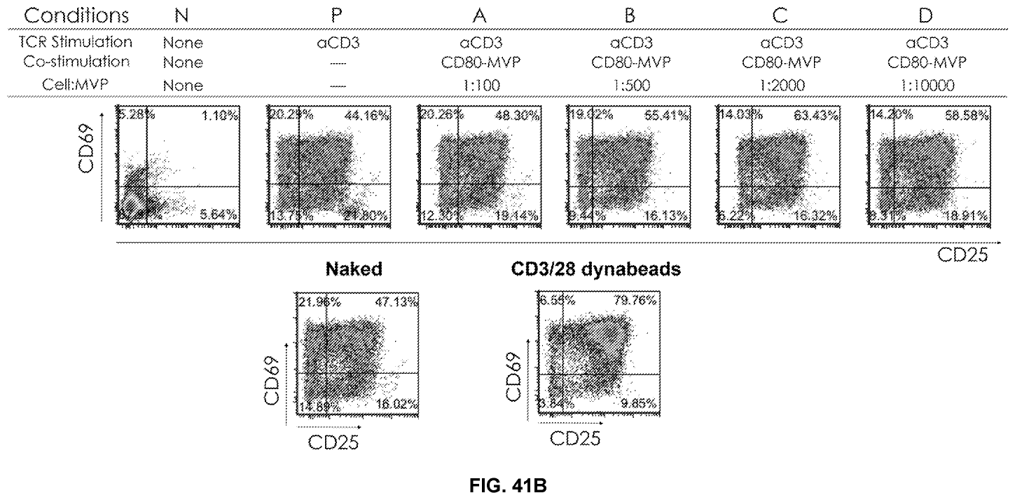

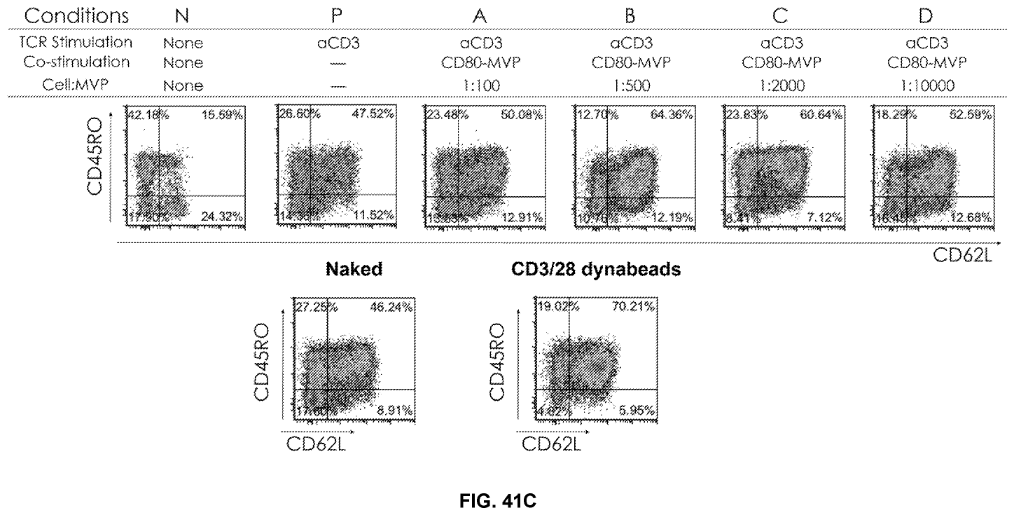

It was examined whether human CD80-MVPs had similar function in T cell activation, proliferation, and differentiation. Human CD80-MVPs were generated by pseudotyping lentiviral VLPs with trimeric CD80 fusion peptides. Specifically, HEK 293T cells were co-transfected with a trimeric CD80 display construct, along with a lentiviral packaging construct expressing essential packaging components, including Gag-Pol and Rev proteins, and a viral genome transfer vector encoding a GFP/luciferase reporter (FIG. 3A). Western-blot analyses demonstrated the expression of CD80 on purified CD80-MVPs (FIG. 41A). Hence, the D4 display construct (FIG. 1B) can effectively present many copies of CD80 in oligomerized form on MVPs. Human peripheral blood T cells were then activated with plate coated anti-human CD3 antibody to provide TCR activation signals and human CD80-MVPs were supplemented as co-stimulatory signals at varied cell to CD86-MVP ratios (FIG. 41B). At day-2 post T cell activation, FACS analyses were carried out to determine the expression of early T cell activation markers CD69 and CD25 on the activated T cells. The results showed that addition of human CD86-MVPs further increased proportion of T cells with CD69+CD25+ phenotype from ˜44% to over 63% (FIG. 41B), demonstrating that CD80-MVPs provided co-stimulatory signals and boosted human T cell activation. Further, the effect of co-stimulatory MVPs on T cell differentiation from naïve T cells (CD62L+CD45RO−) into central memory (CD62L+CD45RO+) and effector memory (CD62L-CD45RO−) T cells were analyzed by FACS. At day-8 post activation, cells were analyzed by FACS to determine the effects of CD80-MVPs on differentiation status based on the expression of CD45RO and CD62L markers. The results showed that addition of human CD80-MVPs to T cell activation also boosted the percent of T cells produced with CD62L+CD45RO+ central memory phenotype (FIG. 41C) from 47% to 60%. Collectively, these results demonstrated that CD80-MVPs provided co-stimulatory signals for T cell activation, proliferation, and differentiation

### 4-1BBL-MVPs as Co-Stimulatory Signals for T Cells

4-1BBL-MVPs were produced displaying either mouse or human 4-1BB ligand using the type II display vector (FIG. 1C) and their function was tested in T cell activation, proliferation, and differentiation. 4-1BBL is a type 2 transmembrane glycoprotein receptor that is found on APCs and binds to 4-1BB (also known as CD137) on T cells. The interaction between 4-1BB and 4-1BBL provides costimulatory signals to a variety of T cells, which have been shown to have anti-tumor effects in some model systems. While CD28 contributes to initial T cell expansion, 4-1BBL may contribute more to memory CD8 T cell survival. Co-stimulation using 4-1BB ligand (4-1BBL) or agonistic anti-4-1BB antibodies could prolong T-cell responses and avoid activation-induced cell death for cancer immunotherapy.

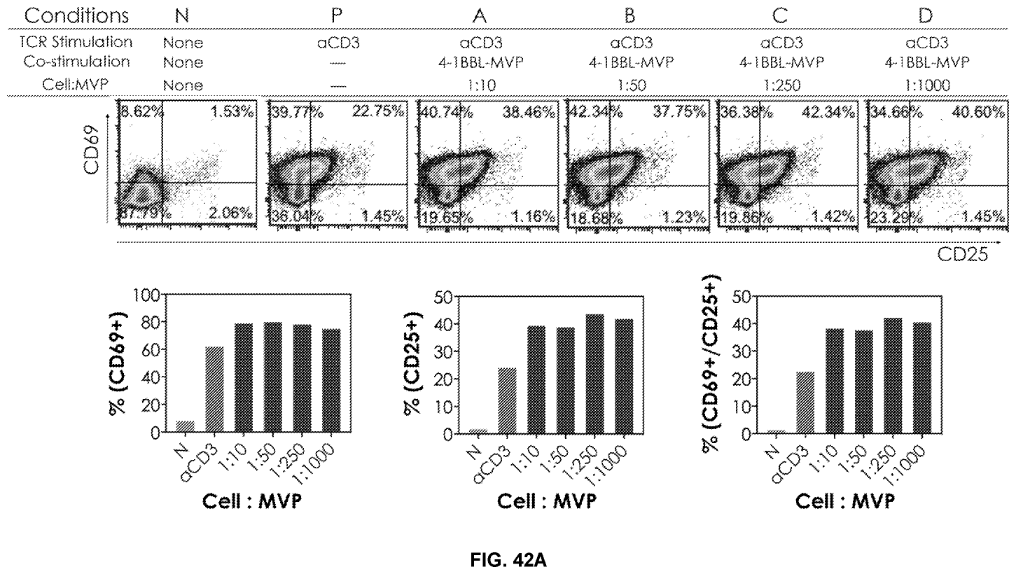

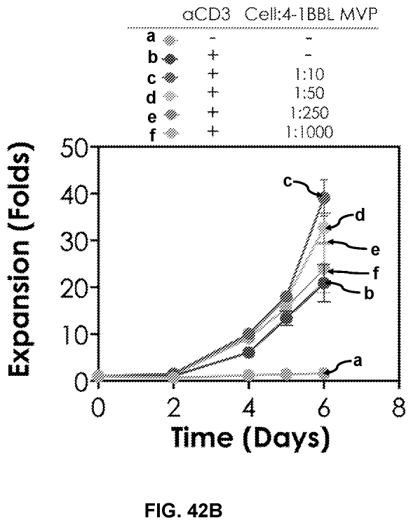

To this end, mouse spleen T cells were activated with plate coated anti-CD3 antibody to provide TCR activation signals and murine 4-1BBL-MVPs were supplemented as co-stimulatory signals at varied cell to 4-1BBL-MVP ratios (FIG. 42A). At day-2 post T cell activation, FACS analyses were carried out to determine the expression of early T cell activation markers CD69 and CD25 on the activated T cells. The results showed that addition of murine 4-1BBL-MVPs further increased proportion of T cells with CD69+CD25+ phenotype from ˜22.75% to over 42% (FIG. 42A), demonstrating that 4-1BBL-MVPs provided co-stimulatory signals and boosted T cell activation. Notably, optimal cell to 4-1BBL-MVPs ratio was at 1:250 and further increase of this ratio would reduce the amount of T cells with CD69+CD25+ phenotype. Furthermore, enhanced T cell activation with the addition of murine 4-1BB-MVPs was translated into increased T cell proliferation as indicated by the fold of expansion (FIG. 42B). Control T cells were activated with only primary or secondary co-stimulatory signals which was not enough to induce full T cell activation and proliferation.

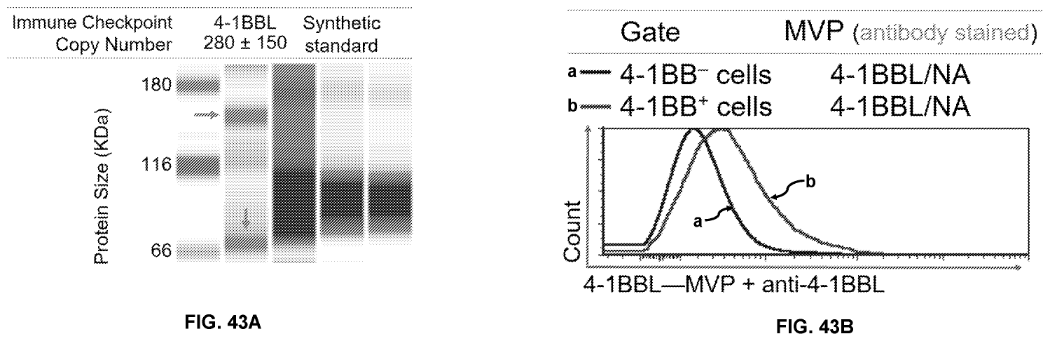

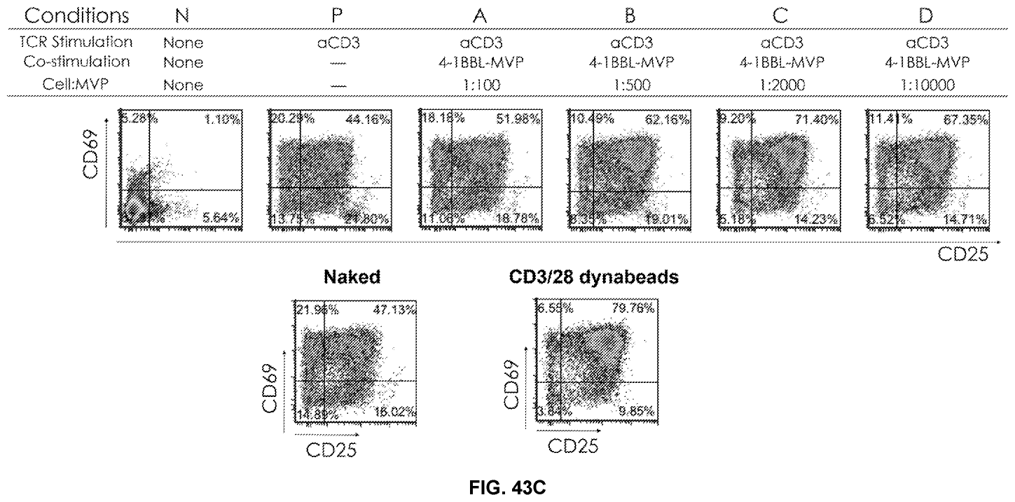

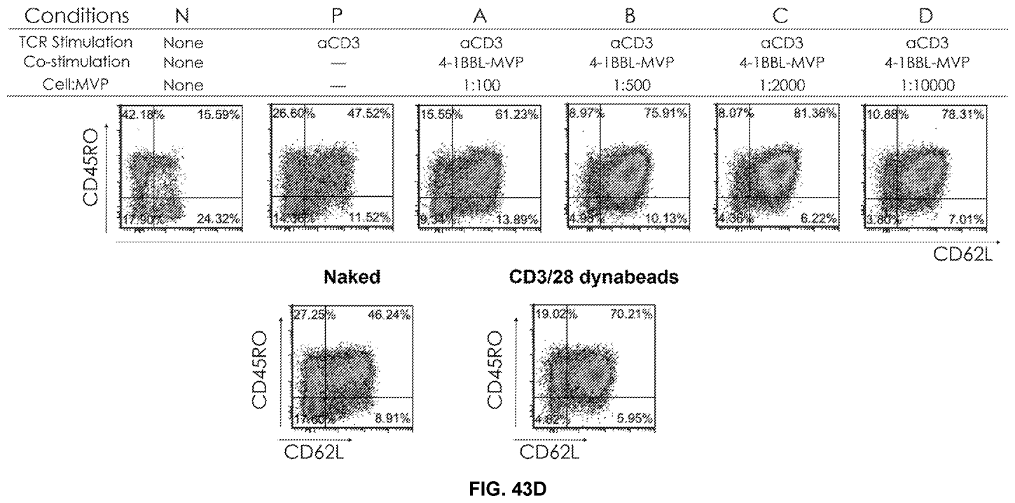

It was examined whether human 4-1BBL-MVPs have similar function in T cell activation, proliferation, and differentiation. Human 4-1BBL-MVPs were generated by pseudotyping lentiviral VLPs with trimeric 4-1BB ligand fusion peptides. Specifically, HEK 293T cells were co-transfected with a trimeric 4-1BB ligand display construct, along with a lentiviral packaging construct expressing essential packaging components, including Gag-Pol and Rev proteins, and a viral genome transfer vector encoding a GFP/luciferase reporter (FIG. 3A). Quantitative Western-blot analyses showed that 4-1BBL-MVPs displayed 280±150 copies of 4-1BB ligand per particle in various oligomerized forms (FIG. 43A). Hence, the D4 display construct (FIG. 1B) can effectively present hundreds of copies of 4-1BB ligand in oligomerized form on MVPs.

To confirm that human 41BBL-MVPs display functional 4-1BB ligand, 4-1BB-transfected cells were first incubated with unlabeled 4-1BBL-MVPs to allow MVPs to bind to target cells. The cell-MVP mixture was then co-stained with fluorescently-labeled anti-4-1BB and anti-4-1BB ligand antibodies. 4-1BB ligand staining pattern on 41BB+ and 41BB− cells was then examined via FACS analysis. The results showed that 4-1BB+ cells were also 4-1BB ligand positive (FIG. 43B). Single staining with anti-4-1BB antibody did not compete with 41BBL-MVP binding to target cells, and 41BB-transfected S293 cells were 4-1BB ligand negative. These results demonstrated that 4-1BBL-MVPs displayed functional 4-1BB ligand. Collectively, 41BBL-MVPs were generated displaying high copy numbers of functional protein, and these MVPs can selectively bind target cells expressing their cognate receptor, 4-1BB.

Human peripheral blood T cells were then activated with plate coated with anti-human CD3 antibody to provide TCR activation signals and human 4-1BBL-MVPs were supplemented as co-stimulatory signals at varied cell to 4-1BBL-MVP ratios (FIG. 43B). At day-2 post T cell activation, FACS analyses were carried out to determine the expression of early T cell activation markers CD69 and CD25 on the activated T cells. The results showed that addition of human 4-1BBL-MVPs further increased proportion of T cells with CD69+CD25+ phenotype from ˜44% to over 71% (FIG. 43C), demonstrating that human 4-1BBL-MVPs provided co-stimulatory signals and boosted T cell activation. Further, the effect of co-stimulatory MVPs on T cell differentiation from naïve T cells (CD62L+CD45RO−) into central memory (CD62L+CD45RO+) and effector memory (CD62L-CD45RO−) T cells was analyzed by FACS. At day-8 post activation, cells were analyzed by FACS to determine the effects of human 4-1BBL-MVPs on differentiation status based on the expression of CD45RO and CD62L markers (FIG. 43D). The results showed that addition of human 4-1BBL-MVPs to T cell activation also boosted the percent of T cells produced with CD62L+CD45RO+ central memory phenotype (FIG. 43D) from ˜47% to ˜81%. Collectively, these results demonstrated that 4-1BBL-MVPs provided co-stimulatory signals for T cell activation, proliferation, and differentiation

### OX40L-MVPs as Co-Stimulatory Signals for T Cells

OX40L-MVPs were produced displaying either mouse or human OX40 ligand using the D4 trimeric display vector to test their function in T cell activation, proliferation, and differentiation. OX40L is the ligand for OX40 (also known as CD134 or TNFRSF4) and is expressed on many antigen-presenting cells such as DC2s (a subtype of dendritic cells), macrophages, and activated B lymphocytes. Costimulatory signals from OX40 to a conventional T cell promote division and survival, augmenting expansion of effector and memory populations. OX40L, when co-expressed with 4-1BBL, could provide a synergistic costimulatory signal to an antigen reacting naïve CD4 T cell to prolong T cell proliferation, as well as increase production of several cytokines.

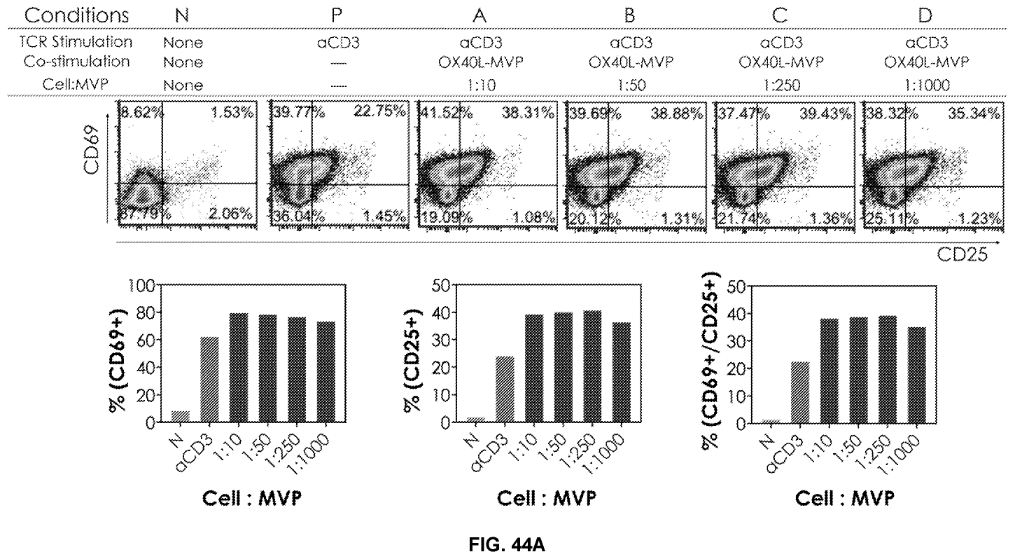

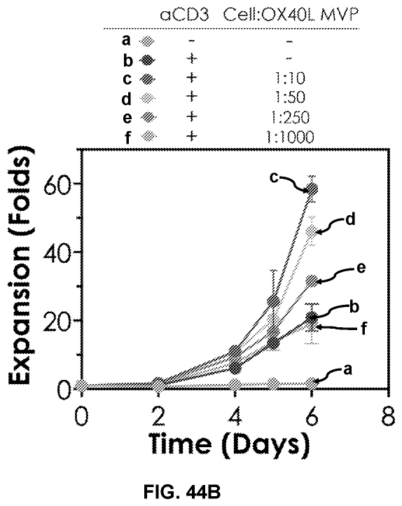

To this end, mouse spleen T cells were activated with plate coated anti-CD3 antibody to provide TCR activation signals and mouse OX40L-MVPs were supplemented as co-stimulatory signals at varied cell to OX40L-MVP ratios (FIG. 44A). At day-2 post T cell activation, FACS analyses were carried out to determine the expression of early T cell activation markers CD69 and CD25 on the activated T cells. The results showed that addition of OX40L-MVPs further increased proportion of T cells with CD69+CD25+ phenotype from ˜22.75% to over 39% (FIG. 44A), demonstrating that mouse OX40L-MVPs provided co-stimulatory signals and boosted T cell activation. Notably, optimal cell to OX40L-MVP was at 1:250 and further increase of this ratio would reduce the amount of T cells with CD69+CD25+ phenotype. Furthermore, enhanced T cell activation with the addition of mouse OX40L-MVPs was translated into increased T cell proliferation as indicated by the fold of expansion (FIG. 44B). The control T cells were activated with only primary or secondary co-stimulatory signals which was not enough to induce full T cell activation and proliferation.

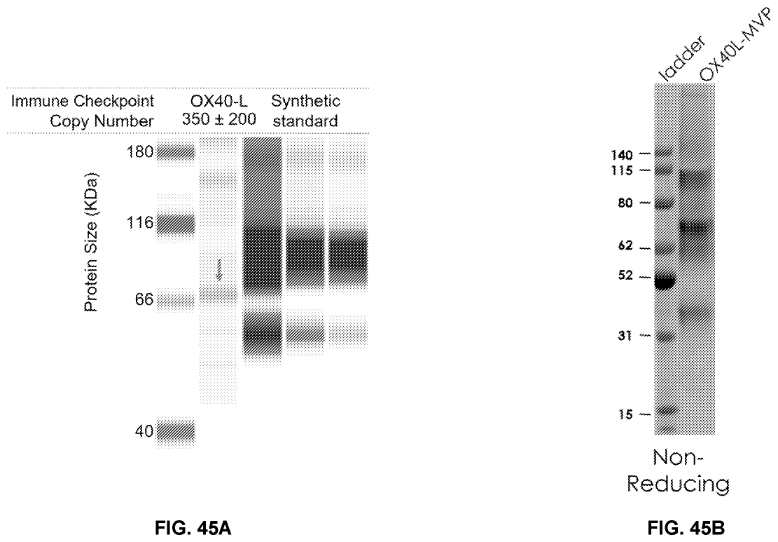

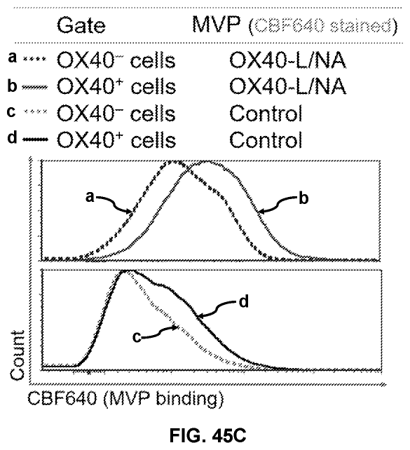

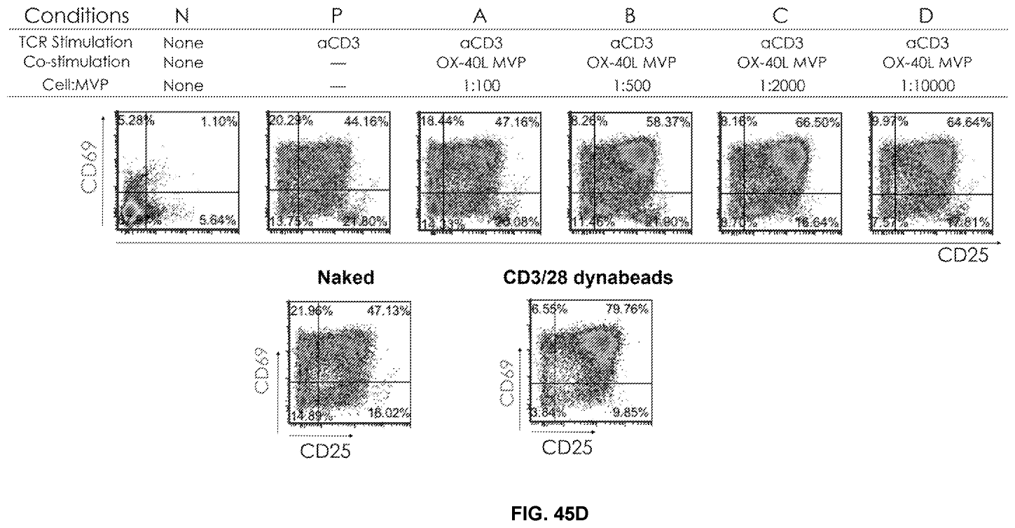

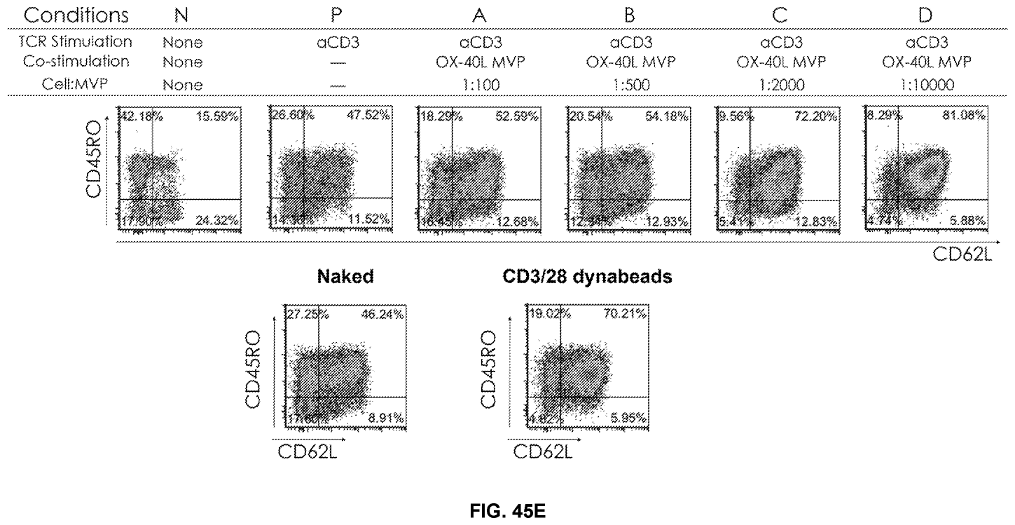

It was examined whether human OX40L-MVPs have similar function in T cell activation, proliferation, and differentiation. Human OX40L-MVPs were generated by pseudotyping lentiviral VLPs with trimeric OX40 ligand fusion peptides. Specifically, HEK 293T cells were co-transfected with a trimeric OX40 ligand display construct, along with a lentiviral packaging construct expressing essential packaging components, including Gag-Pol and Rev proteins, and a viral genome transfer vector encoding a GFP/luciferase reporter (FIG. 3A). Quantitative Western-blot analyses showed that human OX40L-MVPs displayed 350±20 copies of OX40 ligand per particle in various oligomerized forms (FIG. 45A), and western-blot analyses of human OX40L-MVPs in a non-reducing condition showed consistent results (FIG. 45B). Hence, the D4 display construct (FIG. 1B) can effectively present hundreds of copies of OX40 ligand in oligomerized form on MVPs.

To confirm that human OX40L-MVPs display functional OX40 ligand, it was tested whether human OX40L-MVPs can selectively bind to target cells expressing OX40, its cognate receptor. First, target cell lines were established by transfecting S293 cells with a construct expressing OX40. Transfected cells were then stained with anti-OX40 antibody to differentiate OX40+ from OX40− cells. Subsequently, human OX40L-MVPs were labeled with fluorescent dye, transfected cells were stained with labeled MVPs, and selective MVP-cell binding was analyzed via FACS (FIG. 45C). The results showed that labeled human OX40L-MVP binding caused significantly higher fluorescence shift in OX40+ cells as compared to OX40− cells (FIG. 45C, upper panel). Moreover, this shift was higher than the fluorescence shift caused by staining the same cells with control MVPs displaying non-specific ligand (FIG. 45C, lower panel). This result demonstrated that human OX40L-MVPs displayed functional OX40L and can selectively bind to OX40 on target cells.

Human peripheral blood T cells were then activated with plate coated anti-human CD3 antibody to provide TCR activation signals and human OX40L-MVPs were supplemented as co-stimulatory signals at varied cell to OX40L-MVP ratios (FIG. 45D). At day-3 post T cell activation, FACS analyses were carried out to determine the expression of early T cell activation markers CD69 and CD25 on the activated T cells. The results showed that addition of human OX40L-MVPs further increased the proportion of T cells with CD69+CD25+ phenotype from ˜44% to over 66%, demonstrating that OX40L-MVPs provided co-stimulatory signals and boosted T cell activation. Further, the effect of co-stimulatory MVPs on T cell differentiation from naïve T cells (CD62L+CD45RO−) into central memory (CD62L+CD45RO+) and effector memory (CD62L-CD45RO−) T cells was analyzed by FACS. The results showed that addition of human OX40L-MVPs to T cell activation also boosted the percent of T cells produced with CD62L+CD45RO+ central memory phenotype from ˜47% to 81% (FIG. 45E). Collectively, these results demonstrated that OX40L-MVPs provided co-stimulatory signals for T cell activation, proliferation, and differentiation

### Example 7. Characterization of IC-MVPs Displaying Activating Immune Checkpoints

This example illustrates a list of IC-MVPs displaying activating immune checkpoints and characterization of their compositions by determining the copies of immune checkpoint molecules displayed on each of the VLPs. This example also demonstrates their specific binding to target cells expressing cognate ligands or receptors. The list of IC-MVPs exemplified in this example includes: LIGHT-MVP, CD30-MVP, CD30L-MVP, CD48-MVP, CD2-MVP, CD27-MVP, CD70-MVP, ICOS-MVP, ICOSL-MVP, GITR-MVP, GITRL-MVP, 4-1BB-MVP, and OX40-MVP.

### LIGHT-MVP Composition and Selective Binding to Target Cells Expressing HVEM

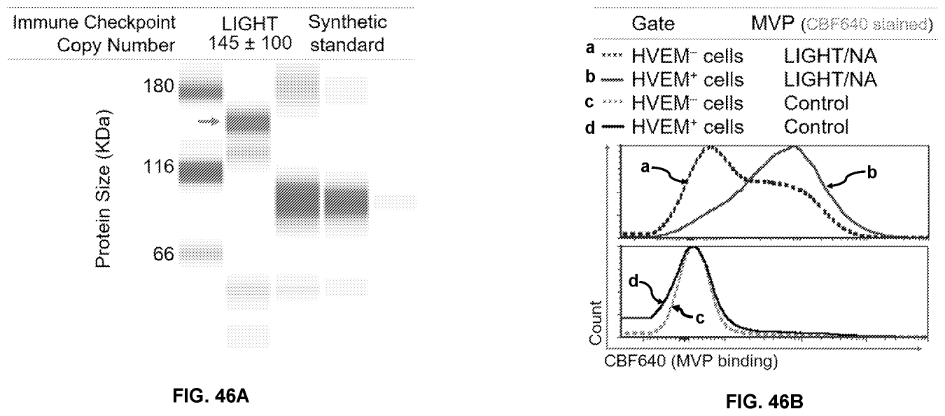

LIGHT-MVPs were generated by pseudotyping lentiviral VLPs with trimeric LIGHT fusion peptides. Specifically, HEK 293T cells were co-transfected with a trimeric LIGHT display construct, along with a lentiviral packaging construct expressing essential packaging components, including Gag-Pol and Rev proteins, and a viral genome transfer vector encoding a GFP/luciferase reporter (FIG. 3A). The concentration of purified LIGHT-MVPs was quantified via P24 ELISA. LIGHT-MVPs displayed 145±100 copies of LIGHT per MVP in oligomerized forms, as determined by quantitative western blot analysis (FIG. 46A). Hence, the D4 display construct (FIG. 1B) can effectively present thousands of copies of LIGHT in oligomerized form on MVPs.

To confirm that LIGHT-MVPs display functional LIGHT, it was tested whether LIGHT-MVPs can selectively bind to target cells expressing HVEM, a cognate receptor of LIGHT. First, target cell lines were established by transfecting S293 cells with a construct expressing HVEM. Transfected cells were then stained with anti-HVEM antibody to differentiate HVEM+ from HVEM− cells. Subsequently, LIGHT-MVPs were labeled with fluorescent dye, transfected cells were stained with labeled MVPs, and selective MVP-cell binding was analyzed via FACS (FIG. 46B). The results showed that labeled LIGHT-MVPs binding caused significantly higher fluorescence shift in HVEM+ cells as compared to HVEM− cells (FIG. 46B, upper panel). Moreover, this shift was at least one log higher than the fluorescence shift caused by staining the same cells with control MVPs displaying non-specific ligand (FIG. 46B, lower panel). This result demonstrated that LIGHT-MVPs displayed functional LIGHT and can selectively bind to target cells expressing HVEM.

### CD30-MVP Composition and Selective Binding to Target Cells Expressing CD30L

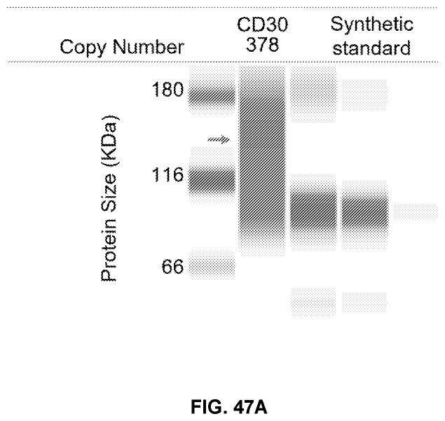

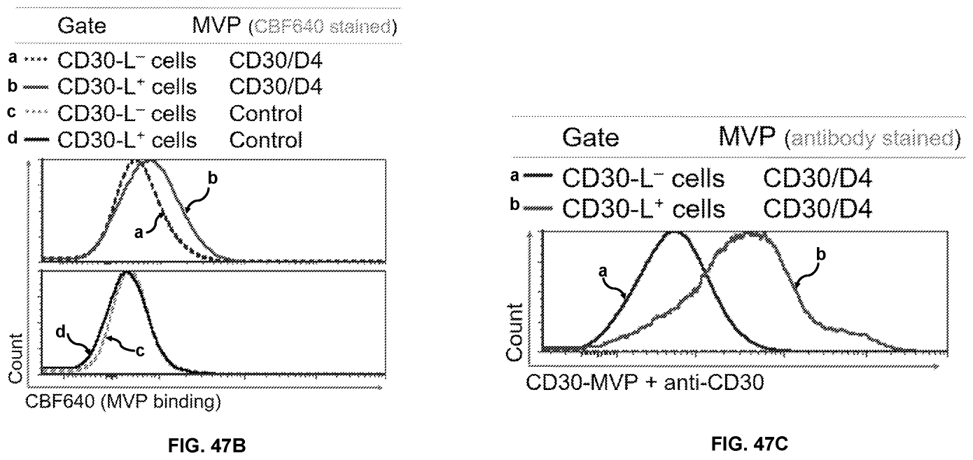

CD30-MVPs were generated by pseudotyping lentiviral VLPs with trimeric CD30 fusion peptides. Specifically, HEK 293T cells were co-transfected with a trimeric CD30 display construct, along with a lentiviral packaging construct expressing essential packaging components, including Gag-Pol and Rev proteins, and a viral genome transfer vector encoding a GFP/luciferase reporter (FIG. 3A). The concentration of purified CD30-MVPs was quantified via P24 ELISA. CD30-MVPs displayed 378 copies of CD30 per MVP in various oligomerized forms, as determined by quantitative western blot analysis (FIG. 47A).

To confirm that CD30-MVPs display functional CD30, it was tested whether CD30-MVPs can selectively bind to target cells expressing CD30 ligand, a cognate ligand of CD30. First, target cell lines were established by transfecting S293 cells with a construct expressing CD30 ligand (CD30L). Transfected cells were then stained with anti-CD30L antibody to differentiate CD30L+ from CD30L− cells. Subsequently, CD30-MVPs were labeled with fluorescent dye, transfected cells were stained with labeled MVPs, and selective MVP-cell binding was analyzed via FACS (FIG. 47B). The results showed that labeled CD30-MVP binding caused significantly higher fluorescence shift in CD30L+ cells as compared to CD30L− cells (FIG. 47B, upper panel). Moreover, this shift was significantly higher than the fluorescence shift caused by staining the same cells with control MVPs displaying non-specific ligand (FIG. 47B, lower panel). This result demonstrated that CD30-MVPs displayed functional CD30 and can selectively bind to CD30 ligand on target cells.

This result was further validated through an alternative staining method (FIG. 47C). In this case, CD30 ligand-transfected cells were first incubated with unlabeled CD30-MVPs to allow MVPs to bind to target cells. The cell-MVP mixture was then co-stained with fluorescently-labeled anti-CD30 and anti-30L antibodies. CD30 staining pattern on CD30L+ and CD30L− cells was then examined via FACS analysis. The results showed that CD30L+ cells were also CD30 positive, as exemplified by more than 1-log CD30 staining shift in CD30L+ cells from CD30L− background cells (FIG. 47C). Single staining with anti-CD30 antibody did not compete with CD30-MVP binding to target cells, and CD30L-transfected S293 cells were CD30 negative. These results demonstrated that CD30-MVPs displayed functional CD30. Collectively, CD30-MVPs were generated displaying high copy numbers of functional protein, and these MVPs can selectively bind target cells expressing their cognate receptor, CD30 ligand.

### CD30L-MVP Composition and Selective Binding to Target Cells Expressing CD30

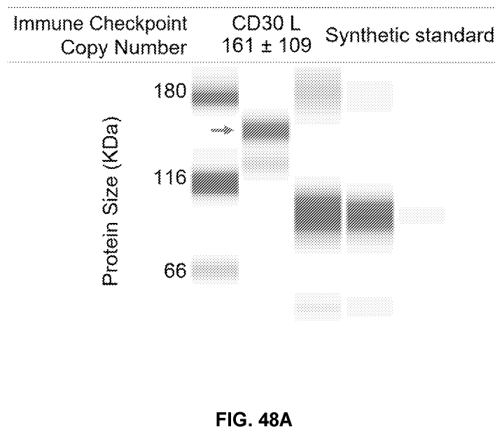

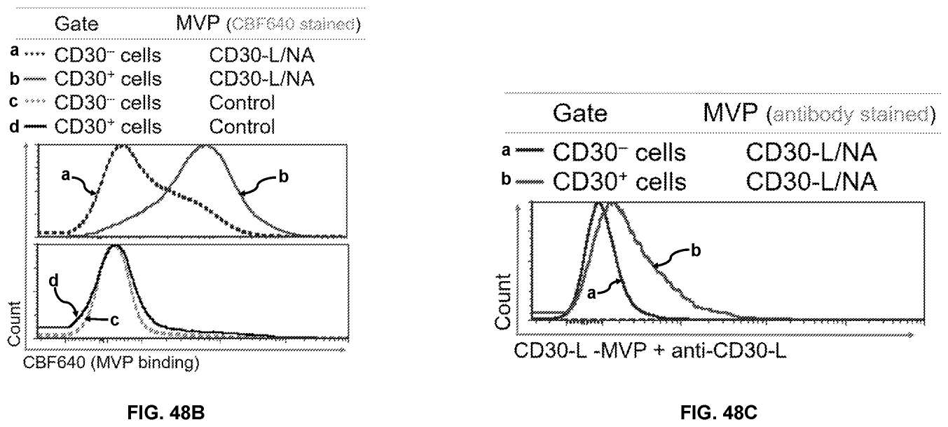

CD30L-MVPs were generated by pseudotyping lentiviral VLPs with trimeric CD30 ligand fusion peptides. Specifically, HEK 293T cells were co-transfected with a trimeric CD30 ligand display construct, along with a lentiviral packaging construct expressing essential packaging components, including Gag-Pol and Rev proteins, and a viral genome transfer vector encoding a GFP/luciferase reporter (FIG. 3A). The concentration of purified CD30L-MVPs was quantified via P24 ELISA. CD30L-MVPs displayed 161±109 copies of CD30 per MVP in various oligomerized forms, as determined by quantitative western blot analysis (FIG. 48A).

To confirm that CD30L-MVPs display functional CD30L, it was tested whether CD30L-MVPs can selectively bind to target cells expressing CD30, a cognate receptor of CD30L. First, target cell lines were established by transfecting S293 cells with a construct expressing CD30. Transfected cells were then stained with anti-CD30 antibody to differentiate CD30+ from CD30-cells. Subsequently, CD30L-MVPs were labeled with fluorescent dye, transfected cells were stained with labeled MVPs, and selective MVP-cell binding was analyzed via FACS (FIG. 48B). The results showed that labeled CD30L-MVP binding caused significantly higher fluorescence shift in CD30+ cells as compared to CD30− cells (FIG. 48B, upper panel). Moreover, this shift was significantly higher than the fluorescence shift caused by staining the same cells with control MVPs displaying non-specific ligand (FIG. 48B, lower panel). This result demonstrated that CD30L-MVPs displayed functional CD30 ligand and can selectively bind to target cells expressing CD30.

This result was further validated through an alternative staining method (FIG. 48C). In this case, CD30-transfected cells were first incubated with unlabeled CD30L-MVPs to allow MVPs to bind to target cells. The cell-MVP mixture was then co-stained with fluorescent-labeled anti-CD30 and anti-30L antibodies. CD30L staining pattern on CD30+ and CD30− cells was then examined via FACS analysis. The results showed that CD30+ cells were also CD30L positive, as exemplified by more than 1-log CD30L staining shift in CD30+ cells from CD30− background cells (FIG. 48C). Single staining with anti-CD30 antibody did not compete with CD30L-MVP binding to target cells, and CD30-transfected S293 cells were CD30L negative. These results demonstrated that CD30L-MVPs displayed functional CD30L. Collectively, CD30L-MVPs were generated displaying high copy numbers of functional protein, and these MVPs can selectively bind target cells expressing their cognate receptor, CD30.

### CD48-MVP Composition and Selective Binding to Target Cells Expressing CD2

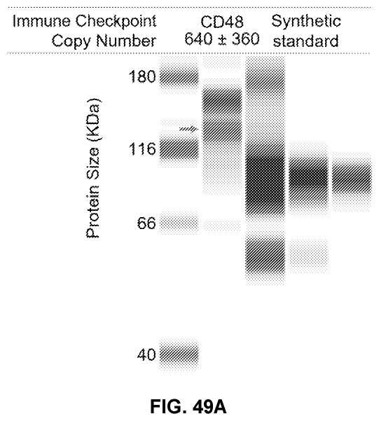

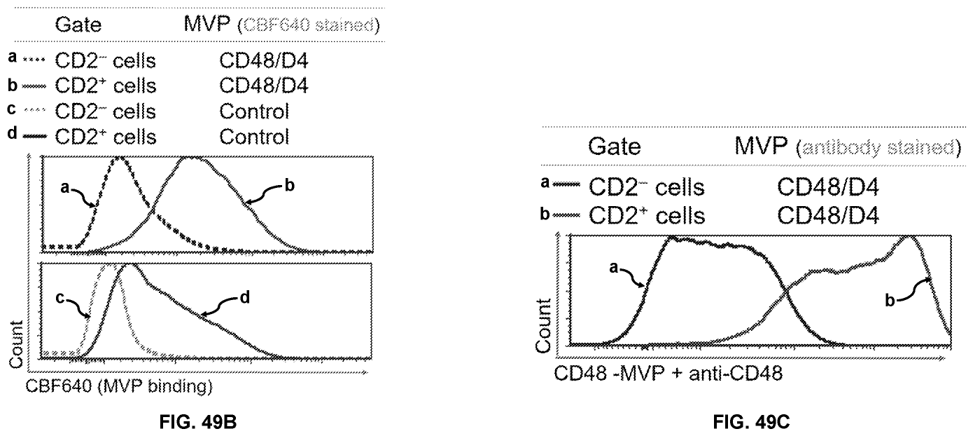

CD48-MVPs were generated by pseudotyping lentiviral VLPs with trimeric CD48 fusion peptides. Specifically, HEK 293T cells were co-transfected with a trimeric CD48 display construct, along with a lentiviral packaging construct expressing essential packaging components, including Gag-Pol and Rev proteins, and a viral genome transfer vector encoding a GFP/luciferase reporter (FIG. 3A). The concentration of purified CD48-MVPs was quantified via P24 ELISA. CD48-MVPs displayed 640±360 copies of CD48 per MVP in various oligomerized forms, as determined by quantitative western blot analysis (FIG. 49A).

To confirm that CD48-MVPs display functional CD48, it was tested whether CD48-MVPs can selectively bind to target cells expressing CD2, its cognate receptor. First, target cell lines were established by transfecting S293 cells with a construct expressing CD2. Transfected cells were then stained with anti-CD2 antibody to differentiate CD2+ from CD2− cells. Subsequently, CD48-MVPs were labeled with fluorescent dye, transfected cells were stained with labeled MVPs, and selective MVP-cell binding was analyzed via FACS (FIG. 49B). The results showed that labeled CD48-MVP binding caused significantly higher fluorescence shift in CD2+ cells as compared to CD2− cells (FIG. 49B, upper panel). Moreover, this shift was significantly higher than the fluorescence shift caused by staining the same cells with control MVPs displaying non-specific ligand (FIG. 49B, lower panel). This result demonstrated that CD48-MVPs displayed functional CD48 and can selectively bind to target cells expressing CD2.

This result was further validated through an alternative staining method (FIG. 49C). In this case, CD2-transfected cells were first incubated with unlabeled CD48-MVPs to allow MVPs to bind to target cells. The cell-MVP mixture was then co-stained with fluorescently-labeled anti-CD2 and anti-CD48 antibodies. CD48 staining pattern on CD2+ and CD2− cells was then examined via FACS analysis. The results showed that CD2+ cells were also CD48 positive, as exemplified by more than 1-log CD48 staining shift in CD2+ cells from CD2− background cells (FIG. 49C). Single staining with anti-CD2 antibody did not compete with CD48-MVP binding to target cells, and CD2-transfected S293 cells were CD48 negative. These results demonstrated that CD48-MVPs displayed functional CD48. Collectively, CD48-MVPs were generated displaying high copy numbers of functional protein, and these MVPs can selectively bind to target cells expressing their cognate receptor, CD2.

### CD2-MVP Composition and Selective Binding to Target Cells Expressing CD48

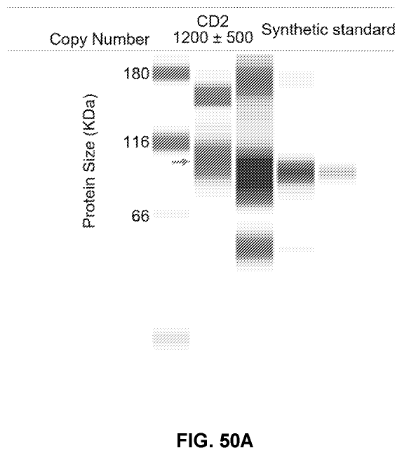

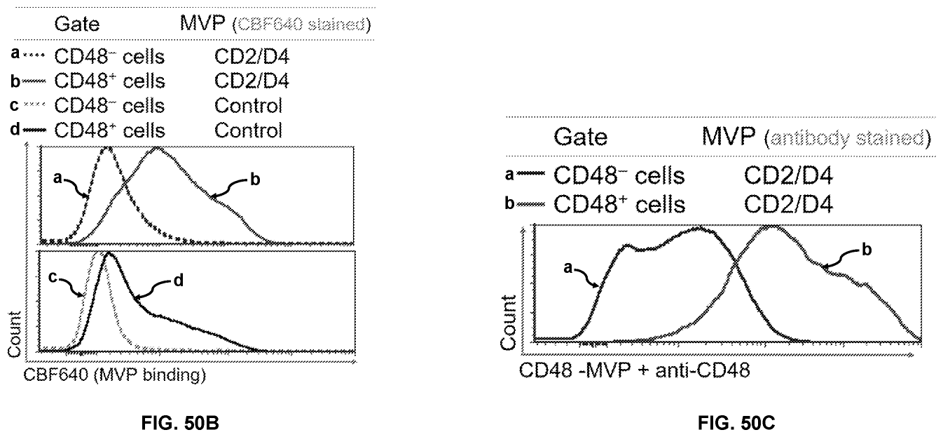

CD2-MVPs were generated by pseudotyping lentiviral VLPs with trimeric CD2 fusion peptides. Specifically, HEK 293T cells were co-transfected with a trimeric CD2 display construct, along with a lentiviral packaging construct expressing essential packaging components, including Gag-Pol and Rev proteins, and a viral genome transfer vector encoding a GFP/luciferase reporter (FIG. 3A). The concentration of purified CD2-MVPs was quantified via P24 ELISA. CD2-MVPs displayed 1200±500 copies of CD2 per MVP in various oligomerized forms, as determined by quantitative western blot analysis (FIG. 50A).

To confirm that CD2-MVPs display functional CD2, it was tested whether CD2-MVPs can selectively bind to target cells expressing CD48, a cognate receptor of CD2. First, target cell lines were established by transfecting S293 cells with a construct expressing CD48. Transfected cells were then stained with anti-CD48 antibody to differentiate CD48+ from CD48− cells. Subsequently, CD2-MVPs were labeled with fluorescent dye, transfected cells were stained with labeled MVPs, and selective MVP-cell binding was analyzed via FACS (FIG. 50B). The results showed that labeled CD2-MVP binding caused significantly higher fluorescence shift in CD48+ cells as compared to CD48− cells (FIG. 50B, upper panel). Moreover, this shift was significantly higher than the fluorescence shift caused by staining the same cells with control MVPs displaying non-specific ligand (FIG. 50B, lower panel). This result demonstrated that CD2-MVPs displayed functional CD2 and can selectively bind to target cells expressing CD48.

This result was further validated through an alternative staining method (FIG. 50C). In this case, CD48-transfected cells were first incubated with unlabeled CD2-MVPs to allow MVPs to bind to target cells. The cell-MVP mixture was then co-stained with fluorescently-labeled anti-CD48 and anti-CD2 antibodies. CD2 staining pattern on CD48+ and CD48− cells was then examined via FACS analysis. The results showed that CD48+ cells were also CD2 positive, as exemplified by more than 1-log CD2 staining shift in CD48+ cells from CD48− background cells (FIG. 50C). Single staining with anti-CD48 antibody did not compete with CD2-MVP binding to target cells, and CD48-transfected S293 cells were CD2 negative. These results demonstrated that CD2-MVPs displayed functional CD48. Collectively, CD2-MVPs were generated displaying high copy numbers of functional protein, and these MVPs can selectively bind target cells expressing their cognate receptor, CD48.

### CD27-MVP Composition and Selective Binding to Target Cells Expressing CD70

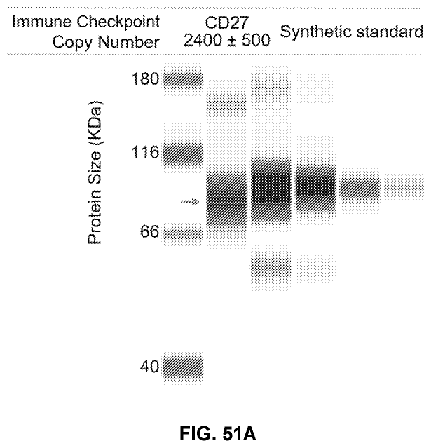

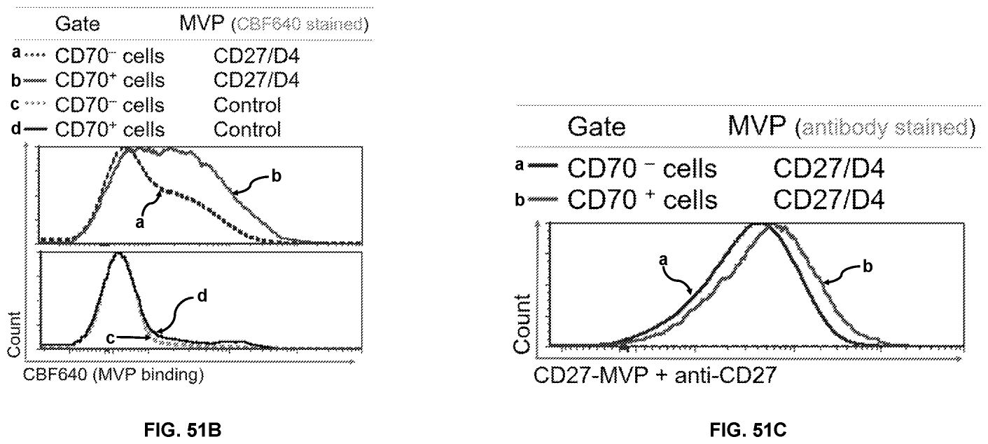

CD27-MVPs were generated by pseudotyping lentiviral VLPs with trimeric CD27 fusion peptides. Specifically, HEK 293T cells were co-transfected with a trimeric CD27 display construct, along with a lentiviral packaging construct expressing essential packaging components, including Gag-Pol and Rev proteins, and a viral genome transfer vector encoding a GFP/luciferase reporter (FIG. 3A). The concentration of purified CD27-MVPs was quantified via P24 ELISA. CD27-MVPs displayed 2400±500 copies of CD27 per MVP in various oligomerized forms, as determined by quantitative western blot analysis (FIG. 51A).

To confirm that CD27-MVPs display functional CD27, it was tested whether CD27-MVPs can selectively bind to target cells expressing CD70, its cognate receptor. First, target cell lines were established by transfecting S293 cells with a construct expressing CD70. Transfected cells were then stained with anti-CD70 antibody to differentiate CD70+ from CD70− cells. Subsequently, CD27-MVPs were labeled with fluorescent dye, transfected cells were stained with labeled MVPs, and selective MVP-cell binding was analyzed via FACS (FIG. 51B). The results showed that labeled CD27-MVP binding caused significantly higher fluorescence shift in CD70+ cells as compared to CD70− cells (FIG. 51B, upper panel). Moreover, this shift was significantly higher than the fluorescence shift caused by staining the same cells with control MVPs displaying non-specific ligand (FIG. 51B, lower panel). This result demonstrated that CD27-MVPs displayed functional CD27 and can selectively bind to target cells expressing CD70.

This result was further validated through an alternative staining method (FIG. 51C). In this case, CD70-transfected cells were first incubated with unlabeled CD27-MVPs to allow MVPs to bind to target cells. The cell-MVP mixture was then co-stained with fluorescently-labeled anti-CD70 and anti-CD27 antibodies. CD27 staining pattern on CD70+ and CD70− cells was then examined via FACS analysis. The results showed that CD70+ cells were also CD27 positive, as exemplified by more than 1-log CD27 staining shift in CD70+ cells from CD70− background cells (FIG. 51C). Single staining with anti-CD70 antibody did not compete with CD27-MVP binding to target cells, and CD70-transfected S293 cells were CD27 negative. These results demonstrated that CD27-MVPs displayed functional CD27. Collectively, CD27-MVPs were generated displaying high copy numbers of functional protein, and these MVPs can selectively bind target cells expressing their cognate receptor, CD70.

### CD70-MVP Composition and Selective Binding to Target Cells Expressing CD27

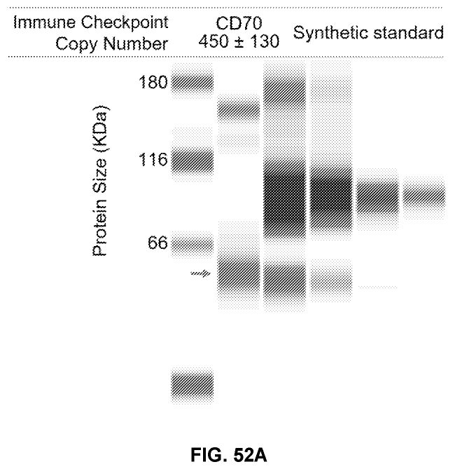

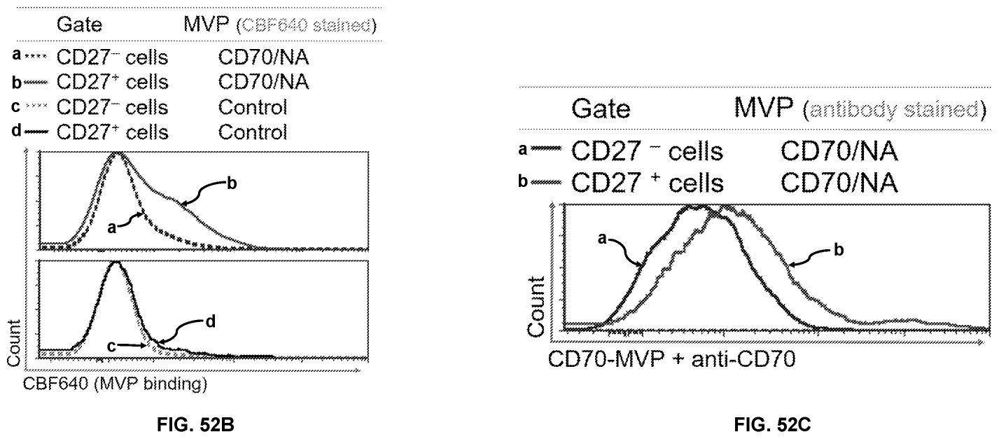

CD70-MVPs were generated by pseudotyping lentiviral VLPs with trimeric CD70 fusion peptides. Specifically, HEK 293T cells were co-transfected with a trimeric CD70 display construct, along with a lentiviral packaging construct expressing essential packaging components, including Gag-Pol and Rev proteins, and a viral genome transfer vector encoding a GFP/luciferase reporter (FIG. 3A). The concentration of purified CD70-MVPs was quantified via P24 ELISA. CD70-MVPs displayed 450±130 copies of CD70 per MVP in various oligomerized forms, as determined by quantitative western blot analysis (FIG. 52A).

To confirm that CD70-MVPs display functional CD70, it was tested whether CD70-MVPs can selectively bind to target cells expressing CD27, a cognate receptor of CD70. First, target cell lines were established by transfecting S293 cells with a construct expressing CD27. Transfected cells were then stained with anti-CD27 antibody to differentiate CD27+ from CD27-cells. Subsequently, CD70-MVPs were labeled with fluorescent dye, transfected cells were stained with labeled MVPs, and selective MVP-cell binding was analyzed via FACS (FIG. 52B). The results showed that labeled CD70-MVP binding caused significantly higher fluorescence shift in CD27+ cells as compared to CD27− cells (FIG. 52B, upper panel). Moreover, this shift was significantly higher than the fluorescence shift caused by staining the same cells with control MVPs displaying non-specific ligand (FIG. 52B, lower panel). This result demonstrated that CD70-MVPs displayed functional CD70 and can selectively bind to target cells expressing CD27.

This result was further validated through an alternative staining method (FIG. 52C). In this case, CD27-transfected cells were first incubated with unlabeled CD70-MVPs to allow MVPs to bind to target cells. The cell-MVP mixture was then co-stained with fluorescent-labeled anti-CD27 and anti-CD70 antibodies. We then examined CD70 staining pattern on CD27+ vs CD27-cells via FACS analysis. The results showed that CD27+ cells were also CD70 positive, as exemplified by significant CD70 staining shift in CD27+ cells from CD27− background cells (FIG. 52C). Single staining with anti-CD27 antibody did not compete with CD70-MVP binding to target cells, and CD27-transfected S293 cells were CD70 negative. These results demonstrated that CD70-MVPs displayed functional CD70. Collectively, CD70-MVPs were generated displaying high copy numbers of functional protein, and these MVPs can selectively bind target cells expressing their cognate receptor, CD27.

### ICOSL-MVP Composition and Selective Binding to Target Cells Expressing ICOS

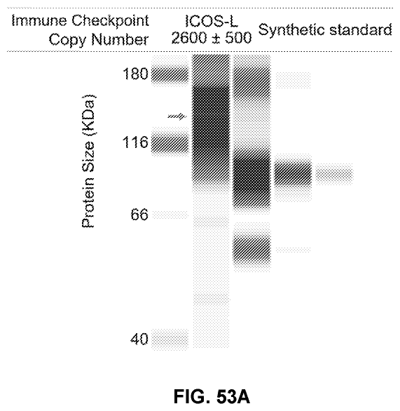

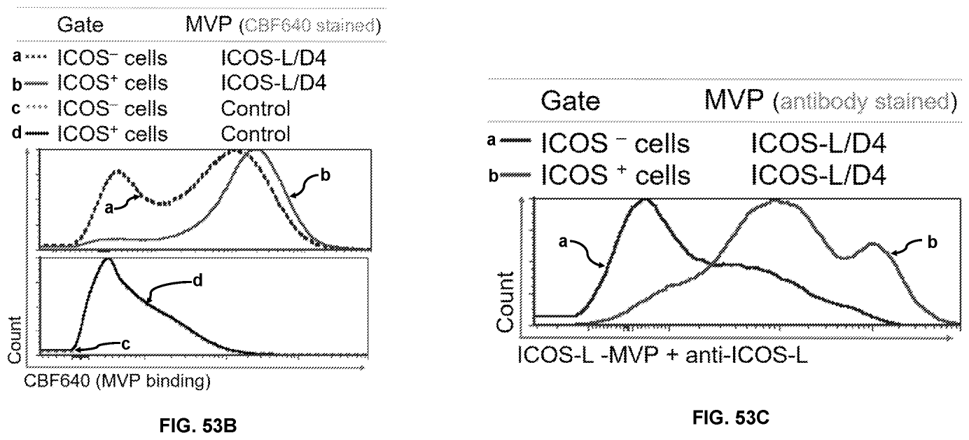

ICOSL-MVPs were generated by pseudotyping lentiviral VLPs with trimeric ICOS-L fusion peptides. Specifically, HEK 293T cells were co-transfected with a trimeric ICOS-L display construct, along with a lentiviral packaging construct expressing essential packaging components, including Gag-Pol and Rev proteins, and a viral genome transfer vector encoding a GFP/luciferase reporter (FIG. 3A). The concentration of purified ICOSL-MVPs was quantified via P24 ELISA. ICOSL-MVPs displayed 2600±500 copies of ICOS-L per MVP in various oligomerized forms, as determined by quantitative western blot analysis (FIG. 53A).

To confirm that ICOSL-MVPs display functional ICOS-L, it was tested whether ICOSL-MVPs can selectively bind to target cells expressing ICOS, a cognate receptor of ICOS-L. First, target cell lines were established by transfecting S293 cells with a construct expressing ICOS. Transfected cells were then stained with anti-ICOS antibody to differentiate ICOS+ from ICOS-cells. Subsequently, ICOSL-MVPs were labeled with fluorescent dye, transfected cells were stained with labeled MVPs, and selective MVP-cell binding was analyzed via FACS (FIG. 53B). The results showed that labeled ICOSL-MVP binding caused significantly higher fluorescence shift in ICOS+ cells as compared to ICOS− cells (FIG. 53B, upper panel). Moreover, this shift was significantly higher than the fluorescence shift caused by staining the same cells with control MVPs displaying non-specific ligand (FIG. 53B, lower panel). This result demonstrated that ICOSL-MVPs displayed functional ICOS-L and can selectively bind to target cells expressing ICOS.

This result was further validated through an alternative staining method (FIG. 53C). In this case, ICOS-transfected cells were first incubated with unlabeled ICOSL-MVPs to allow MVPs to bind to target cells. The cell-MVP mixture was then co-stained with fluorescently-labeled anti-ICOS and anti-ICOSL antibodies. ICOS-L staining pattern on ICOS+ and ICOS− cells was then examined via FACS analysis. The results showed that ICOS+ cells were also ICOS-L positive, as exemplified by two-log ICOS-L staining shift in ICOS+ cells from ICOS− background cells (FIG. 53C). Single staining with anti-ICOS antibody did not compete with ICOSL-MVP binding to target cells, and ICOS-transfected S293 cells were ICOS-L negative. These results demonstrated that ICOS-L-MVPs displayed functional ICOS-L. Collectively, ICOSL-MVPs were generated displaying high copy numbers of functional protein, and these MVPs can selectively bind target cells expressing their cognate receptor, ICOS.

### ICOS-MVP Composition and Selective Binding to Target Cells Expressing ICOS Ligand

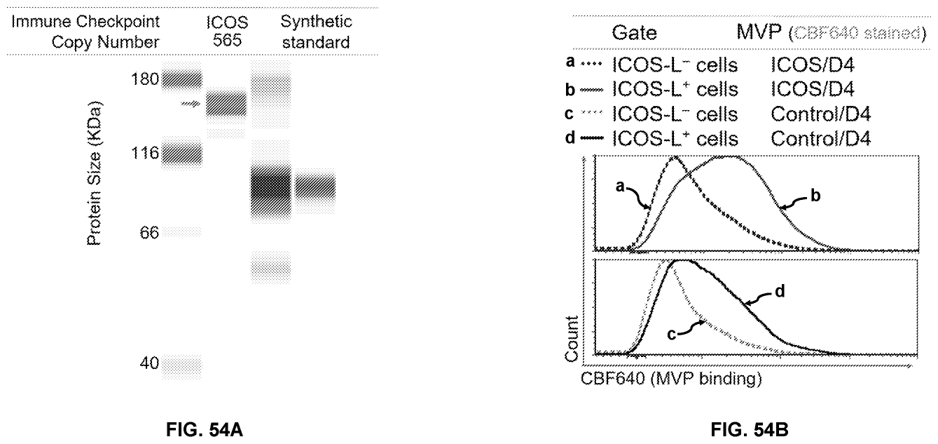

ICOS-MVPs were generated by pseudotyping lentiviral VLPs with trimeric ICOS fusion peptides. Specifically, HEK 293T cells were co-transfected with a trimeric ICOS display construct, along with a lentiviral packaging construct expressing essential packaging components, including Gag-Pol and Rev proteins, and a viral genome transfer vector encoding a GFP/luciferase reporter (FIG. 3A). The concentration of purified ICOS-MVPs was quantified via P24 ELISA. ICOS-MVPs displayed 565 copies of ICOS per MVP in various oligomerized forms, as determined by quantitative western blot analysis (FIG. 54A).

To confirm that ICOS-MVPs display functional ICOS, it was tested whether ICOS-MVPs can selectively bind to target cells expressing ICOS-L, a cognate ligand of ICOS. First, target cell lines were established by transfecting S293 cells with a construct expressing ICOS-L. Transfected cells were then stained with anti-ICOS-L antibody to differentiate ICOS-L+ from ICOS-L− cells. Subsequently, ICOS-MVPs were labeled with fluorescent dye, transfected cells were stained with labeled MVPs, and selective MVP-cell binding was analyzed via FACS (FIG. 54B). The results showed that labeled ICOS-MVP binding caused significantly higher fluorescence shift in ICOS-L+ cells as compared to ICOS-L− cells (FIG. 54B, upper panel). Moreover, this shift was about two times higher than the fluorescence shift caused by staining the same cells with control MVPs displaying non-specific ligand (FIG. 54B, lower panel). This result demonstrated that ICOS-MVPs displayed functional ICOS and can selectively bind to target cells expressing ICOS ligand.

### GITRL-MVP Composition and Selective Binding to Target Cells Expressing GITR

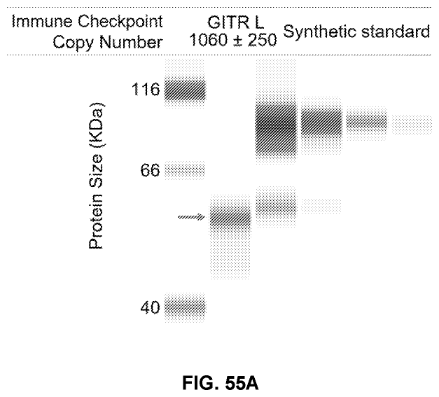

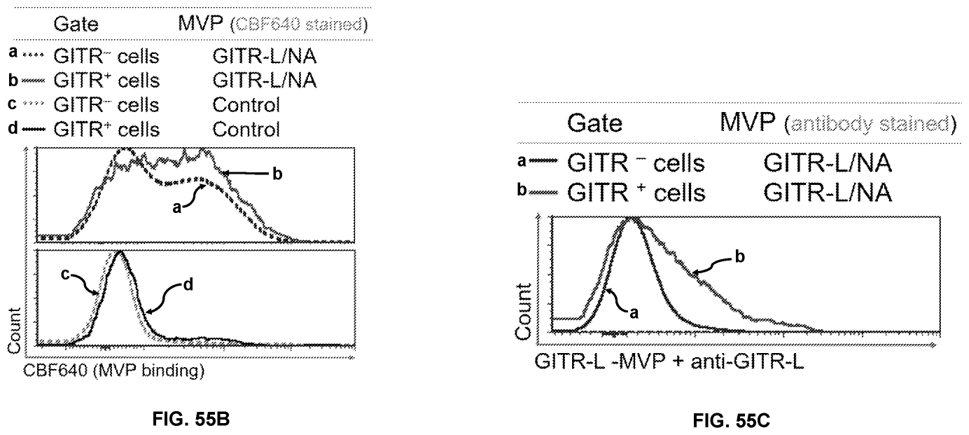

GITRL-MVPs were generated by pseudotyping lentiviral VLPs with trimeric GITR Ligand fusion peptides. Specifically, HEK 293T cells were co-transfected with a trimeric GITR Ligand display construct, along with a lentiviral packaging construct expressing essential packaging components, including Gag-Pol and Rev proteins, and a viral genome transfer vector encoding a GFP/luciferase reporter (FIG. 3A). The concentration of purified GITRL-MVPs was quantified via P24 ELISA. GITRL-MVPs displayed 1060±250 copies of GITR Ligand per MVP in various oligomerized forms, as determined by quantitative western blot analysis (FIG. 55A).

To confirm that GITRL-MVPs display functional GITR Ligand, it was tested whether GITRL-MVPs can selectively bind to target cells expressing GITR, a cognate receptor of GITR-L. First, target cell lines were established by transfecting S293 cells with a construct expressing GITR. Transfected cells were then stained with anti-GITR antibody to differentiate GITR+ from GITR-cells. Subsequently, GITRL-MVPs were labeled with fluorescent dye, transfected cells were stained with labeled MVPs, and selective MVP-cell binding was analyzed via FACS (FIG. 55B). The results showed that labeled GITRL-MVP binding caused slightly higher fluorescence shift in GITR+ cells as compared to GITR− cells (FIG. 55B, upper panel). Moreover, this shift was still higher than the fluorescence shift caused by staining the same cells with control MVPs displaying non-specific ligand (FIG. 55B, lower panel). This result demonstrated that GITRL-MVPs displayed functional GITR Ligand and can selectively bind to target cells expressing GITR.

This result was further validated through an alternative staining method (FIG. 55C). In this case, GITR-transfected cells we first incubated with unlabeled GITR-L-MVPs to allow MVPs to bind to target cells. The cell-MVP mixture was then co-stained with fluorescently-labeled anti-GITR and anti-GITR-L antibodies. GITR-L staining pattern on GITR+ and GITR− cells was then examined via FACS analysis. The results showed that GITR+ cells were also GITR-L positive, as exemplified by around two times of GITR-L staining shift in GITR+ cells from GITR− background cells (FIG. 55C). Single staining with anti-GITR antibody did not compete with GITR-L-MVP binding to target cells, and GITR-transfected S293 cells were GITR-L negative. These results demonstrated that GITRL-MVPs displayed functional GITR Ligand. Collectively, GITRL-MVPs were generated displaying high copy numbers of functional protein, and these MVPs can selectively bind to target cells expressing their cognate receptor, GITR.

### GITR-MVP Composition and Selective Binding to Target Cells Expressing GITR Ligand

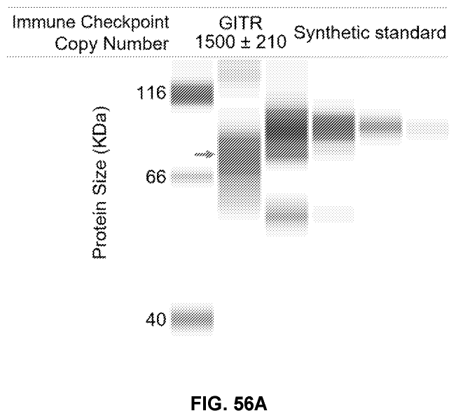

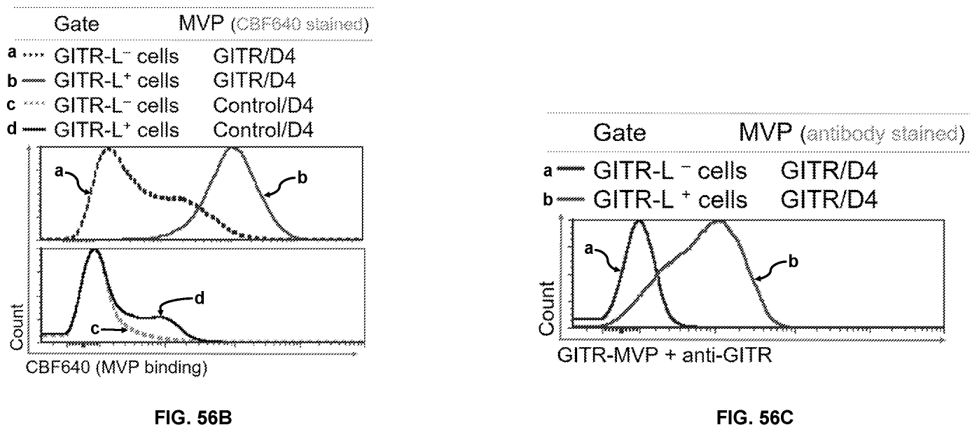

GITR-MVPs were generated by pseudotyping lentiviral VLPs with trimeric GITR fusion peptides. Specifically, HEK 293T cells were co-transfected with a trimeric GITR display construct, along with a lentiviral packaging construct expressing essential packaging components, including Gag-Pol and Rev proteins, and a viral genome transfer vector encoding a GFP/luciferase reporter (FIG. 3A). The concentration of purified GITR-MVPs was quantified via P24 ELISA. GITR-MVPs displayed 1500±210 copies of GITR per MVP in various oligomerized forms, as determined by quantitative western blot analysis (FIG. 56A).

To confirm that GITR-MVPs display functional GITR, it was tested whether GITR-MVPs can selectively bind to target cells expressing GITR-L, a cognate ligand of GITR. First, target cell lines were established by transfecting S293 cells with a construct expressing GITR Ligand. Transfected cells were then stained with anti-GITR-L antibody to differentiate GITRL+ from GITRL− cells. Subsequently, GITR-MVPs were labeled with fluorescent dye, transfected cells were stained with labeled MVPs, and selective MVP-cell binding was analyzed via FACS (FIG. 56B). The results showed that labeled GITR-MVP binding caused significantly higher fluorescence shift in GITR-L+ cells as compared to GITR-L− cells (FIG. 56B, upper panel). Moreover, this shift was at least two-log higher than the fluorescence shift caused by staining the same cells with control MVPs displaying non-specific ligand (FIG. 56B, lower panel). This result demonstrated that GITR-MVPs displayed functional GITR and can selectively bind to target cells expressing GITR Ligand.

This result was further validated through an alternative staining method (FIG. 56C). In this case, GITR-L-transfected cells were first incubated with unlabeled GITR-MVPs to allow MVPs to bind to target cells. The cell-MVP mixture was then co-stained with fluorescently-labeled anti-GITRL and anti-GITR antibodies. GITR staining pattern on GITRL+ and GITRL− cells was then examined via FACS analysis. The results showed that GITRL+ cells were also GITR positive, as exemplified by one-log GITR staining shift in GITRL+ cells from GITRL− background cells (FIG. 56C). Single staining with anti-GITR-L antibody did not compete with GITR-MVP binding to target cells, and GITR-L-transfected S293 cells were GITR negative. These results demonstrated that GITR-MVPs displayed functional GITR. Collectively, GITR-MVPs were generated displaying high copy numbers of functional protein, and these MVPs can selectively bind target cells expressing their cognate receptor.

### 4-1BB-MVP Composition and Selective Binding to Target Cells Expressing 4-1BB Ligand

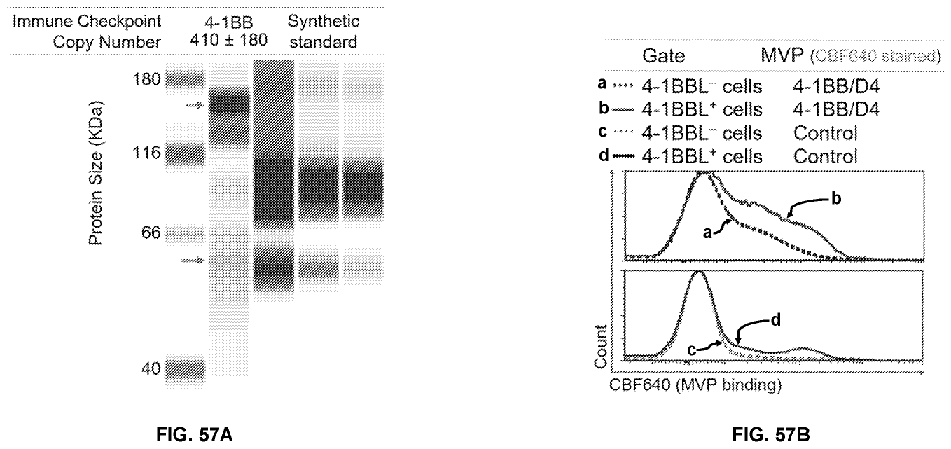

4-1BB-MVPs were generated by pseudotyping lentiviral VLPs with trimeric 4-1BB fusion peptides. Specifically, HEK 293T cells were co-transfected with a trimeric 4-1BB display construct, along with a lentiviral packaging construct expressing essential packaging components, including Gag-Pol and Rev proteins, and a viral genome transfer vector encoding a GFP/luciferase reporter (FIG. 3A). The concentration of purified 4-1BB-MVPs was quantified via P24 ELISA. 4-1BB-MVPs displayed 410±180 copies of 4-1BB per MVP in various oligomerized forms, as determined by quantitative western blot analysis (FIG. 57A).

To confirm that 4-1BB-MVPs display functional 4-1BB, it was tested whether 4-1BB-MVPs can selectively bind to target cells expressing 4-1BBL, its cognate ligand. First, target cell lines were established by transfecting S293 cells with a construct expressing 4-1BBL. Transfected cells were then stained with anti-4-1BBL antibody to differentiate 4-1BBL+ from 41BBL− cells. Subsequently, 4-1BB-MVPs were labeled with fluorescent dye, transfected cells were stained with labeled MVPs, and selective MVP-cell binding was analyzed via FACS (FIG. 57B). The results showed that labeled 4-1BB-MVP binding caused significantly higher fluorescence shift in 4-1BBL+ cells as compared to 4-1BBL− cells (FIG. 57B, upper panel). Moreover, this shift was at least two times higher than the fluorescence shift caused by staining the same cells with control MVPs displaying non-specific ligand (FIG. 57B, lower panel). This result demonstrated that 4-1BB-MVPs displayed functional 4-1BB and can selectively bind to target cells expressing 4-1BBL.

### OX40-MVP Composition and Selective Binding to Target Cells Expressing OX40 Ligand

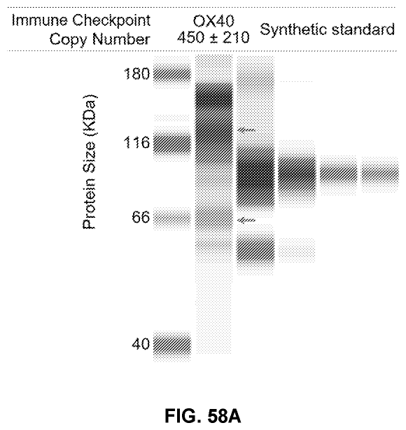

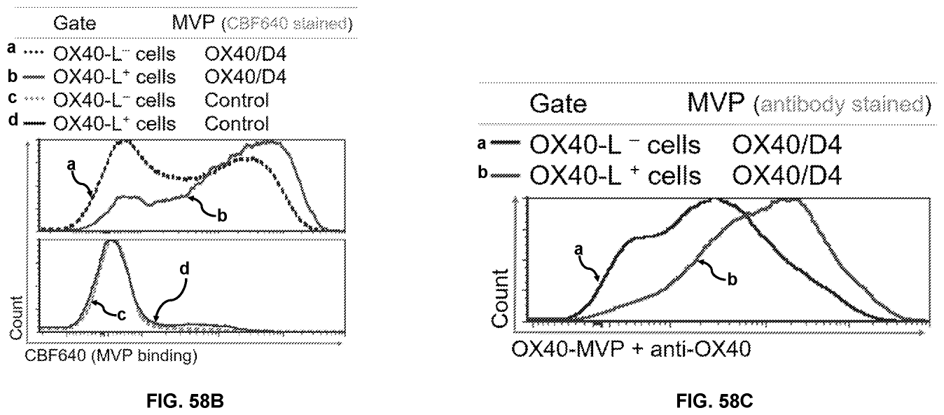

OX40-MVPs were generated by pseudotyping lentiviral VLPs with trimeric OX40 fusion peptides. Specifically, HEK 293T cells were co-transfected with a trimeric OX40 display construct, along with a lentiviral packaging construct expressing essential packaging components, including Gag-Pol and Rev proteins, and a viral genome transfer vector encoding a GFP/luciferase reporter (FIG. 3A). The concentration of purified OX40-MVPs was quantified via P24 ELISA. OX40-MVPs displayed 450±210 copies of OX40 per MVP in various oligomerized forms, as determined by quantitative western blot analysis (FIG. 58A).

To confirm that OX40-MVPs display functional OX40, it was tested whether OX40-MVPs can selectively bind to target cells expressing OX40L, a cognate ligand of OX40. First, target cell lines were established by transfecting S293 cells with a construct expressing OX40L. Transfected cells were then stained with anti-OX40L antibody to differentiate OX40L+ from OX40L− cells. Subsequently, OX40-MVPs were labeled with fluorescent dye, transfected cells were stained with labeled MVPs, and selective MVP-cell binding was analyzed via FACS (FIG. 58B). The results showed that labeled OX40-MVP binding caused significantly higher fluorescence shift in OX40L+ cells as compared to OX40L− cells (FIG. 58B, upper panel). Moreover, this shift was at least two times higher than the fluorescence shift caused by staining the same cells with control MVPs displaying non-specific ligand (FIG. 58B, lower panel). This result demonstrated that OX40-MVPs displayed functional OX40 and can selectively bind to target cells expressing OX40 ligand.

This result was further validated through an alternative staining method (FIG. 58C). In this case, OX40L-transfected cells were first incubated with unlabeled OX40-MVPs to allow MVPs to bind to target cells. The cell-MVP mixture was then co-stained with fluorescently-labeled anti-OX40L and anti-OX40 antibodies. OX40 staining pattern on OX40L+ and OX40L− cells was then examined via FACS analysis. The results showed that OX40L+ cells were also OX40 positive, as exemplified by one-log OX40 staining shift in OX40-L+ cells from OX40-L− background cells (FIG. 58C). Single staining with anti-OX40L antibody did not compete with OX40-MVP binding to target cells, and OX40L-transfected S293 cells were OX40 negative. These results demonstrated that OX40-MVPs displayed functional OX40. Collectively, OX40-MVPs were generated displaying high copy numbers of functional protein, and these MVPs can selectively bind target cells expressing their cognate ligand, OX40 ligand.

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

### EMBODIMENTS

The following non-limiting embodiments provide illustrative examples of the invention, but do not limit the scope of the invention.

Embodiment 1. A multivalent particle comprising a fusion protein that comprises a mammalian immune checkpoint polypeptide and a transmembrane polypeptide wherein the fusion protein is expressed at a valency of at least about 10 copies on a surface of the multivalent particle.

Embodiment 2. The multivalent particle of embodiment 1, wherein the mammalian immune checkpoint polypeptide comprises a polypeptide expressed on T cells.

Embodiment 3. The multivalent particle of embodiment 1, wherein the mammalian immune checkpoint polypeptide comprises an immune inhibitory checkpoint polypeptide.

Embodiment 4. The multivalent particle of embodiment 3, wherein the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9.

Embodiment 5. The multivalent particle of embodiment 3, wherein the immune inhibitory checkpoint polypeptide is expressed on antigen presenting cells, tumor cells, or normal cells.

Embodiment 6. The multivalent particle of embodiment 5, wherein the immune inhibitory checkpoint polypeptide comprises PD-L1, PD-L2, CD80, CD86, HVEM, CD48, CD112, CD155, Ceacam1, FGL1, or Galectin-3.

Embodiment 7. The multivalent particle of embodiment 1, wherein the mammalian immune checkpoint polypeptide comprises an immune stimulatory checkpoint polypeptide.

Embodiment 8. The multivalent particle of embodiment 7, wherein the immune stimulatory checkpoint polypeptide comprises a polypeptide expressed on T cells.

Embodiment 9. The multivalent particle of embodiment 7, wherein the immune stimulatory checkpoint polypeptide comprises CD27, CD28, CD40, CD122, 4-1BB, ICOS, OX40, CD2, CD30, or GITR.

Embodiment 10. The multivalent particle of embodiment 7, wherein the immune stimulatory checkpoint polypeptide comprises CD70, CD80, CD86, CD40L, GITRL, 4-1BBL, OX40L, LIGHT, CD30L, CD48, or ICOSL.

Embodiment 11. The multivalent particle of any one of embodiments 9-10, wherein the immune stimulatory checkpoint polypeptide is expressed on antigen presenting cells, tumor cells, or normal cells.

Embodiment 12. The multivalent particle of embodiment 3, wherein the immune inhibitory checkpoint polypeptide comprises an amino acid sequence of at least 90% sequence identity to an amino acid sequence according to any one of SEQ ID NOs: 1-42, or 96-101.

Embodiment 13. The multivalent particle of embodiment 7, wherein the immune stimulatory checkpoint polypeptide comprises an amino acid sequence of at least 90% sequence identity to an amino acid sequence according to any one of SEQ ID NOs: 43-62, 102-115, or 153-162.

Embodiment 14. The multivalent particle of any one of embodiments 1-13, wherein the transmembrane polypeptide anchors the fusion protein to a bilayer of the multivalent particle.

Embodiment 15. The multivalent particle of any one of embodiments 1-14, wherein the transmembrane polypeptide comprises a spike glycoprotein, a mammalian membrane protein, an envelope protein, a nucleocapsid protein, or a cellular transmembrane protein.

Embodiment 16. The multivalent particle of any one of embodiments 1-14, wherein the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120.

Embodiment 17. The multivalent particle of embodiment 16, wherein the VSVG comprises full length VSVG or a truncated VSVG.

Embodiment 18. The multivalent particle of embodiment 16, wherein the VSVG comprises a transmembrane domain and cytoplasmic tail.

Embodiment 19. The multivalent particle of any one of embodiments 1-18, wherein the fusion protein further comprises an oligomerization domain.

Embodiment 20. The multivalent particle of embodiment 19, wherein the oligomerization domain is a dimerization domain.

Embodiment 21. The multivalent particle of embodiment 20, wherein the dimerization domain comprises a leucine zipper dimerization domain.

Embodiment 22. The multivalent particle of embodiment 20, wherein the oligomerization domain is a trimerization domain.

Embodiment 23. The multivalent particle of embodiment 22, wherein the trimerization domain comprises a post-fusion oligomerization domain of viral surface protein.

Embodiment 24. The multivalent particle of embodiment 22, wherein the trimerization domain comprises a D4 post-fusion trimerization domain of VSV-G protein.

Embodiment 25. The multivalent particle of embodiment 22, wherein the trimerization domain comprises a Dengue E protein post-fusion trimerization domain.

Embodiment 26. The multivalent particle of embodiment 22, wherein the trimerization domain comprises a foldon trimerization domain.

Embodiment 27. The multivalent particle of embodiment 20, wherein the oligomerization domain is a tetramerization domain.

Embodiment 28. The multivalent particle of embodiment 27, wherein the tetramerization domain comprises an influenza neuraminidase stem domain.

Embodiment 29. The multivalent particle of embodiment 20, wherein the oligomerization domain comprises an amino acid sequence that has at least 95% sequence identity to an amino acid sequence according to SEQ ID NOs: 65-78.

Embodiment 30. The multivalent particle of any one of embodiments 20-29, wherein when the fusion protein is expressed on the surface of the multivalent particle, the oligomerization domain is outside of the multivalent particle.

Embodiment 31. The multivalent particle of any one of embodiments 20-29, wherein when the fusion protein is expressed on the surface of the multivalent particle, the oligomerization domain is outside of the multivalent particle and adjacent to a signal peptide.

Embodiment 32. The multivalent particle of any one of embodiments 20-29, wherein when the fusion protein is expressed on the surface of the multivalent particle, the oligomerization domain is inside of the multivalent particle.

Embodiment 33. The multivalent particle of any one of embodiments 20-29, wherein when the fusion protein is expressed on the surface of the multivalent particle, the oligomerization domain is inside of the multivalent particle and adjacent to the transmembrane polypeptide.

Embodiment 34. The multivalent particle of any one of embodiments 1-33, wherein the fusion protein comprises a signal peptide.

Embodiment 35. The multivalent particle of any one of embodiments 1-34, wherein domains of the fusion protein are arranged from the N-terminus to the C-terminus in the following orders:
(a) signal peptide, mammalian immune checkpoint polypeptide, oligomerization domain, transmembrane polypeptide, and cytosolic domain;
(b) signal peptide, mammalian immune checkpoint polypeptide, transmembrane polypeptide, oligomerization domain, and cytosolic domain; or
(c) signal peptide, oligomerization domain, mammalian immune checkpoint polypeptide, transmembrane polypeptide, and cytosolic domain.

Embodiment 36. The multivalent particle of any one of embodiments 1-35, wherein the fusion protein is expressed at a valency of about 10 copies on a surface of the multivalent particle.

Embodiment 37. The multivalent particle of any one of embodiments 1-35, wherein the fusion protein is expressed at a valency of about 10 to about 15 copies on a surface of the multivalent particle.

Embodiment 38. The multivalent particle of any one of embodiments 1-35, wherein the fusion protein is expressed at a valency of at least about 25 copies on a surface of the multivalent particle.

Embodiment 39. The multivalent particle of any one of embodiments 1-35, wherein the fusion protein is expressed at a valency of at least about 50 copies on a surface of the multivalent particle.

Embodiment 40. The multivalent particle of any one of embodiments 1-35, wherein the fusion protein is expressed at a valency of at least about 75 copies on a surface of the multivalent particle.

Embodiment 41. The multivalent particle of any one of embodiments 1-35, wherein the fusion protein is expressed at a valency of at least about 100 copies on a surface of the multivalent particle.

Embodiment 42. The multivalent particle of any one of embodiments 1-35, wherein the fusion protein is expressed at a valency of at least about 150 copies on a surface of the multivalent particle.

Embodiment 43. The multivalent particle of any one of embodiments 1-35, wherein the fusion protein is expressed at a valency of at least about 200 copies on a surface of the multivalent particle.

Embodiment 44. The multivalent particle of any one of embodiments 1-43, wherein the multivalent particle does not comprise viral genetic material.

Embodiment 45. The multivalent particle of any one of embodiments 1-44, wherein the multivalent particle is a viral-like a particle.

Embodiment 46. The multivalent particle of any one of embodiments 1-44, wherein the multivalent particle is an extracellular vesicle.

Embodiment 47. The multivalent particle of any one of embodiments 1-44, wherein the multivalent particle is an exosome.

Embodiment 48. The multivalent particle of any one of embodiments 1-44, wherein the multivalent particle is an ectosome.

Embodiment 49. A composition comprising a first nucleic acid sequence encoding a multivalent particle comprising a fusion protein that comprises a mammalian immune checkpoint polypeptide and a transmembrane polypeptide wherein the fusion protein is expressed at a valency of at least about 10 copies on a surface of the multivalent particle when the multivalent particle is expressed; and an excipient.

Embodiment 50. The composition of embodiment 49, further comprising a second nucleic acid sequence that encodes one or more viral proteins.

Embodiment 51. The composition of embodiment 50, wherein the one or more viral proteins is a lentiviral protein, a retroviral protein, an adenoviral protein, or combinations thereof.

Embodiment 52. The composition of embodiment 50, wherein the one or more viral proteins comprises gag, pol, pre, tat, rev, or combinations thereof.

Embodiment 53. The composition of any one of embodiments 49-52, further comprising a third nucleic acid sequence that encodes a replication incompetent viral genome, a reporter, a therapeutic molecule, or combinations thereof.

Embodiment 54. The composition of embodiment 53, wherein the viral genome is derived from vesicular stomatitis virus, measles virus, Hepatitis virus, influenza virus, or combinations thereof.

Embodiment 55. The composition of embodiment 53, wherein the reporter is a fluorescent protein or luciferase.

Embodiment 56. The composition of embodiment 55, wherein the fluorescent protein is green fluorescent protein.

Embodiment 57. The composition of embodiment 53, wherein the therapeutic molecule is a cellular signal modulating molecule, a proliferation modulating molecule, a cell death modulating molecule, or combinations thereof.

Embodiment 58. The composition of any one of embodiments 49-57, wherein the mammalian immune checkpoint polypeptide comprises a polypeptide expressed on T cells.

Embodiment 59. The composition of any one of embodiments 49-57, wherein the mammalian immune checkpoint polypeptide comprises an immune inhibitory checkpoint polypeptide.

Embodiment 60. The composition of embodiment 59, wherein the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9.

Embodiment 61. The composition of embodiment 59, wherein the immune inhibitory checkpoint polypeptide is expressed on antigen presenting cells, tumor cells, or normal cells.

Embodiment 62. The composition of embodiment 61, wherein the immune inhibitory checkpoint polypeptide comprises PD-L1, PD-L2, CD80, CD86, HVEM, CD48, CD112, CD155, Ceacam1, FGL1, or Galectin-3.

Embodiment 63. The composition of embodiment 49, wherein the mammalian immune checkpoint polypeptide comprises an immune stimulatory checkpoint polypeptide.

Embodiment 64. The composition of embodiment 63, wherein the immune stimulatory checkpoint polypeptide comprises a polypeptide expressed on T cells.

Embodiment 65. The composition of embodiment 63, wherein the immune stimulatory checkpoint polypeptide comprises CD27, CD28, CD40, CD122, 4-1BB, ICOS, OX40, CD2, CD30, or GITR.

Embodiment 66. The composition of embodiment 63, wherein the immune stimulatory checkpoint polypeptide comprises CD70, CD80, CD86, CD40L, GITRL, 4-1BBL, OX40L, LIGHT, CD30L, CD48, or ICOSL.

Embodiment 67. The composition of any one of embodiments 65-66, wherein the immune stimulatory checkpoint polypeptide is expressed on antigen presenting cells, tumor cells, or normal cells.

Embodiment 68. The composition of embodiment 49, wherein the immune inhibitory checkpoint polypeptide comprises an amino acid sequence of at least 90% sequence identity to an amino acid sequence according to any one of SEQ ID NOs: 1-42, or 96-101.

Embodiment 69. The composition of embodiment 63, wherein the immune stimulatory checkpoint polypeptide comprises an amino acid sequence of at least 90% sequence identity to an amino acid sequence according to any one of SEQ ID NOs 43-62, 102-115, or 153-162.

Embodiment 70. The composition of any one of embodiments 49-69, wherein the transmembrane polypeptide anchors the fusion protein to a bilayer of the multivalent particle.

Embodiment 71. The composition of any one of embodiments 49-70, wherein the transmembrane polypeptide comprises a spike glycoprotein, a mammalian membrane protein, an envelope protein, a nucleocapsid protein, or a cellular transmembrane protein.

Embodiment 72. The composition of any one of embodiments 49-70, wherein the transmembrane polypeptide comprises the transmembrane domain of VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120.

Embodiment 73. The composition of embodiment 72, wherein the VSVG comprises full length VSVG or a truncated VSVG.

Embodiment 74. The composition of embodiment 72, wherein the VSVG comprises a transmembrane domain and cytoplasmic tail.

Embodiment 75. The composition of any one of embodiments 49-70, wherein the transmembrane polypeptide comprises an amino acid sequence at least about 90% identical to that set forth in any one of SEQ ID NOs: 63, 64, or 79-95.

Embodiment 76. The composition of any one of embodiments 49-70, wherein the transmembrane polypeptide comprises a nucleic acid sequence at least about 95% identical to that set forth in any one of SEQ ID NOs: 63, 64, or 79-95.

Embodiment 77. The composition of any one of embodiments 49-76, wherein the fusion protein further comprises an oligomerization domain.

Embodiment 78. The composition of embodiment 77, wherein the oligomerization domain is a dimerization domain.

Embodiment 79. The composition of embodiment 78, wherein the dimerization domain comprises a leucine zipper dimerization domain.

Embodiment 80. The composition of embodiment 78, wherein the oligomerization domain is a trimerization domain.

Embodiment 81. The composition of embodiment 80, wherein the trimerization domain comprises a post-fusion oligomerization domain of viral surface protein.

Embodiment 82. The composition of embodiment 80, wherein the trimerization domain comprises a D4 post-fusion trimerization domain of VSV-G protein.

Embodiment 83. The composition of embodiment 80, wherein the trimerization domain comprises a Dengue E protein post-fusion trimerization domain.

Embodiment 84. The composition of embodiment 80, wherein the trimerization domain comprises a foldon trimerization domain.

Embodiment 85. The composition of embodiment 78, wherein the oligomerization domain is a tetramerization domain.

Embodiment 86. The composition of embodiment 85, wherein the tetramerization domain comprises an influenza neuraminidase stem domain.

Embodiment 87. The composition of embodiment 78, wherein the oligomerization domain comprises an amino acid sequence that has at least 95% sequence identity to an amino acid sequence according to SEQ ID NOs: 65-78.

Embodiment 88. The composition of any one of embodiments 78-87, wherein when the fusion protein is expressed on the surface of the multivalent particle, the oligomerization domain is outside of the multivalent particle.

Embodiment 89. The composition of any one of embodiments 78-87, wherein when the fusion protein is expressed on the surface of the multivalent particle, the oligomerization domain is outside of the multivalent particle and adjacent to a signal peptide.

Embodiment 90. The composition of any one of embodiments 78-87, wherein when the fusion protein is expressed on the surface of the multivalent particle, the oligomerization domain is inside of the multivalent particle.

Embodiment 91. The composition of any one of embodiments 78-87, wherein when the fusion protein is expressed on the surface of the multivalent particle, the oligomerization domain is inside of the multivalent particle and adjacent to the transmembrane polypeptide.

Embodiment 92. The composition of any one of embodiments 78-87, wherein the fusion protein comprises a signal peptide.

Embodiment 93. The composition of any one of embodiments 78-92, wherein domains of the fusion protein are arranged from the N-terminus to the C-terminus in the following orders:
(a) signal peptide, mammalian immune checkpoint polypeptide, oligomerization domain, transmembrane polypeptide, and cytosolic domain;
(b) signal peptide, mammalian immune checkpoint polypeptide, transmembrane polypeptide, oligomerization domain, and cytosolic domain; or
(c) signal peptide, oligomerization domain, mammalian immune checkpoint polypeptide, transmembrane polypeptide, and cytosolic domain.

Embodiment 94. The composition of any one of embodiments 49-93, wherein the fusion protein is expressed at a valency of at about 10 copies on a surface of the multivalent particle when the multivalent particle is expressed.

Embodiment 95. The composition of any one of embodiments 49-93, wherein the fusion protein is expressed at a valency of at about 10 copies to about 15 copies on a surface of the multivalent particle when the multivalent particle is expressed.

Embodiment 96. The composition of any one of embodiments 49-93, wherein the fusion protein is expressed at a valency of at least about 25 copies on a surface of the multivalent particle when the multivalent particle is expressed.

Embodiment 97. The composition of any one of embodiments 49-93, wherein the fusion protein is expressed at a valency of at least about 50 copies on a surface of the multivalent particle when the multivalent particle is expressed.

Embodiment 98. The composition of any one of embodiments 49-93, wherein the fusion protein is expressed at a valency of at least about 75 copies on a surface of the multivalent particle when the multivalent particle is expressed.

Embodiment 99. The composition of any one of embodiments 49-93, wherein the fusion protein is expressed at a valency of at least about 100 copies on a surface of the multivalent particle when the multivalent particle is expressed.

Embodiment 100. The composition of any one of embodiments 49-93, wherein the fusion protein is expressed at a valency of at least about 150 copies on a surface of the multivalent particle when the multivalent particle is expressed.

Embodiment 101. The composition of any one of embodiments 49-93, wherein the fusion protein is expressed at a valency of at least about 200 copies on a surface of the multivalent particle when the multivalent particle is expressed.

Embodiment 102. The composition of any one of embodiments 49-101, wherein the multivalent particle does not comprise viral genetic material.

Embodiment 103. The composition of any one of embodiments 49-102, wherein the multivalent particle is a viral-like a particle.

Embodiment 104. The composition of any one of embodiments 49-102, wherein the multivalent particle is an extracellular vesicle.

Embodiment 105. The composition of any one of embodiments 49-102, wherein the multivalent particle is an exosome.

Embodiment 106. The composition of any one of embodiments 49-102, wherein the multivalent particle is an ectosome.

Embodiment 107. The composition of embodiment 53, wherein the first nucleic acid sequence, the second nucleic acid sequence, and the third nucleic acid sequence are within a same vector.

Embodiment 108. The composition of embodiment 53, wherein the first nucleic acid sequence, the second nucleic acid sequence, and the third nucleic acid sequence are within different vectors.

Embodiment 109. The composition of any one of embodiments 107-108, wherein the vector is a lentivirus vector, an adenovirus vector, or an adeno-associated virus vector.

Embodiment 110. A pharmaceutical composition comprising the multivalent particle of any one of embodiments 1-48 and a pharmaceutically acceptable excipient.

Embodiment 111. A method of treating cancer in a subject in need thereof comprising administering the multivalent particle of any one of embodiments 1-48 or the composition of any one of embodiments 49-109.

Embodiment 112. The method of embodiment 111, wherein the multivalent particle is administered intravenously.

Embodiment 113. The method of embodiment 111, wherein the multivalent particle is administered by inhalation.

Embodiment 114. The method of embodiment 111, wherein the multivalent particle is administered by an intraperitoneal injection.

Embodiment 115. The method of embodiment 111, wherein the multivalent particle is administered by a subcutaneous injection.

Embodiment 116. The method of any one of embodiments 111-115, wherein the multivalent particle induces T cell mediated cytotoxicity against tumor cells.

Embodiment 117. The method of any one of embodiments 111-115, wherein the administering to the subject of the multivalent particle is sufficient to reduce or eliminate the cancer.

Embodiment 118. The method of embodiment 117, wherein the reduction is compared to a level of cancer prior to administration of the multivalent particle.

Embodiment 119. The method of embodiment 117, wherein the reduction is at least about 1-fold, 5-fold, 10-fold, 20-fold, 40-fold, 60-fold, 80-fold, or 100-fold.

Embodiment 120. The method of any one of embodiments 111-119, wherein the cancer is a hematological malignancy.

Embodiment 121. The method of any one of embodiments 111-119, wherein the cancer is leukemia or lymphoma.

Embodiment 122. The method of embodiment 121, wherein the lymphoma is B-cell lymphoma.

Embodiment 123. The method of any one of embodiments 111-119, wherein the cancer is a solid tumor.

Embodiment 124. The method of embodiment 123, wherein the solid tumor comprises sarcoma, melanoma, breast cancer, lung cancer, pancreatic cancer, ovarian cancer, gastric cancer, brain cancer or carcinoma.

Embodiment 125. The method of embodiment 124, wherein the lung cancer is non-small cell lung cancer.

Embodiment 126. The method of any one of embodiments 111-115, wherein the multivalent particle inhibits T cell mediated cytotoxicity against normal tissues.

Embodiment 127. A method of treating an autoimmune disease in a subject in need thereof comprising administering the multivalent particle of any one of embodiments 1-47 or the composition of any one of embodiments 49-109.

Embodiment 128. The method of embodiment 127, wherein the multivalent particle is administered intravenously.

Embodiment 129. The method of embodiment 127, wherein the multivalent particle is administered by inhalation.

Embodiment 130. The method of embodiment 127, wherein the multivalent particle is administered by an intraperitoneal injection.

Embodiment 131. The method of embodiment 127, wherein the multivalent particle is administered by a subcutaneous injection.

Embodiment 132. The method of any one of embodiments 127-131, wherein the administering to the subject of the multivalent particle is sufficient to dampen or inhibit an autoimmune response.

Embodiment 133. The method of embodiment 132, wherein the reduction is compared to the autoimmune response prior to administration of the multivalent particle.

Embodiment 134. The method of embodiment 132, wherein the reduction is at least about 1-fold, 5-fold, 10-fold, 20-fold, 40-fold, 60-fold, 80-fold, or 100-fold.

Embodiment 135. The method of any one of embodiments 127-133, wherein the autoimmune disease is rheumatoid arthritis, systemic lupus erythematosus, multiple sclerosis, inflammatory bowel diseases, psoriasis, or aplastic anemia.

Embodiment 136. A method of inducing T cell activation, proliferation, or differentiation, comprising contacting a T cell with the multivalent particle of any one of embodiments 7-48 or the composition of any one of embodiments 49-58, 63-109.

Embodiment 137. The multivalent particle of any one of embodiments 1-35, wherein the fusion protein is expressed at a valency of at least about 400 copies on a surface of the multivalent particle.

Embodiment 138. The multivalent particle of any one of embodiments 1-35, wherein the fusion protein is expressed at a valency of at least about 800 copies on a surface of the multivalent particle.

Embodiment 139. The multivalent particle of any one of embodiments 1-35, wherein the fusion protein is expressed at a valency of at least about 1000 copies on a surface of the multivalent particle.

Embodiment 140. The multivalent particle of any one of embodiments 1-35, wherein the fusion protein is expressed at a valency of at least about 2000 copies on a surface of the multivalent particle.

What is claimed is:

1. A multivalent particle comprising a fusion protein that comprises a mammalian immune checkpoint polypeptide and a transmembrane polypeptide wherein the fusion protein is expressed at a valency of at least about 10 copies on a surface of the multivalent particle.

2. The multivalent particle of claim 1, wherein the mammalian immune checkpoint polypeptide comprises a polypeptide expressed on T cells.

3. The multivalent particle of claim 1, wherein the mammalian immune checkpoint polypeptide comprises an immune inhibitory checkpoint polypeptide.

4. The multivalent particle of claim 3, wherein the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9.

5. The multivalent particle of claim 3, wherein the immune inhibitory checkpoint polypeptide is expressed on antigen presenting cells, tumor cells, or normal cells.

6. The multivalent particle of claim 3, wherein the immune inhibitory checkpoint polypeptide comprises PD-L1, PD-L2, CD80, CD86, HVEM, CD48, CD112, CD155, Ceacam1, FGL1, or Galectin-3.

7. The multivalent particle of claim 1, wherein the mammalian immune checkpoint polypeptide comprises an immune stimulatory checkpoint polypeptide.

8. The multivalent particle of claim 7, wherein the immune stimulatory checkpoint polypeptide comprises a polypeptide expressed on T cells.

9. The multivalent particle of claim 7, wherein the immune stimulatory checkpoint polypeptide comprises CD27, CD28, CD40, CD122, 4-1BB, ICOS, OX40, CD2, CD30, or GITR.

10. The multivalent particle of claim 7, wherein the immune stimulatory checkpoint polypeptide comprises CD70, CD80, CD86, CD40L, GITRL, 4-1BBL, OX40L, LIGHT, CD30L, CD48, or ICOSL.

11. The multivalent particle of claim 7, wherein the immune stimulatory checkpoint polypeptide is expressed on antigen presenting cells, tumor cells, or normal cells.

12. The multivalent particle of claim 3, wherein the immune inhibitory checkpoint polypeptide comprises an amino acid sequence of at least 90% sequence identity to an amino acid sequence according to any one of SEQ ID NOs: 1-42, or 96-101.

13. The multivalent particle of claim 7, wherein the immune stimulatory checkpoint polypeptide comprises an amino acid sequence of at least 90% sequence identity to an amino acid sequence according to any one of SEQ ID NOs: 43-62, 102-115 or 153-162.

14. The multivalent particle of claim 1, wherein the transmembrane polypeptide anchors the fusion protein to a bilayer of the multivalent particle.

15. The multivalent particle of claim 1, wherein the transmembrane polypeptide comprises a spike glycoprotein, a mammalian membrane protein, an envelope protein, a nucleocapsid protein, or a cellular transmembrane protein.

16. The multivalent particle of claim 1, wherein the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120.

17. The multivalent particle of claim 16, wherein the VSVG comprises full length VSVG or a truncated VSVG.

18. The multivalent particle of claim 16, wherein the VSVG comprises a transmembrane domain and cytoplasmic tail.

19. The multivalent particle of claim 1, wherein the fusion protein further comprises an oligomerization domain.

20. The multivalent particle of claim 19, wherein the oligomerization domain comprises a dimerization domain, a trimerization domain, or a tetramerization domain.

21. The multivalent particle of claim 20, wherein the dimerization domain comprises a leucine zipper dimerization domain.

22. The multivalent particle of claim 19, wherein the fusion protein further comprises a cytosolic domain.

23. The multivalent particle of claim 20, wherein the trimerization domain comprises a post-fusion oligomerization domain of viral surface protein.

24. The multivalent particle of claim 20, wherein the trimerization domain comprises a D4 post-fusion trimerization domain of VSV-G protein.

25. The multivalent particle of claim 20, wherein the trimerization domain comprises a Dengue E protein post-fusion trimerization domain.

26. The multivalent particle of claim 20, wherein the trimerization domain comprises a foldon trimerization domain.

27. The multivalent particle of claim 1, wherein the transmembrane polypeptide comprises an amino acid sequence at least about 90% identical to that set forth in any one of SEQ ID NOs: 63, 64, or 79-95.

28. The multivalent particle of claim 20, wherein the tetramerization domain comprises an influenza neuraminidase stem domain.

29. The multivalent particle of claim 19, wherein the oligomerization domain comprises an amino acid sequence that has at least 95% sequence identity to an amino acid sequence according to SEQ ID NOs: 65-78.

30. The multivalent particle of claim 20, wherein when the fusion protein is expressed on the surface of the multivalent particle, the oligomerization domain is outside of the multivalent particle.

31. The multivalent particle of claim 20, wherein when the fusion protein is expressed on the surface of the multivalent particle, the oligomerization domain is outside of the multivalent particle and adjacent to a signal peptide.

32. The multivalent particle of claim 20, wherein when the fusion protein is expressed on the surface of the multivalent particle, the oligomerization domain is inside of the multivalent particle.

33. The multivalent particle of claim 20, wherein when the fusion protein is expressed on the surface of the multivalent particle, the oligomerization domain is inside of the multivalent particle and adjacent to the transmembrane polypeptide.

34. The multivalent particle of claim 22, wherein the fusion protein comprises a signal peptide.

35. The multivalent particle of claim 34, wherein domains of the fusion protein are arranged from the N-terminus to the C-terminus in the following orders:
(a) signal peptide, mammalian immune checkpoint polypeptide, oligomerization domain, transmembrane polypeptide, and cytosolic domain;
(b) signal peptide, mammalian immune checkpoint polypeptide, transmembrane polypeptide, oligomerization domain, and cytosolic domain; or
(c) signal peptide, oligomerization domain, mammalian immune checkpoint polypeptide, transmembrane polypeptide, and cytosolic domain.

36. The multivalent particle of claim 1, wherein the fusion protein is expressed at a valency of about 10 copies on a surface of the multivalent particle.

37. The multivalent particle of claim 1, wherein the fusion protein is expressed at a valency of about 10 to about 15 copies on a surface of the multivalent particle.

38. The multivalent particle of claim 1, wherein the fusion protein is expressed at a valency of at least about 25 copies on a surface of the multivalent particle.

39. The multivalent particle of claim 1, wherein the fusion protein is expressed at a valency of at least about 50 copies on a surface of the multivalent particle.

40. The multivalent particle of claim 1, wherein the fusion protein is expressed at a valency of at least about 75 copies on a surface of the multivalent particle.

41. The multivalent particle of claim 1, wherein the fusion protein is expressed at a valency of at least about 100 copies on a surface of the multivalent particle.

42. The multivalent particle of claim 1, wherein the fusion protein is expressed at a valency of at least about 150 copies on a surface of the multivalent particle.

43. The multivalent particle of claim 1, wherein the fusion protein is expressed at a valency of at least about 200 copies on a surface of the multivalent particle.

44. The multivalent particle of claim 1, wherein the multivalent particle does not comprise viral genetic material.

45. The multivalent particle of claim 1, wherein the multivalent particle is a viral-like a particle.

46. The multivalent particle of claim 1, wherein the multivalent particle is an extracellular vesicle (EV).

47. The multivalent particle of claim 1, wherein the multivalent particle is an exosome.

48. The multivalent particle of claim 1, wherein the multivalent particle is an ectosome.

49. The multivalent particle of claim 1, wherein:
(a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; and
(b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120.

50. The multivalent particle of claim 1, wherein:
(a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; and
(b) the transmembrane polypeptide comprises an amino acid sequence at least about 75%, 80%, 85%, 90%, 95%, or 99% identical to that set forth in any one of SEQ ID NOs: 63, 64, or 79-95.

51. The multivalent particle of claim 1, wherein:
(a) the immune checkpoint polypeptide comprises an amino acid sequence of at least 75%, 80%, 85%, 90%, 95%, or 99% sequence identity to an amino acid sequence according to any one of SEQ ID NOs: 1-62, 96-115, or 153-162; and
(b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120.

52. The multivalent particle of claim 1, wherein:
(a) the immune checkpoint polypeptide comprises an amino acid sequence of at least 75%, 80%, 85%, 90%, 95%, or 99% sequence identity to an amino acid sequence according to any one of SEQ ID NOs: 1-62, 96-115, or 153-162; and
(b) the transmembrane polypeptide comprises an amino acid sequence at least about 75%, 80%, 85%, 90%, 95%, or 99% identical to that set forth in any one of SEQ ID NOs: 63, 64, or 79-95.

53. The multivalent particle of claim 19, wherein:
(a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL;
(b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120; and
(c) the oligomerization domain comprises a leucine zipper dimerization domain, a post-fusion oligomerization domain of viral surface protein, a D4 post-fusion trimerization domain of VSV-G protein, a Dengue E protein post-fusion trimerization domain, a foldon trimerization domain, or an influenza neuraminidase stem domain.

54. The multivalent particle of claim 19, wherein:
(a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL;
(b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120; and
(c) the oligomerization domain comprises an amino acid sequence that has at least 75%, 80%, 85%, 90%, 95%, or 99% sequence identity to an amino acid sequence according to SEQ ID NOs: 65-78.

55. The multivalent particle of claim 19, wherein:
(a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL;
(b) the transmembrane polypeptide comprises an amino acid sequence at least about 75%, 80%, 85%, 90%, 95%, or 99% identical to that set forth in any one of SEQ ID NOs: 63, 64, or 79-95; and
(c) the oligomerization domain comprises a leucine zipper dimerization domain, a post-fusion oligomerization domain of viral surface protein, a D4 post-fusion trimerization domain of VSV-G protein, a Dengue E protein post-fusion trimerization domain, a foldon trimerization domain, or an influenza neuraminidase stem domain.

56. The multivalent particle of claim 19, wherein:
(a) the immune checkpoint polypeptide comprises an amino acid sequence of at least 75%, 80%, 85%, 90%, 95%, or 99% sequence identity to an amino acid sequence according to any one of SEQ ID NOs: 1-62, 96-115, or 153-162;
(b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120; and
(c) the oligomerization domain comprises a leucine zipper dimerization domain, a post-fusion oligomerization domain of viral surface protein, a D4 post-fusion trimerization domain of VSV-G protein, a Dengue E protein post-fusion trimerization domain, a foldon trimerization domain, or an influenza neuraminidase stem domain.

57. The multivalent particle of claim 19, wherein:
(a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL;
(b) the transmembrane polypeptide comprises an amino acid sequence at least about 75%, 80%, 85%, 90%, 95%, or 99% identical to that set forth in any one of SEQ ID NOs: 63, 64, or 79-95; and
(c) the oligomerization domain comprises an amino acid sequence that has at least 75%, 80%, 85%, 90%, 95%, or 99% sequence identity to an amino acid sequence according to SEQ ID NOs: 65-78.

58. A composition comprising a first nucleic acid sequence encoding a multivalent particle comprising a fusion protein that comprises a mammalian immune checkpoint polypeptide and a transmembrane polypeptide wherein the fusion protein is expressed at a valency of at least about 10 copies on a surface of the multivalent particle when the multivalent particle is expressed; and an excipient.

59. The composition of claim 58, further comprising a second nucleic acid sequence that encodes one or more viral proteins.

60. The composition of claim 59, wherein the one or more viral proteins is a lentiviral protein, a retroviral protein, an adenoviral protein, or combinations thereof.

61. The composition of claim 59, wherein the one or more viral proteins comprises gag, pol, pre, tat, rev, or combinations thereof.

62. The composition of claim 59, further comprising a third nucleic acid sequence that encodes a replication incompetent viral genome, a reporter, a therapeutic molecule, or combinations thereof.

63. The composition of claim 62, wherein the viral genome is derived from vesicular stomatitis virus, measles virus, Hepatitis virus, influenza virus, or combinations thereof.

64. The composition of claim 62, wherein the reporter is a fluorescent protein or luciferase.

65. The composition of claim 64, wherein the fluorescent protein is green fluorescent protein.

66. The composition of claim 62, wherein the therapeutic molecule is a cellular signal modulating molecule, a proliferation modulating molecule, a cell death modulating molecule, or combinations thereof.

67. The composition of claim 58, wherein the mammalian immune checkpoint polypeptide comprises a polypeptide expressed on T cells.

68. The composition of claim 58, wherein the mammalian immune checkpoint polypeptide comprises an immune inhibitory checkpoint polypeptide.

69. The composition of claim 68, wherein the immune inhibitory checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, or SIGLEC9.

70. The composition of claim 68, wherein the immune inhibitory checkpoint polypeptide is expressed on antigen presenting cells, tumor cells, or normal cells.

71. The composition of claim 68, wherein the immune inhibitory checkpoint polypeptide comprises PD-L1, PD-L2, CD80, CD86, HVEM, CD48, CD112, CD155, Ceacam1, FGL1, or Galectin-3.

72. The composition of claim 68, wherein the mammalian immune checkpoint polypeptide comprises an immune stimulatory checkpoint polypeptide.

73. The composition of claim 71, wherein the immune stimulatory checkpoint polypeptide comprises a polypeptide expressed on T cells.

74. The composition of claim 71, wherein the immune stimulatory checkpoint polypeptide comprises CD27, CD28, CD40, CD122, 4-1BB, ICOS, OX40, CD2, CD30, or GITR.

75. The composition of claim 71, wherein the immune stimulatory checkpoint polypeptide comprises CD70, CD80, CD86, CD40L, GITRL, 4-1BBL, OX40L, LIGHT, CD30L, CD48, or ICOSL.

76. The composition of claim 71, wherein the immune stimulatory checkpoint polypeptide is expressed on antigen presenting cells, tumor cells, or normal cells.

77. The composition of claim 68, wherein the immune inhibitory checkpoint polypeptide comprises an amino acid sequence of at least 90% sequence identity to an amino acid sequence according to any one of SEQ ID NOs: 1-42, or 96-101.

78. The composition of claim 71, wherein the immune stimulatory checkpoint polypeptide comprises an amino acid sequence of at least 90% sequence identity to an amino acid sequence according to any one of SEQ ID NOs: 43-62, 102-115, or 153-162.

79. The composition of claim 58, wherein the transmembrane polypeptide anchors the fusion protein to a bilayer of the multivalent particle.

80. The composition of claim 58, wherein the transmembrane polypeptide comprises a spike glycoprotein, a mammalian membrane protein, an envelope protein, a nucleocapsid protein, or a cellular transmembrane protein.

81. The composition of claim 58, wherein the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120.

82. The composition of claim 81, wherein the VSVG comprises full length VSVG or a truncated VSVG.

83. The composition of claim 81, wherein the VSVG comprises a transmembrane domain and cytoplasmic tail.

84. The composition of claim 58, wherein the transmembrane polypeptide comprises an amino acid sequence at least about 90% identical to that set forth in any one of SEQ ID NOs: 63, 64, or 79-95.

85. The composition of claim 58, wherein the fusion protein further comprises an oligomerization domain.

86. The composition of claim 85, wherein the oligomerization domain comprises a dimerization domain, a trimerization domain, or a tetramerization domain.

87. The composition of claim 86, wherein the dimerization domain comprises a leucine zipper dimerization domain.

88. The composition of claim 86, wherein the trimerization domain comprises a post-fusion oligomerization domain of viral surface protein.

89. The composition of claim 86, wherein the trimerization domain comprises a D4 post-fusion trimerization domain of VSV-G protein.

90. The composition of claim 86, wherein the trimerization domain comprises a Dengue E protein post-fusion trimerization domain.

91. The composition of claim 86, wherein the trimerization domain comprises a foldon trimerization domain.

92. The composition of claim 86, wherein the fusion protein further comprises a cytosolic domain.

93. The composition of claim 86, wherein the tetramerization domain comprises an influenza neuraminidase stem domain.

94. The composition of claim 86, wherein the oligomerization domain comprises an amino acid sequence that has at least 95% sequence identity to an amino acid sequence according to SEQ ID NOs: 65-78.

95. The composition of claim 85, wherein when the fusion protein is expressed on the surface of the multivalent particle, the oligomerization domain is outside of the multivalent particle.

96. The composition of claim 85, wherein when the fusion protein is expressed on the surface of the multivalent particle, the oligomerization domain is outside of the multivalent particle and adjacent to a signal peptide.

97. The composition of claim 85, wherein when the fusion protein is expressed on the surface of the multivalent particle, the oligomerization domain is inside of the multivalent particle.

98. The composition of claim 85, wherein when the fusion protein is expressed on the surface of the multivalent particle, the oligomerization domain is inside of the multivalent particle and adjacent to the transmembrane polypeptide.

99. The composition of claim 92, wherein the fusion protein comprises a signal peptide.

100. The composition of claim 99, wherein domains of the fusion protein are arranged from the N-terminus to the C-terminus in the following orders:
(a) signal peptide, mammalian immune checkpoint polypeptide, oligomerization domain, transmembrane polypeptide, and cytosolic domain;
(b) signal peptide, mammalian immune checkpoint polypeptide, transmembrane polypeptide, oligomerization domain, and cytosolic domain; or
(c) signal peptide, oligomerization domain, mammalian immune checkpoint polypeptide, transmembrane polypeptide, and cytosolic domain.

101. The composition of claim 58, wherein the fusion protein is expressed at a valency of at about 10 copies on a surface of the multivalent particle when the multivalent particle is expressed.

102. The composition of claim 58, wherein the fusion protein is expressed at a valency of at about 10 copies to about 15 copies on a surface of the multivalent particle when the multivalent particle is expressed.

103. The composition of claim 58, wherein the fusion protein is expressed at a valency of at least about 25 copies on a surface of the multivalent particle when the multivalent particle is expressed.

104. The composition of claim 58, wherein the fusion protein is expressed at a valency of at least about 50 copies on a surface of the multivalent particle when the multivalent particle is expressed.

105. The composition of claim 58, wherein the fusion protein is expressed at a valency of at least about 75 copies on a surface of the multivalent particle when the multivalent particle is expressed.

106. The composition of claim 58, wherein the fusion protein is expressed at a valency of at least about 100 copies on a surface of the multivalent particle when the multivalent particle is expressed.

107. The composition of claim 58, wherein the fusion protein is expressed at a valency of at least about 150 copies on a surface of the multivalent particle when the multivalent particle is expressed.

108. The composition of claim 58, wherein the fusion protein is expressed at a valency of at least about 200 copies on a surface of the multivalent particle when the multivalent particle is expressed.

109. The composition of claim 58, wherein the fusion protein is expressed at a valency of at least about 500 copies on a surface of the multivalent particle when the multivalent particle is expressed.

110. The composition of claim 58, wherein the fusion protein is expressed at a valency of at least about 1000 copies on a surface of the multivalent particle when the multivalent particle is expressed.

111. The composition of claim 58, wherein the fusion protein is expressed at a valency of at least about 2000 copies on a surface of the multivalent particle when the multivalent particle is expressed.

112. The composition of claim 58, wherein the multivalent particle does not comprise viral genetic material.

113. The composition of claim 58, wherein the multivalent particle is a viral-like a particle.

114. The composition of claim 58, wherein the multivalent particle is an extracellular vesicle (EV).

115. The composition of claim 58, wherein the multivalent particle is an exosome.

116. The composition of claim 58, wherein the multivalent particle is an ectosome.

117. The composition of claim 62, wherein the first nucleic acid sequence, the second nucleic acid sequence, and the third nucleic acid sequence are within a same vector.

118. The composition of claim 62, wherein the first nucleic acid sequence, the second nucleic acid sequence, and the third nucleic acid sequence are within different vectors.

119. The composition of claim 117, wherein the vector is a lentivirus vector, an adenovirus vector, or an adeno-associated virus vector.

120. The composition of claim 118, wherein the vectors comprise a lentivirus vector, an adenovirus vector, or an adeno-associated virus vector.

121. The composition of claim 58, wherein:
(a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; and
(b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120.

122. The composition of claim 58, wherein:
(a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL; and
(b) the transmembrane polypeptide comprises an amino acid sequence at least about 75%, 80%, 85%, 90%, 95%, or 99% identical to that set forth in any one of SEQ ID NOs: 63, 64, or 79-95.

123. The composition of claim 58, wherein:
(a) the immune checkpoint polypeptide comprises an amino acid sequence of at least 75%, 80%, 85%, 90%, 95%, or 99% sequence identity to an amino acid sequence according to any one of SEQ ID NOs: 1-62, 96-115, or 153-162; and
(b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120.

124. The composition of claim 58, wherein:
(a) the immune checkpoint polypeptide comprises an amino acid sequence of at least 75%, 80%, 85%, 90%, 95%, or 99% sequence identity to an amino acid sequence according to any one of SEQ ID NOs: 1-62, 96-115, or 153-162; and
(b) the transmembrane polypeptide comprises an amino acid sequence at least about 75%, 80%, 85%, 90%, 95%, or 99% identical to that set forth in any one of SEQ ID NOs: 63, 64, or 79-95.

125. The composition of claim 85, wherein:
(a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL;
(b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120; and
(c) the oligomerization domain comprises a leucine zipper dimerization domain, a post-fusion oligomerization domain of viral surface protein, a D4 post-fusion trimerization domain of VSV-G protein, a Dengue E protein post-fusion trimerization domain, a foldon trimerization domain, or an influenza neuraminidase stem domain.

126. The composition of claim 85, wherein:
(a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL;
(b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120; and
(c) the oligomerization domain comprises an amino acid sequence that has at least 75%, 80%, 85%, 90%, 95%, or 99% sequence identity to an amino acid sequence according to SEQ ID NOs: 65-78.

127. The composition of claim 85, wherein:
(a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL;
(b) the transmembrane polypeptide comprises an amino acid sequence at least about 75%, 80%, 85%, 90%, 95%, or 99% identical to that set forth in any one of SEQ ID NOs: 63, 64, or 79-95; and
(c) the oligomerization domain comprises a leucine zipper dimerization domain, a post-fusion oligomerization domain of viral surface protein, a D4 post-fusion trimerization domain of VSV-G protein, a Dengue E protein post-fusion trimerization domain, a foldon trimerization domain, or an influenza neuraminidase stem domain.

128. The composition of claim 85, wherein:
(a) the immune checkpoint polypeptide comprises an amino acid sequence of at least 75%, 80%, 85%, 90%, 95%, or 99% sequence identity to an amino acid sequence according to any one of SEQ ID NOs: 1-62, 96-115, or 153-162;
(b) the transmembrane polypeptide comprises VSVG, Dengue E protein, influenza hemagglutinin, influenza neuraminidase, spike protein S1, spike protein S2, Sindbis virus envelope (SINDBIS) protein, hemagglutinin envelope protein from measles virus, envelope glycoprotein of measles virus fusion (F) protein, RD114, BaEV, GP41, or GP120; and
(c) the oligomerization domain comprises a leucine zipper dimerization domain, a post-fusion oligomerization domain of viral surface protein, a D4 post-fusion trimerization domain of VSV-G protein, a Dengue E protein post-fusion trimerization domain, a foldon trimerization domain, or an influenza neuraminidase stem domain.

129. The composition of claim 85, wherein:
(a) the immune checkpoint polypeptide comprises PD-1, CTLA4, LAG3, BTLA, CD160, 2B4, CD226, TIGIT, CD96, B7-H3, B7-H4, VISTA, TIM3, SIGLEC7, KLRG1, SIGLEC9, PD-L1, PD-L2, CD80, CD86, HVEM, CD48, CD112, CD155, Ceacam1, FGL1, Galectin-3, CD27, CD28, CD40, CD122, 4-1BB, ICOS, OX40, CD2, CD30, GITR, CD70, CD80, CD86, CD40L, GITRL, 4-1BBL, OX40L, LIGHT, CD30L, CD48, or ICOSL;
(b) the transmembrane polypeptide comprises an amino acid sequence at least about 75%, 80%, 85%, 90%, 95%, or 99% identical to that set forth in any one of SEQ ID NOs: 63, 64, or 79-95; and
(c) the oligomerization domain comprises an amino acid sequence that has at least 75%, 80%, 85%, 90%, 95%, or 99% sequence identity to an amino acid sequence according to SEQ ID NOs: 65-78.

130. A pharmaceutical composition comprising the multivalent particle of claim 1 and a pharmaceutically acceptable excipient.

131. A method of treating a cancer, an autoimmune disease, an infection, or an inflammatory disease, comprising administering the multivalent particle of claim 1.

132. The method of claim 131, wherein the multivalent particle is administered intravenously.

133. The method of claim 131, wherein the multivalent particle is administered through inhalation.

134. The method of claim 131, wherein the multivalent particle is administered by intraperitoneal injection.

135. The method of claim 131, wherein the multivalent particle is administered by subcutaneous injection.

136. A composition comprising a multivalent particle (MVP) wherein the MVP comprises an enveloped particle that displays at least about 10 copies of an immune checkpoint polypeptide on a surface of the MVP, wherein the immune checkpoint polypeptide forms multivalent interactions with a ligand on a target immune cell when displayed on the surface of the enveloped particle.

137. A method of using a multivalent particle (MVP) displaying an immune checkpoint polypeptide to mimic multivalent interactions between a first immune cell expressing the immune checkpoint polypeptide and a second immune cell expressing a target of the immune checkpoint polypeptide, wherein the immune checkpoint polypeptide is displayed at least about 10 copies on a surface of the MVP.
